SEQUENCE LISTING (544 sequences)

SEQ ID NO: 1 - DNA, 62 bp, Homo sapiens
  misc_feature 1..62: note = HSD17B4 5'-UTR
  source 1..62: mol_type = genomic DNA
gtcccgcagtcggcgtccagcggctctgcttgttcgtgtgtgtgtcgttgcaggccttattc

SEQ ID NO: 2 - RNA, 62 nt, Homo sapiens
  misc_feature 1..62: note = HSD17B4 5'-UTR
  source 1..62: mol_type = mRNA
gtcccgcagtcggcgtccagcggctctgcttgttcgtgtgtgtgtcgttgcaggccttattc

SEQ ID NO: 3 - DNA, 42 bp, Homo sapiens
  misc_feature 1..42: note = RPL32_(32L4,32L3) 5'-UTR
  source 1..42: mol_type = genomic DNA
ggcgctgcctacggaggtggcagccatctccttctcggcatc

SEQ ID NO: 4 - RNA, 42 nt, Homo sapiens
  misc_feature 1..42: note = RPL32_(32L4,32L3) 5'-UTR
  source 1..42: mol_type = mRNA
ggcgctgcctacggaggtggcagccatctccttctcggcatc

SEQ ID NO: 5 - DNA, 31 bp, Homo sapiens
  misc_feature 1..31: note = AIG1 5'-UTR
  source 1..31: mol_type = genomic DNA
gccgcccagccggtccaggcctctggcgaac

SEQ ID NO: 6 - RNA, 31 nt, Homo sapiens
  misc_feature 1..31: note = AIG1 5'-UTR
  source 1..31: mol_type = mRNA
gccgcccagccggtccaggcctctggcgaac

SEQ ID NO: 7 - DNA, 42 bp, Homo sapiens
  misc_feature 1..42: note = ASAH1 5'-UTR
  source 1..42: mol_type = genomic DNA
gcctctgctggagtccggggagtggcgttggctgctagagcg

SEQ ID NO: 8 - RNA, 42 nt, Homo sapiens
  misc_feature 1..42: note = ASAH1 5'-UTR
  source 1..42: mol_type = mRNA
gcctctgctggagtccggggagtggcgttggctgctagagcg

SEQ ID NO: 9 - DNA, 75 bp, Homo sapiens
  misc_feature 1..75: note = ATP5A1 5'-UTR
  source 1..75: mol_type = genomic DNA
gcggctcggccattttgtcccagtcagtccggaggctgcggctgcagaagtaccgcctgcggagtaactgcaaag

SEQ ID NO: 10 - RNA, 75 nt, Homo sapiens
  misc_feature 1..75: note = ATP5A1 5'-UTR
  source 1..75: mol_type = mRNA
gcggctcggccattttgtcccagtcagtccggaggctgcggctgcagaagtaccgcctgcggagtaactgcaaag

SEQ ID NO: 11 - DNA, 56 bp, Homo sapiens
  misc_feature 1..56: note = COX6C 5'-UTR
  source 1..56: mol_type = genomic DNA
agtcaggaaggacgttggtgttgaggttagcatacgtatcaaggacagtaactacc

SEQ ID NO: 12 - RNA, 56 nt, Homo sapiens
  misc_feature 1..56: note = COX6C 5'-UTR
  source 1..56: mol_type = mRNA
agtcaggaaggacgttggtgttgaggttagcatacgtatcaaggacagtaactacc

SEQ ID NO: 13 - DNA, 232 bp, Mus musculus
  misc_feature 1..232: note = Dpysl2 5'-UTR
  source 1..232: mol_type = genomic DNA
ctctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagag

SEQ ID NO: 14 - RNA, 232 nt, Mus musculus
  misc_feature 1..232: note = Dpysl2 5'-UTR
  source 1..232: mol_type = mRNA
ctctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagag

SEQ ID NO: 15 - DNA, 22 bp, Homo sapiens
  misc_feature 1..22: note = MDR (ABCB7) 5'-UTR
  source 1..22: mol_type = genomic DNA
ggttcctctttcctcgctcaag

SEQ ID NO: 16 - RNA, 22 nt, Homo sapiens
  misc_feature 1..22: note = MDR (ABCB7) 5'-UTR
  source 1..22: mol_type = mRNA
ggttcctctttcctcgctcaag

SEQ ID NO: 17 - DNA, 54 bp, Mus musculus
  misc_feature 1..54: note = Mp68_(2010107E04Rik) 5'-UTR
  source 1..54: mol_type = genomic DNA
ctttcccattctgtagcagaatttggtgttgcctgtggtcttggtcccgcggag

SEQ ID NO: 18 - RNA, 54 nt, Mus musculus
  misc_feature 1..54: note = Mp68_(2010107E04Rik) 5'-UTR
  source 1..54: mol_type = mRNA
ctttcccattctgtagcagaatttggtgttgcctgtggtcttggtcccgcggag

SEQ ID NO: 19 - DNA, 81 bp, Mus musculus
  misc_feature 1..81: note = Ndufa4 5'-UTR
  source 1..81: mol_type = genomic DNA
gtccgctcagccaggttgcagaagcggcttagcgtgtgtcctaatcttctctctgcgtgtaggtaggcctgtgccgcaaac

SEQ ID NO: 20 - RNA, 81 nt, Mus musculus
  misc_feature 1..81: note = Ndufa4 5'-UTR
  source 1..81: mol_type = mRNA
gtccgctcagccaggttgcagaagcggcttagcgtgtgtcctaatcttctctctgcgtgtaggtaggcctgtgccgcaaac

SEQ ID NO: 21 - DNA, 108 bp, Mus musculus
  misc_feature 1..108: note = Nosip 5'-UTR
  source 1..108: mol_type = genomic DNA
ctcctgtcgggcggaagtaggaggagtagagtttaaaaacagtactctttttccggttcgggacgtagttgaagcaacgacaagccggataaccgctcttgagacagg

SEQ ID NO: 22 - RNA, 108 nt, Mus musculus
  misc_feature 1..108: note = Nosip 5'-UTR
  source 1..108: mol_type = mRNA
ctcctgtcgggcggaagtaggaggagtagagtttaaaaacagtactctttttccggttcgggacgtagttgaagcaacgacaagccggataaccgctcttgagacagg

SEQ ID NO: 23 - DNA, 84 bp, Mus musculus
  misc_feature 1..84: note = Rpl31 5'-UTR
  source 1..84: mol_type = genomic DNA
cccgtgacccggaagttgtacggctacgcgactttccctcccacaaaccctcgcgcccttcctttcctacttgggcccggcaga

SEQ ID NO: 24 - RNA, 84 nt, Mus musculus
  misc_feature 1..84: note = Rpl31 5'-UTR
  source 1..84: mol_type = mRNA
cccgtgacccggaagttgtacggctacgcgactttccctcccacaaaccctcgcgcccttcctttcctacttgggcccggcaga

SEQ ID NO: 25 - DNA, 56 bp, Homo sapiens
  misc_feature 1..56: note = RPL35A 5'-UTR
  source 1..56: mol_type = genomic DNA
accgccatcttggctcctgtggaggcctgctgggaacgggacttctaaaaggaact

SEQ ID NO: 26 - RNA, 56 nt, Homo sapiens
  misc_feature 1..56: note = RPL35A 5'-UTR
  source 1..56: mol_type = mRNA
accgccatcttggctcctgtggaggcctgctgggaacgggacttctaaaaggaact

SEQ ID NO: 27 - DNA, 159 bp, Mus musculus
  misc_feature 1..159: note = Slc7a3 5'-UTR
  source 1..159: mol_type = genomic DNA
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 28 - RNA, 159 nt, Mus musculus
  misc_feature 1..159: note = Slc7a3 5'-UTR
  source 1..159: mol_type = mRNA
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 29 - DNA, 102 bp, Homo sapiens
  misc_feature 1..102: note = TUBB4B 5'-UTR
  source 1..102: mol_type = genomic DNA
atataagcgttggcggagcgtcggttgtagcactctgcgcgcccgctcttctgctgctgtttgtctacttcctcctgcttccccgccgccgccgccgccatc

SEQ ID NO: 30 - RNA, 102 nt, Homo sapiens
  misc_feature 1..102: note = TUBB4B 5'-UTR
  source 1..102: mol_type = mRNA
atataagcgttggcggagcgtcggttgtagcactctgcgcgcccgctcttctgctgctgtttgtctacttcctcctgcttccccgccgccgccgccgccatc

SEQ ID NO: 31 - DNA, 222 bp, Mus musculus
  misc_feature 1..222: note = Ubqln2 5'-UTR
  source 1..222: mol_type = genomic DNA
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 32 - RNA, 222 nt, Mus musculus
  misc_feature 1..222: note = Ubqln2 5'-UTR
  source 1..222: mol_type = mRNA
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 33 - DNA, 57 bp, synthetic construct
  misc_feature 1..57: note = synthetic-v1 5'-UTR
  source 1..57: mol_type = other DNA
gggaaataagagagaaaagaagagtaagaagaaatataagaccccggcgccgccacc

SEQ ID NO: 34 - RNA, 57 nt, synthetic construct
  misc_feature 1..57: note = synthetic-v1 5'-UTR
  source 1..57: mol_type = other RNA
gggaaataagagagaaaagaagagtaagaagaaatataagaccccggcgccgccacc

SEQ ID NO: 35 - DNA, 57 bp, synthetic construct
  misc_feature 1..57: note = synthetic-v2 5'-UTR
  source 1..57: mol_type = other DNA
aggagataagagagaaaagaagagtaagaagaaatataagaccccggcgccgccacc

SEQ ID NO: 36 - RNA, 57 nt, synthetic construct
  misc_feature 1..57: note = synthetic-v2 5'-UTR
  source 1..57: mol_type = other RNA
aggagataagagagaaaagaagagtaagaagaaatataagaccccggcgccgccacc

SEQ ID NO: 37 - DNA, 47 bp, synthetic construct
  misc_feature 1..47: note = synthetic-v3 5'-UTR
  source 1..47: mol_type = other DNA
gggaaataagagagaaaagaagagtaagaagaaatataagagccacc

SEQ ID NO: 38 - RNA, 47 nt, synthetic construct
  misc_feature 1..47: note = synthetic-v3 5'-UTR
  source 1..47: mol_type = other RNA
gggaaataagagagaaaagaagagtaagaagaaatataagagccacc

SEQ ID NO: 39 - DNA, 47 bp, synthetic construct
  misc_feature 1..47: note = synthetic-v4 5'-UTR
  source 1..47: mol_type = other DNA
aggagataagagagaaaagaagagtaagaagaaatataagagccacc

SEQ ID NO: 40 - RNA, 47 nt, synthetic construct
  misc_feature 1..47: note = synthetic-v4 5'-UTR
  source 1..47: mol_type = other RNA
aggagataagagagaaaagaagagtaagaagaaatataagagccacc

SEQ ID NO: 41 - DNA, 54 bp, synthetic construct
  misc_feature 1..54: note = synthetic-v5 5'-UTR
  source 1..54: mol_type = other DNA
gggaaataagagagaaaagaagagtaagaagaaatataagaccccggcgccacc

SEQ ID NO: 42 - RNA, 54 nt, synthetic construct
  misc_feature 1..54: note = synthetic-v5 5'-UTR
  source 1..54: mol_type = other RNA
gggaaataagagagaaaagaagagtaagaagaaatataagaccccggcgccacc

SEQ ID NO: 43 - DNA, 54 bp, synthetic construct
  misc_feature 1..54: note = synthetic-v6 5'-UTR
  source 1..54: mol_type = other DNA
aggagataagagagaaaagaagagtaagaagaaatataagaccccggcgccacc

SEQ ID NO: 44 - RNA, 54 nt, synthetic construct
  misc_feature 1..54: note = synthetic-v6 5'-UTR
  source 1..54: mol_type = other RNA
aggagataagagagaaaagaagagtaagaagaaatataagaccccggcgccacc

SEQ ID NO: 45 - DNA, 67 bp, synthetic construct
  misc_feature 1..67: note = synthetic-v7 5'-UTR
  source 1..67: mol_type = other DNA
gggaaataagagagaaaagaagagtaagaagaaatataagagcgccccgcggcgccccgcggccacc

SEQ ID NO: 46 - RNA, 67 nt, synthetic construct
  misc_feature 1..67: note = synthetic-v7 5'-UTR
  source 1..67: mol_type = other RNA
gggaaataagagagaaaagaagagtaagaagaaatataagagcgccccgcggcgccccgcggccacc

SEQ ID NO: 47 - DNA, 67 bp, synthetic construct
  misc_feature 1..67: note = synthetic-v8 5'-UTR
  source 1..67: mol_type = other DNA
aggagataagagagaaaagaagagtaagaagaaatataagagcgccccgcggcgccccgcggccacc

SEQ ID NO: 48 - RNA, 67 nt, synthetic construct
  misc_feature 1..67: note = synthetic-v8 5'-UTR
  source 1..67: mol_type = other RNA
aggagataagagagaaaagaagagtaagaagaaatataagagcgccccgcggcgccccgcggccacc

SEQ ID NO: 49 - DNA, 53 bp, synthetic construct
  misc_feature 1..53: note = HBA1-v1 5'-UTR
  source 1..53: mol_type = other DNA
agaataaactagtattcttctggtccccacagactcagagagaacccgccacc

SEQ ID NO: 50 - RNA, 53 nt, synthetic construct
  misc_feature 1..53: note = HBA1-v1 5'-UTR
  source 1..53: mol_type = other RNA
agaataaactagtattcttctggtccccacagactcagagagaacccgccacc

SEQ ID NO: 51 - DNA, 56 bp, synthetic construct
  misc_feature 1..56: note = HBA1-v2 5'-UTR
  source 1..56: mol_type = other DNA
aggagaataaactagtattcttctggtccccacagactcagagagaacccgccacc

SEQ ID NO: 52 - RNA, 56 nt, synthetic construct
  misc_feature 1..56: note = HBA1-v2 5'-UTR
  source 1..56: mol_type = other RNA
aggagaataaactagtattcttctggtccccacagactcagagagaacccgccacc

SEQ ID NO: 53 - DNA, 53 bp, synthetic construct
  misc_feature 1..53: note = HBA1-v3 5'-UTR
  source 1..53: mol_type = other DNA
aggagaaactagtattcttctggtccccacagactcagagagaacccgccacc

SEQ ID NO: 54 - RNA, 53 nt, synthetic construct
  misc_feature 1..53: note = HBA1-v3 5'-UTR
  source 1..53: mol_type = other RNA
aggagaaactagtattcttctggtccccacagactcagagagaacccgccacc

SEQ ID NO: 55 - DNA, 53 bp, synthetic construct
  misc_feature 1..53: note = HBA1-v4 5'-UTR
  source 1..53: mol_type = other DNA
agattaaactagtattcttctggtccccacagactcagagagaacccgccacc

SEQ ID NO: 56 - RNA, 53 nt, synthetic construct
  misc_feature 1..53: note = HBA1-v4 5'-UTR
  source 1..53: mol_type = other RNA
agattaaactagtattcttctggtccccacagactcagagagaacccgccacc

SEQ ID NO: 57 - DNA, 52 bp, synthetic construct
  misc_feature 1..52: note = HBA1-v5 5'-UTR
  source 1..52: mol_type = other DNA
agacgaactagtattcttctggtccccacagactcagagagaacccgccacc

SEQ ID NO: 58 - RNA, 52 nt, synthetic construct
  misc_feature 1..52: note = HBA1-v5 5'-UTR
  source 1..52: mol_type = other RNA
agacgaactagtattcttctggtccccacagactcagagagaacccgccacc

SEQ ID NO: 59 - DNA, 52 bp, synthetic construct
  misc_feature 1..52: note = HBA1-v6 5'-UTR
  source 1..52: mol_type = other DNA
gggcgaactagtattcttctggtccccacagactcagagagaacccgccacc

SEQ ID NO: 60 - RNA, 52 nt, synthetic construct
  misc_feature 1..52: note = HBA1-v6 5'-UTR
  source 1..52: mol_type = other RNA
gggcgaactagtattcttctggtccccacagactcagagagaacccgccacc

SEQ ID NO: 61 - DNA, 15 bp, synthetic construct
  misc_feature 1..15: note = minimal 5'-UTR
  source 1..15: mol_type = other DNA
aggagaaagcttacc

SEQ ID NO: 62 - RNA, 15 nt, synthetic construct
  misc_feature 1..15: note = minimal 5'-UTR
  source 1..15: mol_type = other RNA
aggagaaagcttacc

SEQ ID NO: 63 - DNA, 77 bp, synthetic construct
  misc_feature 1..77: note = HSD17B4-v2 5'-UTR
  source 1..77: mol_type = other DNA
aggagagtcccgcagtcggcgtccagcggctctgcttgttcgtgtgtgtgtcgttgcaggccttattcaagcttacc

SEQ ID NO: 64 - RNA, 77 nt, synthetic construct
  misc_feature 1..77: note = HSD17B4-v2 5'-UTR
  source 1..77: mol_type = other RNA
aggagagtcccgcagtcggcgtccagcggctctgcttgttcgtgtgtgtgtcgttgcaggccttattcaagcttacc

SEQ ID NO: 65 - DNA, 57 bp, synthetic construct
  misc_feature 1..57: note = RPL32_(32L4,32L3)-v2 5'-UTR
  source 1..57: mol_type = other DNA
aggagaggcgctgcctacggaggtggcagccatctccttctcggcatcaagcttacc

SEQ ID NO: 66 - RNA, 57 nt, synthetic construct
  misc_feature 1..57: note = RPL32_(32L4, 32L3)-v2 5'-UTR
  source 1..57: mol_type = other RNA
aggagaggcgctgcctacggaggtggcagccatctccttctcggcatcaagcttacc

SEQ ID NO: 67 - DNA, 46 bp, synthetic construct
  misc_feature 1..46: note = AIG1-v2 5'-UTR
  source 1..46: mol_type = other DNA
aggagagccgcccagccggtccaggcctctggcgaacaagcttacc

SEQ ID NO: 68 - RNA, 46 nt, synthetic construct
  misc_feature 1..46: note = AIG1-v2 5'-UTR
  source 1..46: mol_type = other RNA
aggagagccgcccagccggtccaggcctctggcgaacaagcttacc

SEQ ID NO: 69 - DNA, 57 bp, synthetic construct
  misc_feature 1..57: note = ASAH1-v2 5'-UTR
  source 1..57: mol_type = other DNA
aggagagcctctgctggagtccggggagtggcgttggctgctagagcgaagcttacc

SEQ ID NO: 70 - RNA, 57 nt, synthetic construct
  misc_feature 1..57: note = ASAH1-v2 5'-UTR
  source 1..57: mol_type = other RNA
aggagagcctctgctggagtccggggagtggcgttggctgctagagcgaagcttacc

SEQ ID NO: 71 - DNA, 90 bp, synthetic construct
  misc_feature 1..90: note = ATP5A1-v2 5'-UTR
  source 1..90: mol_type = other DNA
aggagagcggctcggccattttgtcccagtcagtccggaggctgcggctgcagaagtaccgcctgcggagtaactgcaaagaagcttacc

SEQ ID NO: 72 - RNA, 90 nt, synthetic construct
  misc_feature 1..90: note = ATP5A1-v2 5'-UTR
  source 1..90: mol_type = other RNA
aggagagcggctcggccattttgtcccagtcagtccggaggctgcggctgcagaagtaccgcctgcggagtaactgcaaagaagcttacc

SEQ ID NO: 73 - DNA, 71 bp, synthetic construct
  misc_feature 1..71: note = COX6C-v2 5'-UTR
  source 1..71: mol_type = other DNA
aggagaagtcaggaaggacgttggtgttgaggttagcatacgtatcaaggacagtaactaccaagcttacc

SEQ ID NO: 74 - RNA, 71 nt, synthetic construct
  misc_feature 1..71: note = COX6C-v2 5'-UTR
  source 1..71: mol_type = other RNA
aggagaagtcaggaaggacgttggtgttgaggttagcatacgtatcaaggacagtaactaccaagcttacc

SEQ ID NO: 75 - DNA, 247 bp, synthetic construct
  misc_feature 1..247: note = Dpysl2-v2 5'-UTR
  source 1..247: mol_type = other DNA
aggagactctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagagaagcttacc

SEQ ID NO: 76 - RNA, 247 nt, synthetic construct
  misc_feature 1..247: note = Dpysl2-v2 5'-UTR
  source 1..247: mol_type = other RNA
aggagactctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagagaagcttacc

SEQ ID NO: 77 - DNA, 37 bp, synthetic construct
  misc_feature 1..37: note = MDR (ABCB7)-v2 5'-UTR
  source 1..37: mol_type = other DNA
aggagaggttcctctttcctcgctcaagaagcttacc

SEQ ID NO: 78 - RNA, 37 nt, synthetic construct
  misc_feature 1..37: note = MDR (ABCB7)-v2 5'-UTR
  source 1..37: mol_type = other RNA
aggagaggttcctctttcctcgctcaagaagcttacc

SEQ ID NO: 79 - DNA, 69 bp, synthetic construct
  misc_feature 1..69: note = Mp68_(2010107E04Rik)-v2 5'-UTR
  source 1..69: mol_type = other DNA
aggagactttcccattctgtagcagaatttggtgttgcctgtggtcttggtcccgcggagaagcttacc

SEQ ID NO: 80 - RNA, 69 nt, synthetic construct
  misc_feature 1..69: note = Mp68_(2010107E04Rik)-v2 5'-UTR
  source 1..69: mol_type = other RNA
aggagactttcccattctgtagcagaatttggtgttgcctgtggtcttggtcccgcggagaagcttacc

SEQ ID NO: 81 - DNA, 96 bp, synthetic construct
  misc_feature 1..96: note = Ndufa4-v2 5'-UTR
  source 1..96: mol_type = other DNA
aggagagtccgctcagccaggttgcagaagcggcttagcgtgtgtcctaatcttctctctgcgtgtaggtaggcctgtgccgcaaacaagcttacc

SEQ ID NO: 82 - RNA, 96 nt, synthetic construct
  misc_feature 1..96: note = Ndufa4-v2 5'-UTR
  source 1..96: mol_type = other RNA
aggagagtccgctcagccaggttgcagaagcggcttagcgtgtgtcctaatcttctctctgcgtgtaggtaggcctgtgccgcaaacaagcttacc

SEQ ID NO: 83 - DNA, 123 bp, synthetic construct
  misc_feature 1..123: note = Nosip-v2 5'-UTR
  source 1..123: mol_type = other DNA
aggagactcctgtcgggcggaagtaggaggagtagagtttaaaaacagtactctttttccggttcgggacgtagttgaagcaacgacaagccggataaccgctcttgagacaggaagcttacc

SEQ ID NO: 84 - RNA, 123 nt, synthetic construct
  misc_feature 1..123: note = Nosip-v2 5'-UTR
  source 1..123: mol_type = other RNA
aggagactcctgtcgggcggaagtaggaggagtagagtttaaaaacagtactctttttccggttcgggacgtagttgaagcaacgacaagccggataaccgctcttgagacaggaagcttacc

SEQ ID NO: 85 - DNA, 99 bp, synthetic construct
  misc_feature 1..99: note = Rpl31-v2 5'-UTR
  source 1..99: mol_type = other DNA
aggagacccgtgacccggaagttgtacggctacgcgactttccctcccacaaaccctcgcgcccttcctttcctacttgggcccggcagaaagcttacc

SEQ ID NO: 86 - RNA, 99 nt, synthetic construct
  misc_feature 1..99: note = Rpl31-v2 5'-UTR
  source 1..99: mol_type = other RNA
aggagacccgtgacccggaagttgtacggctacgcgactttccctcccacaaaccctcgcgcccttcctttcctacttgggcccggcagaaagcttacc

SEQ ID NO: 87 - DNA, 71 bp, synthetic construct
  misc_feature 1..71: note = RPL35A-v2 5'-UTR
  source 1..71: mol_type = other DNA
aggagaaccgccatcttggctcctgtggaggcctgctgggaacgggacttctaaaaggaactaagcttacc

SEQ ID NO: 88 - RNA, 71 nt, synthetic construct
  misc_feature 1..71: note = RPL35A-v2 5'-UTR
  source 1..71: mol_type = other RNA
aggagaaccgccatcttggctcctgtggaggcctgctgggaacgggacttctaaaaggaactaagcttacc

SEQ ID NO: 89 - DNA, 174 bp, synthetic construct
  misc_feature 1..174: note = Slc7a3-v2 5'-UTR
  source 1..174: mol_type = other DNA
aggagagggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactagaaagcttacc

SEQ ID NO: 90 - RNA, 174 nt, synthetic construct
  misc_feature 1..174: note = Slc7a3-v2 5'-UTR
  source 1..174: mol_type = other RNA
aggagagggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactagaaagcttacc

SEQ ID NO: 91 - DNA, 117 bp, synthetic construct
  misc_feature 1..117: note = TUBB4B-v2 5'-UTR
  source 1..117: mol_type = other DNA
aggagaatataagcgttggcggagcgtcggttgtagcactctgcgcgcccgctcttctgctgctgtttgtctacttcctcctgcttccccgccgccgccgccgccatcaagcttacc

SEQ ID NO: 92 - RNA, 117 nt, synthetic construct
  misc_feature 1..117: note = TUBB4B-v2 5'-UTR
  source 1..117: mol_type = other RNA
aggagaatataagcgttggcggagcgtcggttgtagcactctgcgcgcccgctcttctgctgctgtttgtctacttcctcctgcttccccgccgccgccgccgccatcaagcttacc

SEQ ID NO: 93 - DNA, 237 bp, synthetic construct
  misc_feature 1..237: note = Ubqln2-v2 5'-UTR
  source 1..237: mol_type = other DNA
aggagacggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgccaagcttacc

SEQ ID NO: 94 - RNA, 237 nt, synthetic construct
  misc_feature 1..237: note = Ubqln2-v2 5'-UTR
  source 1..237: mol_type = other RNA
aggagacggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgccaagcttacc

SEQ ID NO: 95 - DNA, 57 bp, Homo sapiens
  misc_feature 1..57: note = PSMB3 3'-UTR
  source 1..57: mol_type = genomic DNA
ccctgttcccagagcccacttttttttctttttttgaaataaaatagcctgtctttc

SEQ ID NO: 96 - RNA, 57 nt, Homo sapiens
  misc_feature 1..57: note = PSMB3 3'-UTR
  source 1..57: mol_type = mRNA
ccctgttcccagagcccacttttttttctttttttgaaataaaatagcctgtctttc

SEQ ID NO: 97 - DNA, 44 bp, Homo sapiens
  misc_feature 1..44: note = muag 3'-UTR
  source 1..44: mol_type = genomic DNA
gcccgatgggcctcccaacgggccctcctcccctccttgcaccg

SEQ ID NO: 98 - RNA, 44 nt, Homo sapiens
  misc_feature 1..44: note = muag 3'-UTR
  source 1..44: mol_type = mRNA
gcccgatgggcctcccaacgggccctcctcccctccttgcaccg

SEQ ID NO: 99 - DNA, 187 bp, Homo sapiens
  misc_feature 1..187: note = ALB7 3'-UTR
  source 1..187: mol_type = genomic DNA
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 100 - RNA, 187 nt, Homo sapiens
  misc_feature 1..187: note = ALB7 3'-UTR
  source 1..187: mol_type = mRNA
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 101 - DNA, 266 bp, Homo sapiens
  misc_feature 1..266: note = ANXA4 3'-UTR
  source 1..266: mol_type = genomic DNA
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 102 - RNA, 266 nt, Homo sapiens
  misc_feature 1..266: note = ANXA4 3'-UTR
  source 1..266: mol_type = mRNA
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 103 - DNA, 121 bp, Homo sapiens
  misc_feature 1..121: note = CASP1 3'-UTR
  source 1..121: mol_type = genomic DNA
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 104 - RNA, 121 nt, Homo sapiens
  misc_feature 1..121: note = CASP1 3'-UTR
  source 1..121: mol_type = mRNA
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 105 - DNA, 137 bp, Homo sapiens
  misc_feature 1..137: note = COX6B1 3'-UTR
  source 1..137: mol_type = genomic DNA
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 106 - RNA, 137 nt, Homo sapiens
  misc_feature 1..137: note = COX6B1 3'-UTR
  source 1..137: mol_type = mRNA
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 107 - DNA, 166 bp, Homo sapiens
  misc_feature 1..166: note = FIG4 3'-UTR
  source 1..166: mol_type = genomic DNA
aaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgc

SEQ ID NO: 108 - RNA, 166 nt, Homo sapiens
  misc_feature 1..166: note = FIG4 3'-UTR
  source 1..166: mol_type = mRNA
aaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgc

SEQ ID NO: 109 - DNA, 353 bp, Mus musculus
  misc_feature 1..353: note = Gnas 3'-UTR
  source 1..353: mol_type = genomic DNA
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 110 - RNA, 353 nt, Mus musculus
  misc_feature 1..353: note = Gnas 3'-UTR
  source 1..353: mol_type = mRNA
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 111 - DNA, 133 bp, Mus musculus
  misc_feature 1..133: note = Ndufa1 3'-UTR
  source 1..133: mol_type = genomic DNA
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 112 - RNA, 133 nt, Mus musculus
  misc_feature 1..133: note = Ndufa1 3'-UTR
  source 1..133: mol_type = mRNA
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 113 - DNA, 70 bp, Homo sapiens
  misc_feature 1..70: note = RPS9 3'-UTR
  source 1..70: mol_type = genomic DNA
gtccacctgtccctcctgggctgctggattgtctcgttttcctgccaaataaacaggatcagcgctttac

SEQ ID NO: 114 - RNA, 70 nt, Homo sapiens
  misc_feature 1..70: note = RPS9 3'-UTR
  source 1..70: mol_type = mRNA
gtccacctgtccctcctgggctgctggattgtctcgttttcctgccaaataaacaggatcagcgctttac

SEQ ID NO: 115 - DNA, 240 bp, Mus musculus
  misc_feature 1..240: note = Slc7a3 3'-UTR
  source 1..240: mol_type = genomic DNA
cctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgt

SEQ ID NO: 116 - RNA, 240 nt, Mus musculus
  misc_feature 1..240: note = Slc7a3 3'-UTR
  source 1..240: mol_type = mRNA
cctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgt

SEQ ID NO: 117 - DNA, 151 bp, Homo sapiens
  misc_feature 1..151: note = TUBB4B 3'-UTR
  source 1..151: mol_type = genomic DNA
agccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtg

SEQ ID NO: 118 - RNA, 151 nt, Homo sapiens
  misc_feature 1..151: note = TUBB4B 3'-UTR
  source 1..151: mol_type = mRNA
agccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtg

SEQ ID NO: 119 - DNA, 110 bp, Homo sapiens
  misc_feature 1..110: note = HBA1-v1 3'-UTR
  source 1..110: mol_type = genomic DNA
gctggagcctcggtggccatgcttcttgccccttgggcctccccccagcccctcctccccttcctgcacccgtacccccgtggtctttgaataaagtctgagtgggcggc

SEQ ID NO: 120 - RNA, 110 nt, Homo sapiens
  misc_feature 1..110: note = HBA1-v1 3'-UTR
  source 1..110: mol_type = mRNA
gctggagcctcggtggccatgcttcttgccccttgggcctccccccagcccctcctccccttcctgcacccgtacccccgtggtctttgaataaagtctgagtgggcggc

SEQ ID NO: 121 - DNA, 119 bp, synthetic construct
  misc_feature 1..119: note = HBA1-v2 3'-UTR
  source 1..119: mol_type = other DNA
tgataataggctggagcctcggtggcctagcttcttgccccttgggcctccccccagcccctcctccccttcctgcacccgtacccccgtggtctttgaataaagtctgagtgggcggc

SEQ ID NO: 122 - RNA, 119 nt, synthetic construct
  misc_feature 1..119: note = HBA1-v2 3'-UTR
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 123 - DNA, 119 bp, synthetic construct
  misc_feature 1..119: note = HBA1-v3 3'-UTR
  source 1..119: mol_type = other DNA
tgataataggctggagcctcggtggccatgcttcttgccccttgggcctccccccagcccctcctccccttcctgcacccgtacccccgtggtctttgaataaagtctgagtgggcggc

SEQ ID NO: 124 - RNA, 119 nt, synthetic construct
  misc_feature 1..119: note = HBA1-v3 3'-UTR
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 125 - DNA, 108 bp, Homo sapiens
  misc_feature 1..108: note = HBA2 3'-UTR
  source 1..108: mol_type = genomic DNA
gctggagcctcggtagccgttcctcctgcccgctgggcctcccaacgggccctcctcccctccttgcaccggcccttcctggtctttgaataaagtctgagtgggcag

SEQ ID NO: 126 - RNA, 108 nt, Homo sapiens
  misc_feature 1..108: note = HBA2 3'-UTR
  source 1..108: mol_type = mRNA
gctggagcctcggtagccgttcctcctgcccgctgggcctcccaacgggccctcctcccctccttgcaccggcccttcctggtctttgaataaagtctgagtgggcag

SEQ ID NO: 127 - DNA, 132 bp, Homo sapiens
  misc_feature 1..132: note = HBB-v1 3'-UTR
  source 1..132: mol_type = genomic DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 128 - RNA, 132 nt, Homo sapiens
  misc_feature 1..132: note = HBB-v1 3'-UTR
  source 1..132: mol_type = mRNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 129 - DNA, 311 bp, synthetic construct
  misc_feature 1..311: note = HBB-v2 3'-UTR
  source 1..311: mol_type = other DNA
ctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagc

SEQ ID NO: 130 - RNA, 311 nt, synthetic construct
  misc_feature 1..311: note = HBB-v2 3'-UTR
  source 1..311: mol_type = other RNA
ctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagc

SEQ ID NO: 131 - DNA, 295 bp, synthetic construct
  misc_feature 1..295: note = AES-12S-v1 3'-UTR
  source 1..295: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 132 - RNA, 295 nt, synthetic construct
  misc_feature 1..295: note = AES-12S-v1 3'-UTR
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 133 - DNA, 317 bp, synthetic construct
  misc_feature 1..317: note = AES-12S-v2 3'-UTR
  source 1..317: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 134 - RNA, 317 nt, synthetic construct
  misc_feature 1..317: note = AES-12S-v2 3'-UTR
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 135 - DNA, 71 bp, synthetic construct
  misc_feature 1..71: note = PSMB3-v3 3'-UTR
  source 1..71: mol_type = other DNA
ggactagtccctgttcccagagcccacttttttttctttttttgaaataaaatagcctgtctttcagatct

SEQ ID NO: 136 - RNA, 71 nt, synthetic construct
  misc_feature 1..71: note = PSMB3-v3 3'-UTR
  source 1..71: mol_type = other RNA
ggactagtccctgttcccagagcccacttttttttctttttttgaaataaaatagcctgtctttcagatct

SEQ ID NO: 137 - DNA, 74 bp, synthetic construct
  misc_feature 1..74: note = muag-v5 3'-UTR
  source 1..74: mol_type = other DNA
ggactagttataagactgactagcccgatgggcctcccaacgggccctcctcccctccttgcaccgagattaat

SEQ ID NO: 138 - RNA, 74 nt, synthetic construct
  misc_feature 1..74: note = muag-v5 3'-UTR
  source 1..74: mol_type = other RNA
ggactagttataagactgactagcccgatgggcctcccaacgggccctcctcccctccttgcaccgagattaat

SEQ ID NO: 139 - DNA, 80 bp, synthetic construct
  misc_feature 1..80: note = muag-v6 3'-UTR
  source 1..80: mol_type = other DNA
ggactagttataagactgactagcccgatgggcctcccaacgggccctcctcccctccttgcaccgagattaatagatct

SEQ ID NO: 140 - RNA, 80 nt, synthetic construct
  misc_feature 1..80: note = muag-v6 3'-UTR
  source 1..80: mol_type = other RNA
ggactagttataagactgactagcccgatgggcctcccaacgggccctcctcccctccttgcaccgagattaatagatct

SEQ ID NO: 141 - DNA, 74 bp, synthetic construct
  misc_feature 1..74: note = muag-v7 3'-UTR
  source 1..74: mol_type = other DNA
ggactagttataagactgactagcccgatgggcctcccaacgggccctcctcccctccttgcaccgagagatct

SEQ ID NO: 142 - RNA, 74 nt, synthetic construct
  misc_feature 1..74: note = muag-v7 3'-UTR
  source 1..74: mol_type = other RNA
ggactagttataagactgactagcccgatgggcctcccaacgggccctcctcccctccttgcaccgagagatct

SEQ ID NO: 143 - DNA, 201 bp, synthetic construct
  misc_feature 1..201: note = ALB7-v3 3'-UTR
  source 1..201: mol_type = other DNA
ggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatct

SEQ ID NO: 144 - RNA, 201 nt, synthetic construct
  misc_feature 1..201: note = ALB7-v3 3'-UTR
  source 1..201: mol_type = other RNA
ggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatct

SEQ ID NO: 145 - DNA, 280 bp, synthetic construct
  misc_feature 1..280: note = ANXA4-v3 3'-UTR
  source 1..280: mol_type = other DNA
ggactagtaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaaagatct

SEQ ID NO: 146 - RNA, 280 nt, synthetic construct
  misc_feature 1..280: note = ANXA4-v3 3'-UTR
  source 1..280: mol_type = other RNA
ggactagtaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaaagatct

SEQ ID NO: 147 - DNA, 135 bp, synthetic construct
  misc_feature 1..135: note = CASP1-v3 3'-UTR
  source 1..135: mol_type = other DNA
ggactagtaataaggaaactgtatgaatgtctgtgggcaggaagtgaagagatccttctgtaaaggtttttggaattatgtctgctgaataataaacttttttgaaataataaatctggtagaaaaatgagatct

SEQ ID NO: 148 - RNA, 135 nt, synthetic construct
  misc_feature 1..135: note = CASP1-v3 3'-UTR
  source 1..135: mol_type = other RNA
ggactagtaataaggaaactgtatgaatgtctgtgggcaggaagtgaagagatccttctgtaaaggtttttggaattatgtctgctgaataataaacttttttgaaataataaatctggtagaaaaatgagatct

SEQ ID NO: 149 - DNA, 151 bp, synthetic construct
  misc_feature 1..151: note = COX6B1-v3 3'-UTR
  source 1..151: mol_type = other DNA
ggactagtactggctgcatctccctttcctctgtcctccatccttctcccaggatggtgaagggggacctggtacccagtgatccccaccccaggatcctaaatcatgacttacctgctaataaaaactcattggaaaagtgagaagatct

SEQ ID NO: 150 - RNA, 151 nt, synthetic construct
  misc_feature 1..151: note = COX6B1-v3 3'-UTR
  source 1..151: mol_type = other RNA
ggactagtactggctgcatctccctttcctctgtcctccatccttctcccaggatggtgaagggggacctggtacccagtgatccccaccccaggatcctaaatcatgacttacctgctaataaaaactcattggaaaagtgagaagatct

SEQ ID NO: 151 - DNA, 180 bp, synthetic construct
  misc_feature 1..180: note = FIG4-v3 3'-UTR
  source 1..180: mol_type = other DNA
ggactagtaaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgcagatct

SEQ ID NO: 152 - RNA, 180 nt, synthetic construct
  misc_feature 1..180: note = FIG4-v3 3'-UTR
  source 1..180: mol_type = other RNA
ggactagtaaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgcagatct

SEQ ID NO: 153 - DNA, 367 bp, synthetic construct
  misc_feature 1..367: note = Gnas-v3 3'-UTR
  source 1..367: mol_type = other DNA
ggactagtgaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaagatct

SEQ ID NO: 154 - RNA, 367 nt, synthetic construct
  misc_feature 1..367: note = Gnas-v3 3'-UTR
  source 1..367: mol_type = other RNA
ggactagtgaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaagatct

SEQ ID NO: 155 - DNA, 147 bp, synthetic construct
  misc_feature 1..147: note = Ndufa1-v3 3'-UTR
  source 1..147: mol_type = other DNA
ggactagtggaagcattttcctggctgattaaaagaaattactcagctatggtcatctgttcctgttagaaggctatgcagcatattatatactatgcgcatgttatgaaatgcataataaaaaattttaaaaaatctaaaagatct

SEQ ID NO: 156 - RNA, 147 nt, synthetic construct
  misc_feature 1..147: note = Ndufa1-v3 3'-UTR
  source 1..147: mol_type = other RNA
ggactagtggaagcattttcctggctgattaaaagaaattactcagctatggtcatctgttcctgttagaaggctatgcagcatattatatactatgcgcatgttatgaaatgcataataaaaaattttaaaaaatctaaaagatct

SEQ ID NO: 157 - DNA, 84 bp, synthetic construct
  misc_feature 1..84: note = RPS9-v3 3'-UTR
  source 1..84: mol_type = other DNA
ggactagtgtccacctgtccctcctgggctgctggattgtctcgttttcctgccaaataaacaggatcagcgctttacagatct

SEQ ID NO: 158 - RNA, 84 nt, synthetic construct
  misc_feature 1..84: note = RPS9-v3 3'-UTR
  source 1..84: mol_type = other RNA
ggactagtgtccacctgtccctcctgggctgctggattgtctcgttttcctgccaaataaacaggatcagcgctttacagatct

SEQ ID NO: 159 - DNA, 254 bp, synthetic construct
  misc_feature 1..254: note = Slc7a3-v3 3'-UTR
  source 1..254: mol_type = other DNA
ggactagtcctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgtagatct

SEQ ID NO: 160 - RNA, 254 nt, synthetic construct
  misc_feature 1..254: note = Slc7a3-v3 3'-UTR
  source 1..254: mol_type = other RNA
ggactagtcctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgtagatct

SEQ ID NO: 161 - DNA, 165 bp, synthetic construct
  misc_feature 1..165: note = TUBB4B-v3 3'-UTR
  source 1..165: mol_type = other DNA
ggactagtagccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtgagatct

SEQ ID NO: 162 - RNA, 165 nt, synthetic construct
  misc_feature 1..165: note = TUBB4B-v3 3'-UTR
  source 1..165: mol_type = other RNA
ggactagtagccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtgagatct

SEQ ID NO: 163 - DNA, 124 bp, synthetic construct
  misc_feature 1..124: note = HBA1-v3 3'-UTR
  source 1..124: mol_type = other DNA
ggactagtgctggagcctcggtggccatgcttcttgccccttgggcctccccccagcccctcctccccttcctgcacccgtacccccgtggtctttgaataaagtctgagtgggcggcagatct

SEQ ID NO: 164 - RNA, 124 nt, synthetic construct
  misc_feature 1..124: note = HBA1-v3 3'-UTR
  source 1..124: mol_type = other RNA
ggactagtgctggagcctcggtggccatgcttcttgccccttgggcctccccccagcccctcctccccttcctgcacccgtacccccgtggtctttgaataaagtctgagtgggcggcagatct

SEQ ID NO: 165 - DNA, 122 bp, synthetic construct
  misc_feature 1..122: note = HBA2-v3 3'-UTR
  source 1..122: mol_type = other DNA
ggactagtgctggagcctcggtagccgttcctcctgcccgctgggcctcccaacgggccctcctcccctccttgcaccggcccttcctggtctttgaataaagtctgagtgggcagagatct

SEQ ID NO: 166 - RNA, 122 nt, synthetic construct
  misc_feature 1..122: note = HBA2-v3 3'-UTR
  source 1..122: mol_type = other RNA
ggactagtgctggagcctcggtagccgttcctcctgcccgctgggcctcccaacgggccctcctcccctccttgcaccggcccttcctggtctttgaataaagtctgagtgggcagagatct

SEQ ID NO: 167 - DNA, 146 bp, synthetic construct
  misc_feature 1..146: note = HBB-v3 3'-UTR
  source 1..146: mol_type = other DNA
ggactagtgctcgctttcttgctgtccaatttctattaaaggttcctttgttccctaagtccaactactaaactgggggatattatgaagggccttgagcatctggattctgcctaataaaaaacatttattttcattgcagatct

SEQ ID NO: 168 - RNA, 146 nt, synthetic construct
  misc_feature 1..146: note = HBB-v3 3'-UTR
  source 1..146: mol_type = other RNA
ggactagtgctcgctttcttgctgtccaatttctattaaaggttcctttgttccctaagtccaactactaaactgggggatattatgaagggccttgagcatctggattctgcctaataaaaaacatttattttcattgcagatct

SEQ ID NO: 169 - DNA, 13 bp, synthetic construct
  misc_feature 1..13: note = Kozak sequence
  source 1..13: mol_type = other DNA
gccgccaccatgg

SEQ ID NO: 170 - RNA, 13 nt, synthetic construct
  misc_feature 1..13: note = Kozak sequence
  source 1..13: mol_type = other RNA
gccgccaccatgg

SEQ ID NO: 171-176 - skipped (6 entries)

SEQ ID NO: 177 - DNA, 24 bp, synthetic construct
  misc_feature 1..24: note = histone stem-loop (histoneSL,hSL)
  source 1..24: mol_type = other DNA
caaaggctcttttcagagccacca

SEQ ID NO: 178 - RNA, 24 nt, synthetic construct
  misc_feature 1..24: note = histone stem-loop (histoneSL,hSL)
  source 1..24: mol_type = other RNA
caaaggctcttttcagagccacca

SEQ ID NO: 179 - protein, 14 aa, synthetic construct
  REGION 1..14: note = CR12C carrier peptide
CRRRRRRRRRRRRC

SEQ ID NO: 180 - protein, 13 aa, synthetic construct
  REGION 1..13: note = CR12 carrier peptide
CRRRRRRRRRRRR

SEQ ID NO: 181 - protein, 14 aa, synthetic construct
  REGION 1..14: note = WR12C carrier peptide
WRRRRRRRRRRRRC

SEQ ID NO: 182 - protein, 18 aa, synthetic construct
  REGION 1..18: note = CH6R4H6C carrier peptide
CHHHHHHRRRRHHHHHHC

SEQ ID NO: 183 - protein, 18 aa, synthetic construct
  REGION 1..18: note = CGH5R4H5GC carrier peptide
CGHHHHHRRRRHHHHHGC

SEQ ID NO: 184 - RNA, 164 nt, synthetic construct
  misc_feature 1..164: note = released intron-1 circRNA
  source 1..164: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 185 - RNA, 146 nt, synthetic construct
  misc_feature 1..146: note = released intron-2 circRNA
  source 1..146: mol_type = other RNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 186 - RNA, 50 nt, synthetic construct
  misc_feature 1..50: note = AC linker circRNA
  source 1..50: mol_type = other RNA
aaaaacaaaaaacaaaaaaaacaaaaaaaaaaccaaaaaaacaaaacaca

SEQ ID NO: 187 - RNA, 71 nt, synthetic construct
  misc_feature 1..71: note = 5' spliced circRNA wo AC Linker wo IRES
  source 1..71: mol_type = other RNA
aaaatccgttgaccttaaacggtcgtgtgggttcaagtccctccacccccacgccggaaacgcaatagccg

SEQ ID NO: 188 - RNA, 121 nt, synthetic construct
  misc_feature 1..121: note = 5' spliced circRNA with AC Linker wo IRES
  source 1..121: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacaca

SEQ ID NO: 189 - RNA, 37 nt, synthetic construct
  misc_feature 1..37: note = 3' spliced circRNA wo UTR wo PolyA
  source 1..37: mol_type = other RNA
cggctattatgcgttaccggcgagacgctacggactt

SEQ ID NO: 190 - RNA, 73 nt, synthetic construct
  misc_feature 1..73: note = 3' spliced circRNA with PolyA wo UTR
  source 1..73: mol_type = other RNA
aaaaaaaaaaaaaaaaaaaaaaaaaaaaaaaaaaaacggctattatgcgttaccggcgagacgctacggactt

SEQ ID NO: 191 - RNA, 131 nt, synthetic construct
  misc_feature 1..131: note = 3' spliced circRNA with PolyA with UTR
  source 1..131: mol_type = other RNA
tccctgttcccagagcccacttttttttctttttttgaaataaaatagcctgtctttcaaaaaaaaaaaaaaaaaaaaaaaaaaaaaaaaaaaacggctattatgcgttaccggcgagacgctacggactt

SEQ ID NO: 192 - RNA, 58 nt, synthetic construct
  misc_feature 1..58: note = PSMB 3'UTR
  source 1..58: mol_type = other RNA
tccctgttcccagagcccacttttttttctttttttgaaataaaatagcctgtctttc

SEQ ID NO: 193 - RNA, 36 nt, synthetic construct
  misc_feature 1..36: note = PolyA36
  source 1..36: mol_type = other RNA
aaaaaaaaaaaaaaaaaaaaaaaaaaaaaaaaaaaa

SEQ ID NO: 194 - RNA, 60 nt, synthetic construct
  misc_feature 1..60: note = PolyA60
  source 1..60: mol_type = other RNA
aaaaaaaaaaaaaaaaaaaaaaaaaaaaaaaaaaaaaaaaaaaaaaaaaaaaaaaaaaaa

SEQ ID NO: 195 - RNA, 100 nt, synthetic construct
  misc_feature 1..100: note = PolyA100
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 196 - RNA, 1785 nt, synthetic construct
  misc_feature 1..1785: note = precursor circRNA-01
  source 1..1785: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 197 - RNA, 1861 nt, synthetic construct
  misc_feature 1..1861: note = precursor circRNA-02
  source 1..1861: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgatccctgttcccagagcccacttttttttctttttttgaaataaaatagcctgtctttcaaaaaaaaaaaaaaaaaaaaaaaaaaaaaaaaaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 198 - RNA, 1897 nt, synthetic construct
  misc_feature 1..1897: note = precursor circRNA-03
  source 1..1897: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgatccctgttcccagagcccacttttttttctttttttgaaataaaatagcctgtctttcggatccaaaaaaaaaaaaaaaaaaaaaaaaaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 199 - RNA, 1937 nt, synthetic construct
  misc_feature 1..1937: note = precursor circRNA-04
  source 1..1937: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgatccctgttcccagagcccacttttttttctttttttgaaataaaatagcctgtctttcggatccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 200 - RNA, 2968 nt, synthetic construct
  misc_feature 1..2968: note = precursor circRNA-05
  source 1..2968: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 201 - RNA, 1475 nt, synthetic construct
  misc_feature 1475^1: note = spliced circRNA-01
  source 1..1475: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggactt

SEQ ID NO: 202 - RNA, 1551 nt, synthetic construct
  misc_feature 1551^1: note = spliced circRNA-02
  source 1..1551: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgatccctgttcccagagcccacttttttttctttttttgaaataaaatagcctgtctttcaaaaaaaaaaaaaaaaaaaaaaaaaaaaaaaaaaaacggctattatgcgttaccggcgagacgctacggactt

SEQ ID NO: 203 - RNA, 1587 nt, synthetic construct
  misc_feature 1587^1: note = spliced circRNA-03
  source 1..1587: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgatccctgttcccagagcccacttttttttctttttttgaaataaaatagcctgtctttcggatccaaaaaaaaaaaaaaaaaaaaaaaaaaaaaaaaaaaaaaaaaaaaaaaaaaaaaaaaaaaactcgagcggctattatgcgttaccggcgagacgctacggactt

SEQ ID NO: 204 - RNA, 1627 nt, synthetic construct
  misc_feature 1627^1: note = spliced circRNA-04
  source 1..1627: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgatccctgttcccagagcccacttttttttctttttttgaaataaaatagcctgtctttcggatccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcgagcggctattatgcgttaccggcgagacgctacggactt

SEQ ID NO: 205 - RNA, 2658 nt, synthetic construct
  misc_feature 2658^1: note = spliced circRNA-05
  source 1..2658: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

SEQ ID NO: 206 - RNA, 830 nt, synthetic construct
  misc_feature 1..830: note = R8732 (Gluc(GC))
  source 1..830: mol_type = other RNA
aggagagtcccgcagtcggcgtccagcggctctgcttgttcgtgtgtgtgtcgttgcaggccttattcaagcttaccatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 207 - RNA, 1925 nt, synthetic construct
  misc_feature 1..1925: note = R8730 (PpLuc(GC))
  source 1..1925: mol_type = other RNA
aggagagtcccgcagtcggcgtccagcggctctgcttgttcgtgtgtgtgtcgttgcaggccttattcaagcttaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 208 - RNA, 20 nt, synthetic construct
  misc_feature 1..20: note = AS1-5'end
  source 1..20: mol_type = other RNA
tctttaccctcgtcttgacg

SEQ ID NO: 209 - RNA, 20 nt, synthetic construct
  misc_feature 1..20: note = AS2-3'end
  source 1..20: mol_type = other RNA
tatgctgttatccgtcgatt

SEQ ID NO: 210 - DNA, 741 bp, synthetic construct
  misc_feature 1..741: note = IRES CVB3
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 211 - DNA, 585 bp, synthetic construct
  misc_feature 1..585: note = IRES-1
  source 1..585: mol_type = other DNA
cccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 212 - DNA, 568 bp, synthetic construct
  misc_feature 1..568: note = IRES-2
  source 1..568: mol_type = other DNA
ctccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 213 - DNA, 696 bp, synthetic construct
  misc_feature 1..696: note = IRES-3
  source 1..696: mol_type = other DNA
ttgccagtctgctcgatatcgcaggctgggtccgtgactacccactccccctttcaacgtgaaggctacgatagtgccagggcgggtactgccgtaagtgccaccccaaacaacaacaacaaaacaaactccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 214 - DNA, 697 bp, synthetic construct
  misc_feature 1..697: note = IRES-4
  source 1..697: mol_type = other DNA
ttgccagtctgctcgatatcgcaggctgggtccgtgactacccactccccctttcaacgtgaaggctacgatagtgccagggcgggtactgccgtaagtgccaccccaaaacaacaacaaccccccctctccctcctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 215 - DNA, 562 bp, synthetic construct
  misc_feature 1..562: note = IRES-5
  source 1..562: mol_type = other DNA
ccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 216 - DNA, 156 bp, synthetic construct
  misc_feature 1..156: note = IRES-6
  source 1..156: mol_type = other DNA
gtaaattaaatgctatttacaaaatttaaacagaaaggagagatgttatgaaccggttttacaaggtttcatacatcgaaaatagcactacctggggcagccgacacactaacatcgtctgtttaaccagaagtgttactgaaaggaggttattta

SEQ ID NO: 217 - DNA, 340 bp, synthetic construct
  misc_feature 1..340: note = IRES-7
  source 1..340: mol_type = other DNA
acctgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggacatttgggcgtgcccccgcaagactgctagccgagtagcgttgggttgcgaaaggccttgtggtactgcctgatagggtgcttgcgagtgccccgggaggtctcgtagaccgtgcatc

SEQ ID NO: 218 - DNA, 315 bp, synthetic construct
  misc_feature 1..315: note = IRES-8
  source 1..315: mol_type = other DNA
ctacaagctttgtgtaaacaaacttttgtttggcttttctcaagcttctctcacatcaggccccaaagatgtcctgaaggtaccccgtgtatctgaggatgagcaccatcgactacccggacctgcaaaattttgcaaacgcatgtggtatcccagccccctcctctcggggagggggctttgctcactcagcacaggatctgatcaggagatccacctccggtgctttacaccggggcgtggatttaaaaattgcccaaggcctggcgcacaacctaggggactaggttttccttatattttaaagctgtcaat

SEQ ID NO: 219 - DNA, 182 bp, synthetic construct
  misc_feature 1..182: note = IRES-9
  source 1..182: mol_type = other DNA
gtcttaggacgacgcatgtggtatcccagcccccgcctacattggcgggggcttttgaagcaccagacactggatctgatcaggaggagggtagctgctttacagcccctcttaaaaattgcccaaggtccggccacccaacctaggggactaggttttccttttatttttaaattgtcatt

SEQ ID NO: 220 - DNA, 759 bp, synthetic construct
  misc_feature 1..759: note = IRES-10
  source 1..759: mol_type = other DNA
acatgggggagactgcatgtggcagtcttgaaacgtgtggtttgacgtctaccttatatggcagtgggtggagtactgcaaagatgtcaccgtgctttacacggtttttgaaccccacaccggctgtttgacgctcgtagggcagcaggtttattttcattaaaattcttactttctagctgcatgagttctattcatgcagacggagtgatactcccgttccttcttggacaggttgcctccacgccctttgtggatcttaaggtgaccaagtcactggtgttggaggtgaagatagagagtcctcttgggaatgtcatgtggctgtgccaggggttgtagcgatgccattcgtgtgtgcggatttcctctcgtggtgacacgagcctcacaggccaaaagccccgtccgaaaggacccgaatggtggagtgaccctgactcccccctgcatagttttgtgattaggaacttgaggaatttctgtcataaatctctatcacatcaggccccaaagatgtcctgaaggtaccctgtgtatctgaggatgagcaccaccgactacccggacttgcattagcagacacatgtggttgcccagccccacctcttcagaggtggggctttgctcactcagcacaggatctgatcaggagccccgctcgtgtgctttacactcgacgcggggttaaaaattgcccaaggcctggcacaacaacctaggggactaggttttcctatttttgtaaattatgtcaat

SEQ ID NO: 221 - DNA, 478 bp, synthetic construct
  misc_feature 1..478: note = IRES-11
  source 1..478: mol_type = other DNA
gtgacaatcagccagattgttaacggtcaagcacttctgtttccccggtacccttgtatacgcttcacccgaggcgaaaagtgaggttatcgttatccgcaaagtgcctacgagaagcctagtagcacttttgaagcctatggctggtcgctcaactgtttacccagcagtagacctggcagatgaggctagatgttccccaccagcgatggtgatctagcctgcgtggctgcctgcacactctattgagtgtgaagccagaaagtggacaaggtgtgaagagcctattgtgctcactttgagtcctccggcccctgaatgtggctaatcctaaccccgtagctgttgcatgtaatccaacatgtctgcagtcgtaatgggcaactatgggatggaaccaactactttgggtgtccgtgtttcttgtttttctttatgcttgcttatggtgacaactgtagttattacatttgttacc

SEQ ID NO: 222 - DNA, 628 bp, synthetic construct
  misc_feature 1..628: note = IRES-12
  source 1..628: mol_type = other DNA
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatc

SEQ ID NO: 223 - DNA, 618 bp, synthetic construct
  misc_feature 1..618: note = IRES-13
  source 1..618: mol_type = other DNA
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatc

SEQ ID NO: 224 - DNA, 610 bp, synthetic construct
  misc_feature 1..610: note = IRES-14
  source 1..610: mol_type = other DNA
ttaaaactgggtacaggttgttcccacctgtatcacccacgtggtgtggtgctcttgtattccggtacacttgcacgccagtttgccacccctcacccgtcgtaacttagaagctaacaactcgaccaacaggcggtggtaaaccataccacttacggtcaagcactcctgtttccccggtatgcgaggaatagactcctacagggttgaagcctcaagtatcgttatccgcattggtactacgcaaagcttagtagtgccttgaaagtcccttggttggtcgctccgctagtttcccctagtagacctggcagatgaggcaggacactccccactggcgacagtggtcctgcctgcgtggctgcctgcgcacccttaggggtgcgaagccaagtgacagacaaggtgtgaagagccccgtgtgctaccaatgagtcctccggcccctgaatgcggctaatccaaccccacagctattgcacacaagccagtgtgtatgtagtcgtaatgagcaattgtgggacggaaccgactactttgggtgtccgtgtttccttttattcttatcattctgcttatggtgacaatactgtgaaatagtgttgttacc

SEQ ID NO: 225 - DNA, 614 bp, synthetic construct
  misc_feature 1..614: note = IRES-15
  source 1..614: mol_type = other DNA
taaaactggatccaggttgttcccacctggatctcctattgggagttgtactctattattccggtaattttgtacgccagttttatcttccccctccccaattgtaacttagaaggttatcaatacgaccaataggtggtagttagccaaactaccaaaggtcaagcacttctgtttccccggtcaaagttgatatgctccaacagggcaaaaacaactgagatcgttatccgcaaagtgcctacgcaaagcctagtaacacctttgaagatttatggttggtcgttccgctatttcccatagtagacctggcagatgaggctagaaatcccccactggcgacagtgctctagcctgcgtggctgcctgcgcaccccttgggtgcgaagccatacattggacaaggtgtgaagagccccgtgtgctcactttgagtcctccggcccctgaatgtggctaaccttaaccctgcagctagtgcatgtaatccaacatgttgctagtcgtaatgagtaattgcgggacgggaccaactactttgggtgtccgtgtttcactttttccttttaatattgcttatggtgacaatatatatagctatatatattgacacc

SEQ ID NO: 226 - DNA, 624 bp, synthetic construct
  misc_feature 1..624: note = IRES-16
  source 1..624: mol_type = other DNA
ttcccctgcaaccattacgcttactcgcatgtgcattgagtggtgcatgtgttgaacaaacagctacactcacatgggggcgggttttcccgccctacggcttctcgcgaggcccacccctcccctttctcccataactacagtgctttggtaggtaagcatcctgatcccccgcggaagctgctcacgtggcaactgtggggacccagacaggttatcaaaggcacccggtctttccgccttcaggagtatccctgctagcgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactcaggggatacctcaaggcacccaggaatgccagggaggtaccccgcttcacagcgggatctgaccctggggtaaatgtctgcggggggtcttcttggcccacttctcagtacttttcagg

SEQ ID NO: 227 - DNA, 730 bp, synthetic construct
  misc_feature 1..730: note = IRES-17
  source 1..730: mol_type = other DNA
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgaattccggtagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatg

SEQ ID NO: 228 - DNA, 709 bp, synthetic construct
  misc_feature 1..709: note = IRES-18
  source 1..709: mol_type = other DNA
tatggcaggcgggcttgtggacggcttcggcccacccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 229 - DNA, 708 bp, synthetic construct
  misc_feature 1..708: note = IRES-19
  source 1..708: mol_type = other DNA
tttgaaaggggtctcctagagagcttggccgtcgggccttataccccgacttgctgagtttctctaggagagcccttttcccagccctgaggcggctggtcaataaaagcctcaaacgtaactaacacctaagaagatcatgtaaaccctatgcctggtctccactattcgaaggcaacttgcaataagaagagtgggatcaagacgcttaaagcatagagacagttttcttttctaacccacatttgtgtggggtggcagatggcgtgccataactctaatagtgagataccacgcttgtggaccttatgctcacacagccatcctctagtaagtttgtgagacgtctggtgacgtgtgggaacttattggaaacaacattttgctgcaaagcatcctactgccagcggaaaaacacctggtaacaggtgcctctggggccaaaagccaaggtttaacagaccctttaggattggttctaaacctgagatgttgtggaagatatttagtacctgctgatctggtagttatgcaaacactagttgtaaggcccatgaaggatgcccagaaggtacccgtaggtaacaagtgacactatggatctgatttggggccagatacctctatcttggtgatctggttaaaaaacatctaatgggccaaacccgggggggatccccggtttcctcttattctatcaatgccact

SEQ ID NO: 230 - DNA, 508 bp, synthetic construct
  misc_feature 1..508: note = IRES-20
  source 1..508: mol_type = other DNA
gtataagagacaggtgtttgccttgtcttcggactggcatcttgggaccaaccccccttttccccagccatgggttaaatggcaataaaggacgtaacaactttgtaaccattaagctttgtaattttgtaaccactaagctttgtgcacataatgtaaccatcaagcttgttagtcccagcaggaggtttgcatgcttgtagccgaaatggggctcgaccccccatagtaggatacttgattttgcattccattgtggacctgcaaactctacacatagaggctttgtcttgcatctaaacacctgagtacagtgtgtacctagaccctatagtacgggaggaccgtttgtttcctcaataaccctacataataggctaggtgggcatgcccaatttgcaagatcccagactgggggtcggtctgggcagggttagatccctgttagctactgcctgatagggtggtgctcaaccatgtgtagtttaaattgagctgttcatatacc

SEQ ID NO: 231 - DNA, 248 bp, synthetic construct
  misc_feature 1..248: note = IRES-21
  source 1..248: mol_type = other DNA
actgaagatcctacagtaactactgccccaatgaacgccacagatgggtctgctgatgactacctatcttagtgctagttgaggtttgaagtgagccggtttttagaagaaccagtttctgaacattatcatccccagcatctattctatacgcacaagatagatagtcatcagcagacacatctgtgctactgcttgatagagttgcggctggtcaacttagattggtataaccagttgagtggcaa

SEQ ID NO: 232 - DNA, 516 bp, synthetic construct
  misc_feature 1..516: note = IRES-22
  source 1..516: mol_type = other DNA
tatgcatcactggacggcctaacctcggtcgtggcttcttgccgatttcagcgctaccaggctttctggtctcgccaggcgttgattagtaggtgcactgtctaagtgaagacagcagtgctctctgtgaaaagttgatgacactcttcaggtttgtagcgatcactcaaggctagcggatttccccgtgtggtaacacacgcctctaggcccagaaggcacggtgttgacagcaccccttgagtggctggtcttccccaccagcacctgatttgtggattcttcctagtaacggacaagcatggctgctcttaagcattcagtgcgtccggggctgaaggatgcccagaaggtacccgcaggtaacgataagctcactgtggatctgatctggggctgcgggctgggtgtctttccacccagccaaaacccgtaaaacggtagtcgcagttaaaaaacgtctaggccccacccccccagggatggggggttcccttaaaccctcacaagttcaac

SEQ ID NO: 233 - DNA, 478 bp, synthetic construct
  misc_feature 1..478: note = IRES-23
  source 1..478: mol_type = other DNA
tgaaaagggggcgcagggtggtggtggttactaaatacccaccatcgccctgcacttcccttttcccctgtggctcagggtcacttagccccctctttgggttaccagtagttttctacccctgggcacagggttaactatgcaagacggaacaacaatctcttagtccccctcgccgatagtgggctcgacccccatgtgtaggagtggataagggacggagtgagccgatacggggaagagtgtgcggtcacaccttaattccatgagcgctgcgaagaaggaagctgtgaacaatggcgacctgaaccgtacacatggagctccacaggcatggtactcgttagactacgcagcctggttgggagtgggtataccctgggtgagccgccagtgaatgggagttcactggttaacacacactgcctgatagggtcagggcctcctgtccccgccgtaatgaggtagaccatatgcc

SEQ ID NO: 234 - DNA, 403 bp, synthetic construct
  misc_feature 1..403: note = IRES-24
  source 1..403: mol_type = other DNA
gcggctggatattctggccgtgcaactgcttttgaccagtggctctgggtaacttagccaaagtgtccttctccctttccctattatatgttttatggctttgtctggtcttgtttagtttatatataagatcctttccgccgatatagacctcgacagtctagtgtaggaggattggtgatattaatttgccccagaagagtgaccgtgacacatagaaaccatgagtacatgtgtatccgtggaggatcgcccgggactggattccatatcccattgccatcccaacaagcggagggtatacccactatgtgcacgtctgcagtgggagtctgcagatttagtcatactgcctgatagggtgtgggcctgcactctggggtactcaggctgtttatataat

SEQ ID NO: 235 - DNA, 404 bp, synthetic construct
  misc_feature 1..404: note = IRES-25
  source 1..404: mol_type = other DNA
gctggactttctggctgcgcaactgcttttaaccagtggctctgggttacttagccaaaaccccctttccccgtaccctagtttgtgtgtgtattattattttgttgttgttttgtaaatttttatataagatcctttccgccgatatagacctcgacagtctagtgtaggaggattggtgatattaatatgccccagaagagtgaccgtgacacatagaaaccatgagtacatgtgtatccgtggaggatcgcccgggactggattccatatcccattgccatcccaacaaacggagggtatacccgctatgtgcgcgtctacagtgggaatctgtagatttagtcatactgcctgatagggtgtgggcctgcactctggggtactcaggctgtttatataat

SEQ ID NO: 236 - DNA, 744 bp, synthetic construct
  misc_feature 1..744: note = IRES-26
  source 1..744: mol_type = other DNA
ttaaaacagcctgtgggttgttcccatccacagggcccactgggcgccagcactctggtattgcggtaccttagtgcgcctgttttatatacccgtcccccaaacgtaacttagacgcatgtcaacgaagaccaatagtaagcgcagcacaccagctgtgttccggtcaagcacttctgttaccccggaccgagtatcaataagctactcacgtggctgaaggagaaaacgttcgttacccgaccaattacttcaagaaacctagtaacaccatgaaggttgcgcagtgtttcgctccgcacaaccccagtgtagatcaggtcgatgagtcaccgcattccccacgggtgaccgtggcggtggctgcgctggcggcctgcccatggggaaacccatgggacgcttcaatactgacatggtgcgaagagtctattgagctaattggtagtcctccggcccctgaatgcggctaatcctaactgcggagcagatacccacacaccagtgggcagtctgtcgtaacgggcaactctgcagcggaaccgactactttgggtgtccgtgtttctctttatccttatactggctgcttatggtgacaattgagagattgttaccatatagctattggattggccatccggtgacaaatagagcaattgtgtatttgtttgttggtttcgtgccattaaattacaaggttctaaacacccttaatcttattatagcattcaacacaacaaa

SEQ ID NO: 237 - DNA, 663 bp, synthetic construct
  misc_feature 1..663: note = IRES-27
  source 1..663: mol_type = other DNA
gtacattagatgcgtcatctgcaactttagtcaataaattacctccaatgtcattaccaacattccctaccttttcactaacacctaagacaacaagtacctatgcctggtctccactattcgaaggcaacttgcaataagaagagtggaattaagacgcttaaagcatagagctagttatcttttctaacccacaaagttttgtggggtggcagatggcgtgccataactctattagtgagataccatgcttgtggatcttatgctcacacagccatcctctagtaagttgataaggtgtctggtgatatgtgggaactcacatgaaccattaatttaccgtaaggtatcctatagccagcggaatcacatctggtgacagatgcctctggggccgaaagccaaggtttaacagaccctataggattggtttcaaaacctgaattgatgtggattgtgtatagtacctgttgatctggtaacagtgtcaacactagttgtaaggcccacgaaggatgcccagaaggtacccgtaggtaacaagtgacactatggatctgatctggggccagctacctctatcatggtgagttggttaaaaaacgtctagtgggccaaacccaggggggatccctggtttccttttacctaatcaaagccact

SEQ ID NO: 238 - DNA, 744 bp, synthetic construct
  misc_feature 1..744: note = IRES-28
  source 1..744: mol_type = other DNA
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaattcacgttgggcaactagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 239 - DNA, 734 bp, synthetic construct
  misc_feature 1..734: note = IRES-29
  source 1..734: mol_type = other DNA
ttcaagaggggtctccggagttttccggaacccctcttggaagtccatggtgaggggacttgatacctcaccgccgtttgcctaggctataggctaaatttccctttccctgtccttcccctatttccttttgttttgtttgtaaatattaattcctgcaggttcagggttctttaatctgtttctctataagaacactcaatttttcacgctttctgtctcctttcttccagggctctccccttgccctaggctctggccgttgcgcccggcggggtcaactccatgattagcatggagctgtaggagtctaaattggggacgcagatgtttgggacgtcgccttgcagtgttaacttggctttcatgaacctctttgatcttccacaaggggtaggctacgggtgaaacctcttaggctaatacttcaatgaagagatgccttggatagggtaacagcggcggatattggtgagttgttaagacaaaaaccattcaacgccggaggactggctctcatccagtggatgcattgagggaattgattgtcagggctgtctctaggtttaatctcagacctctctgtgcttagggcaaacactatttggccttaaatgggatcctgtgagagggggtccctccattgacagctggactgttctttggggccttatgtggtgtttgcctctgaggtactcaggggcatttaggtttttcctcattcttaaataata

SEQ ID NO: 240 - DNA, 638 bp, synthetic construct
  misc_feature 1..638: note = IRES-30
  source 1..638: mol_type = other DNA
gggagtaaacctcaccaccgtttgccgtggtttacggctacctatttttggatgtaaatattaattcctgcaggttcaggtctcttgaattatgtccacgctagtggcactctcttacccataagtgacgccttagcggaacctttctacacttgatgtggttaggggttacattatttccctgggccttctttggccctttttcccctgcactatcattctttcttccgggctctcagcatgccaatgttccgaccggtgcgcccgccggggttaactccatggttagcatggagctgtaggccctaaaagtgctgacactggaactggactattgaagcatacactgttaactgaaacatgtaactccaatcgatcttctacaaggggtaggctacgggtgaaaccccttaggttaatactcatattgagagatacttctgataggttaaggttgctggataatggtgagtttaacgacaaaaaccattcaacagctgtgggccaacctcatcaggtagatgcttttggagccaagtgcgtaggggtgtgtgtggaaatgcttcagtggaaggtgccctcccgaaaggtcgtaggggtaatcaggggcagttaggtttccacaattacaatttgaa

SEQ ID NO: 241 - DNA, 743 bp, synthetic construct
  misc_feature 1..743: note = IRES-31
  source 1..743: mol_type = other DNA
gctcttccgatctgggttgttcccacccacagggcccactgggcgccagcactctgattccacggaatctttgtgcgcctgttttacaacccttcccaatttgtaacgtagaagcaatacacactactgatcaatagtaggcatggcgcgccagtcatgtcatgatcaagcacttctgttcccccggactgagtatcaatagactgctcacgcggttgaaggagaaaacgttcgttacccggctaactacttcgagaaacctagtagcaccatggaagctgcggagtgtttcgctcagcactttccccgtgtagatcaggtcgatgagtcactgcaatccccacgggcgaccgtggcagtggctgcgttggcggcctgcctatggggcaacccataggacgctctaatgtggacatggtgcgaagagtctattgagctagttagtagtcctccggcccctgaatgcggctaatcctaactgcggagcacatgccttcaacccaggaggtggtgtgtcgtaacgggtaactctgcagcggaaccgactactttgggtgtccgtgtttccttttatccttatattggctgcttatggtgacaatcacggaattgttgccatatagctattggattggccatccggtgtctaacagagctattgtatacctatttgttggatttactcccctatcatacaaatctctgaacactttgtgctttatactgaacttaaacacacgaaa

SEQ ID NO: 242 - DNA, 746 bp, synthetic construct
  misc_feature 1..746: note = IRES-32
  source 1..746: mol_type = other DNA
ttaaaacagctctggggttgttcccaccccagaggcccacgtggcggccagtacaccggtaccacggtacccttgtacgcctgttttatactcccctccccgtaaactagaagcacgaaacacaagttcaatagaagggggtacagaccagtaccaccacgaacaagcacttctgttcccccggtgaggtcacatagactgtccccacggtcaaaagtgactgatccgttatccgctcacgtacttcggaaagcctagtaccaccttggaatctacgatgcgttgcgctcagcactcgaccccggagtgtagcttaggctgatgagtctggacgttccccactggtgacagtggtccaggctgcgttggcggcctacctgtggtccaaaaccacaggacgctagtagtgaacaaggtgtgaagagcccactgagctacctgagaatcctccggcccctgaatgcggctaatcccaaccacggagcaggtaatcgcaaaccagcggtcagcctgtcgtaacgcgtaagtctgtggcggaaccgactactttgggtgtccgtgtttccttttatttttatggtggctgcttatggtgacaatcatagattgttatcataaagcaaattggattggccatccggagtgagctaaactatctatttctctgagtgttggattcgtttcacccacattctgaacaatcagcctcattagtgttaccctgttaataagacgatatcatcacg

SEQ ID NO: 243 - DNA, 726 bp, synthetic construct
  misc_feature 1..726: note = IRES-33
  source 1..726: mol_type = other DNA
ttaaaacagctctggggttgttcccaccccagaggcccacgtggcggctagtactccggtaccccggtacccttgtacgcctgttttatactccctttcccaagtaactttagaagaaataaactaatgttcaacaggagggggtacaaaccagtaccaccacgaacacacacttctgtttccccggtgaagttgcatagactgtacccacggttgaaagcgatgaatccgttacccgcttaggtacttcgagaagcctagtatcatcttggaatcttcgatgcgttgcgatcagcactctaccccgagtgtagcttgggtcgatgagtctggacaccccacaccggcgacgtggtccaggctgcgttggcggcctacccatggctagcaccatgggacgctagttgtgaacaaggtgcgaagagcctattgagctacctgagagtcctccggcccctgaatgcggctaatcccaaccacggagcaaatgctcacaatccagtgagtggtttgtcgtaatgcgcaagtctgtggcggaaccgactactttgggtgtccgtgtttccttttatttttattatggctgcttatggtgacaatctgagattgttatcatatagctattggattagccatccggtgatatcttgaaattttgccataactttttcacaaatcctacaacattacactacactttctcttgaataattgagacaactcata

SEQ ID NO: 244 - DNA, 737 bp, synthetic construct
  misc_feature 1..737: note = IRES-34
  source 1..737: mol_type = other DNA
ttaaaatagcctcagggttgttcccaccctgagggcccacgtggtgtagtactctggtattacggtacctttgtacgcctattttatacccccttccccaagtaatttagaagcaagcacaaaccagttcagtagtaagcagtacaatccagtactgtaatgaacaagtacttctgttaccccggaagggtctatcggtaagctgtacccacggctgaagaatgacctaccgttaaccggctacctacttcgagaagcctagtaatgccgttgaagttttattgacgttacgctcagcacactaccccgtgtgtagttttggctgatgagtcacggcactccccacgggcgaccgtggccgtggctgcgttggcggccaaccaaggagtgcaagctccttggacgtcatattacagacatggtgtgaagagcctattgagctaggtggtagtcctccggcccctgaatgcggctaatcctaactccggagcatatcggtgcgaaccagcacttggtgtgttgtaatacgtaagtctggagcggaaccgactactttgggtgtccgtgtttcctgttttaacttttatggctgcttatggtgacaatttaacattgttaccatatagctgttgggttggccatccggattttgttataaaaccatttcctcgtgccttgacctttaacacatttgtgaacttctttaaatcccttttattagtccttaaatactaaga

SEQ ID NO: 245 - DNA, 327 bp, synthetic construct
  misc_feature 1..327: note = IRES-35
  source 1..327: mol_type = other DNA
aactgttgttgtagcaatgcgcatattgctacttcggtacgcctaattggtaggcgcccggccgaccggccccgcaagggcctagtaggacgtgtgacaatgccatgagggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 246 - DNA, 281 bp, synthetic construct
  misc_feature 1..281: note = IRES-36
  source 1..281: mol_type = other DNA
tgacgtgggggggttgattttccccccccggcactgggtgcaagccccagaaaccgacgcctatctaagtagacgcaatgactcggcgccgactcggcgaccggccaaaaggtggtggatgggtgatgacagggttggtaggtcgtaaatcccggtcatcctggtagccactataggtgggtcttaagagaaggtcaagattcctcttacgcctgcggcgagaccgcgcacggtccacaggtgttggccctaccggtgtgaataagggcccgacatcaggc

SEQ ID NO: 247 - DNA, 459 bp, synthetic construct
  misc_feature 1..459: note = IRES-37
  source 1..459: mol_type = other DNA
gacgtgggggggttgatcccccccctttggcactgggtgcaagccccagaaaccgacgcctatttaaacagacgttaagaaccggcgccgacccggcgaccggccaaaaggtggtggatgggtgatgccagggttggtaggtcgtaaatcccggtcatcttggtagccactataggtgggtcttaagggttggttaaggtccctctggcgcttgtggcgagaaagcgcacggtccacaggtgttggccctaccggtgtgaataagggcccgacgtcaggctcgtcgttaaaccgagcccactacccacctgggcaaacaacgcccacgtacggtccacgtcgcccttcaatgtctctcttgaccaataggcttagccggcgagttgacaaggaccagtgggggctgggcggtaggggaaggacccctgccgctgcccttcccggtggagtgggaaatgc

SEQ ID NO: 248 - DNA, 350 bp, synthetic construct
  misc_feature 1..350: note = IRES-38
  source 1..350: mol_type = other DNA
tgacgtgggggggttgatccgccccccccggcactcggtgcaagccccataaaccgacgcctatctaagtagacgcaatgactcggcgccgactcggcgaccggccaaaaggtggtggatgggtggtgacagggttggtaggtcgtaaatcccggtcatcctggtagccactataggtgggtcttaagagaaggtcaagactcctcttgtgcctgcggcgagaccgcgcacggtccacaggtgctggccctaccggtgtgaataagggcccgacgtcaggctcgtcgttaaaccgagcccgtcacccacctgggcaaacgacgcccacgtacggtccacgtcgcccttca

SEQ ID NO: 249 - DNA, 318 bp, synthetic construct
  misc_feature 1..318: note = IRES-39
  source 1..318: mol_type = other DNA
tgtagcaatgcgcatattgctacttcggtacgcctaattggtaggcgcccggccgaccggccccgcaagggcctagtaggacgtgtgacaatgccatgcgggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 250 - DNA, 399 bp, synthetic construct
  misc_feature 1..399: note = IRES-40
  source 1..399: mol_type = other DNA
attttctggccgtgtagctgcttttgaccagtggctctgggttacttagccaaatcccccttccttcacccttttaaatttgatggtctgtgttgtttgttttgtcttgtctaaataatatataagatccttcccgccgatacagacctcgacagtctggtgtaggagggttggtgttattaatttgccccagaagagtgaccgtgacacatagaaaccatgagtacatgtgtatccgtggaggatcgcccgggactggattccatatcccattgccatcccaacaagcggagggtatacccactatgtgcgcgtttgcagtgggaatctgcaaatttagtcatactgcctgatagggtgtgggcctgcactctggggtactcaggctgttcatataat

SEQ ID NO: 251 - DNA, 430 bp, synthetic construct
  misc_feature 1..430: note = IRES-41
  source 1..430: mol_type = other DNA
cccctccacccttaaggtggttgtatcccacataccccaccctcccttccaaagtggacggacaactggattttgactaacggcaagtctgaatggtatgatttggatacgtttaaacggcagtagcgtggcgagctatggaaaaatcgcaattgtcgatagccatgttagtgacgcgcttcggcgtgctcctttggtgattcggcgactggttacaggagagtaggcagtgagctatgggcaaacctctacagtattacttagagggaatgtgcaattgagacttgacgagcgtctctttgagatgtggcgcatgctcttggcattaccatagtgagcttccaggttgggaaacctggactgggcctatactacctgatagggtcgcggctggccgcctgtaactagtatagtcagttgaaaccccccc

SEQ ID NO: 252 - DNA, 504 bp, synthetic construct
  misc_feature 1..504: note = IRES-42
  source 1..504: mol_type = other DNA
gtctctttagtgtctatgcttcagagagcggtgaactgacaccgttgcttcttgcacagcccttcgtgccggtctttccggttctcgacagcgttgggcatcatggctagttaggctaagatagtggatgatctagtgaacagttttggattgtttggagttttgtagcgatgctagtagtgtgtgtggacctccccacgtggtaacacgtgccccacaggccaaaagccaaggtgttgaaagcacccctactagtcccagactcacccatctgggaactcctctcatgaaaaatcttagtaacttttgattcggctattcatcaacctctctagtcaagggctgaaggatgcccggaaggtacccgcaggtaacgataagctcactgtggatctgatccggggctttggtgcgaccgtctgtccggcgtagccagagttaaaaaacgtctaggcccttccaccccaagggattggggtttccccaatcatttgaaagttcact

SEQ ID NO: 253 - DNA, 532 bp, synthetic construct
  misc_feature 1..532: note = IRES-43
  source 1..532: mol_type = other DNA
ttttgaacgccacctcggagcgatatccggggaccccctcccctttttccttcctaccttcttcccaaatttccctcttcccttgttattttggtttggatttcctggacatgactcggacggatctatctcatttgctttgtgtctgctccaccagtggcatggtcgaaagatcatcaacactggacgtgtactgtaatggccaaacgtgcccacaggggaaaccatgccggtcgctgtagcggcgggtggacgtggtggacccctctccctgctcataaactttgggtaggtgaagggttcaagcgacgcttgccgtgagggcgcatccggatggtgggaaccaacaaactaggctgtaatggccgacctcaggtggatgagctagggctgctgcaccaaaagggactcgattcgatatcccggcctggtagcctagtgcagtggactcgtagttgggaatctacgactggcctagtacagggtgatagccccgtttcccacgcccacctgttgtagggacacccccccc

SEQ ID NO: 254 - DNA, 494 bp, synthetic construct
  misc_feature 1..494: note = IRES-44
  source 1..494: mol_type = other DNA
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 255 - DNA, 378 bp, synthetic construct
  misc_feature 1..378: note = IRES-45
  source 1..378: mol_type = other DNA
gcttttgaccagtggctctgggttacttagccaagtccctttctcttattttcactagtttatgttgtgtgttgtctgttttgttttgtttaaattgtatacaagatccttcccgccgacacagacctcgacagtctggtgtaggagggttggtgatattaatttgccccaaaagagtgaccgtgatacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgttcatataat

SEQ ID NO: 256 - DNA, 319 bp, synthetic construct
  misc_feature 1..319: note = IRES-46
  source 1..319: mol_type = other DNA
acatggggtatgttgtctgtcctgttttgttgaaacaatatataagatcctttccgccgatatagacctcgacagtctagtgtaggaggattggtgatagtaacttgccccagaagagtgaccgtgacacatagaaaccatgagtacatgtgtatccgtggaggatcgcccgggactggattccatatcccattgccatcccaacaaacggagggtatacccactatgtgcgcgtttgcagtgggagcctgcaaatttagtcatactgcctgatagggtgtgggcctgcactctggggtactcaggctgtttatataat

SEQ ID NO: 257 - DNA, 420 bp, synthetic construct
  misc_feature 1..420: note = IRES-47
  source 1..420: mol_type = other DNA
tgaaaaagtggttgtgcagctggattttccggctgtgcaactgcttttgaccagtggctctgggttacttagccaaattcctttcccttatccctattggtttgtgttgtgtgttgtttgttttgttttgtcttaactatatacaagatccttcccgccgatacagacctcgacagtctggtgtaggagggttggtgttattaatttgccccaaaagagtgaccgtgacacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgtttatataat

SEQ ID NO: 258 - DNA, 373 bp, synthetic construct
  misc_feature 1..373: note = IRES-48
  source 1..373: mol_type = other DNA
gtgtcattaaggtgtgtttggaagttcgaattagctggtttgtggtgattagtagaccccctggaggtacccaattcggatctgaccagggacccgtgactataccgctccggtaattcgggtttaaaacaatgaacgtcaccacacaattacttttctcattttattttcatcattgtcttcctatttaccgattacactcgatttccttggatgttcctggagatttccctggttacctggaccctcattattgttgttgtttcacccagcgagctgtcccaattgcttattatttgcgcttacaacttcgtcctaatatttttctggttgatcgggttgattgagctcccgggctatcctgccattcaac

SEQ ID NO: 259 - DNA, 280 bp, synthetic construct
  misc_feature 1..280: note = IRES-49
  source 1..280: mol_type = other DNA
gggaacaatggtccgtccgcggaacgactctagccatgagtctagtacgagtgcgtgccacccattagcacaaaaaccactgactgagccacacccctcccggaatcctgagtacaggacattcgctcggacgacgcatgagcctccatgccgagaaaattgggtatacccacgggtaaggggtggccacccagcgggaatctgggggctggtcactgactatggtacagcctgatagggtgctgccgcagcgtcagtggtatgcggctgttcatggaac

SEQ ID NO: 260 - DNA, 384 bp, synthetic construct
  misc_feature 1..384: note = IRES-50
  source 1..384: mol_type = other DNA
acctccgtgctaggcacggtgcgttgtcagcgttttgcgcttgcatgcgctacacgcgtcgtccaacgcggagggaacttcacatcaccatgtgtcactccccctatggagggttccaccccgcttacacggaaatgggttaaccatacccaaagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggac

SEQ ID NO: 261 - DNA, 384 bp, synthetic construct
  misc_feature 1..384: note = IRES-51
  source 1..384: mol_type = other DNA
gtatacgagaatttgcctaggacctcgtttacaatatgggcaatctaaaattataattaggcctaagggacaaatcctcctcagcgaaggccgaaaagaggctagccatgcccttagtaggactagcaaaataaggggggtagcaacagtggtgagttcgttggatggctgaagccctgagtacagggtagtcgtcagtggttcgacgcttcggaggacaagcctcgagataccacgtggacgagggcatgcccacagcacatcttaacctggacgggggtcgttcaggtgaaaacggtttaaccaaccgctacgaatacagcctgatagggtgctgcagaggcccactgtattgctactgaaaatctctgctgtacatggcac

SEQ ID NO: 262 - DNA, 372 bp, synthetic construct
  misc_feature 1..372: note = IRES-52
  source 1..372: mol_type = other DNA
gtatacgggagtagctcatgcccgtatacaaaattggatattccaaaactcgattgggttagggagccctcctagcgacggccgaaccgtgttaaccatacacgtagtaggactagcagacgggaggactagccatcgtggtgagatccctgagcagtctaaatcctgagtacaggatagtcgtcagtagttcaacgcaggcacggttctgccttgagatgctacgtggacgagggcatgcccaagacttgctttaatctcggcgggggtcgccgaggtgaaaacacctaacggtgttggggttacagcctgatagggtgctgcagaggcccacgaataggctagtataaaaatctctgctgtacatggcac

SEQ ID NO: 263 - DNA, 388 bp, synthetic construct
  misc_feature 1..388: note = IRES-53
  source 1..388: mol_type = other DNA
gtatacgagattagctaaagtactcgtatatggattggacgtcaacaaatttttaattggcaacgtagggaaccttcccctcagcgaaggccgaaaagaggctagccatgccctttagtaggactagcaaaagtagggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctaatcgtggcgttatggacacagcctgatagggtgtagcagagacctgctattccgctagtaaaaaactctgctgtacatggcac

SEQ ID NO: 264 - DNA, 373 bp, synthetic construct
  misc_feature 1..373: note = IRES-54
  source 1..373: mol_type = other DNA
gtatacgaggttagttcattctcgtatgcattattggacaaatcaaaatttcaatttggttcagggcctccctccagcgacggccgaactgggctagccatgcccatagtaggactagcaaacggagggactagccgtagtggcgagctccctgggtgttctaagtcctgagtacaggacagtcgtcagtagttcgacgtgagcagaagcccacctcgagatgctatgtggacgagggcatgcccaagacgcaccttaaccctagcgggggtcgctagggtgaaatcacaccacgtgatgggagtccgacctgatagggtgctgcagaggctcactattaggctagtataaaaatctctgctgtacatggcac

SEQ ID NO: 265 - DNA, 191 bp, synthetic construct
  misc_feature 1..191: note = IRES-55
  source 1..191: mol_type = other DNA
aaaaccgaccccagagatcagaaagtcgttgacgcgatcttttattagaggacgttgcgctggcgcgagctttaattagcagacgccaaaaataaacaacaaaatgctgatcgcgagacttaattgtcagacgattggccaaatccgatgtgatctttgctgctcccagattgccgaaataggagtagtag

SEQ ID NO: 266 - DNA, 438 bp, synthetic construct
  misc_feature 1..438: note = IRES-56
  source 1..438: mol_type = other DNA
ccccaaaaccccccccttaaactcaacactgtagtggattcattttccgttgcaaaacaaaacattactacccgcatttatgtaggctctgtgttttctatgcgaccgttacattaatctctactctgacccactagtttataaaaccgaagacctgaatgaaacgattttccttcttttcaacctctaacgaacctctgacggcttgagaaacctgaagttagtaattatgtttaaaagaaaggaaagtcaaacgcgatgactcttacatccctattccataccgttgctccacaatgtgagcgatgcgaggtcgggactgcagtattaggggaacgagctacatggagagttaattatctctcccctcctacgggagtctcatgtgagctgtagaaagcggttggcacctctcgttacctcgcctgtacatgatcc

SEQ ID NO: 267 - DNA, 193 bp, synthetic construct
  misc_feature 1..193: note = IRES-57
  source 1..193: mol_type = other DNA
aaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 268 - DNA, 752 bp, synthetic construct
  misc_feature 1..752: note = IRES-58
  source 1..752: mol_type = other DNA
tttcctcctttcgaccgccttacggcaggcgggtccgcggacggcttcggcctacccgcgacaagaatgccgtcatctgtccttatcacccatattctttcccttcccccgcaaccatcacgcttactcgcgcacgtgttgagtggcacgtgcgttgtccaaacagttacactcacacccttggggcgggtttgtcccgccctcgggttcctcgcggaaccctccctcttctctctccctttctatccgccttcactttccataactacagtgctttggtaggtaagcatcctgaccccccgcggaagctgccaacgtggcaactgtggggatccaggcaggttatcaaaggcacccggtctttccgccttcaggagtatccctgccggtgaattccgacagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggtttcccggcgcatgtttggcgaaacgctgaggtaaggttggtgtgcccaatgcccgtaatttggtgacacctcaagaccacccaggaatgccagggaggtaccccacttcggtgggatctgaccctgggctaattgtctacggtggttcttcttgcttccacttctcttttttctggcatg

SEQ ID NO: 269 - DNA, 708 bp, synthetic construct
  misc_feature 1..708: note = IRES-59
  source 1..708: mol_type = other DNA
tatggcaggcgggcttgtggacggcttcggcccacccacagcaagaatgccatcatctgtcctcacccccatgtttcccctttctttccctgcaaccgttacgcttactcgcaggtgcatttgagtggtgcacgtgttgaataaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttttccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttc

SEQ ID NO: 270 - DNA, 718 bp, synthetic construct
  misc_feature 1..718: note = IRES-60
  source 1..718: mol_type = other DNA
tttcgaccgccttatggcaggcgggcttgtggacggcttcggcccacccacagcaagaatgccatcatctgtcctcacccccatttctcccctccttcccctgcaaccattacgcttactcgcatgtgcattgagtggtgcacgtgttgaacaaacagctacactcacgtgggggcgggttttcccgcccttcggcctctcgcgaggcccacccttccccttcctcccataactacagtgctttggtaggtaagcatcctgatcccccgcggaagctgctcgcgtggcaactgtggggacccagacaggttatcaaaggcacccggtctttccgcctccaggagtatccctgctagtgaattctagtggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagcaatggacgggccagtgtgcgtagtcgtcttccggcttgtccggcgcatgtttggtgaaccggtggggtaaggttggtgtgcccaacgcccgtactttggtgacaactcaagaccacccaggaatgccagggaggtaccccgcctcacggcgggatctgaccctgggctaattgtctacggtggttcttcttgcttccatttctttcttctgttc

SEQ ID NO: 271 - DNA, 699 bp, synthetic construct
  misc_feature 1..699: note = IRES-61
  source 1..699: mol_type = other DNA
tatggcaggcgggcttgtggacggtttcggcccacccacagcaagaatgccatcatctgtcctcacccccaattttccctttcttcccctgcaatcatcacgcttactcgcatgtgcattgagtggtgcatgtgttgaacaaacagctacactcacatgggggcgggttttcccgccctacggcctctcgcgaggcccacccttcccctccccttataactacagtgctttggcaggtaagcatcctgatcccccgcggaagctgctcacgtggcaactgtggggacccagacaggttatcaaaggcacccggtctttccgccttcaggagcatccccactagtgaattctagtggggctctgcttggtgccaacctcccccaaatgcgcgctgcgggagtgctcttccccaacccatcctagtatcctctcatgtgtgtgcttggtcagcatatctgagacgacgttccgctgtcccagaccagtccagtaatggacgggccagtgtgcgtagtcgtcttccggcttgtccggcgcatgtttggtgaaccggtggggtaaggttggtgtgcccaacgcccgtactttggtgacacctcaagaccacccaggaatgccagggaggtaccccgcctcacggcgggatctgaccctgggctaattgtctacggtggttcttcttgcttccacttctttctt

SEQ ID NO: 272 - DNA, 624 bp, synthetic construct
  misc_feature 1..624: note = IRES-62
  source 1..624: mol_type = other DNA
ttctcctgcaaccattacgcttaatcgcatgtgcattgagtggtgcatgtgttgaacaaacagctacaatcacatgggggcgggttttcccgccccacggcttctcgcgaggcccatccctcccttttctcccataactacagtgctttggtaggtaagcatcccgatctcccgcggaagctgctcacgtggcaactgtggggacccagacaggttatcaaaggcacccggtctttccgccttcaggagtatccctgctagcgaattctagtagggctctgcttggtgccaacctctcccaaatgcgcgctgcgggagtgctcttccccaaatcaccccagtatcctctcatgtgtgtgcctggtcagcatatctgagacgatgttccgctgtcccagaccagtccagtaatggacgggccagtgtgcgtagtcgtcctccggcttgtccggcgcatgtttggtgaaccggtggggtaaggttggtgtgcccaacgcccgtaatcaggggatacctcaaggcacccaggaatgccagggaggtatcccgcctcacagcgggatctgaccctggggtaaatgtctgcggggggtcctcttggcccaattctcagtaattttcagg

SEQ ID NO: 273 - DNA, 650 bp, synthetic construct
  misc_feature 1..650: note = IRES-63
  source 1..650: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatg

SEQ ID NO: 274 - DNA, 727 bp, synthetic construct
  misc_feature 1..727: note = IRES-64
  source 1..727: mol_type = other DNA
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggc

SEQ ID NO: 275 - DNA, 741 bp, synthetic construct; identical to SEQ ID NO: 210
  misc_feature 1..741: note = IRES-65
  source 1..741: mol_type = other DNA

SEQ ID NO: 276 - DNA, 741 bp, synthetic construct
  misc_feature 1..741: note = IRES-66
  source 1..741: mol_type = other DNA
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaa

SEQ ID NO: 277 - DNA, 742 bp, synthetic construct
  misc_feature 1..742: note = IRES-67
  source 1..742: mol_type = other DNA
ttaaaacagcctgtgggttgttcccacccacagggcccattgggcgtcagcaccctggtatcacggtacctttgtgcgcctgttttatatcccttcccccaattgtaacttagaagaaacacacaccgatcaacagcaagcgtggcacaccagccatgttttggtcaagcacttctgttaccccggactgagtatcaatagactgctcacgcggttgaaggagaaagcgtccgttatccggccagctacttcgagaaacctagtaacaccatggaagttgcggagtgtttcgctcagcactaccccagtgtagatcaggtcgatgagtcaccgctttccccacgggcgaccgtggcggtggctgcgttggcggcctgcctatgggggaacccataggacgctctaatacagacatggtgcgaagagtctattgagctagctggtattcctccggcccctgaatgcggctaatcctaactgtggagcacatgcccctaatccaaggggtagtgtgtcgtaatgagcaattccgcagcggaaccgactactttgggtgtccgtgtttcctcttattcttgtactggctgcttatggtgacaattgagagattgttaccatatagctattggattggccatccggtgactaatagagctattgtgtatctctttgttggatttgtaccacttaatttgaaagaaatcaggacactacgctacattttactattgaacaccgcaaa

SEQ ID NO: 278 - DNA, 743 bp, synthetic construct
  misc_feature 1..743: note = IRES-68
  source 1..743: mol_type = other DNA
ttaaaacagcctgtgggttgtacccacccacagggcccactgggcgctagcactctggtatcacggtacctttgtgcgcctgttttatgcccccttcccccaattgaaacttagaagttacacacaccgatcaacagcgggcgtggcataccagccgcgtcttgatcaagcactcctgtttccccggaccgagtatcaatagactgctcacgcggttgaaggagaaaacgttcgttacccggctaactacttcgagaaacctagtagcatcatgaaagttgcgaagcgtttcgctcagcacatccccagtgtagatcaggtcgatgagtcaccgcattccccacgggcgaccgtggcggtggctgcgttggcggcctgcctacggggcaacccgtaggacgcttcaatacagacatggtgcgaagagtcgattgagctagttagtagtcctccggcccctgaatccggctaatcctaactgcggagcacataccctcaacccagggggcattgtgtcgtaacgggtaactctgcagcggaaccgactactttgggtgtccgtgtttccttttattcttataatggctgcttatggtgacaattgaaagattgttaccatatagctattggattggccatccggtgtctaacagagctattatatacctctttgttggatttgtaccacttgatctaaaggaagtcaagacactacaattcatcatacaattgaacacagcaaa

SEQ ID NO: 279 - DNA, 743 bp, synthetic construct
  misc_feature 1..743: note = IRES-69
  source 1..743: mol_type = other DNA
ttaaaacagcctgtgggttgcacccactcacagggcccactgggcgcaagcactctggcacttcggtacctttgtgcgcctgttttatatcccctcccccaatgaaatttagaagcagcaaaccccgatcaatagcaggcataacgctccagttatgtcttgatcaagcacttctgtttccccggactgagtatcaatagactgctcacgcggttgaaggagaaaacgttcgttatccggctaactacttcggaaagcctagtaacaccatggaagttgcggagagtttcgttcagcacttccccagtgtagatcaggtcgatgagtcaccgcattccccacgggcgaccgtggcggtggctgcgttggcggcctgcccatggggtaacccatgggacgctctaatacggacatggtgtgaagagtctactgagctagttagtagtcctccggcccctgaatgcggctaatcccaactgcggagcacacgcccacaagccagtgggtagtgtgtcgtaacgggcaactctgcagcggaaccgactactttgggtgtccgtgtttccttttattcttatgttggctgcttatggtgacaattaaagagttgttaccatatagctattggattggccatccggtgtgcaacagagcgatcgtttacctatttattggttttgtaccattgacactgaagtctgtgatcacccttaattttatcttaaccctcaacacagccaaac

SEQ ID NO: 280 - DNA, 742 bp, synthetic construct
  misc_feature 1..742: note = IRES-70
  source 1..742: mol_type = other DNA
ttaaaacagcctgtgggttgtacccacccacagggcccactgggcgctagcacactggtattacggtacctttgtgcgcctgttttataccccccccaacctcgaaacttagaagtaaagcaaacccgatcaatagcaggtgcggcgcaccagtcgcatcttgatcaagcacttctgtaaccccggaccgagtatcaatagactgctcacgcggttgaaggagaaaacgttcgttacccggctaactacttcgagaaacccagtagcatcatgaaagttgcagagtgtttcgctcagcactacccccgtgtagatcaggccgatgagtcaccgcacttccccacgggcgaccgtggcggtggctgcgttggcggcctgcctatggggcaacccataggacgctctaatacggacatggtgcgaagagtctattgagctagttagtagtcctccggcccctgaatgcggctaatcctaactgcggagcacatacccttaatccaaagggcagtgtgtcgtaacgggtaactctgcagcggaaccgactactttgggtgtccgtgtttccttttaatttttactggctgcttatggtgacaattgaggaattgttgccatatagctattggattggccatccggtgactaacagagctattgtgttccaatttgttggatttaccccgctcacactcacagtcgtaagaacccttcattacgtgttatttctcaactcaagaaa

SEQ ID NO: 281 - DNA, 745 bp, synthetic construct
  misc_feature 1..745: note = IRES-71
  source 1..745: mol_type = other DNA
ttaaaacagcctgtgggttgtacccacccacagggcccactgggcgctagcactctggtactacggtacctttgtgtgcctgttttaagcccctaccccccactcgtaacttagaaggcttctcacactcgatcaatagtaggtgtggcacgccagtcacaccgtgatcaagcacttctgttaccccggtctgagtaccaataagctgctaacgcggctgaaggggaaaacgatcgttatccggctaactacttcgagaaacccagtaccaccatgaacgttgcagggtgtttcgctcggcacaaccccagtgtagatcaggtcgatgagtcaccgtattccccacgggcgaccgtggcggtggctgcgttggcggcctgcccatggggtgacccatgggacgctctaatactgacatggtgcgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttccttttattcttacattggctgcttatggtgacaattgaaaagttgttaccatatagctattggattggccatccggtgacaaatagagctattgtatatctttttgttggttacgtaccccttaattacaaagtggtttcaactttgaaatacatcctaacactaaattgtagaaa

SEQ ID NO: 282 - DNA, 742 bp, synthetic construct
  misc_feature 1..742: note = IRES-72
  source 1..742: mol_type = other DNA
ttaaaacagcctgtgggttgcacccacccacagggcccacagggcgctagcactctggtatcacggtacctttgtgcgcctgttttattaccccttccccaattgaaaattagaagcaatgcacaccgatcaacagcaggcgtggcgcaccagtcacgtctcgatcaagcacttctgtttccccggaccgagtatcaatagactgctcacgcggttgaaggagaaagtgttcgttatccggctaaccacttcgagaaacccagtaacaccatgaaagttgcagggtgtttcgctcagcacttccccagtgtagatcaggtcgatgagtcaccgcgttccccacgggcgaccgtggcggtggctgcgttggcggcctgcctatgggttaacccataggacgctctaatacagacatggtgcgaagagtttattgagctggttagtatccctccggcccctgaatgcggctaatcctaactgcggagcacgtgcctccaatccagggggttgcatgtcgtaacgggtaactctgcagcggaaccgactactttgggtgtccgtgtttccttttattcttatactggctgcttatggtgacaatcgaggaattgttaccatatagctattggattggccatccggtgtctaacagagcgattatatacctctttgttggatttatgcagctcaataccaccaactttaacacattgaaatatatcttaaagttaaacacagcaaa

SEQ ID NO: 283 - DNA, 585 bp, synthetic construct; identical to SEQ ID NO: 211
  misc_feature 1..585: note = IRES-73
  source 1..585: mol_type = other DNA

SEQ ID NO: 284 - DNA, 568 bp, synthetic construct; identical to SEQ ID NO: 212
  misc_feature 1..568: note = IRES-74
  source 1..568: mol_type = other DNA

SEQ ID NO: 285 - DNA, 696 bp, synthetic construct; identical to SEQ ID NO: 213
  misc_feature 1..696: note = IRES-75
  source 1..696: mol_type = other DNA

SEQ ID NO: 286 - DNA, 697 bp, synthetic construct; identical to SEQ ID NO: 214
  misc_feature 1..697: note = IRES-76
  source 1..697: mol_type = other DNA

SEQ ID NO: 287 - DNA, 562 bp, synthetic construct; identical to SEQ ID NO: 215
  misc_feature 1..562: note = IRES-77
  source 1..562: mol_type = other DNA

SEQ ID NO: 288 - DNA, 340 bp, synthetic construct; identical to SEQ ID NO: 217
  misc_feature 1..340: note = IRES-78
  source 1..340: mol_type = other DNA

SEQ ID NO: 289 - DNA, 315 bp, synthetic construct; identical to SEQ ID NO: 218
  misc_feature 1..315: note = IRES-79
  source 1..315: mol_type = other DNA

SEQ ID NO: 290 - DNA, 759 bp, synthetic construct; identical to SEQ ID NO: 220
  misc_feature 1..759: note = IRES-80
  source 1..759: mol_type = other DNA

SEQ ID NO: 291 - DNA, 478 bp, synthetic construct; identical to SEQ ID NO: 221
  misc_feature 1..478: note = IRES-81
  source 1..478: mol_type = other DNA

SEQ ID NO: 292 - DNA, 628 bp, synthetic construct; identical to SEQ ID NO: 222
  misc_feature 1..628: note = IRES-82
  source 1..628: mol_type = other DNA

SEQ ID NO: 293 - DNA, 618 bp, synthetic construct; identical to SEQ ID NO: 223
  misc_feature 1..618: note = IRES-83
  source 1..618: mol_type = other DNA

SEQ ID NO: 294 - DNA, 610 bp, synthetic construct; identical to SEQ ID NO: 224
  misc_feature 1..610: note = IRES-84
  source 1..610: mol_type = other DNA

SEQ ID NO: 295 - DNA, 614 bp, synthetic construct; identical to SEQ ID NO: 225
  misc_feature 1..614: note = IRES-85
  source 1..614: mol_type = other DNA

SEQ ID NO: 296 - DNA, 624 bp, synthetic construct; identical to SEQ ID NO: 226
  misc_feature 1..624: note = IRES-86
  source 1..624: mol_type = other DNA

SEQ ID NO: 297 - DNA, 737 bp, synthetic construct
  misc_feature 1..737: note = IRES-87
  source 1..737: mol_type = other DNA
ttaaaacagcctgtgggttgatcccacccacagggcccattgggcgctagcactctggtatcacggtacccttgtgcgcctgttttatgtcccttccctcaactgtaacttagaagtaacgcacaccgatcaacagtcagcgtggcacaccagccatgttttgatcaagcacttctgttaccccggaccgagtatcaacagactgctcacgcggttgaaggagaaagtgttcgttatccggccaactacttcgaaaaacctagtaacaccatggaagttgcagagtgtttcgctcagcactaccccagtgtagatcaggtcgatgagtcaccgcatcccccacgggcgaccgtggcggtggctgcgttggcggcctgcctatgggggaacccataggacgctctaatacagacatggtgcgaagagtccattgagctagttggtagtcctccggcccctgaatgcggctaatcctaactgcggagcacacaccttcaagccagagggcagtgtgtcgtaacgggcaactctgcagcggaaccgactactttgggtgtccgtgtttcattttattcttatactggctgcttatggtgacaattgagagattgttaccatatagctattggattggccatccagtgactagcagagctattatatacctctttgttgggtttataccacctaatttgaaagaagttaaaacattagaattcattattaaattgaataca

SEQ ID NO: 298 - DNA, 709 bp, synthetic construct; identical to SEQ ID NO: 228
  misc_feature 1..709: note = IRES-88
  source 1..709: mol_type = other DNA

SEQ ID NO: 299 - DNA, 708 bp, synthetic construct; identical to SEQ ID NO: 229
  misc_feature 1..708: note = IRES-89
  source 1..708: mol_type = other DNA

SEQ ID NO: 300 - DNA, 508 bp, synthetic construct; identical to SEQ ID NO: 230
  misc_feature 1..508: note = IRES-90
  source 1..508: mol_type = other DNA

SEQ ID NO: 301 - DNA, 516 bp, synthetic construct; identical to SEQ ID NO: 232
  misc_feature 1..516: note = IRES-91
  source 1..516: mol_type = other DNA

SEQ ID NO: 302 - DNA, 478 bp, synthetic construct; identical to SEQ ID NO: 233
  misc_feature 1..478: note = IRES-92
  source 1..478: mol_type = other DNA

SEQ ID NO: 303 - DNA, 403 bp, synthetic construct; identical to SEQ ID NO: 234
  misc_feature 1..403: note = IRES-93
  source 1..403: mol_type = other DNA

SEQ ID NO: 304 - DNA, 404 bp, synthetic construct; identical to SEQ ID NO: 235
  misc_feature 1..404: note = IRES-94
  source 1..404: mol_type = other DNA

SEQ ID NO: 305 - DNA, 744 bp, synthetic construct; identical to SEQ ID NO: 236
  misc_feature 1..744: note = IRES-95
  source 1..744: mol_type = other DNA

SEQ ID NO: 306 - DNA, 663 bp, synthetic construct; identical to SEQ ID NO: 237
  misc_feature 1..663: note = IRES-96
  source 1..663: mol_type = other DNA

SEQ ID NO: 307 - DNA, 744 bp, synthetic construct
  misc_feature 1..744: note = IRES-97
  source 1..744: mol_type = other DNA
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 308 - DNA, 734 bp, synthetic construct; identical to SEQ ID NO: 239
  misc_feature 1..734: note = IRES-98
  source 1..734: mol_type = other DNA

SEQ ID NO: 309 - DNA, 638 bp, synthetic construct; identical to SEQ ID NO: 240
  misc_feature 1..638: note = IRES-99
  source 1..638: mol_type = other DNA

SEQ ID NO: 310 - DNA, 743 bp, synthetic construct; identical to SEQ ID NO: 241
  misc_feature 1..743: note = IRES-100
  source 1..743: mol_type = other DNA

SEQ ID NO: 311 - DNA, 746 bp, synthetic construct; identical to SEQ ID NO: 242
  misc_feature 1..746: note = IRES-101
  source 1..746: mol_type = other DNA

SEQ ID NO: 312 - DNA, 726 bp, synthetic construct; identical to SEQ ID NO: 243
  misc_feature 1..726: note = IRES-102
  source 1..726: mol_type = other DNA

SEQ ID NO: 313 - DNA, 737 bp, synthetic construct; identical to SEQ ID NO: 244
  misc_feature 1..737: note = IRES-103
  source 1..737: mol_type = other DNA

SEQ ID NO: 314 - DNA, 327 bp, synthetic construct; identical to SEQ ID NO: 245
  misc_feature 1..327: note = IRES-104
  source 1..327: mol_type = other DNA

SEQ ID NO: 315 - DNA, 281 bp, synthetic construct; identical to SEQ ID NO: 246
  misc_feature 1..281: note = IRES-105
  source 1..281: mol_type = other DNA

SEQ ID NO: 316 - DNA, 459 bp, synthetic construct; identical to SEQ ID NO: 247
  misc_feature 1..459: note = IRES-106
  source 1..459: mol_type = other DNA

SEQ ID NO: 317 - DNA, 350 bp, synthetic construct; identical to SEQ ID NO: 248
  misc_feature 1..350: note = IRES-107
  source 1..350: mol_type = other DNA

SEQ ID NO: 318 - DNA, 318 bp, synthetic construct; identical to SEQ ID NO: 249
  misc_feature 1..318: note = IRES-108
  source 1..318: mol_type = other DNA

SEQ ID NO: 319 - DNA, 399 bp, synthetic construct; identical to SEQ ID NO: 250
  misc_feature 1..399: note = IRES-109
  source 1..399: mol_type = other DNA

SEQ ID NO: 320 - DNA, 430 bp, synthetic construct; identical to SEQ ID NO: 251
  misc_feature 1..430: note = IRES-110
  source 1..430: mol_type = other DNA

SEQ ID NO: 321 - DNA, 504 bp, synthetic construct; identical to SEQ ID NO: 252
  misc_feature 1..504: note = IRES-111
  source 1..504: mol_type = other DNA

SEQ ID NO: 322 - DNA, 532 bp, synthetic construct; identical to SEQ ID NO: 253
  misc_feature 1..532: note = IRES-112
  source 1..532: mol_type = other DNA

SEQ ID NO: 323 - DNA, 494 bp, synthetic construct; identical to SEQ ID NO: 254
  misc_feature 1..494: note = IRES-113
  source 1..494: mol_type = other DNA

SEQ ID NO: 324 - DNA, 378 bp, synthetic construct; identical to SEQ ID NO: 255
  misc_feature 1..378: note = IRES-114
  source 1..378: mol_type = other DNA

SEQ ID NO: 325 - DNA, 319 bp, synthetic construct; identical to SEQ ID NO: 256
  misc_feature 1..319: note = IRES-115
  source 1..319: mol_type = other DNA

SEQ ID NO: 326 - DNA, 420 bp, synthetic construct; identical to SEQ ID NO: 257
  misc_feature 1..420: note = IRES-116
  source 1..420: mol_type = other DNA

SEQ ID NO: 327 - DNA, 373 bp, synthetic construct; identical to SEQ ID NO: 258
  misc_feature 1..373: note = IRES-117
  source 1..373: mol_type = other DNA

SEQ ID NO: 328 - DNA, 280 bp, synthetic construct; identical to SEQ ID NO: 259
  misc_feature 1..280: note = IRES-118
  source 1..280: mol_type = other DNA

SEQ ID NO: 329 - DNA, 384 bp, synthetic construct; identical to SEQ ID NO: 260
  misc_feature 1..384: note = IRES-119
  source 1..384: mol_type = other DNA

SEQ ID NO: 330 - DNA, 384 bp, synthetic construct; identical to SEQ ID NO: 261
  misc_feature 1..384: note = IRES-120
  source 1..384: mol_type = other DNA

SEQ ID NO: 331 - DNA, 372 bp, synthetic construct; identical to SEQ ID NO: 262
  misc_feature 1..372: note = IRES-121
  source 1..372: mol_type = other DNA

SEQ ID NO: 332 - DNA, 388 bp, synthetic construct; identical to SEQ ID NO: 263
  misc_feature 1..388: note = IRES-122
  source 1..388: mol_type = other DNA

SEQ ID NO: 333 - DNA, 373 bp, synthetic construct; identical to SEQ ID NO: 264
  misc_feature 1..373: note = IRES-123
  source 1..373: mol_type = other DNA

SEQ ID NO: 334 - DNA, 438 bp, synthetic construct; identical to SEQ ID NO: 266
  misc_feature 1..438: note = IRES-124
  source 1..438: mol_type = other DNA

SEQ ID NO: 335 - DNA, 752 bp, synthetic construct; identical to SEQ ID NO: 268
  misc_feature 1..752: note = IRES-125
  source 1..752: mol_type = other DNA

SEQ ID NO: 336 - DNA, 708 bp, synthetic construct; identical to SEQ ID NO: 269
  misc_feature 1..708: note = IRES-126
  source 1..708: mol_type = other DNA

SEQ ID NO: 337 - DNA, 718 bp, synthetic construct; identical to SEQ ID NO: 270
  misc_feature 1..718: note = IRES-127
  source 1..718: mol_type = other DNA

SEQ ID NO: 338 - DNA, 699 bp, synthetic construct; identical to SEQ ID NO: 271
  misc_feature 1..699: note = IRES-128
  source 1..699: mol_type = other DNA

SEQ ID NO: 339 - DNA, 624 bp, synthetic construct; identical to SEQ ID NO: 272
  misc_feature 1..624: note = IRES-129
  source 1..624: mol_type = other DNA

SEQ ID NO: 340 - DNA, 650 bp, synthetic construct; identical to SEQ ID NO: 273
  misc_feature 1..650: note = IRES-130
  source 1..650: mol_type = other DNA

SEQ ID NO: 341 - DNA, 742 bp, synthetic construct
  misc_feature 1..742: note = IRES-131
  source 1..742: mol_type = other DNA
ttaaaacagcctgtgggttgatcccacccacagggcccactgggcgctagcactctggtatcacggtacctttgtgcgcctgttttatacttcctcccccaactgcaacttagaagtaacacaaaccgatcaacagtcagcgtggcacaccagccacgttttgatcaaacacttctgttaccccggactgagtatcaatagactgctcacgcggttgaaggagaaaacgttcgttatccggccaactacttcgagaaacctagtaacgccatggaagttgtggagtgtttcgctcagcactaccccagtgtagatcaggttgatgagtcaccgcattccccacgggtgaccgtggcggtggctgcgttggcggcctgcccatggggaaacccatgggacgctcttatacagacatggtgcgaagagtctattgagctagttggtagtcctccggcccctgaatgcggctaatcccaactgcggagcatacactctcaagccagagggtagtgtgtcgtaatgggcaactctgcagcggaaccgactactttgggtgtccgtgtttcattttattcctatactggctgcttatggtgacaattgagagattgttaccatatagctattggattggccatccggtgactaacagagctattatatatctttttgttgggtttataccacttagcttgaaagaggttaaaactctacattacattttaatactgaacaccgcaaa

SEQ ID NO: 342 - DNA, 741 bp, synthetic construct; identical to SEQ ID NO: 276
  misc_feature 1..741: note = IRES-132
  source 1..741: mol_type = other DNA

SEQ ID NO: 343 - DNA, 742 bp, synthetic construct; identical to SEQ ID NO: 277
  misc_feature 1..742: note = IRES-133
  source 1..742: mol_type = other DNA

SEQ ID NO: 344 - DNA, 743 bp, synthetic construct; identical to SEQ ID NO: 278
  misc_feature 1..743: note = IRES-134
  source 1..743: mol_type = other DNA

SEQ ID NO: 345 - DNA, 743 bp, synthetic construct; identical to SEQ ID NO: 279
  misc_feature 1..743: note = IRES-135
  source 1..743: mol_type = other DNA

SEQ ID NO: 346 - DNA, 742 bp, synthetic construct; identical to SEQ ID NO: 280
  misc_feature 1..742: note = IRES-136
  source 1..742: mol_type = other DNA

SEQ ID NO: 347 - DNA, 745 bp, synthetic construct; identical to SEQ ID NO: 281
  misc_feature 1..745: note = IRES-137
  source 1..745: mol_type = other DNA

SEQ ID NO: 348 - DNA, 742 bp, synthetic construct; identical to SEQ ID NO: 282
  misc_feature 1..742: note = IRES-138
  source 1..742: mol_type = other DNA

SEQ ID NO: 349 - DNA, 625 bp, synthetic construct
  misc_feature 1..625: note = IRES-139
  source 1..625: mol_type = other DNA
attctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 350 - DNA, 549 bp, synthetic construct
  misc_feature 1..549: note = IRES-140
  source 1..549: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacg

SEQ ID NO: 351 - DNA, 630 bp, synthetic construct
  misc_feature 1..630: note = IRES-141
  source 1..630: mol_type = other DNA
aacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaacc

SEQ ID NO: 352 - DNA, 653 bp, synthetic construct
  misc_feature 1..653: note = IRES-142
  source 1..653: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcgccacc

SEQ ID NO: 353 - RNA, 741 nt, synthetic construct
  misc_feature 1..741: note = IRES CVB3
  source 1..741: mol_type = other RNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 354 - RNA, 585 nt, synthetic construct
  misc_feature 1..585: note = IRES-1
  source 1..585: mol_type = other RNA
cccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 355 - RNA, 568 nt, synthetic construct
  misc_feature 1..568: note = IRES-2
  source 1..568: mol_type = other RNA
ctccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 356 - RNA, 696 nt, synthetic construct
  misc_feature 1..696: note = IRES-3
  source 1..696: mol_type = other RNA
ttgccagtctgctcgatatcgcaggctgggtccgtgactacccactccccctttcaacgtgaaggctacgatagtgccagggcgggtactgccgtaagtgccaccccaaacaacaacaacaaaacaaactccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 357 - RNA, 697 nt, synthetic construct
  misc_feature 1..697: note = IRES-4
  source 1..697: mol_type = other RNA
ttgccagtctgctcgatatcgcaggctgggtccgtgactacccactccccctttcaacgtgaaggctacgatagtgccagggcgggtactgccgtaagtgccaccccaaaacaacaacaaccccccctctccctcctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 358 - RNA, 562 nt, synthetic construct
  misc_feature 1..562: note = IRES-5
  source 1..562: mol_type = other RNA
ccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 359 - RNA, 156 nt, synthetic construct
  misc_feature 1..156: note = IRES-6
  source 1..156: mol_type = other RNA
gtaaattaaatgctatttacaaaatttaaacagaaaggagagatgttatgaaccggttttacaaggtttcatacatcgaaaatagcactacctggggcagccgacacactaacatcgtctgtttaaccagaagtgttactgaaaggaggttattta

SEQ ID NO: 360 - RNA, 340 nt, synthetic construct
  misc_feature 1..340: note = IRES-7
  source 1..340: mol_type = other RNA
acctgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggacatttgggcgtgcccccgcaagactgctagccgagtagcgttgggttgcgaaaggccttgtggtactgcctgatagggtgcttgcgagtgccccgggaggtctcgtagaccgtgcatc

SEQ ID NO: 361 - RNA, 315 nt, synthetic construct
  misc_feature 1..315: note = IRES-8
  source 1..315: mol_type = other RNA
ctacaagctttgtgtaaacaaacttttgtttggcttttctcaagcttctctcacatcaggccccaaagatgtcctgaaggtaccccgtgtatctgaggatgagcaccatcgactacccggacctgcaaaattttgcaaacgcatgtggtatcccagccccctcctctcggggagggggctttgctcactcagcacaggatctgatcaggagatccacctccggtgctttacaccggggcgtggatttaaaaattgcccaaggcctggcgcacaacctaggggactaggttttccttatattttaaagctgtcaat

SEQ ID NO: 362 - RNA, 182 nt, synthetic construct
  misc_feature 1..182: note = IRES-9
  source 1..182: mol_type = other RNA
gtcttaggacgacgcatgtggtatcccagcccccgcctacattggcgggggcttttgaagcaccagacactggatctgatcaggaggagggtagctgctttacagcccctcttaaaaattgcccaaggtccggccacccaacctaggggactaggttttccttttatttttaaattgtcatt

SEQ ID NO: 363 - RNA, 759 nt, synthetic construct
  misc_feature 1..759: note = IRES-10
  source 1..759: mol_type = other RNA
acatgggggagactgcatgtggcagtcttgaaacgtgtggtttgacgtctaccttatatggcagtgggtggagtactgcaaagatgtcaccgtgctttacacggtttttgaaccccacaccggctgtttgacgctcgtagggcagcaggtttattttcattaaaattcttactttctagctgcatgagttctattcatgcagacggagtgatactcccgttccttcttggacaggttgcctccacgccctttgtggatcttaaggtgaccaagtcactggtgttggaggtgaagatagagagtcctcttgggaatgtcatgtggctgtgccaggggttgtagcgatgccattcgtgtgtgcggatttcctctcgtggtgacacgagcctcacaggccaaaagccccgtccgaaaggacccgaatggtggagtgaccctgactcccccctgcatagttttgtgattaggaacttgaggaatttctgtcataaatctctatcacatcaggccccaaagatgtcctgaaggtaccctgtgtatctgaggatgagcaccaccgactacccggacttgcattagcagacacatgtggttgcccagccccacctcttcagaggtggggctttgctcactcagcacaggatctgatcaggagccccgctcgtgtgctttacactcgacgcggggttaaaaattgcccaaggcctggcacaacaacctaggggactaggttttcctatttttgtaaattatgtcaat

SEQ ID NO: 364 - RNA, 478 nt, synthetic construct
  misc_feature 1..478: note = IRES-11
  source 1..478: mol_type = other RNA
gtgacaatcagccagattgttaacggtcaagcacttctgtttccccggtacccttgtatacgcttcacccgaggcgaaaagtgaggttatcgttatccgcaaagtgcctacgagaagcctagtagcacttttgaagcctatggctggtcgctcaactgtttacccagcagtagacctggcagatgaggctagatgttccccaccagcgatggtgatctagcctgcgtggctgcctgcacactctattgagtgtgaagccagaaagtggacaaggtgtgaagagcctattgtgctcactttgagtcctccggcccctgaatgtggctaatcctaaccccgtagctgttgcatgtaatccaacatgtctgcagtcgtaatgggcaactatgggatggaaccaactactttgggtgtccgtgtttcttgtttttctttatgcttgcttatggtgacaactgtagttattacatttgttacc

SEQ ID NO: 365 - RNA, 628 nt, synthetic construct
  misc_feature 1..628: note = IRES-12
  source 1..628: mol_type = other RNA
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatc

SEQ ID NO: 366 - RNA, 618 nt, synthetic construct
  misc_feature 1..618: note = IRES-13
  source 1..618: mol_type = other RNA
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatc

SEQ ID NO: 367 - RNA, 610 nt, synthetic construct
  misc_feature 1..610: note = IRES-14
  source 1..610: mol_type = other RNA
ttaaaactgggtacaggttgttcccacctgtatcacccacgtggtgtggtgctcttgtattccggtacacttgcacgccagtttgccacccctcacccgtcgtaacttagaagctaacaactcgaccaacaggcggtggtaaaccataccacttacggtcaagcactcctgtttccccggtatgcgaggaatagactcctacagggttgaagcctcaagtatcgttatccgcattggtactacgcaaagcttagtagtgccttgaaagtcccttggttggtcgctccgctagtttcccctagtagacctggcagatgaggcaggacactccccactggcgacagtggtcctgcctgcgtggctgcctgcgcacccttaggggtgcgaagccaagtgacagacaaggtgtgaagagccccgtgtgctaccaatgagtcctccggcccctgaatgcggctaatccaaccccacagctattgcacacaagccagtgtgtatgtagtcgtaatgagcaattgtgggacggaaccgactactttgggtgtccgtgtttccttttattcttatcattctgcttatggtgacaatactgtgaaatagtgttgttacc

SEQ ID NO: 368 - RNA, 614 nt, synthetic construct
  misc_feature 1..614: note = IRES-15
  source 1..614: mol_type = other RNA
taaaactggatccaggttgttcccacctggatctcctattgggagttgtactctattattccggtaattttgtacgccagttttatcttccccctccccaattgtaacttagaaggttatcaatacgaccaataggtggtagttagccaaactaccaaaggtcaagcacttctgtttccccggtcaaagttgatatgctccaacagggcaaaaacaactgagatcgttatccgcaaagtgcctacgcaaagcctagtaacacctttgaagatttatggttggtcgttccgctatttcccatagtagacctggcagatgaggctagaaatcccccactggcgacagtgctctagcctgcgtggctgcctgcgcaccccttgggtgcgaagccatacattggacaaggtgtgaagagccccgtgtgctcactttgagtcctccggcccctgaatgtggctaaccttaaccctgcagctagtgcatgtaatccaacatgttgctagtcgtaatgagtaattgcgggacgggaccaactactttgggtgtccgtgtttcactttttccttttaatattgcttatggtgacaatatatatagctatatatattgacacc

SEQ ID NO: 369 - RNA, 624 nt, synthetic construct
  misc_feature 1..624: note = IRES-16
  source 1..624: mol_type = other RNA
ttcccctgcaaccattacgcttactcgcatgtgcattgagtggtgcatgtgttgaacaaacagctacactcacatgggggcgggttttcccgccctacggcttctcgcgaggcccacccctcccctttctcccataactacagtgctttggtaggtaagcatcctgatcccccgcggaagctgctcacgtggcaactgtggggacccagacaggttatcaaaggcacccggtctttccgccttcaggagtatccctgctagcgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactcaggggatacctcaaggcacccaggaatgccagggaggtaccccgcttcacagcgggatctgaccctggggtaaatgtctgcggggggtcttcttggcccacttctcagtacttttcagg

SEQ ID NO: 370 - RNA, 730 nt, synthetic construct
  misc_feature 1..730: note = IRES-17
  source 1..730: mol_type = other RNA
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgaattccggtagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatg

SEQ ID NO: 371 - RNA, 709 nt, synthetic construct
  misc_feature 1..709: note = IRES-18
  source 1..709: mol_type = other RNA
tatggcaggcgggcttgtggacggcttcggcccacccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 372 - RNA, 708 nt, synthetic construct
  misc_feature 1..708: note = IRES-19
  source 1..708: mol_type = other RNA
tttgaaaggggtctcctagagagcttggccgtcgggccttataccccgacttgctgagtttctctaggagagcccttttcccagccctgaggcggctggtcaataaaagcctcaaacgtaactaacacctaagaagatcatgtaaaccctatgcctggtctccactattcgaaggcaacttgcaataagaagagtgggatcaagacgcttaaagcatagagacagttttcttttctaacccacatttgtgtggggtggcagatggcgtgccataactctaatagtgagataccacgcttgtggaccttatgctcacacagccatcctctagtaagtttgtgagacgtctggtgacgtgtgggaacttattggaaacaacattttgctgcaaagcatcctactgccagcggaaaaacacctggtaacaggtgcctctggggccaaaagccaaggtttaacagaccctttaggattggttctaaacctgagatgttgtggaagatatttagtacctgctgatctggtagttatgcaaacactagttgtaaggcccatgaaggatgcccagaaggtacccgtaggtaacaagtgacactatggatctgatttggggccagatacctctatcttggtgatctggttaaaaaacatctaatgggccaaacccgggggggatccccggtttcctcttattctatcaatgccact

SEQ ID NO: 373 - RNA, 508 nt, synthetic construct
  misc_feature 1..508: note = IRES-20
  source 1..508: mol_type = other RNA
gtataagagacaggtgtttgccttgtcttcggactggcatcttgggaccaaccccccttttccccagccatgggttaaatggcaataaaggacgtaacaactttgtaaccattaagctttgtaattttgtaaccactaagctttgtgcacataatgtaaccatcaagcttgttagtcccagcaggaggtttgcatgcttgtagccgaaatggggctcgaccccccatagtaggatacttgattttgcattccattgtggacctgcaaactctacacatagaggctttgtcttgcatctaaacacctgagtacagtgtgtacctagaccctatagtacgggaggaccgtttgtttcctcaataaccctacataataggctaggtgggcatgcccaatttgcaagatcccagactgggggtcggtctgggcagggttagatccctgttagctactgcctgatagggtggtgctcaaccatgtgtagtttaaattgagctgttcatatacc

SEQ ID NO: 374 - RNA, 248 nt, synthetic construct
  misc_feature 1..248: note = IRES-21
  source 1..248: mol_type = other RNA
actgaagatcctacagtaactactgccccaatgaacgccacagatgggtctgctgatgactacctatcttagtgctagttgaggtttgaagtgagccggtttttagaagaaccagtttctgaacattatcatccccagcatctattctatacgcacaagatagatagtcatcagcagacacatctgtgctactgcttgatagagttgcggctggtcaacttagattggtataaccagttgagtggcaa

SEQ ID NO: 375 - RNA, 516 nt, synthetic construct
  misc_feature 1..516: note = IRES-22
  source 1..516: mol_type = other RNA
tatgcatcactggacggcctaacctcggtcgtggcttcttgccgatttcagcgctaccaggctttctggtctcgccaggcgttgattagtaggtgcactgtctaagtgaagacagcagtgctctctgtgaaaagttgatgacactcttcaggtttgtagcgatcactcaaggctagcggatttccccgtgtggtaacacacgcctctaggcccagaaggcacggtgttgacagcaccccttgagtggctggtcttccccaccagcacctgatttgtggattcttcctagtaacggacaagcatggctgctcttaagcattcagtgcgtccggggctgaaggatgcccagaaggtacccgcaggtaacgataagctcactgtggatctgatctggggctgcgggctgggtgtctttccacccagccaaaacccgtaaaacggtagtcgcagttaaaaaacgtctaggccccacccccccagggatggggggttcccttaaaccctcacaagttcaac

SEQ ID NO: 376 - RNA, 478 nt, synthetic construct
  misc_feature 1..478: note = IRES-23
  source 1..478: mol_type = other RNA
tgaaaagggggcgcagggtggtggtggttactaaatacccaccatcgccctgcacttcccttttcccctgtggctcagggtcacttagccccctctttgggttaccagtagttttctacccctgggcacagggttaactatgcaagacggaacaacaatctcttagtccccctcgccgatagtgggctcgacccccatgtgtaggagtggataagggacggagtgagccgatacggggaagagtgtgcggtcacaccttaattccatgagcgctgcgaagaaggaagctgtgaacaatggcgacctgaaccgtacacatggagctccacaggcatggtactcgttagactacgcagcctggttgggagtgggtataccctgggtgagccgccagtgaatgggagttcactggttaacacacactgcctgatagggtcagggcctcctgtccccgccgtaatgaggtagaccatatgcc

SEQ ID NO: 377 - RNA, 403 nt, synthetic construct
  misc_feature 1..403: note = IRES-24
  source 1..403: mol_type = other RNA
gcggctggatattctggccgtgcaactgcttttgaccagtggctctgggtaacttagccaaagtgtccttctccctttccctattatatgttttatggctttgtctggtcttgtttagtttatatataagatcctttccgccgatatagacctcgacagtctagtgtaggaggattggtgatattaatttgccccagaagagtgaccgtgacacatagaaaccatgagtacatgtgtatccgtggaggatcgcccgggactggattccatatcccattgccatcccaacaagcggagggtatacccactatgtgcacgtctgcagtgggagtctgcagatttagtcatactgcctgatagggtgtgggcctgcactctggggtactcaggctgtttatataat

SEQ ID NO: 378 - RNA, 404 nt, synthetic construct
  misc_feature 1..404: note = IRES-25
  source 1..404: mol_type = other RNA
gctggactttctggctgcgcaactgcttttaaccagtggctctgggttacttagccaaaaccccctttccccgtaccctagtttgtgtgtgtattattattttgttgttgttttgtaaatttttatataagatcctttccgccgatatagacctcgacagtctagtgtaggaggattggtgatattaatatgccccagaagagtgaccgtgacacatagaaaccatgagtacatgtgtatccgtggaggatcgcccgggactggattccatatcccattgccatcccaacaaacggagggtatacccgctatgtgcgcgtctacagtgggaatctgtagatttagtcatactgcctgatagggtgtgggcctgcactctggggtactcaggctgtttatataat

SEQ ID NO: 379 - RNA, 744 nt, synthetic construct
  misc_feature 1..744: note = IRES-26
  source 1..744: mol_type = other RNA
ttaaaacagcctgtgggttgttcccatccacagggcccactgggcgccagcactctggtattgcggtaccttagtgcgcctgttttatatacccgtcccccaaacgtaacttagacgcatgtcaacgaagaccaatagtaagcgcagcacaccagctgtgttccggtcaagcacttctgttaccccggaccgagtatcaataagctactcacgtggctgaaggagaaaacgttcgttacccgaccaattacttcaagaaacctagtaacaccatgaaggttgcgcagtgtttcgctccgcacaaccccagtgtagatcaggtcgatgagtcaccgcattccccacgggtgaccgtggcggtggctgcgctggcggcctgcccatggggaaacccatgggacgcttcaatactgacatggtgcgaagagtctattgagctaattggtagtcctccggcccctgaatgcggctaatcctaactgcggagcagatacccacacaccagtgggcagtctgtcgtaacgggcaactctgcagcggaaccgactactttgggtgtccgtgtttctctttatccttatactggctgcttatggtgacaattgagagattgttaccatatagctattggattggccatccggtgacaaatagagcaattgtgtatttgtttgttggtttcgtgccattaaattacaaggttctaaacacccttaatcttattatagcattcaacacaacaaa

SEQ ID NO: 380 - RNA, 663 nt, synthetic construct
  misc_feature 1..663: note = IRES-27
  source 1..663: mol_type = other RNA
gtacattagatgcgtcatctgcaactttagtcaataaattacctccaatgtcattaccaacattccctaccttttcactaacacctaagacaacaagtacctatgcctggtctccactattcgaaggcaacttgcaataagaagagtggaattaagacgcttaaagcatagagctagttatcttttctaacccacaaagttttgtggggtggcagatggcgtgccataactctattagtgagataccatgcttgtggatcttatgctcacacagccatcctctagtaagttgataaggtgtctggtgatatgtgggaactcacatgaaccattaatttaccgtaaggtatcctatagccagcggaatcacatctggtgacagatgcctctggggccgaaagccaaggtttaacagaccctataggattggtttcaaaacctgaattgatgtggattgtgtatagtacctgttgatctggtaacagtgtcaacactagttgtaaggcccacgaaggatgcccagaaggtacccgtaggtaacaagtgacactatggatctgatctggggccagctacctctatcatggtgagttggttaaaaaacgtctagtgggccaaacccaggggggatccctggtttccttttacctaatcaaagccact

SEQ ID NO: 381 - RNA, 744 nt, synthetic construct
  misc_feature 1..744: note = IRES-28
  source 1..744: mol_type = other RNA
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaattcacgttgggcaactagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 382 - RNA, 734 nt, synthetic construct
  misc_feature 1..734: note = IRES-29
  source 1..734: mol_type = other RNA
ttcaagaggggtctccggagttttccggaacccctcttggaagtccatggtgaggggacttgatacctcaccgccgtttgcctaggctataggctaaatttccctttccctgtccttcccctatttccttttgttttgtttgtaaatattaattcctgcaggttcagggttctttaatctgtttctctataagaacactcaatttttcacgctttctgtctcctttcttccagggctctccccttgccctaggctctggccgttgcgcccggcggggtcaactccatgattagcatggagctgtaggagtctaaattggggacgcagatgtttgggacgtcgccttgcagtgttaacttggctttcatgaacctctttgatcttccacaaggggtaggctacgggtgaaacctcttaggctaatacttcaatgaagagatgccttggatagggtaacagcggcggatattggtgagttgttaagacaaaaaccattcaacgccggaggactggctctcatccagtggatgcattgagggaattgattgtcagggctgtctctaggtttaatctcagacctctctgtgcttagggcaaacactatttggccttaaatgggatcctgtgagagggggtccctccattgacagctggactgttctttggggccttatgtggtgtttgcctctgaggtactcaggggcatttaggtttttcctcattcttaaataata

SEQ ID NO: 383 - RNA, 638 nt, synthetic construct
  misc_feature 1..638: note = IRES-30
  source 1..638: mol_type = other RNA
gggagtaaacctcaccaccgtttgccgtggtttacggctacctatttttggatgtaaatattaattcctgcaggttcaggtctcttgaattatgtccacgctagtggcactctcttacccataagtgacgccttagcggaacctttctacacttgatgtggttaggggttacattatttccctgggccttctttggccctttttcccctgcactatcattctttcttccgggctctcagcatgccaatgttccgaccggtgcgcccgccggggttaactccatggttagcatggagctgtaggccctaaaagtgctgacactggaactggactattgaagcatacactgttaactgaaacatgtaactccaatcgatcttctacaaggggtaggctacgggtgaaaccccttaggttaatactcatattgagagatacttctgataggttaaggttgctggataatggtgagtttaacgacaaaaaccattcaacagctgtgggccaacctcatcaggtagatgcttttggagccaagtgcgtaggggtgtgtgtggaaatgcttcagtggaaggtgccctcccgaaaggtcgtaggggtaatcaggggcagttaggtttccacaattacaatttgaa

SEQ ID NO: 384 - RNA, 743 nt, synthetic construct
  misc_feature 1..743: note = IRES-31
  source 1..743: mol_type = other RNA
gctcttccgatctgggttgttcccacccacagggcccactgggcgccagcactctgattccacggaatctttgtgcgcctgttttacaacccttcccaatttgtaacgtagaagcaatacacactactgatcaatagtaggcatggcgcgccagtcatgtcatgatcaagcacttctgttcccccggactgagtatcaatagactgctcacgcggttgaaggagaaaacgttcgttacccggctaactacttcgagaaacctagtagcaccatggaagctgcggagtgtttcgctcagcactttccccgtgtagatcaggtcgatgagtcactgcaatccccacgggcgaccgtggcagtggctgcgttggcggcctgcctatggggcaacccataggacgctctaatgtggacatggtgcgaagagtctattgagctagttagtagtcctccggcccctgaatgcggctaatcctaactgcggagcacatgccttcaacccaggaggtggtgtgtcgtaacgggtaactctgcagcggaaccgactactttgggtgtccgtgtttccttttatccttatattggctgcttatggtgacaatcacggaattgttgccatatagctattggattggccatccggtgtctaacagagctattgtatacctatttgttggatttactcccctatcatacaaatctctgaacactttgtgctttatactgaacttaaacacacgaaa

SEQ ID NO: 385 - RNA, 746 nt, synthetic construct
  misc_feature 1..746: note = IRES-32
  source 1..746: mol_type = other RNA
ttaaaacagctctggggttgttcccaccccagaggcccacgtggcggccagtacaccggtaccacggtacccttgtacgcctgttttatactcccctccccgtaaactagaagcacgaaacacaagttcaatagaagggggtacagaccagtaccaccacgaacaagcacttctgttcccccggtgaggtcacatagactgtccccacggtcaaaagtgactgatccgttatccgctcacgtacttcggaaagcctagtaccaccttggaatctacgatgcgttgcgctcagcactcgaccccggagtgtagcttaggctgatgagtctggacgttccccactggtgacagtggtccaggctgcgttggcggcctacctgtggtccaaaaccacaggacgctagtagtgaacaaggtgtgaagagcccactgagctacctgagaatcctccggcccctgaatgcggctaatcccaaccacggagcaggtaatcgcaaaccagcggtcagcctgtcgtaacgcgtaagtctgtggcggaaccgactactttgggtgtccgtgtttccttttatttttatggtggctgcttatggtgacaatcatagattgttatcataaagcaaattggattggccatccggagtgagctaaactatctatttctctgagtgttggattcgtttcacccacattctgaacaatcagcctcattagtgttaccctgttaataagacgatatcatcacg

SEQ ID NO: 386 - RNA, 726 nt, synthetic construct
  misc_feature 1..726: note = IRES-33
  source 1..726: mol_type = other RNA
ttaaaacagctctggggttgttcccaccccagaggcccacgtggcggctagtactccggtaccccggtacccttgtacgcctgttttatactccctttcccaagtaactttagaagaaataaactaatgttcaacaggagggggtacaaaccagtaccaccacgaacacacacttctgtttccccggtgaagttgcatagactgtacccacggttgaaagcgatgaatccgttacccgcttaggtacttcgagaagcctagtatcatcttggaatcttcgatgcgttgcgatcagcactctaccccgagtgtagcttgggtcgatgagtctggacaccccacaccggcgacgtggtccaggctgcgttggcggcctacccatggctagcaccatgggacgctagttgtgaacaaggtgcgaagagcctattgagctacctgagagtcctccggcccctgaatgcggctaatcccaaccacggagcaaatgctcacaatccagtgagtggtttgtcgtaatgcgcaagtctgtggcggaaccgactactttgggtgtccgtgtttccttttatttttattatggctgcttatggtgacaatctgagattgttatcatatagctattggattagccatccggtgatatcttgaaattttgccataactttttcacaaatcctacaacattacactacactttctcttgaataattgagacaactcata

SEQ ID NO: 387 - RNA, 737 nt, synthetic construct
  misc_feature 1..737: note = IRES-34
  source 1..737: mol_type = other RNA
ttaaaatagcctcagggttgttcccaccctgagggcccacgtggtgtagtactctggtattacggtacctttgtacgcctattttatacccccttccccaagtaatttagaagcaagcacaaaccagttcagtagtaagcagtacaatccagtactgtaatgaacaagtacttctgttaccccggaagggtctatcggtaagctgtacccacggctgaagaatgacctaccgttaaccggctacctacttcgagaagcctagtaatgccgttgaagttttattgacgttacgctcagcacactaccccgtgtgtagttttggctgatgagtcacggcactccccacgggcgaccgtggccgtggctgcgttggcggccaaccaaggagtgcaagctccttggacgtcatattacagacatggtgtgaagagcctattgagctaggtggtagtcctccggcccctgaatgcggctaatcctaactccggagcatatcggtgcgaaccagcacttggtgtgttgtaatacgtaagtctggagcggaaccgactactttgggtgtccgtgtttcctgttttaacttttatggctgcttatggtgacaatttaacattgttaccatatagctgttgggttggccatccggattttgttataaaaccatttcctcgtgccttgacctttaacacatttgtgaacttctttaaatcccttttattagtccttaaatactaaga

SEQ ID NO: 388 - RNA, 327 nt, synthetic construct
  misc_feature 1..327: note = IRES-35
  source 1..327: mol_type = other RNA
aactgttgttgtagcaatgcgcatattgctacttcggtacgcctaattggtaggcgcccggccgaccggccccgcaagggcctagtaggacgtgtgacaatgccatgagggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 389 - RNA, 281 nt, synthetic construct
  misc_feature 1..281: note = IRES-36
  source 1..281: mol_type = other RNA
tgacgtgggggggttgattttccccccccggcactgggtgcaagccccagaaaccgacgcctatctaagtagacgcaatgactcggcgccgactcggcgaccggccaaaaggtggtggatgggtgatgacagggttggtaggtcgtaaatcccggtcatcctggtagccactataggtgggtcttaagagaaggtcaagattcctcttacgcctgcggcgagaccgcgcacggtccacaggtgttggccctaccggtgtgaataagggcccgacatcaggc

SEQ ID NO: 390 - RNA, 459 nt, synthetic construct
  misc_feature 1..459: note = IRES-37
  source 1..459: mol_type = other RNA
gacgtgggggggttgatcccccccctttggcactgggtgcaagccccagaaaccgacgcctatttaaacagacgttaagaaccggcgccgacccggcgaccggccaaaaggtggtggatgggtgatgccagggttggtaggtcgtaaatcccggtcatcttggtagccactataggtgggtcttaagggttggttaaggtccctctggcgcttgtggcgagaaagcgcacggtccacaggtgttggccctaccggtgtgaataagggcccgacgtcaggctcgtcgttaaaccgagcccactacccacctgggcaaacaacgcccacgtacggtccacgtcgcccttcaatgtctctcttgaccaataggcttagccggcgagttgacaaggaccagtgggggctgggcggtaggggaaggacccctgccgctgcccttcccggtggagtgggaaatgc

SEQ ID NO: 391 - RNA, 350 nt, synthetic construct
  misc_feature 1..350: note = IRES-38
  source 1..350: mol_type = other RNA
tgacgtgggggggttgatccgccccccccggcactcggtgcaagccccataaaccgacgcctatctaagtagacgcaatgactcggcgccgactcggcgaccggccaaaaggtggtggatgggtggtgacagggttggtaggtcgtaaatcccggtcatcctggtagccactataggtgggtcttaagagaaggtcaagactcctcttgtgcctgcggcgagaccgcgcacggtccacaggtgctggccctaccggtgtgaataagggcccgacgtcaggctcgtcgttaaaccgagcccgtcacccacctgggcaaacgacgcccacgtacggtccacgtcgcccttca

SEQ ID NO: 392 - RNA, 318 nt, synthetic construct
  misc_feature 1..318: note = IRES-39
  source 1..318: mol_type = other RNA
tgtagcaatgcgcatattgctacttcggtacgcctaattggtaggcgcccggccgaccggccccgcaagggcctagtaggacgtgtgacaatgccatgcgggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 393 - RNA, 399 nt, synthetic construct
  misc_feature 1..399: note = IRES-40
  source 1..399: mol_type = other RNA
attttctggccgtgtagctgcttttgaccagtggctctgggttacttagccaaatcccccttccttcacccttttaaatttgatggtctgtgttgtttgttttgtcttgtctaaataatatataagatccttcccgccgatacagacctcgacagtctggtgtaggagggttggtgttattaatttgccccagaagagtgaccgtgacacatagaaaccatgagtacatgtgtatccgtggaggatcgcccgggactggattccatatcccattgccatcccaacaagcggagggtatacccactatgtgcgcgtttgcagtgggaatctgcaaatttagtcatactgcctgatagggtgtgggcctgcactctggggtactcaggctgttcatataat

SEQ ID NO: 394 - RNA, 430 nt, synthetic construct
  misc_feature 1..430: note = IRES-41
  source 1..430: mol_type = other RNA
cccctccacccttaaggtggttgtatcccacataccccaccctcccttccaaagtggacggacaactggattttgactaacggcaagtctgaatggtatgatttggatacgtttaaacggcagtagcgtggcgagctatggaaaaatcgcaattgtcgatagccatgttagtgacgcgcttcggcgtgctcctttggtgattcggcgactggttacaggagagtaggcagtgagctatgggcaaacctctacagtattacttagagggaatgtgcaattgagacttgacgagcgtctctttgagatgtggcgcatgctcttggcattaccatagtgagcttccaggttgggaaacctggactgggcctatactacctgatagggtcgcggctggccgcctgtaactagtatagtcagttgaaaccccccc

SEQ ID NO: 395 - RNA, 504 nt, synthetic construct
  misc_feature 1..504: note = IRES-42
  source 1..504: mol_type = other RNA
gtctctttagtgtctatgcttcagagagcggtgaactgacaccgttgcttcttgcacagcccttcgtgccggtctttccggttctcgacagcgttgggcatcatggctagttaggctaagatagtggatgatctagtgaacagttttggattgtttggagttttgtagcgatgctagtagtgtgtgtggacctccccacgtggtaacacgtgccccacaggccaaaagccaaggtgttgaaagcacccctactagtcccagactcacccatctgggaactcctctcatgaaaaatcttagtaacttttgattcggctattcatcaacctctctagtcaagggctgaaggatgcccggaaggtacccgcaggtaacgataagctcactgtggatctgatccggggctttggtgcgaccgtctgtccggcgtagccagagttaaaaaacgtctaggcccttccaccccaagggattggggtttccccaatcatttgaaagttcact

SEQ ID NO: 396 - RNA, 532 nt, synthetic construct
  misc_feature 1..532: note = IRES-43
  source 1..532: mol_type = other RNA
ttttgaacgccacctcggagcgatatccggggaccccctcccctttttccttcctaccttcttcccaaatttccctcttcccttgttattttggtttggatttcctggacatgactcggacggatctatctcatttgctttgtgtctgctccaccagtggcatggtcgaaagatcatcaacactggacgtgtactgtaatggccaaacgtgcccacaggggaaaccatgccggtcgctgtagcggcgggtggacgtggtggacccctctccctgctcataaactttgggtaggtgaagggttcaagcgacgcttgccgtgagggcgcatccggatggtgggaaccaacaaactaggctgtaatggccgacctcaggtggatgagctagggctgctgcaccaaaagggactcgattcgatatcccggcctggtagcctagtgcagtggactcgtagttgggaatctacgactggcctagtacagggtgatagccccgtttcccacgcccacctgttgtagggacacccccccc

SEQ ID NO: 397 - RNA, 494 nt, synthetic construct
  misc_feature 1..494: note = IRES-44
  source 1..494: mol_type = other RNA
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 398 - RNA, 378 nt, synthetic construct
  misc_feature 1..378: note = IRES-45
  source 1..378: mol_type = other RNA
gcttttgaccagtggctctgggttacttagccaagtccctttctcttattttcactagtttatgttgtgtgttgtctgttttgttttgtttaaattgtatacaagatccttcccgccgacacagacctcgacagtctggtgtaggagggttggtgatattaatttgccccaaaagagtgaccgtgatacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgttcatataat

SEQ ID NO: 399 - RNA, 319 nt, synthetic construct
  misc_feature 1..319: note = IRES-46
  source 1..319: mol_type = other RNA
acatggggtatgttgtctgtcctgttttgttgaaacaatatataagatcctttccgccgatatagacctcgacagtctagtgtaggaggattggtgatagtaacttgccccagaagagtgaccgtgacacatagaaaccatgagtacatgtgtatccgtggaggatcgcccgggactggattccatatcccattgccatcccaacaaacggagggtatacccactatgtgcgcgtttgcagtgggagcctgcaaatttagtcatactgcctgatagggtgtgggcctgcactctggggtactcaggctgtttatataat

SEQ ID NO: 400 - RNA, 420 nt, synthetic construct
  misc_feature 1..420: note = IRES-47
  source 1..420: mol_type = other RNA
tgaaaaagtggttgtgcagctggattttccggctgtgcaactgcttttgaccagtggctctgggttacttagccaaattcctttcccttatccctattggtttgtgttgtgtgttgtttgttttgttttgtcttaactatatacaagatccttcccgccgatacagacctcgacagtctggtgtaggagggttggtgttattaatttgccccaaaagagtgaccgtgacacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgtttatataat

SEQ ID NO: 401 - RNA, 373 nt, synthetic construct
  misc_feature 1..373: note = IRES-48
  source 1..373: mol_type = other RNA
gtgtcattaaggtgtgtttggaagttcgaattagctggtttgtggtgattagtagaccccctggaggtacccaattcggatctgaccagggacccgtgactataccgctccggtaattcgggtttaaaacaatgaacgtcaccacacaattacttttctcattttattttcatcattgtcttcctatttaccgattacactcgatttccttggatgttcctggagatttccctggttacctggaccctcattattgttgttgtttcacccagcgagctgtcccaattgcttattatttgcgcttacaacttcgtcctaatatttttctggttgatcgggttgattgagctcccgggctatcctgccattcaac

SEQ ID NO: 402 - RNA, 280 nt, synthetic construct
  misc_feature 1..280: note = IRES-49
  source 1..280: mol_type = other RNA
gggaacaatggtccgtccgcggaacgactctagccatgagtctagtacgagtgcgtgccacccattagcacaaaaaccactgactgagccacacccctcccggaatcctgagtacaggacattcgctcggacgacgcatgagcctccatgccgagaaaattgggtatacccacgggtaaggggtggccacccagcgggaatctgggggctggtcactgactatggtacagcctgatagggtgctgccgcagcgtcagtggtatgcggctgttcatggaac

SEQ ID NO: 403 - RNA, 384 nt, synthetic construct
  misc_feature 1..384: note = IRES-50
  source 1..384: mol_type = other RNA
acctccgtgctaggcacggtgcgttgtcagcgttttgcgcttgcatgcgctacacgcgtcgtccaacgcggagggaacttcacatcaccatgtgtcactccccctatggagggttccaccccgcttacacggaaatgggttaaccatacccaaagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggac

SEQ ID NO: 404 - RNA, 384 nt, synthetic construct
  misc_feature 1..384: note = IRES-51
  source 1..384: mol_type = other RNA
gtatacgagaatttgcctaggacctcgtttacaatatgggcaatctaaaattataattaggcctaagggacaaatcctcctcagcgaaggccgaaaagaggctagccatgcccttagtaggactagcaaaataaggggggtagcaacagtggtgagttcgttggatggctgaagccctgagtacagggtagtcgtcagtggttcgacgcttcggaggacaagcctcgagataccacgtggacgagggcatgcccacagcacatcttaacctggacgggggtcgttcaggtgaaaacggtttaaccaaccgctacgaatacagcctgatagggtgctgcagaggcccactgtattgctactgaaaatctctgctgtacatggcac

SEQ ID NO: 405 - RNA, 372 nt, synthetic construct
  misc_feature 1..372: note = IRES-52
  source 1..372: mol_type = other RNA
gtatacgggagtagctcatgcccgtatacaaaattggatattccaaaactcgattgggttagggagccctcctagcgacggccgaaccgtgttaaccatacacgtagtaggactagcagacgggaggactagccatcgtggtgagatccctgagcagtctaaatcctgagtacaggatagtcgtcagtagttcaacgcaggcacggttctgccttgagatgctacgtggacgagggcatgcccaagacttgctttaatctcggcgggggtcgccgaggtgaaaacacctaacggtgttggggttacagcctgatagggtgctgcagaggcccacgaataggctagtataaaaatctctgctgtacatggcac

SEQ ID NO: 406 - RNA, 388 nt, synthetic construct
  misc_feature 1..388: note = IRES-53
  source 1..388: mol_type = other RNA
gtatacgagattagctaaagtactcgtatatggattggacgtcaacaaatttttaattggcaacgtagggaaccttcccctcagcgaaggccgaaaagaggctagccatgccctttagtaggactagcaaaagtagggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctaatcgtggcgttatggacacagcctgatagggtgtagcagagacctgctattccgctagtaaaaaactctgctgtacatggcac

SEQ ID NO: 407 - RNA, 373 nt, synthetic construct
  misc_feature 1..373: note = IRES-54
  source 1..373: mol_type = other RNA
gtatacgaggttagttcattctcgtatgcattattggacaaatcaaaatttcaatttggttcagggcctccctccagcgacggccgaactgggctagccatgcccatagtaggactagcaaacggagggactagccgtagtggcgagctccctgggtgttctaagtcctgagtacaggacagtcgtcagtagttcgacgtgagcagaagcccacctcgagatgctatgtggacgagggcatgcccaagacgcaccttaaccctagcgggggtcgctagggtgaaatcacaccacgtgatgggagtccgacctgatagggtgctgcagaggctcactattaggctagtataaaaatctctgctgtacatggcac

SEQ ID NO: 408 - RNA, 191 nt, synthetic construct
  misc_feature 1..191: note = IRES-55
  source 1..191: mol_type = other RNA
aaaaccgaccccagagatcagaaagtcgttgacgcgatcttttattagaggacgttgcgctggcgcgagctttaattagcagacgccaaaaataaacaacaaaatgctgatcgcgagacttaattgtcagacgattggccaaatccgatgtgatctttgctgctcccagattgccgaaataggagtagtag

SEQ ID NO: 409 - RNA, 438 nt, synthetic construct
  misc_feature 1..438: note = IRES-56
  source 1..438: mol_type = other RNA
ccccaaaaccccccccttaaactcaacactgtagtggattcattttccgttgcaaaacaaaacattactacccgcatttatgtaggctctgtgttttctatgcgaccgttacattaatctctactctgacccactagtttataaaaccgaagacctgaatgaaacgattttccttcttttcaacctctaacgaacctctgacggcttgagaaacctgaagttagtaattatgtttaaaagaaaggaaagtcaaacgcgatgactcttacatccctattccataccgttgctccacaatgtgagcgatgcgaggtcgggactgcagtattaggggaacgagctacatggagagttaattatctctcccctcctacgggagtctcatgtgagctgtagaaagcggttggcacctctcgttacctcgcctgtacatgatcc

SEQ ID NO: 410 - RNA, 193 nt, synthetic construct
  misc_feature 1..193: note = IRES-57
  source 1..193: mol_type = other RNA
aaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 411 - RNA, 752 nt, synthetic construct
  misc_feature 1..752: note = IRES-58
  source 1..752: mol_type = other RNA
tttcctcctttcgaccgccttacggcaggcgggtccgcggacggcttcggcctacccgcgacaagaatgccgtcatctgtccttatcacccatattctttcccttcccccgcaaccatcacgcttactcgcgcacgtgttgagtggcacgtgcgttgtccaaacagttacactcacacccttggggcgggtttgtcccgccctcgggttcctcgcggaaccctccctcttctctctccctttctatccgccttcactttccataactacagtgctttggtaggtaagcatcctgaccccccgcggaagctgccaacgtggcaactgtggggatccaggcaggttatcaaaggcacccggtctttccgccttcaggagtatccctgccggtgaattccgacagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggtttcccggcgcatgtttggcgaaacgctgaggtaaggttggtgtgcccaatgcccgtaatttggtgacacctcaagaccacccaggaatgccagggaggtaccccacttcggtgggatctgaccctgggctaattgtctacggtggttcttcttgcttccacttctcttttttctggcatg

SEQ ID NO: 412 - RNA, 708 nt, synthetic construct
  misc_feature 1..708: note = IRES-59
  source 1..708: mol_type = other RNA
tatggcaggcgggcttgtggacggcttcggcccacccacagcaagaatgccatcatctgtcctcacccccatgtttcccctttctttccctgcaaccgttacgcttactcgcaggtgcatttgagtggtgcacgtgttgaataaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttttccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttc

SEQ ID NO: 413 - RNA, 718 nt, synthetic construct
  misc_feature 1..718: note = IRES-60
  source 1..718: mol_type = other RNA
tttcgaccgccttatggcaggcgggcttgtggacggcttcggcccacccacagcaagaatgccatcatctgtcctcacccccatttctcccctccttcccctgcaaccattacgcttactcgcatgtgcattgagtggtgcacgtgttgaacaaacagctacactcacgtgggggcgggttttcccgcccttcggcctctcgcgaggcccacccttccccttcctcccataactacagtgctttggtaggtaagcatcctgatcccccgcggaagctgctcgcgtggcaactgtggggacccagacaggttatcaaaggcacccggtctttccgcctccaggagtatccctgctagtgaattctagtggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagcaatggacgggccagtgtgcgtagtcgtcttccggcttgtccggcgcatgtttggtgaaccggtggggtaaggttggtgtgcccaacgcccgtactttggtgacaactcaagaccacccaggaatgccagggaggtaccccgcctcacggcgggatctgaccctgggctaattgtctacggtggttcttcttgcttccatttctttcttctgttc

SEQ ID NO: 414 - RNA, 699 nt, synthetic construct
  misc_feature 1..699: note = IRES-61
  source 1..699: mol_type = other RNA
tatggcaggcgggcttgtggacggtttcggcccacccacagcaagaatgccatcatctgtcctcacccccaattttccctttcttcccctgcaatcatcacgcttactcgcatgtgcattgagtggtgcatgtgttgaacaaacagctacactcacatgggggcgggttttcccgccctacggcctctcgcgaggcccacccttcccctccccttataactacagtgctttggcaggtaagcatcctgatcccccgcggaagctgctcacgtggcaactgtggggacccagacaggttatcaaaggcacccggtctttccgccttcaggagcatccccactagtgaattctagtggggctctgcttggtgccaacctcccccaaatgcgcgctgcgggagtgctcttccccaacccatcctagtatcctctcatgtgtgtgcttggtcagcatatctgagacgacgttccgctgtcccagaccagtccagtaatggacgggccagtgtgcgtagtcgtcttccggcttgtccggcgcatgtttggtgaaccggtggggtaaggttggtgtgcccaacgcccgtactttggtgacacctcaagaccacccaggaatgccagggaggtaccccgcctcacggcgggatctgaccctgggctaattgtctacggtggttcttcttgcttccacttctttctt

SEQ ID NO: 415 - RNA, 624 nt, synthetic construct
  misc_feature 1..624: note = IRES-62
  source 1..624: mol_type = other RNA
ttctcctgcaaccattacgcttaatcgcatgtgcattgagtggtgcatgtgttgaacaaacagctacaatcacatgggggcgggttttcccgccccacggcttctcgcgaggcccatccctcccttttctcccataactacagtgctttggtaggtaagcatcccgatctcccgcggaagctgctcacgtggcaactgtggggacccagacaggttatcaaaggcacccggtctttccgccttcaggagtatccctgctagcgaattctagtagggctctgcttggtgccaacctctcccaaatgcgcgctgcgggagtgctcttccccaaatcaccccagtatcctctcatgtgtgtgcctggtcagcatatctgagacgatgttccgctgtcccagaccagtccagtaatggacgggccagtgtgcgtagtcgtcctccggcttgtccggcgcatgtttggtgaaccggtggggtaaggttggtgtgcccaacgcccgtaatcaggggatacctcaaggcacccaggaatgccagggaggtatcccgcctcacagcgggatctgaccctggggtaaatgtctgcggggggtcctcttggcccaattctcagtaattttcagg

SEQ ID NO: 416 - RNA, 650 nt, synthetic construct
  misc_feature 1..650: note = IRES-63
  source 1..650: mol_type = other RNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatg

SEQ ID NO: 417 - RNA, 727 nt, synthetic construct
  misc_feature 1..727: note = IRES-64
  source 1..727: mol_type = other RNA
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggc

SEQ ID NO: 418 - RNA, 741 nt, synthetic construct; identical to SEQ ID NO: 353
  misc_feature 1..741: note = IRES-65
  source 1..741: mol_type = other RNA

SEQ ID NO: 419 - RNA, 741 nt, synthetic construct
  misc_feature 1..741: note = IRES-66
  source 1..741: mol_type = other RNA
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaa

SEQ ID NO: 420 - RNA, 742 nt, synthetic construct
  misc_feature 1..742: note = IRES-67
  source 1..742: mol_type = other RNA
ttaaaacagcctgtgggttgttcccacccacagggcccattgggcgtcagcaccctggtatcacggtacctttgtgcgcctgttttatatcccttcccccaattgtaacttagaagaaacacacaccgatcaacagcaagcgtggcacaccagccatgttttggtcaagcacttctgttaccccggactgagtatcaatagactgctcacgcggttgaaggagaaagcgtccgttatccggccagctacttcgagaaacctagtaacaccatggaagttgcggagtgtttcgctcagcactaccccagtgtagatcaggtcgatgagtcaccgctttccccacgggcgaccgtggcggtggctgcgttggcggcctgcctatgggggaacccataggacgctctaatacagacatggtgcgaagagtctattgagctagctggtattcctccggcccctgaatgcggctaatcctaactgtggagcacatgcccctaatccaaggggtagtgtgtcgtaatgagcaattccgcagcggaaccgactactttgggtgtccgtgtttcctcttattcttgtactggctgcttatggtgacaattgagagattgttaccatatagctattggattggccatccggtgactaatagagctattgtgtatctctttgttggatttgtaccacttaatttgaaagaaatcaggacactacgctacattttactattgaacaccgcaaa

SEQ ID NO: 421 - RNA, 743 nt, synthetic construct
  misc_feature 1..743: note = IRES-68
  source 1..743: mol_type = other RNA
ttaaaacagcctgtgggttgtacccacccacagggcccactgggcgctagcactctggtatcacggtacctttgtgcgcctgttttatgcccccttcccccaattgaaacttagaagttacacacaccgatcaacagcgggcgtggcataccagccgcgtcttgatcaagcactcctgtttccccggaccgagtatcaatagactgctcacgcggttgaaggagaaaacgttcgttacccggctaactacttcgagaaacctagtagcatcatgaaagttgcgaagcgtttcgctcagcacatccccagtgtagatcaggtcgatgagtcaccgcattccccacgggcgaccgtggcggtggctgcgttggcggcctgcctacggggcaacccgtaggacgcttcaatacagacatggtgcgaagagtcgattgagctagttagtagtcctccggcccctgaatccggctaatcctaactgcggagcacataccctcaacccagggggcattgtgtcgtaacgggtaactctgcagcggaaccgactactttgggtgtccgtgtttccttttattcttataatggctgcttatggtgacaattgaaagattgttaccatatagctattggattggccatccggtgtctaacagagctattatatacctctttgttggatttgtaccacttgatctaaaggaagtcaagacactacaattcatcatacaattgaacacagcaaa

SEQ ID NO: 422 - RNA, 743 nt, synthetic construct
  misc_feature 1..743: note = IRES-69
  source 1..743: mol_type = other RNA
ttaaaacagcctgtgggttgcacccactcacagggcccactgggcgcaagcactctggcacttcggtacctttgtgcgcctgttttatatcccctcccccaatgaaatttagaagcagcaaaccccgatcaatagcaggcataacgctccagttatgtcttgatcaagcacttctgtttccccggactgagtatcaatagactgctcacgcggttgaaggagaaaacgttcgttatccggctaactacttcggaaagcctagtaacaccatggaagttgcggagagtttcgttcagcacttccccagtgtagatcaggtcgatgagtcaccgcattccccacgggcgaccgtggcggtggctgcgttggcggcctgcccatggggtaacccatgggacgctctaatacggacatggtgtgaagagtctactgagctagttagtagtcctccggcccctgaatgcggctaatcccaactgcggagcacacgcccacaagccagtgggtagtgtgtcgtaacgggcaactctgcagcggaaccgactactttgggtgtccgtgtttccttttattcttatgttggctgcttatggtgacaattaaagagttgttaccatatagctattggattggccatccggtgtgcaacagagcgatcgtttacctatttattggttttgtaccattgacactgaagtctgtgatcacccttaattttatcttaaccctcaacacagccaaac

SEQ ID NO: 423 - RNA, 742 nt, synthetic construct
  misc_feature 1..742: note = IRES-70
  source 1..742: mol_type = other RNA
ttaaaacagcctgtgggttgtacccacccacagggcccactgggcgctagcacactggtattacggtacctttgtgcgcctgttttataccccccccaacctcgaaacttagaagtaaagcaaacccgatcaatagcaggtgcggcgcaccagtcgcatcttgatcaagcacttctgtaaccccggaccgagtatcaatagactgctcacgcggttgaaggagaaaacgttcgttacccggctaactacttcgagaaacccagtagcatcatgaaagttgcagagtgtttcgctcagcactacccccgtgtagatcaggccgatgagtcaccgcacttccccacgggcgaccgtggcggtggctgcgttggcggcctgcctatggggcaacccataggacgctctaatacggacatggtgcgaagagtctattgagctagttagtagtcctccggcccctgaatgcggctaatcctaactgcggagcacatacccttaatccaaagggcagtgtgtcgtaacgggtaactctgcagcggaaccgactactttgggtgtccgtgtttccttttaatttttactggctgcttatggtgacaattgaggaattgttgccatatagctattggattggccatccggtgactaacagagctattgtgttccaatttgttggatttaccccgctcacactcacagtcgtaagaacccttcattacgtgttatttctcaactcaagaaa

SEQ ID NO: 424 - RNA, 745 nt, synthetic construct
  misc_feature 1..745: note = IRES-71
  source 1..745: mol_type = other RNA
ttaaaacagcctgtgggttgtacccacccacagggcccactgggcgctagcactctggtactacggtacctttgtgtgcctgttttaagcccctaccccccactcgtaacttagaaggcttctcacactcgatcaatagtaggtgtggcacgccagtcacaccgtgatcaagcacttctgttaccccggtctgagtaccaataagctgctaacgcggctgaaggggaaaacgatcgttatccggctaactacttcgagaaacccagtaccaccatgaacgttgcagggtgtttcgctcggcacaaccccagtgtagatcaggtcgatgagtcaccgtattccccacgggcgaccgtggcggtggctgcgttggcggcctgcccatggggtgacccatgggacgctctaatactgacatggtgcgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttccttttattcttacattggctgcttatggtgacaattgaaaagttgttaccatatagctattggattggccatccggtgacaaatagagctattgtatatctttttgttggttacgtaccccttaattacaaagtggtttcaactttgaaatacatcctaacactaaattgtagaaa

SEQ ID NO: 425 - RNA, 742 nt, synthetic construct
  misc_feature 1..742: note = IRES-72
  source 1..742: mol_type = other RNA
ttaaaacagcctgtgggttgcacccacccacagggcccacagggcgctagcactctggtatcacggtacctttgtgcgcctgttttattaccccttccccaattgaaaattagaagcaatgcacaccgatcaacagcaggcgtggcgcaccagtcacgtctcgatcaagcacttctgtttccccggaccgagtatcaatagactgctcacgcggttgaaggagaaagtgttcgttatccggctaaccacttcgagaaacccagtaacaccatgaaagttgcagggtgtttcgctcagcacttccccagtgtagatcaggtcgatgagtcaccgcgttccccacgggcgaccgtggcggtggctgcgttggcggcctgcctatgggttaacccataggacgctctaatacagacatggtgcgaagagtttattgagctggttagtatccctccggcccctgaatgcggctaatcctaactgcggagcacgtgcctccaatccagggggttgcatgtcgtaacgggtaactctgcagcggaaccgactactttgggtgtccgtgtttccttttattcttatactggctgcttatggtgacaatcgaggaattgttaccatatagctattggattggccatccggtgtctaacagagcgattatatacctctttgttggatttatgcagctcaataccaccaactttaacacattgaaatatatcttaaagttaaacacagcaaa

SEQ ID NO: 426 - RNA, 585 nt, synthetic construct; identical to SEQ ID NO: 354
  misc_feature 1..585: note = IRES-73
  source 1..585: mol_type = other RNA

SEQ ID NO: 427 - RNA, 568 nt, synthetic construct; identical to SEQ ID NO: 355
  misc_feature 1..568: note = IRES-74
  source 1..568: mol_type = other RNA

SEQ ID NO: 428 - RNA, 696 nt, synthetic construct; identical to SEQ ID NO: 356
  misc_feature 1..696: note = IRES-75
  source 1..696: mol_type = other RNA

SEQ ID NO: 429 - RNA, 697 nt, synthetic construct; identical to SEQ ID NO: 357
  misc_feature 1..697: note = IRES-76
  source 1..697: mol_type = other RNA

SEQ ID NO: 430 - RNA, 562 nt, synthetic construct; identical to SEQ ID NO: 358
  misc_feature 1..562: note = IRES-77
  source 1..562: mol_type = other RNA

SEQ ID NO: 431 - RNA, 340 nt, synthetic construct; identical to SEQ ID NO: 360
  misc_feature 1..340: note = IRES-78
  source 1..340: mol_type = other RNA

SEQ ID NO: 432 - RNA, 315 nt, synthetic construct; identical to SEQ ID NO: 361
  misc_feature 1..315: note = IRES-79
  source 1..315: mol_type = other RNA

SEQ ID NO: 433 - RNA, 759 nt, synthetic construct; identical to SEQ ID NO: 363
  misc_feature 1..759: note = IRES-80
  source 1..759: mol_type = other RNA

SEQ ID NO: 434 - RNA, 478 nt, synthetic construct; identical to SEQ ID NO: 364
  misc_feature 1..478: note = IRES-81
  source 1..478: mol_type = other RNA

SEQ ID NO: 435 - RNA, 628 nt, synthetic construct; identical to SEQ ID NO: 365
  misc_feature 1..628: note = IRES-82
  source 1..628: mol_type = other RNA

SEQ ID NO: 436 - RNA, 618 nt, synthetic construct; identical to SEQ ID NO: 366
  misc_feature 1..618: note = IRES-83
  source 1..618: mol_type = other RNA

SEQ ID NO: 437 - RNA, 610 nt, synthetic construct; identical to SEQ ID NO: 367
  misc_feature 1..610: note = IRES-84
  source 1..610: mol_type = other RNA

SEQ ID NO: 438 - RNA, 614 nt, synthetic construct; identical to SEQ ID NO: 368
  misc_feature 1..614: note = IRES-85
  source 1..614: mol_type = other RNA

SEQ ID NO: 439 - RNA, 624 nt, synthetic construct; identical to SEQ ID NO: 369
  misc_feature 1..624: note = IRES-86
  source 1..624: mol_type = other RNA

SEQ ID NO: 440 - RNA, 737 nt, synthetic construct
  misc_feature 1..737: note = IRES-87
  source 1..737: mol_type = other RNA
ttaaaacagcctgtgggttgatcccacccacagggcccattgggcgctagcactctggtatcacggtacccttgtgcgcctgttttatgtcccttccctcaactgtaacttagaagtaacgcacaccgatcaacagtcagcgtggcacaccagccatgttttgatcaagcacttctgttaccccggaccgagtatcaacagactgctcacgcggttgaaggagaaagtgttcgttatccggccaactacttcgaaaaacctagtaacaccatggaagttgcagagtgtttcgctcagcactaccccagtgtagatcaggtcgatgagtcaccgcatcccccacgggcgaccgtggcggtggctgcgttggcggcctgcctatgggggaacccataggacgctctaatacagacatggtgcgaagagtccattgagctagttggtagtcctccggcccctgaatgcggctaatcctaactgcggagcacacaccttcaagccagagggcagtgtgtcgtaacgggcaactctgcagcggaaccgactactttgggtgtccgtgtttcattttattcttatactggctgcttatggtgacaattgagagattgttaccatatagctattggattggccatccagtgactagcagagctattatatacctctttgttgggtttataccacctaatttgaaagaagttaaaacattagaattcattattaaattgaataca

SEQ ID NO: 441 - RNA, 709 nt, synthetic construct; identical to SEQ ID NO: 371
  misc_feature 1..709: note = IRES-88
  source 1..709: mol_type = other RNA

SEQ ID NO: 442 - RNA, 708 nt, synthetic construct; identical to SEQ ID NO: 372
  misc_feature 1..708: note = IRES-89
  source 1..708: mol_type = other RNA

SEQ ID NO: 443 - RNA, 508 nt, synthetic construct; identical to SEQ ID NO: 373
  misc_feature 1..508: note = IRES-90
  source 1..508: mol_type = other RNA

SEQ ID NO: 444 - RNA, 516 nt, synthetic construct; identical to SEQ ID NO: 375
  misc_feature 1..516: note = IRES-91
  source 1..516: mol_type = other RNA

SEQ ID NO: 445 - RNA, 478 nt, synthetic construct; identical to SEQ ID NO: 376
  misc_feature 1..478: note = IRES-92
  source 1..478: mol_type = other RNA

SEQ ID NO: 446 - RNA, 403 nt, synthetic construct; identical to SEQ ID NO: 377
  misc_feature 1..403: note = IRES-93
  source 1..403: mol_type = other RNA

SEQ ID NO: 447 - RNA, 404 nt, synthetic construct; identical to SEQ ID NO: 378
  misc_feature 1..404: note = IRES-94
  source 1..404: mol_type = other RNA

SEQ ID NO: 448 - RNA, 744 nt, synthetic construct; identical to SEQ ID NO: 379
  misc_feature 1..744: note = IRES-95
  source 1..744: mol_type = other RNA

SEQ ID NO: 449 - RNA, 663 nt, synthetic construct; identical to SEQ ID NO: 380
  misc_feature 1..663: note = IRES-96
  source 1..663: mol_type = other RNA

SEQ ID NO: 450 - RNA, 744 nt, synthetic construct
  misc_feature 1..744: note = IRES-97
  source 1..744: mol_type = other RNA
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 451 - RNA, 734 nt, synthetic construct; identical to SEQ ID NO: 382
  misc_feature 1..734: note = IRES-98
  source 1..734: mol_type = other RNA

SEQ ID NO: 452 - RNA, 638 nt, synthetic construct; identical to SEQ ID NO: 383
  misc_feature 1..638: note = IRES-99
  source 1..638: mol_type = other RNA

SEQ ID NO: 453 - RNA, 743 nt, synthetic construct; identical to SEQ ID NO: 384
  misc_feature 1..743: note = IRES-100
  source 1..743: mol_type = other RNA

SEQ ID NO: 454 - RNA, 746 nt, synthetic construct; identical to SEQ ID NO: 385
  misc_feature 1..746: note = IRES-101
  source 1..746: mol_type = other RNA

SEQ ID NO: 455 - RNA, 726 nt, synthetic construct; identical to SEQ ID NO: 386
  misc_feature 1..726: note = IRES-102
  source 1..726: mol_type = other RNA

SEQ ID NO: 456 - RNA, 737 nt, synthetic construct; identical to SEQ ID NO: 387
  misc_feature 1..737: note = IRES-103
  source 1..737: mol_type = other RNA

SEQ ID NO: 457 - RNA, 327 nt, synthetic construct; identical to SEQ ID NO: 388
  misc_feature 1..327: note = IRES-104
  source 1..327: mol_type = other RNA

SEQ ID NO: 458 - RNA, 281 nt, synthetic construct; identical to SEQ ID NO: 389
  misc_feature 1..281: note = IRES-105
  source 1..281: mol_type = other RNA

SEQ ID NO: 459 - RNA, 459 nt, synthetic construct; identical to SEQ ID NO: 390
  misc_feature 1..459: note = IRES-106
  source 1..459: mol_type = other RNA

SEQ ID NO: 460 - RNA, 350 nt, synthetic construct; identical to SEQ ID NO: 391
  misc_feature 1..350: note = IRES-107
  source 1..350: mol_type = other RNA

SEQ ID NO: 461 - RNA, 318 nt, synthetic construct; identical to SEQ ID NO: 392
  misc_feature 1..318: note = IRES-108
  source 1..318: mol_type = other RNA

SEQ ID NO: 462 - RNA, 399 nt, synthetic construct; identical to SEQ ID NO: 393
  misc_feature 1..399: note = IRES-109
  source 1..399: mol_type = other RNA

SEQ ID NO: 463 - RNA, 430 nt, synthetic construct; identical to SEQ ID NO: 394
  misc_feature 1..430: note = IRES-110
  source 1..430: mol_type = other RNA

SEQ ID NO: 464 - RNA, 504 nt, synthetic construct; identical to SEQ ID NO: 395
  misc_feature 1..504: note = IRES-111
  source 1..504: mol_type = other RNA

SEQ ID NO: 465 - RNA, 532 nt, synthetic construct; identical to SEQ ID NO: 396
  misc_feature 1..532: note = IRES-112
  source 1..532: mol_type = other RNA

SEQ ID NO: 466 - RNA, 494 nt, synthetic construct; identical to SEQ ID NO: 397
  misc_feature 1..494: note = IRES-113
  source 1..494: mol_type = other RNA

SEQ ID NO: 467 - RNA, 378 nt, synthetic construct; identical to SEQ ID NO: 398
  misc_feature 1..378: note = IRES-114
  source 1..378: mol_type = other RNA

SEQ ID NO: 468 - RNA, 319 nt, synthetic construct; identical to SEQ ID NO: 399
  misc_feature 1..319: note = IRES-115
  source 1..319: mol_type = other RNA

SEQ ID NO: 469 - RNA, 420 nt, synthetic construct; identical to SEQ ID NO: 400
  misc_feature 1..420: note = IRES-116
  source 1..420: mol_type = other RNA

SEQ ID NO: 470 - RNA, 373 nt, synthetic construct; identical to SEQ ID NO: 401
  misc_feature 1..373: note = IRES-117
  source 1..373: mol_type = other RNA

SEQ ID NO: 471 - RNA, 280 nt, synthetic construct; identical to SEQ ID NO: 402
  misc_feature 1..280: note = IRES-118
  source 1..280: mol_type = other RNA

SEQ ID NO: 472 - RNA, 384 nt, synthetic construct; identical to SEQ ID NO: 403
  misc_feature 1..384: note = IRES-119
  source 1..384: mol_type = other RNA

SEQ ID NO: 473 - RNA, 384 nt, synthetic construct; identical to SEQ ID NO: 404
  misc_feature 1..384: note = IRES-120
  source 1..384: mol_type = other RNA

SEQ ID NO: 474 - RNA, 372 nt, synthetic construct; identical to SEQ ID NO: 405
  misc_feature 1..372: note = IRES-121
  source 1..372: mol_type = other RNA

SEQ ID NO: 475 - RNA, 388 nt, synthetic construct; identical to SEQ ID NO: 406
  misc_feature 1..388: note = IRES-122
  source 1..388: mol_type = other RNA

SEQ ID NO: 476 - RNA, 373 nt, synthetic construct; identical to SEQ ID NO: 407
  misc_feature 1..373: note = IRES-123
  source 1..373: mol_type = other RNA

SEQ ID NO: 477 - RNA, 438 nt, synthetic construct; identical to SEQ ID NO: 409
  misc_feature 1..438: note = IRES-124
  source 1..438: mol_type = other RNA

SEQ ID NO: 478 - RNA, 752 nt, synthetic construct; identical to SEQ ID NO: 411
  misc_feature 1..752: note = IRES-125
  source 1..752: mol_type = other RNA

SEQ ID NO: 479 - RNA, 708 nt, synthetic construct; identical to SEQ ID NO: 412
  misc_feature 1..708: note = IRES-126
  source 1..708: mol_type = other RNA

SEQ ID NO: 480 - RNA, 718 nt, synthetic construct; identical to SEQ ID NO: 413
  misc_feature 1..718: note = IRES-127
  source 1..718: mol_type = other RNA

SEQ ID NO: 481 - RNA, 699 nt, synthetic construct; identical to SEQ ID NO: 414
  misc_feature 1..699: note = IRES-128
  source 1..699: mol_type = other RNA

SEQ ID NO: 482 - RNA, 624 nt, synthetic construct; identical to SEQ ID NO: 415
  misc_feature 1..624: note = IRES-129
  source 1..624: mol_type = other RNA

SEQ ID NO: 483 - RNA, 650 nt, synthetic construct; identical to SEQ ID NO: 416
  misc_feature 1..650: note = IRES-130
  source 1..650: mol_type = other RNA

SEQ ID NO: 484 - RNA, 742 nt, synthetic construct
  misc_feature 1..742: note = IRES-131
  source 1..742: mol_type = other RNA
ttaaaacagcctgtgggttgatcccacccacagggcccactgggcgctagcactctggtatcacggtacctttgtgcgcctgttttatacttcctcccccaactgcaacttagaagtaacacaaaccgatcaacagtcagcgtggcacaccagccacgttttgatcaaacacttctgttaccccggactgagtatcaatagactgctcacgcggttgaaggagaaaacgttcgttatccggccaactacttcgagaaacctagtaacgccatggaagttgtggagtgtttcgctcagcactaccccagtgtagatcaggttgatgagtcaccgcattccccacgggtgaccgtggcggtggctgcgttggcggcctgcccatggggaaacccatgggacgctcttatacagacatggtgcgaagagtctattgagctagttggtagtcctccggcccctgaatgcggctaatcccaactgcggagcatacactctcaagccagagggtagtgtgtcgtaatgggcaactctgcagcggaaccgactactttgggtgtccgtgtttcattttattcctatactggctgcttatggtgacaattgagagattgttaccatatagctattggattggccatccggtgactaacagagctattatatatctttttgttgggtttataccacttagcttgaaagaggttaaaactctacattacattttaatactgaacaccgcaaa

SEQ ID NO: 485 - RNA, 741 nt, synthetic construct; identical to SEQ ID NO: 419
  misc_feature 1..741: note = IRES-132
  source 1..741: mol_type = other RNA

SEQ ID NO: 486 - RNA, 742 nt, synthetic construct; identical to SEQ ID NO: 420
  misc_feature 1..742: note = IRES-133
  source 1..742: mol_type = other RNA

SEQ ID NO: 487 - RNA, 743 nt, synthetic construct; identical to SEQ ID NO: 421
  misc_feature 1..743: note = IRES-134
  source 1..743: mol_type = other RNA

SEQ ID NO: 488 - RNA, 743 nt, synthetic construct; identical to SEQ ID NO: 422
  misc_feature 1..743: note = IRES-135
  source 1..743: mol_type = other RNA

SEQ ID NO: 489 - RNA, 742 nt, synthetic construct; identical to SEQ ID NO: 423
  misc_feature 1..742: note = IRES-136
  source 1..742: mol_type = other RNA

SEQ ID NO: 490 - RNA, 745 nt, synthetic construct; identical to SEQ ID NO: 424
  misc_feature 1..745: note = IRES-137
  source 1..745: mol_type = other RNA

SEQ ID NO: 491 - RNA, 742 nt, synthetic construct; identical to SEQ ID NO: 425
  misc_feature 1..742: note = IRES-138
  source 1..742: mol_type = other RNA

SEQ ID NO: 492 - RNA, 625 nt, synthetic construct
  misc_feature 1..625: note = IRES-139
  source 1..625: mol_type = other RNA
attctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 493 - RNA, 549 nt, synthetic construct
  misc_feature 1..549: note = IRES-140
  source 1..549: mol_type = other RNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacg

SEQ ID NO: 494 - RNA, 630 nt, synthetic construct
  misc_feature 1..630: note = IRES-141
  source 1..630: mol_type = other RNA
aacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaacc

SEQ ID NO: 495 - RNA, 653 nt, synthetic construct
  misc_feature 1..653: note = IRES-142
  source 1..653: mol_type = other RNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcgccacc

SEQ ID NO: 496 - DNA, 86 bp, synthetic construct
  misc_feature 1..86: note = Aptamer-01
  source 1..86: mol_type = other DNA
gggagacaagaataaacgctcaatgttcaaccagagtgaaaccactaacgggtcagagccccttcgacaggaggctcacaacaggc

SEQ ID NO: 497 - DNA, 40 bp, synthetic construct
  misc_feature 1..40: note = Aptamer-02
  source 1..40: mol_type = other DNA
actcactatttgttttcgcgcccagttgcaaaaagtgtcg

SEQ ID NO: 498 - DNA, 40 bp, synthetic construct
  misc_feature 1..40: note = Aptamer-03
  source 1..40: mol_type = other DNA
tccgcggcgccatctcatgtttagttgtcctatgtcgacg

SEQ ID NO: 499 - DNA, 41 bp, synthetic construct
  misc_feature 1..41: note = Aptamer-04
  source 1..41: mol_type = other DNA
tccgtagaaacgcgttaaggtgaaagtttgagggctcctca

SEQ ID NO: 500 - RNA, 86 nt, synthetic construct
  misc_feature 1..86: note = Aptamer-01
  source 1..86: mol_type = other RNA
gggagacaagaataaacgctcaatgttcaaccagagtgaaaccactaacgggtcagagccccttcgacaggaggctcacaacaggc

SEQ ID NO: 501 - RNA, 40 nt, synthetic construct
  misc_feature 1..40: note = Aptamer-02
  source 1..40: mol_type = other RNA
actcactatttgttttcgcgcccagttgcaaaaagtgtcg

SEQ ID NO: 502 - RNA, 40 nt, synthetic construct
  misc_feature 1..40: note = Aptamer-03
  source 1..40: mol_type = other RNA
tccgcggcgccatctcatgtttagttgtcctatgtcgacg

SEQ ID NO: 503 - RNA, 41 nt, synthetic construct
  misc_feature 1..41: note = Aptamer-04
  source 1..41: mol_type = other RNA
tccgtagaaacgcgttaaggtgaaagtttgagggctcctca

SEQ ID NO: 504 - DNA, 17 bp, synthetic construct
  misc_feature 1..17: note = BTE consensus sequence CITE
  source 1..17: mol_type = other DNA
ggatcctgggaaacagg

SEQ ID NO: 505 - RNA, 17 nt, synthetic construct
  misc_feature 1..17: note = BTE consensus sequence CITE
  source 1..17: mol_type = other RNA
ggatcctgggaaacagg

SEQ ID NO: 506 - RNA, 235 nt, synthetic construct
  misc_feature 1..235: note = 5'PIE wo spacer
  source 1..235: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccg

SEQ ID NO: 507 - RNA, 285 nt, synthetic construct
  misc_feature 1..285: note = 5'PIE with spacer
  source 1..285: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacaca

SEQ ID NO: 508 - RNA, 183 nt, synthetic construct
  misc_feature 1..183: note = 3'PIE wo spacer
  source 1..183: mol_type = other RNA
cggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 509 - RNA, 20 nt, synthetic construct
  misc_feature 1..20: note = 5'binding sequence for oligo purification (binding sequence for AS1)
  source 1..20: mol_type = other RNA
cgtcaagacgagggtaaaga

SEQ ID NO: 510 - RNA, 20 nt, synthetic construct
  misc_feature 1..20: note = 3'binding sequence for oligo purification (binding sequence for AS2)
  source 1..20: mol_type = other RNA
aatcgacggataacagcata

SEQ ID NO: 511-512 - skipped (2 entries)

SEQ ID NO: 513 - DNA, 575 bp, synthetic construct
  source 1..575: mol_type = other DNA
  misc_feature 1..575: note = EMCV IRES
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 514 - RNA, 575 nt, synthetic construct
  source 1..575: mol_type = other RNA
  misc_feature 1..575: note = EMCV IRES
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 515 - DNA, 733 bp, synthetic construct
  source 1..733: mol_type = other DNA
  misc_feature 1..733: note = SaV IRES-Kozak
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcgccacc

SEQ ID NO: 516 - DNA, 750 bp, synthetic construct
  source 1..750: mol_type = other DNA
  misc_feature 1..750: note = AiV IRES-Kozak
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtcgccacc

SEQ ID NO: 517 - DNA, 747 bp, synthetic construct
  source 1..747: mol_type = other DNA
  misc_feature 1..747: note = CVB3 IRES-Kozak
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccacc

SEQ ID NO: 518 - RNA, 733 nt, synthetic construct
  source 1..733: mol_type = other RNA
  misc_feature 1..733: note = SaV IRES-Kozak
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcgccacc

SEQ ID NO: 519 - RNA, 750 nt, synthetic construct
  source 1..750: mol_type = other RNA
  misc_feature 1..750: note = AiV IRES-Kozak
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtcgccacc

SEQ ID NO: 520 - RNA, 747 nt, synthetic construct
  source 1..747: mol_type = other RNA
  misc_feature 1..747: note = CVB3 IRES-Kozak
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccacc

SEQ ID NO: 521 - RNA, 2758 nt, synthetic construct
  misc_feature 1..2758: note = precursor circRNA-06
  source 1..2758: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggactggacctggcgcttcctgttcgtggtcgccgccgcgaccggcgtgcagagccaggtgcagctcgtccagtccggggccgaggtgaagaagcccggcagctccgtgaaggtcagctgcaaggcctccgggggcacgttcaaccggtacaccgtgaactgggtgcgccaggccccggggcagggcctggagtggatgggcgggatcatccccatcttcggcaccgccaactacgcgcagcggttccaggggcgcctgaccatcacggccgacgagagcacctccaccgcctacatggagctcagctccctgcggagcgacgacaccgccgtctacttctgcgcgcgcgagaacctggacaactccggcacgtactactacttcagcgggtggttcgacccctggggccagggcaccctcgtgaccgtgtccagcgcctccaccaaggggcccagcgtcttcccgctggccccctccagcaagtccacgagcggcgggaccgccgccctgggctgcctcgtgaaggactacttccccgagcccgtgaccgtcagctggaactccggcgcgctgaccagcggggtgcacacgttcccggccgtgctgcagtccagcggcctctactccctgagctccgtcgtgaccgtgcccagctccagcctggggacccagacctacatctgcaacgtcaaccacaagccctccaacacgaaggtggacaagcgggtggagcccaagagctgcgacaagacccacacctgcccgccctgccccgccccggagctcctgggcgggccgtccgtcttcctgttccctcccaagcccaaggacaccctcatgatcagccgcacgccggaggtgacctgcgtcgtggtggacgtgtcccacgaggaccccgaggtgaagttcaactggtacgtggacggcgtcgaggtgcacaacgccaagaccaagccccgggaggagcagtacaacagcacctaccgcgtggtctccgtgctgacggtgctgcaccaggactggctcaacggcaaggagtacaagtgcaaggtcagcaacaaggccctgcccgcgccgatcgagaagaccatctccaaggccaaggggcagccccgggagccccaggtgtacaccctgcccccgagccgcgaggagatgaccaagaaccaggtgagcctcacgtgcctggtcaagggcttctacccctccgacatcgccgtggagtgggagagcaacgggcagcccgagaacaactacaagaccaccccgcccgtgctggactccgacggcagcttcttcctctactccaagctgaccgtcgacaagagccggtggcagcaggggaacgtgttctcctgcagcgtgatgcacgaggccctgcacaaccactacacgcagaagtccctcagcctgtcccccggcaagtgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcaaagaagagcgaatt

SEQ ID NO: 522 - RNA, 2056 nt, synthetic construct
  misc_feature 1..2056: note = precursor circRNA-07
  source 1..2056: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagcgtgcccaccatggcctgggccctgctcctgctgtccctcctgacccagggcacggggagctgggcgcagtccgccctgacccagccgcgcagcgtctccggcagccccgggcagtccgtgaccatcagctgcaccggcacgtccagcgacatcgggggctacaacttcgtgtcctggtaccagcagcaccccggcaaggcccccaagctcatgatctacgacgccaccaagcggccgagcggggtccccgaccgcttctccggcagcaagtccgggaacaccgccagcctgaccatctccggcctgcaggcggaggacgaggccgactactactgctgcagctacgccggggactacacgcccggcgtggtgttcggcgggggcaccaagctcaccgtcctggggcagccgaaggccgcgccgtccgtgaccctgttccctcccagcagcgaggagctccaggccaacaaggccacgctggtgtgcctgatctccgacttctaccccggcgccgtcaccgtggcctggaaggcggacagctccccggtgaaggccggcgtcgagaccaccacgcccagcaagcagtccaacaacaagtacgccgccagctcctacctcagcctgacccccgagcagtggaagtcccaccggagctactcctgccaggtgacccacgaggggagcaccgtggagaagacggtcgcccccaccgagtgctcctgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcaaagaagagcgaatt

SEQ ID NO: 523 - RNA, 3562 nt, synthetic construct
  misc_feature 1..3562: note = precursor circRNA-08
  source 1..3562: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggactggacctggcgcttcctgttcgtggtcgccgccgcgaccggcgtgcagagccaggtgcagctcgtccagtccggggccgaggtgaagaagcccggcagctccgtgaaggtcagctgcaaggcctccgggggcacgttcaaccggtacaccgtgaactgggtgcgccaggccccggggcagggcctggagtggatgggcgggatcatccccatcttcggcaccgccaactacgcgcagcggttccaggggcgcctgaccatcacggccgacgagagcacctccaccgcctacatggagctcagctccctgcggagcgacgacaccgccgtctacttctgcgcgcgcgagaacctggacaactccggcacgtactactacttcagcgggtggttcgacccctggggccagggcaccctcgtgaccgtgtccagcgcctccaccaaggggcccagcgtcttcccgctggccccctccagcaagtccacgagcggcgggaccgccgccctgggctgcctcgtgaaggactacttccccgagcccgtgaccgtcagctggaactccggcgcgctgaccagcggggtgcacacgttcccggccgtgctgcagtccagcggcctctactccctgagctccgtcgtgaccgtgcccagctccagcctggggacccagacctacatctgcaacgtcaaccacaagccctccaacacgaaggtggacaagcgggtggagcccaagagctgcgacaagacccacacctgcccgccctgccccgccccggagctcctgggcgggccgtccgtcttcctgttccctcccaagcccaaggacaccctcatgatcagccgcacgccggaggtgacctgcgtcgtggtggacgtgtcccacgaggaccccgaggtgaagttcaactggtacgtggacggcgtcgaggtgcacaacgccaagaccaagccccgggaggagcagtacaacagcacctaccgcgtggtctccgtgctgacggtgctgcaccaggactggctcaacggcaaggagtacaagtgcaaggtcagcaacaaggccctgcccgcgccgatcgagaagaccatctccaaggccaaggggcagccccgggagccccaggtgtacaccctgcccccgagccgcgaggagatgaccaagaaccaggtgagcctcacgtgcctggtcaagggcttctacccctccgacatcgccgtggagtgggagagcaacgggcagcccgagaacaactacaagaccaccccgcccgtgctggactccgacggcagcttcttcctctactccaagctgaccgtcgacaagagccggtggcagcaggggaacgtgttctcctgcagcgtgatgcacgaggccctgcacaaccactacacgcagaagtccctcagcctgtcccccggcaagcgcgccaagcggggaagcggagctactaacttcagcctgctgaagcaggctggcgacgtggaggagaaccctggacctatgagcgtgcccaccatggcctgggccctgctcctgctgtccctcctgacccagggcacggggagctgggcgcagtccgccctgacccagccgcgcagcgtgtccggcagccccgggcagtccgtgaccatcagctgcaccggcacgtccagcgacatcgggggctacaacttcgtgtcctggtaccagcagcaccccggcaaggcccccaagctcatgatctacgacgccaccaagcggccgagcggggtccccgaccgcttctccggcagcaagtccgggaacaccgccagcctgaccatctccggcctgcaggcggaggacgaggccgactactactgctgcagctacgccggggactacacgcccggcgtggtgttcggcgggggcaccaagctcaccgtcctggggcagccgaaggccgcgccgtccgtgaccctgttccctcccagcagcgaggagctccaggccaacaaggccacgctggtgtgcctgatctccgacttctaccccggcgccgtcaccgtggcctggaaggcggacagctccccggtgaaggccggcgtcgagaccaccacgcccagcaagcagtccaacaacaagtacgccgccagctcctacctcagcctgacccccgagcagtggaagtcccaccggagctactcctgccaggtgacccacgaggggagcaccgtggagaagacggtcgcccccaccgagtgctcctgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagaggctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcaaagaagagcgaatt

SEQ ID NO: 524 - RNA, 3028 nt, synthetic construct
  misc_feature 1..3028: note = precursor circRNA-09
  source 1..3028: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaaggccatcctggtggtcctcctgtacaccttcgccaccgcgaacgccgacacgctgtgcatcggctaccacgccaacaacagcaccgacaccgtggacaccgtgctcgagaagaacgtcacggtgacccactccgtgaacctgctggaggacaagcacaacgggaagctctgcaagctgcggggcgtcgccccgctgcacctcgggaagtgcaacatcgccggctggatcctggggaacccggagtgcgagagcctgtccaccgcgagctcctggagctacatcgtggagacctccagctccgacaacggcacgtgctaccccggcgacttcatcgactacgaggagctccgcgagcagctgagctccgtgagctccttcgagcggttcgagatcttccccaagaccagctcctggcccaaccacgacagcaacaagggggtcaccgccgcctgcccgcacgccggcgcgaagtccttctacaagaacctgatctggctcgtgaagaaggggaacagctaccccaagctgtccaagagctacatcaacgacaagggcaaggaggtgctggtcctctgggggatccaccaccccagcacctccgccgaccagcagagcctgtaccagaacgccgacgcctacgtgttcgtgggctccagccgctactccaagaagttcaagcccgagatcgccatccggccgaaggtccgcgaccaggagggccggatgaactactactggacgctggtggagcccggggacaagatcaccttcgaggcgaccggcaacctcgtggtcccccgctacgccttcgccatggagcggaacgccgggagcggcatcatcatctccgacacccccgtgcacgactgcaacacgacctgccagaccccgaagggcgccatcaacaccagcctgcccttccagaacatccaccccatcacgatcgggaagtgccccaagtacgtgaagtccaccaagctgcgcctcgcgaccggcctgcggaacgtcccgagcatccagtcccgcgggctgttcggcgccatcgccgggttcatcgagggcggctggaccgggatggtggacggctggtacgggtaccaccaccagaacgagcagggcagcgggtacgccgccgacctcaagtccacgcagaacgcgatcgacgagatcaccaacaaggtgaacagcgtcatcgagaagatgaacacccagttcaccgccgtgggcaaggagttcaaccacctggagaagcggatcgagaacctgaacaagaaggtcgacgacggcttcctcgacatctggacgtacaacgccgagctgctggtgctcctggagaacgagcgcaccctggactaccacgactccaacgtgaagaacctctacgagaaggtccggagccagctgaagaacaacgccaaggagatcgggaacggctgcttcgagttctaccacaagtgcgacaacacctgcatggagtccgtgaagaacgggacctacgactaccccaagtacagcgaggaggccaagctgaaccgcgaggagatcgacggcgtgaagctcgagtccacgcggatctaccagatcctggcgatctacagcaccgtcgccagctccctggtgctcgtggtcagcctgggggccatctccttctggatgtgcagcaacggctccctgcagtgccgcatctgcatctgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcaaagaagagcgaatt

SEQ ID NO: 525 - RNA, 2979 nt, synthetic construct
  misc_feature 1..2979: note = precursor circRNA-10
  source 1..2979: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 526 - RNA, 2959 nt, synthetic construct
  misc_feature 1..2959: note = precursor circRNA-11
  source 1..2959: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 527 - RNA, 2976 nt, synthetic construct
  misc_feature 1..2976: note = precursor circRNA-12
  source 1..2976: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacatttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtcatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 528 - RNA, 2965 nt, synthetic construct
  misc_feature 1..2965: note = precursor circRNA-13
  source 1..2965: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcgccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 529 - RNA, 2982 nt, synthetic construct
  misc_feature 1..2982: note = precursor circRNA-14
  source 1..2982: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacatttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtcgccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 530 - RNA, 1878 nt, synthetic construct
  misc_feature 1..1878: note = precursor circRNA-15
  source 1..1878: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgagtccacctgtccctcctgggctgctggattgtctcgttttcctgccaaataaacaggatcagcgctttacaaaaaaaaaaaaaaaaaaaaaaaaaaaaaaaaaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 531 - RNA, 3027 nt, synthetic construct
  misc_feature 1..3027: note = precursor circRNA-16
  source 1..3027: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatggcctgcctgggcttccagcgccacaaggcccagctcaacctggcgacccggacctggccctgcacgctgctgttcttcctgctgttcatccccgtgttctgcggcgggggcgggagcggcgggggcggctcccagttcccccgcgtctgcatgaccctcgacggcgtgctgaacaaggagtgctgcccgcccctgggccccgaggcgaccaacatctgcgggttcctcgagggccgggggcagtgcgccgaggtgcagacggacacccgcccctggagcggcccgtacatcctgcggaaccaggacgaccgcgagcagtggccccggaagttcttcaaccggacctgcaagtgcaccgggaacttcgccggctacaactgcggcgggtgcaagttcggctggacggggcccgactgcaaccggaagaagcccgccatcctgcgccggaacatccactccctcaccgcccaggagcgcgagcagttcctgggcgcgctggacctcgccaagaagtccatccacccggactacgtcatcaccacccagcactggctgggcctgctcgggcccaacggcacgcagccccagatcgccaactgctccgtgtacgacttcttcgtgtggctgcactactacagcgtccgggacaccctgctcgggcccggccgcccgtacaaggccatcgacttctcccaccaggggcccgcgttcgtgacctggcaccggtaccacctcctgtggctcgagcgcgagctgcagcggctgaccggcaacgagagcttcgccctcccctactggaacttcgccacgggcaagaacgagtgcgacgtgtgcaccgacgagctgctgggggccgcccgccaggacgaccccaccctgatctcccggaacagccgcttctccacctgggagatcgtctgcgacagcctcgacgactacaaccggcgcgtgacgctgtgcaacggcacctacgaggggctgctccgccggaacaaggtgggccgcaacaacgagaagctgccgaccctgaagaacgtccaggactgcctctccctgcagaagttcgacagcccgcccttcttccagaactccaccttcagcttccggaacgcgctggaggggttcgacaaggccgacggcacgctcgactcccaggtgatgaacctgcacaacctggcccacagcttcctcaacggcaccaacgccctgccccactccgccgcgaacgacccggtgttcgtcgtgctgcacagcttcaccgacgccatcttcgacgagtggctcaagcgcaacaacccctccaccgacgcctggccccaggagctggcccccatcgggcacaaccggatgtacaacatggtgccgttcttcccgcccgtcacgaacgaggagctgttcctgaccgccgagcagctcggctacaactacgcggtggacctgagcgaggaggaggcccccgtgtggagcaccaccctgtccggcgggggcgggagcggcgggggcggctccttcctcctgtggatcctggccgccgtctccagcggcctcttcttctactccttcctgctgaccgcggtgagcctctccaagatgctgaagaagcgcagccccctgacgaccggggtgtacgtcaagatgccgcccaccgagcccgagtgcgagaagcagttccagccctacttcatccccatcaactgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 532 - RNA, 2094 nt, synthetic construct
  misc_feature 1..2094: note = precursor circRNA-17
  source 1..2094: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatggcctgcctgggcttccagcgccacaaggcccagctcaacctggcgacccggacctggccctgcacgctcctgttcttcctgctgttcatcccggtgttctgcctgtccgccctcctggccgtgggcgcgctggaggggccccgcaaccaggactggctcggcgtcccgcggcagctggtgaccaagaccaacggcacccagccccagatcgccaactgcagcgtgtacgacttcttcgtctggctgcactactactccgtgcgcgacaccctcctgaaggacagcggctcccccttcccggccgccgtgatcctgcgcgacgcgctccacatggcccgggggctgaagtacctgcaccaggagcccccggtgaccaacaccgagatgttcgtcacggcccccgacaacctgggctacatgtacgaggtgcagtggcccgggcaggagttcatgcgcgagggcgtggagctgtgccccgggaacaagtacgagatgcggcgccacggcaccacccacagcctcgtcatccacgacgtggccaagttcgtggccgcgtggaccctgaaggccgccgccttcctgctctggatcctggccgccgtgagctccggcctgttcttctacagcttcctcctgaccgcggtctccctgagcaagatgctcaagaagcgcagccccctgaccacgggggtgtacgtgaagatgccgcccaccgagcccgagtgcgagaagcagttccagccctacttcatcccgatcaactgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatagaatt

SEQ ID NO: 533 - RNA, 2436 nt, synthetic construct
  misc_feature 2436^1: note = spliced circRNA-06
  source 1..2436: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggactggacctggcgcttcctgttcgtggtcgccgccgcgaccggcgtgcagagccaggtgcagctcgtccagtccggggccgaggtgaagaagcccggcagctccgtgaaggtcagctgcaaggcctccgggggcacgttcaaccggtacaccgtgaactgggtgcgccaggccccggggcagggcctggagtggatgggcgggatcatccccatcttcggcaccgccaactacgcgcagcggttccaggggcgcctgaccatcacggccgacgagagcacctccaccgcctacatggagctcagctccctgcggagcgacgacaccgccgtctacttctgcgcgcgcgagaacctggacaactccggcacgtactactacttcagcgggtggttcgacccctggggccagggcaccctcgtgaccgtgtccagcgcctccaccaaggggcccagcgtcttcccgctggccccctccagcaagtccacgagcggcgggaccgccgccctgggctgcctcgtgaaggactacttccccgagcccgtgaccgtcagctggaactccggcgcgctgaccagcggggtgcacacgttcccggccgtgctgcagtccagcggcctctactccctgagctccgtcgtgaccgtgcccagctccagcctggggacccagacctacatctgcaacgtcaaccacaagccctccaacacgaaggtggacaagcgggtggagcccaagagctgcgacaagacccacacctgcccgccctgccccgccccggagctcctgggcgggccgtccgtcttcctgttccctcccaagcccaaggacaccctcatgatcagccgcacgccggaggtgacctgcgtcgtggtggacgtgtcccacgaggaccccgaggtgaagttcaactggtacgtggacggcgtcgaggtgcacaacgccaagaccaagccccgggaggagcagtacaacagcacctaccgcgtggtctccgtgctgacggtgctgcaccaggactggctcaacggcaaggagtacaagtgcaaggtcagcaacaaggccctgcccgcgccgatcgagaagaccatctccaaggccaaggggcagccccgggagccccaggtgtacaccctgcccccgagccgcgaggagatgaccaagaaccaggtgagcctcacgtgcctggtcaagggcttctacccctccgacatcgccgtggagtgggagagcaacgggcagcccgagaacaactacaagaccaccccgcccgtgctggactccgacggcagcttcttcctctactccaagctgaccgtcgacaagagccggtggcagcaggggaacgtgttctcctgcagcgtgatgcacgaggccctgcacaaccactacacgcagaagtccctcagcctgtcccccggcaagtgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

SEQ ID NO: 534 - RNA, 1734 nt, synthetic construct
  misc_feature 1734^1: note = spliced circRNA-07
  source 1..1734: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagcgtgcccaccatggcctgggccctgctcctgctgtccctcctgacccagggcacggggagctgggcgcagtccgccctgacccagccgcgcagcgtctccggcagccccgggcagtccgtgaccatcagctgcaccggcacgtccagcgacatcgggggctacaacttcgtgtcctggtaccagcagcaccccggcaaggcccccaagctcatgatctacgacgccaccaagcggccgagcggggtccccgaccgcttctccggcagcaagtccgggaacaccgccagcctgaccatctccggcctgcaggcggaggacgaggccgactactactgctgcagctacgccggggactacacgcccggcgtggtgttcggcgggggcaccaagctcaccgtcctggggcagccgaaggccgcgccgtccgtgaccctgttccctcccagcagcgaggagctccaggccaacaaggccacgctggtgtgcctgatctccgacttctaccccggcgccgtcaccgtggcctggaaggcggacagctccccggtgaaggccggcgtcgagaccaccacgcccagcaagcagtccaacaacaagtacgccgccagctcctacctcagcctgacccccgagcagtggaagtcccaccggagctactcctgccaggtgacccacgaggggagcaccgtggagaagacggtcgcccccaccgagtgctcctgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

SEQ ID NO: 535 - RNA, 3240 nt, synthetic construct
  misc_feature 3240^1: note = spliced circRNA-08
  source 1..3240: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggactggacctggcgcttcctgttcgtggtcgccgccgcgaccggcgtgcagagccaggtgcagctcgtccagtccggggccgaggtgaagaagcccggcagctccgtgaaggtcagctgcaaggcctccgggggcacgttcaaccggtacaccgtgaactgggtgcgccaggccccggggcagggcctggagtggatgggcgggatcatccccatcttcggcaccgccaactacgcgcagcggttccaggggcgcctgaccatcacggccgacgagagcacctccaccgcctacatggagctcagctccctgcggagcgacgacaccgccgtctacttctgcgcgcgcgagaacctggacaactccggcacgtactactacttcagcgggtggttcgacccctggggccagggcaccctcgtgaccgtgtccagcgcctccaccaaggggcccagcgtcttcccgctggccccctccagcaagtccacgagcggcgggaccgccgccctgggctgcctcgtgaaggactacttccccgagcccgtgaccgtcagctggaactccggcgcgctgaccagcggggtgcacacgttcccggccgtgctgcagtccagcggcctctactccctgagctccgtcgtgaccgtgcccagctccagcctggggacccagacctacatctgcaacgtcaaccacaagccctccaacacgaaggtggacaagcgggtggagcccaagagctgcgacaagacccacacctgcccgccctgccccgccccggagctcctgggcgggccgtccgtcttcctgttccctcccaagcccaaggacaccctcatgatcagccgcacgccggaggtgacctgcgtcgtggtggacgtgtcccacgaggaccccgaggtgaagttcaactggtacgtggacggcgtcgaggtgcacaacgccaagaccaagccccgggaggagcagtacaacagcacctaccgcgtggtctccgtgctgacggtgctgcaccaggactggctcaacggcaaggagtacaagtgcaaggtcagcaacaaggccctgcccgcgccgatcgagaagaccatctccaaggccaaggggcagccccgggagccccaggtgtacaccctgcccccgagccgcgaggagatgaccaagaaccaggtgagcctcacgtgcctggtcaagggcttctacccctccgacatcgccgtggagtgggagagcaacgggcagcccgagaacaactacaagaccaccccgcccgtgctggactccgacggcagcttcttcctctactccaagctgaccgtcgacaagagccggtggcagcaggggaacgtgttctcctgcagcgtgatgcacgaggccctgcacaaccactacacgcagaagtccctcagcctgtcccccggcaagcgcgccaagcggggaagcggagctactaacttcagcctgctgaagcaggctggcgacgtggaggagaaccctggacctatgagcgtgcccaccatggcctgggccctgctcctgctgtccctcctgacccagggcacggggagctgggcgcagtccgccctgacccagccgcgcagcgtgtccggcagccccgggcagtccgtgaccatcagctgcaccggcacgtccagcgacatcgggggctacaacttcgtgtcctggtaccagcagcaccccggcaaggcccccaagctcatgatctacgacgccaccaagcggccgagcggggtccccgaccgcttctccggcagcaagtccgggaacaccgccagcctgaccatctccggcctgcaggcggaggacgaggccgactactactgctgcagctacgccggggactacacgcccggcgtggtgttcggcgggggcaccaagctcaccgtcctggggcagccgaaggccgcgccgtccgtgaccctgttccctcccagcagcgaggagctccaggccaacaaggccacgctggtgtgcctgatctccgacttctaccccggcgccgtcaccgtggcctggaaggcggacagctccccggtgaaggccggcgtcgagaccaccacgcccagcaagcagtccaacaacaagtacgccgccagctcctacctcagcctgacccccgagcagtggaagtcccaccggagctactcctgccaggtgacccacgaggggagcaccgtggagaagacggtcgcccccaccgagtgctcctgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagaggctacggactt

SEQ ID NO: 536 - RNA, 2706 nt, synthetic construct
  misc_feature 2706^1: note = spliced circRNA-09
  source 1..2706: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaaggccatcctggtggtcctcctgtacaccttcgccaccgcgaacgccgacacgctgtgcatcggctaccacgccaacaacagcaccgacaccgtggacaccgtgctcgagaagaacgtcacggtgacccactccgtgaacctgctggaggacaagcacaacgggaagctctgcaagctgcggggcgtcgccccgctgcacctcgggaagtgcaacatcgccggctggatcctggggaacccggagtgcgagagcctgtccaccgcgagctcctggagctacatcgtggagacctccagctccgacaacggcacgtgctaccccggcgacttcatcgactacgaggagctccgcgagcagctgagctccgtgagctccttcgagcggttcgagatcttccccaagaccagctcctggcccaaccacgacagcaacaagggggtcaccgccgcctgcccgcacgccggcgcgaagtccttctacaagaacctgatctggctcgtgaagaaggggaacagctaccccaagctgtccaagagctacatcaacgacaagggcaaggaggtgctggtcctctgggggatccaccaccccagcacctccgccgaccagcagagcctgtaccagaacgccgacgcctacgtgttcgtgggctccagccgctactccaagaagttcaagcccgagatcgccatccggccgaaggtccgcgaccaggagggccggatgaactactactggacgctggtggagcccggggacaagatcaccttcgaggcgaccggcaacctcgtggtcccccgctacgccttcgccatggagcggaacgccgggagcggcatcatcatctccgacacccccgtgcacgactgcaacacgacctgccagaccccgaagggcgccatcaacaccagcctgcccttccagaacatccaccccatcacgatcgggaagtgccccaagtacgtgaagtccaccaagctgcgcctcgcgaccggcctgcggaacgtcccgagcatccagtcccgcgggctgttcggcgccatcgccgggttcatcgagggcggctggaccgggatggtggacggctggtacgggtaccaccaccagaacgagcagggcagcgggtacgccgccgacctcaagtccacgcagaacgcgatcgacgagatcaccaacaaggtgaacagcgtcatcgagaagatgaacacccagttcaccgccgtgggcaaggagttcaaccacctggagaagcggatcgagaacctgaacaagaaggtcgacgacggcttcctcgacatctggacgtacaacgccgagctgctggtgctcctggagaacgagcgcaccctggactaccacgactccaacgtgaagaacctctacgagaaggtccggagccagctgaagaacaacgccaaggagatcgggaacggctgcttcgagttctaccacaagtgcgacaacacctgcatggagtccgtgaagaacgggacctacgactaccccaagtacagcgaggaggccaagctgaaccgcgaggagatcgacggcgtgaagctcgagtccacgcggatctaccagatcctggcgatctacagcaccgtcgccagctccctggtgctcgtggtcagcctgggggccatctccttctggatgtgcagcaacggctccctgcagtgccgcatctgcatctgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

SEQ ID NO: 537 - RNA, 2664 nt, synthetic construct
  misc_feature 2664^1: note = spliced circRNA-10
  source 1..2664: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

SEQ ID NO: 538 - RNA, 2644 nt, synthetic construct
  misc_feature 2644^1: note = spliced circRNA-11
  source 1..2644: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggactt

SEQ ID NO: 539 - RNA, 2661 nt, synthetic construct
  misc_feature 2661^1: note = spliced circRNA-12
  source 1..2661: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacatttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtcatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggactt

SEQ ID NO: 540 - RNA, 2650 nt, synthetic construct
  misc_feature 2650^1: note = spliced circRNA-13
  source 1..2650: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgtattccggtagggctctgcttggtgccaacctcccccaaatgcgcgctgcgggagtgcccttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcgccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggactt

SEQ ID NO: 541 - RNA, 2667 nt, synthetic construct
  misc_feature 2667^1: note = spliced circRNA-14
  source 1..2667: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacatttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaaatcacgttgggcacctagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtcgccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgatccctgttcccagagcccacttttttttctttttttgaaataaaatagcctgtctttcaagcttaaaaaaaaaaaaaaaaaaaaaaaaaaaaaaaaaaaactcgagcggctattatgcgttaccggcgagacgctacggactt

SEQ ID NO: 542 - RNA, 1563 nt, synthetic construct
  misc_feature 1563^1: note = spliced circRNA-15
  source 1..1563: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggcgtgaaggtcctgttcgccctcatctgcatcgccgtggcggaggccaagcccaccgagaacaacgaggacttcaacatcgtggccgtcgccagcaacttcgccaccacggacctggacgcggaccgggggaagctgccgggcaagaagctccccctggaggtgctgaaggagatggaggccaacgcccgcaaggccgggtgcacccggggctgcctcatctgcctgtcccacatcaagtgcacccccaagatgaagaagttcatccccgggcgctgccacacctacgagggcgacaaggagagcgcgcagggcgggatcggcgaggccatcgtggacatcccggagatccccgggttcaaggacctggagcccatggagcagttcatcgcccaggtcgacctctgcgtggactgcacgaccggctgcctgaaggggctggccaacgtgcagtgctccgacctcctgaagaagtggctgccccagcggtgcgccaccttcgcgagcaagatccagggccaggtcgacaagatcaagggcgccgggggcgactgagtccacctgtccctcctgggctgctggattgtctcgttttcctgccaaataaacaggatcagcgctttacaaaaaaaaaaaaaaaaaaaaaaaaaaaaaaaaaaaacggctattatgcgttaccggcgagacgctacggactt

SEQ ID NO: 543 - RNA, 2712 nt, synthetic construct
  misc_feature 2712^1: note = spliced circRNA-16
  source 1..2712: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatggcctgcctgggcttccagcgccacaaggcccagctcaacctggcgacccggacctggccctgcacgctgctgttcttcctgctgttcatccccgtgttctgcggcgggggcgggagcggcgggggcggctcccagttcccccgcgtctgcatgaccctcgacggcgtgctgaacaaggagtgctgcccgcccctgggccccgaggcgaccaacatctgcgggttcctcgagggccgggggcagtgcgccgaggtgcagacggacacccgcccctggagcggcccgtacatcctgcggaaccaggacgaccgcgagcagtggccccggaagttcttcaaccggacctgcaagtgcaccgggaacttcgccggctacaactgcggcgggtgcaagttcggctggacggggcccgactgcaaccggaagaagcccgccatcctgcgccggaacatccactccctcaccgcccaggagcgcgagcagttcctgggcgcgctggacctcgccaagaagtccatccacccggactacgtcatcaccacccagcactggctgggcctgctcgggcccaacggcacgcagccccagatcgccaactgctccgtgtacgacttcttcgtgtggctgcactactacagcgtccgggacaccctgctcgggcccggccgcccgtacaaggccatcgacttctcccaccaggggcccgcgttcgtgacctggcaccggtaccacctcctgtggctcgagcgcgagctgcagcggctgaccggcaacgagagcttcgccctcccctactggaacttcgccacgggcaagaacgagtgcgacgtgtgcaccgacgagctgctgggggccgcccgccaggacgaccccaccctgatctcccggaacagccgcttctccacctgggagatcgtctgcgacagcctcgacgactacaaccggcgcgtgacgctgtgcaacggcacctacgaggggctgctccgccggaacaaggtgggccgcaacaacgagaagctgccgaccctgaagaacgtccaggactgcctctccctgcagaagttcgacagcccgcccttcttccagaactccaccttcagcttccggaacgcgctggaggggttcgacaaggccgacggcacgctcgactcccaggtgatgaacctgcacaacctggcccacagcttcctcaacggcaccaacgccctgccccactccgccgcgaacgacccggtgttcgtcgtgctgcacagcttcaccgacgccatcttcgacgagtggctcaagcgcaacaacccctccaccgacgcctggccccaggagctggcccccatcgggcacaaccggatgtacaacatggtgccgttcttcccgcccgtcacgaacgaggagctgttcctgaccgccgagcagctcggctacaactacgcggtggacctgagcgaggaggaggcccccgtgtggagcaccaccctgtccggcgggggcgggagcggcgggggcggctccttcctcctgtggatcctggccgccgtctccagcggcctcttcttctactccttcctgctgaccgcggtgagcctctccaagatgctgaagaagcgcagccccctgacgaccggggtgtacgtcaagatgccgcccaccgagcccgagtgcgagaagcagttccagccctacttcatccccatcaactgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

SEQ ID NO: 544 - RNA, 1779 nt, synthetic construct
  misc_feature 1779^1: note = spliced circRNA-17
  source 1..1779: mol_type = other RNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatggcctgcctgggcttccagcgccacaaggcccagctcaacctggcgacccggacctggccctgcacgctcctgttcttcctgctgttcatcccggtgttctgcctgtccgccctcctggccgtgggcgcgctggaggggccccgcaaccaggactggctcggcgtcccgcggcagctggtgaccaagaccaacggcacccagccccagatcgccaactgcagcgtgtacgacttcttcgtctggctgcactactactccgtgcgcgacaccctcctgaaggacagcggctcccccttcccggccgccgtgatcctgcgcgacgcgctccacatggcccgggggctgaagtacctgcaccaggagcccccggtgaccaacaccgagatgttcgtcacggcccccgacaacctgggctacatgtacgaggtgcagtggcccgggcaggagttcatgcgcgagggcgtggagctgtgccccgggaacaagtacgagatgcggcgccacggcaccacccacagcctcgtcatccacgacgtggccaagttcgtggccgcgtggaccctgaaggccgccgccttcctgctctggatcctggccgccgtgagctccggcctgttcttctacagcttcctcctgaccgcggtctccctgagcaagatgctcaagaagcgcagccccctgaccacgggggtgtacgtgaagatgccgcccaccgagcccgagtgcgagaagcagttccagccctacttcatcccgatcaactgatccctgttcccagagcccacttttttttctttttttgaaataaaatagcctgtctttcggatccaaaaaaaaaaaaaaaaaaaaaaaaaaaaaaaaaaaactcgagcggctattatgcgttaccggcgagacgctacggactt

(19) United States
(12) Patent Application Publication
(10) Pub. No.: US 2025/0027108 A1
(43) Pub. Date: Jan. 23, 2025

(54) IMPROVED CIRCULAR RNA FOR EXPRESSING THERAPEUTIC PROTEINS

(71) Applicant: CureVac SE, Tübingen (DE)

(72) Inventor: Dipankar BHANDARI, Tübingen (DE)

(73) Assignee: CureVac SE, Tübingen (DE)

(21) Appl. No.: 18/705,499

(22) PCT Filed: Oct. 31, 2022
(86) PCT No.: PCT/EP2022/080358

§ 371 Date: Apr. 26, 2024

(30) Foreign Application Priority Data
Oct. 29, 2021 (WO) PCT/EP2021/080187

(51) Int. Cl. C12N 15/85; A61K 39/00; A61K 39/35; A61K 48/00; C07H 1/06; C07H 21/02; C12N 15/64; C12N 15/88

(52) U.S. Cl. CPC C12N 15/85; A61K 39/0011; A61K 39/35; A61K 48/0033; A61K 48/0041; A61K 48/0066; C07H 1/06; C07H 21/02; C12N 15/64; C12N 15/88; A61K 2039/53; A61K 2039/6018; A61K 2039/6093; C12N 2830/50

(57) ABSTRACT

The present invention is inter alia directed to improved circular RNA constructs comprising (i) at least one translation initiation sequence, (ii) at least one coding sequence (cds), (iii) at least one UTR sequence, and (iv) at least one poly(A) sequence. Further provided is linear precursor RNA for making such an improved circular RNA. The invention also relates to improved methods for preparing circular RNA and to improved methods of purifying circular RNA. Moreover, the invention relates to pharmaceutical compositions, vaccines, combinations, and kit or kit of parts comprising the circular RNA of the invention. Also provided are methods of treating or preventing disorders or diseases, and first, second, and further medical uses.

### INTRODUCTION

The present invention is interalia directed to improved circular RNA constructs comprising (i) at least one translation initiation sequence, (ii) at least one coding sequence (cds), (iii) at least one UTR sequence, and (iv) at least one poly(A) sequence. Further provided is linear precursor RNA for making such an improved circular RNA. The invention also relates to improved methods for preparing circular RNA and to improved methods of purifying circular RNA. Moreover, the invention relates to pharmaceutical compositions, vaccines, combinations, and kit or kit of parts comprising the circular RNA of the invention. Also provided are methods of treating or preventing disorders or diseases, and first, second, and further medical uses.

Therapeutic RNA molecules represent an emerging class of drugs. RNA-based therapeutics include RNA molecules encoding antigens for use as vaccines. In addition, it is envisioned to use RNA molecules for replacement therapies, e.g. providing missing proteins such as growth factors or enzymes to patients. Accordingly, RNA-based therapeutics with the use in immunotherapy, gene therapy, and vaccination belong to the most promising and quickly developing therapeutics in modem medicine.

Typically, RNA molecules encoding antigens or therapeutic proteins are provided as linear mRNA constructs. However, one fundamental limitation to the use of linear mRNA is interalia its relatively short half-life in biological systems, and the potential immunostimulation induced by linear mRNA molecules.

Circular RNA may be useful in the design and production of more stable forms of therapeutic RNA. In circular RNA molecules, the 5′ and 3′ ends are joined together and may therefor display certain advantageous properties. Various examples of protein coding circular RNA molecules exist in the art, but as yet circular RNA technology is still in an early developmental stage.

To exploit the full therapeutic potential of circular RNA, further optimizations are needed on sequence level to interalla reduce unwanted immunostimulation, to increase the duration of protein expression, or to further extend the half-life. In addition, advances on manufacturing level are needed (e.g. purification of circular RNA) to ensure high-quality manufacturing of circular RNA therapeutics to enable an industrial scale production.

Thus, the underlying object of the invention is to provide optimized circular RNA molecules and manufacturing methods to obtain optimized circular RNA molecules.

The objects mentioned above are solved by the underlying description and the accompanying claims.

### Short Description of the Invention

In experiments underlying the invention, the inventors discovered that certain elements e.g. certain Poly(A), Kozak and UTR sequences are advantageous in the context of circular RNA (see Example section).

Furthermore, the inventors developed methods for manufacturing and purifying circular RNA at high quality. These methods can be used to generate circular RNA with high purity levels, e.g. circular RNA preparation suitable for industrial scale manufacturing (see Example section).

In a first aspect, the invention provides circular RNA comprising at least one coding sequence and at least one translation initiation sequence.

In particular, the circular RNA comprises
(i) at least one translation initiation sequence;
(ii) at least one coding sequence (cds);
(iii) at least one UTR sequence; and
(iv) at least one poly(A) sequence.

In preferred embodiments, the circular RNA comprises the following sequence elements in the following order
(i) at least one translation initiation sequence;
(ii) at least one coding sequence (cds);
(iii) at least one UTR sequence; and
(iv) at least one poly(A) sequence.

The circular RNA may comprise at least one poly(A) sequence comprising about 30 to about 200 consecutive adenosine nucleotides, e.g. about 60 consecutive adenosine nucleotides.

Suitably, the at least one UTR comprises or consists of a nucleic acid sequence derived from a 3′-UTR of a gene selected from PSMB3, or from a homolog, a fragment or a variant of that gene.

The circular RNA may comprise at least one codon modified coding sequence which is suitably a G/C optimized coding sequence, a human codon usage adapted coding sequence, or a G/C content modified coding sequence.

In a second aspect, the invention provides a linear precursor RNA for making a circular RNA, said linear precursor RNA comprising the following elements operably connected to each other and arranged in the following sequence, preferably in the following order:
a 3′ permuted intron-exon element;
at least one translation initiation sequence;
at least one coding sequence;
at least one UTR sequence;
at least one poly(A) sequence; and
a 5′ permuted intron-exon element.

In preferred embodiments, the linear precursor RNA is for making a circular RNA of the first aspect. In a third aspect, the invention relates to a pharmaceutical composition comprising the circular RNA as defined in the first aspect.

Suitably, the circular RNA of the pharmaceutical composition is formulated in lipid-based carriers, preferably wherein the lipid-based carriers encapsulate the circular RNA.

The pharmaceutical composition may additionally comprise at least one linear 5′ capped messenger RNA comprising at least one coding sequence encoding a peptide or protein.

Suitably, the pharmaceutical composition is a vaccine.

In a fourth aspect, the invention relates to a combination comprising (A) at least one circular RNA of the invention and (B) at least one linear coding RNA, e.g. a linear 5′ capped messenger RNA.

In an fifth aspect, the invention relates a kit or kit of parts, comprising at least one circular RNA as defined herein, and/or at least one pharmaceutical composition as defined herein.

In further aspects, the invention relates to the medical use and further medical uses of the circular RNA as defined herein, and/or the pharmaceutical composition as defined herein, and/or the combination as defined herein, and/or the kit or kit of parts as defined herein.

In further aspects, the invention provides a method of treating or preventing a disease, disorder or condition, wherein the method comprises applying or administering to a subject in need thereof the circular RNA as defined herein, and/or the pharmaceutical composition as defined herein, and/or the combination as defined herein, and/or the kit or kit of parts as defined herein.

In a further aspect, the invention relates to a method for preparing circular RNA comprising the steps of
A) providing a linear precursor RNA;
B) Incubating the precursor RNA in a buffer to allow circularization of the RNA; and
C) Obtaining the circular RNA product.

In a further aspect, the invention relates to a method of purifying a circular RNA from a preparation comprising non-circularized precursor RNA and circular RNA comprising a step of affinity-based removal of linear precursor RNA e.g. using a specific antisense Oligo and obtaining a preparation comprising purified circular RNA.

In a further aspect, the invention relates to a method of purifying a circular RNA from a preparation comprising non-circularized precursor RNA and circular RNA comprising a step of affinity-based capturing of circular RNA e.g. using a specific antisense Oligo, and obtaining a preparation comprising purified circular RNA.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as 15 percentages by weight (wt-%).

About The term “about” is used when determinants or values do not need to be identical, i.e. 100% the same. Accordingly, “about” means, that a determinant or values may diverge by 1% to 20%, preferably by 1% to 10%; in particular, by 1%, 2%, 3%, 4%, 5%, 6%, 7%, 8%, 9%, 10%, 11%, 12%, 13%, 14%, 15%, 16%, 17%, 18%, 19%, 20%. The skilled person knows that e.g. certain parameters or determinants can slightly vary based on the method how the parameter has been determined. For example, if a certain determinants or value is defined herein to have e.g. a length of “about 100 adenosine nucleotides”, the length may diverge by 1% to 20%. Accordingly, the skilled person knows that in that specific example, the length may diverge by 1 to 20 nucleotides. Accordingly, a length of “about 100 adenosine nucleotides” may encompass sequences ranging from exactly 80 to exactly 120 adenosine nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells).

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens. An antigen comprises may be or may comprises at least one mutation, insertion, deletion, or polymorphism.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic[immunogenic peptide or protein comprises at least one epitope (as defined herein) or antigen (as defined herein) of the protein it is derived from.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”. The term “permanently cationic” means, e.g., that the respective compound, or group, or atom, is positively charged at any pH value or hydrogen ion activity of its environment. Typically, the positive charge results from the presence of a quatemary nitrogen atom.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be an RNA sequence consisting of a number of nucleotides that may be divided by three, which starts with a start codon and which preferably terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment typically consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. a virus protein). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of proteins or peptides may comprise at least one epitope of those proteins or peptides.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, Immunogen: The terms “immunogen” or “immunogenic” will be recognized and understood by the person of ordinary skill in the art, and are e.g. intended to refer to a compound that is able to stimulate/induce an (adaptive) immune response. An immunogen may be a peptide, polypeptide, or protein. An immunogen may be the product of translation of a provided circular RNA.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Nucleic acid, nucleic acid molecule: The terms “nucleic acid” or “nucleic acid molecule” as used herein, will be recognized and understood by the person of ordinary skill in the art. The terms “nucleic acid” or “nucleic acid molecule” preferably refers to DNA (molecules) or RNA (molecules). The term is used synonymously with the term polynucleotide. Preferably, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Nucleic acid species, DNA species, RNA species: In the context of the invention, the term “nucleic acid species”, “DNA species”, “RNA species” is not restricted to mean one single molecule but is understood to comprise an ensemble of essentially identical nucleic acid, DNA or RNA molecules. Accordingly, it may relate to a plurality of essentially identical nucleic acid molecules, e.g. DNA or RNA molecules.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is typically formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. In general, RNA can be obtained by transcription of a DNA sequence, e.g., inside a cell or in vitro. In the context of the invention, the circular RNA, the linear precursor RNA, or the linear capped mRNA may be obtained by RNA in vitro transcription. Alternatively, RNA may be obtained by chemical synthesis.

RNA in vitro transcription: The terms “RNA in vitro transcription” or “in vitro transcription” relate to a process wherein RNA is synthesized in a cell-free system (in vitro). RNA may be obtained by DNA-dependent in vitro transcription of an appropriate DNA template, which is typically a linear DNA template (e.g. linearized plasmid DNA or PCR product). The promoter for controlling RNA in vitro transcription can be any promoter for any DNA-dependent RNA polymerase (e.g. T7, SP6, T3). Reagents used in RNA in vitro transcription typically include a DNA template, ribonucleotide triphosphates, a cap analog, a DNA-dependent RNA polymerase, a ribonuclease (RNase) inhibitor, MgCl₂, a buffer (e.g. TRIS or HEPES) which can also contain antioxidants, and/or polyamines such as spermidine at optimal concentrations.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid 35 sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A‘variant’ of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

### DETAILED DESCRIPTION OF THE INVENTION

Where reference is made to “SEQ ID NOs” of other patent applications or patents, said sequences, e.g. amino acid sequences or nucleic acid sequences, are explicitly incorporated herein by reference. For “SEQ ID NOs” provided herein, information under identifier <223>(in the sequence protocol) is also explicitly included herein in its entirety. Where reference is made to “SEQ ID NOs” in the context of RNA sequences, the skilled person will understand and be able to derive RNA sequences from the referenced SEQ ID NOs also in cases where DNA sequences are provided. Where reference is made to “SEQ ID NOs” in the context of DNA sequences, the skilled person will understand and will be able to derive respective DNA sequences from the referenced SEQ ID NOs also in cases where RNA sequences are provided.

The present application is filed together with a sequence listing in electronic format, which is part of the description of the present application (WIPO standard ST.26). The information contained in the sequence listing is incorporated herein by reference in its entirety. Where reference is made herein to a “SEQ ID NO”, the corresponding nucleic acid sequence or amino acid (aa) sequence in the sequence listing having the respective identifier is referred to. For many sequences, the sequence listing also provides additional detailed information, e.g. regarding certain structural features, sequence optimizations, GenBank (NCBI) or GISAID (epi) identifiers, or additional detailed information regarding its coding capacity. In particular, such information on the specific sequences is provided under “feature key”, i.e. “source” (for nucleic acids or proteins) or “misc_feature” (for nucleic acids) or “REGION” (for proteins).

### 1: Circular RNA for Expressing Therapeutic Proteins

In a first aspect, the invention provides circular RNA for expressing a therapeutic protein.

The term “circular RNA” has to be understood as an RNA molecule that does not comprise a 5′ and a 3′ terminus that are typically present in linear RNA molecules (e.g. mRNA). In contrast to mRNA, a circular RNA does not comprise a 5′ Cap structure or a 3′ terminal tail. In circular RNA, the 3′ and 5′ ends that are normally present in a linear RNA molecule are joined together. Accordingly, a circular RNA can be considered as an RNA having a closed continuous loop. In the context of the invention, the circular RNA for expressing a therapeutic protein comprises at least one coding sequence.

Notably, whenever certain features of the circular RNA relate to the 5′ location, a position upstream of the coding sequence is referred to, wherein “upstream” has to be understood as “in the opposing direction of translation”. Likewise, whenever certain features of the circular RNA relate to the 3′ location (e.g. 3′ UTR), a position downstream of the coding sequence is referred to, wherein “downstream” has to be understood as “in the direction of translation”. Whenever an element of the circular RNA is described in relation to another element, the relation to each other has to be understood as the relation to each other in the direction of translation. For example, if an “element X” is defined as being located between an “element Y” and an “element Z”, that has to be interpreted in the direction of translation, e.g. the order of the elements would be X, Y, Z in the direction of translation.

In particularly preferred embodiments, the circular RNA comprises at least one coding sequence and at least one translation initiation sequence. The translation of the at least one coding sequence is driven by said translation initiation sequence. Further elements that may be comprised in the circular RNA of the invention are selected from but not limited to at least one UTR sequence, at least one Kozak sequence, at least one poly(A) sequence, at least one poly(C) sequence, at least one histone stem loop (hsl).

In preferred embodiments, the circular RNA of the invention is an artificial circular RNA.

The term “artificial circular RNA” as used herein is intended to refer to a circular RNA that does not occur naturally. In other words, an artificial circular RNA may be understood as a non-natural RNA molecule. Such RNA molecules may be non-natural due to its individual sequence (e.g. G/C content modified coding sequence, UTRs) and/or due to other modifications, e.g. structural modifications of nucleotides. Typically, artificial circular RNA may be designed and/or generated by genetic engineering to correspond to a desired artificial sequence of nucleotides. In this context, an artificial circular RNA is a sequence that may not occur naturally, i.e. a sequence that differs from the wild type sequence/the naturally occurring sequence by at least one nucleotide (via e.g. codon modification as further specified below), or by a structural features such as its circularity. The term “artificial circular RNA” is not restricted to mean “one single molecule” but is understood to comprise an ensemble of essentially identical RNA molecules. Accordingly, the term may relate to a plurality of essentially identical circular RNA molecules.

In preferred embodiments, the circular RNA of the invention comprises
(i) at least one translation initiation sequence;
(ii) at least one coding sequence (cds);
(iii) at least one UTR sequence; and
(iv) at least one poly(A) sequence.

In preferred embodiments, the circular RNA of the invention comprises, preferably following sequence elements in the following order
(i) at least one translation initiation sequence;
(ii) at least one coding sequence (cds);
(iii) at least one UTR sequence; and
(iv) at least one poly(A) sequence.

In various embodiments, the circular RNA of the invention comprises at least one poly(N) sequence, e.g. at least one poly(A) sequence, at least one poly(U) sequence, at least one poly(C) sequence, or combinations thereof.

In preferred embodiments, the circular RNA comprises at least one poly(A) sequence.

The terms “poly(A) sequence” as used herein will be recognized and understood by the person of ordinary skill in the art, and is e.g. intended to be a sequence of adenosine nucleotides, typically comprising about 30 to up to about 500 adenosine nucleotides. Preferably, said poly(A) sequence is essentially homopolymeric, e.g. a poly(A) sequence of e.g. 100 adenosine nucleotides has essentially the length of 100 nucleotides. A poly(A)sequence in the context of linear mRNA molecules is typically located close or at the 3′ terminal region (often called Poly(A)tail). A poly(A)sequence in the context of circular RNA can be located at any position in the RNA circle.

In linear mRNA molecules, the 3′ Poly(A) sequence typically protects the mRNA from 3′ terminal degradation and also plays an important role in Cap-dependent protein translation. Typically, poly A binding proteins (PABP) recruit translation initiation factors (e.g. eIF4F) to interact with eIF4E that is recruited by the 5′ cap structure of the mRNA. Accordingly, the mRNA forms a loop structure by bridging the cap to the poly(A) tail via the cap-binding protein eIF4E and the PABP, both of which interact with eIF4G. That so called translation initiation complex is thought to promote an efficient translation. Due to the lack of a 5′ cap structure in circular RNA molecules, it is unknown whether poly(A) sequences can have an impact on protein translation and/or RNA stability.

The inventors surprisingly found that the insertion of at least one poly(A)sequence element into circular RNA molecules have certain advantageous effects including but not limited to e.g. an increase of protein translation, and/or a prolongation of protein translation and/or a reduction of unwanted immunostimulation (reactogenicity), and/or an increase in the half-life of the circular RNA.

In preferred embodiments, the at least one poly(A) sequence of the circular RNA comprises at least about 30 consecutive adenosine nucleotides, at least about 40 consecutive adenosine nucleotides, at least about 50 consecutive adenosine nucleotides, at least about 60 consecutive adenosine nucleotides, at least about 70 consecutive adenosine nucleotides, at least about 80 consecutive adenosine nucleotides, at least about 90 consecutive adenosine nucleotides, at least about 100 consecutive adenosine nucleotides. In these embodiments, each upper limit is defined as not more than 300 consecutive adenosine nucleotides.

In preferred embodiments, the at least one poly(A) sequence of the circular RNA comprises about 30 to about 150 adenosine nucleotides, preferably about 30 to about 150 consecutive adenosine nucleotides. In preferred embodiments, the at least one poly(A) sequence of the circular RNA comprises about 40 to about 150 adenosine nucleotides, preferably about 40 to about 150 consecutive adenosine nucleotides. In preferred embodiments, the at least one poly(A) sequence of the circular RNA comprises about 50 to about 150 adenosine nucleotides, preferably about 50 to about 150 consecutive adenosine nucleotides.

In preferred embodiments, the at least one poly(A) sequence of the circular RNA comprises about 50 to about 140 adenosine nucleotides, preferably about 50 to about 140 consecutive adenosine nucleotides.

In preferred embodiments, the at least one poly(A) sequence of the circular RNA comprises about 50 to about 130 adenosine nucleotides, preferably about 50 to about 130 consecutive adenosine nucleotides.

In preferred embodiments, the at least one poly(A) sequence of the circular RNA comprises about 50 to about 120 adenosine nucleotides, preferably about 50 to about 120 consecutive adenosine nucleotides.

In preferred embodiments, the at least one poly(A) sequence of the circular RNA comprises about 50 to about 110 adenosine nucleotides, preferably about 50 to about 110 consecutive adenosine nucleotides.

In preferred embodiments, the at least one poly(A) sequence of the circular RNA comprises about 50 to about 100 adenosine nucleotides, preferably about 50 to about 100 consecutive adenosine nucleotides.

In particularly preferred embodiments, the at least one poly(A) sequence comprises about 40 to about 150 consecutive adenosine nucleotides, preferably about 40 to about 120 consecutive adenosine nucleotides, more preferably about 40 to about 100 consecutive adenosine.

In particularly preferred embodiments, the at least one poly(A) sequence comprises about 30 to about 150 consecutive adenosine nucleotides, preferably about 30 to about 120 consecutive adenosine nucleotides, more preferably about 30 to about 100 consecutive adenosine.

Accordingly, the at least one poly(A) sequence of the circular RNA comprises about or more than 30, 35, 36, 40, 45, 50, 55, 60, 65, 70, 75, 80, 85, 90, 95,100, 105, 110, 115, 120, 125, 130, 135, 140, 145, 150, 155,160, 165, 170, 175, 180, 185, 190,195, 200, 205, 210, 215, 220, 225, 230, 235, 240, 245, 250, 255, 260, 265, 270, 275, 280, 285, 290, 295, or about or more than 300 adenosine nucleotides, preferably about or more than 30, 35, 36, 40, 45, 50, 55, 60, 65, 70, 75, 80, 85, 90, 95, 100, 105, 110, 115, 120, 125, 130, 135, 140, 145, 150,155, 160, 165, 170, 175, 180, 185,190, 195, 200, 205, 210, 215, 220, 225, 230, 235, 240, 245, 250, 255, 260, 265, 270, 275, 280, 285, 290, 295, or about or more than 300 consecutive adenosine nucleotides.

Preferably, the at least one poly(A) sequence of the circular RNA comprises about 30, 35, 36, 40, 45, 50, 55, 60, 65, 70, 75, 80, 85, 90, 95, or about 100 consecutive adenosine nucleotides.

In particularly preferred and specific embodiments, the at least one poly(A) sequence of the circular RNA comprises 36 consecutive adenosine nucleotides. In other particularly preferred and specific embodiments, the at least one poly(A) sequence of the circular RNA comprises 60 consecutive adenosine nucleotides.

In particularly preferred embodiments, the at least one poly(A) sequence of the circular RNA comprises about (that is, +/−20%) 60 consecutive adenosine nucleotides (e.g. 48, 49, 50, 51, 52, 53, 54, 55, 56, 57, 58, 59, 60, 61, 62, 63, 64, 65, 66, 67, 68, 69, 70, 71, or 72 consecutive adenosine nucleotides).

In particularly preferred embodiments, the at least one poly(A) sequence of the circular RNA comprises about (that is, +/−20%) 35 consecutive adenosine nucleotides (e.g. 28, 29, 30, 31, 32, 33, 34, 35, 36, 37, 38, 39, 40, 41 or 42 consecutive adenosine nucleotides).

In preferred embodiments, the circular RNA comprises at least poly(A)sequences, wherein the at least one poly(A)sequences comprises a nucleic acid sequence derived or selected from nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 193 to 195, or a fragment or a variant of any of these.

In preferred embodiments, the circular RNA comprises at least two, three, or more poly(A) sequences. These two, three, or more poly(A) sequences may be located at different positions in the circular RNA molecule.

Accordingly, the circular RNA may comprise 2, 3, 4, 5, 6, 7, 8, or more poly(A) sequences as defined herein, preferably wherein each of the 2, 3, 4, 5, 6, 7, 8, or more poly(A) sequences comprise about 30 to about 150 adenosine nucleotides.

In some embodiments, the poly(A) sequence of the circular RNA may be interrupted by at least one nucleotide that is different from an adenosine nucleotide, e.g. a poly(A) sequence of e.g. 100 adenosine nucleotides may 30 have a length of more than 100 nucleotides (comprising 100 adenosine nucleotides and in addition said at least one nucleotide—or a stretch of nucleotides—different from an adenosine nucleotide). For example, the poly(A) sequence may comprise about 100 A nucleotides being interrupted by at least one nucleotide different from A (e.g. a linker (L), typically about 2 to 20 nucleotides in length), e.g. A30-L-A70 or A70-L-A30.

In embodiments, a suitable linker (L) sequence located between at least two poly(A)sequences is derived from a restriction endonudease recognition site, e.g. a restriction endonuclease recognition site (RERS). For example, the linker (L) sequence located between at least two poly(A)sequences may be derived from a Nodl restriction endonudease recognition site, for example: about A30-RERS—about A30; or about A60-RERS—about A60.

In preferred embodiments, the at least one poly(A) sequence of the circular RNA is located downstream of the coding sequence (that is in particular downstream of the stop codon) in the direction of translation.

In preferred embodiments, at least one poly(A) sequence of the circular RNA is located downstream of the UTR in the direction of translation.

In preferred embodiments, at least one poly(A) sequence of the circular RNA is located upstream (in opposite direction of translation) of the translation initiation sequence.

Accordingly, the at least one poly(A) sequence is located between coding sequence (or UTR) and translation initiation sequence. “Located between” in that context has to be understood as in the direction of translation of the coding sequence.

In preferred embodiments, the distance between the at least one poly(A) sequence as defined herein and the translation initiation sequence as defined herein is less than about 200 nucleotides, preferably less than about 160 nucleotides. In preferred embodiments, the distance between the at least one poly(A) sequence as defined herein and the translation initiation sequence as defined herein is between 200 and 100 nucleotides.

In preferred embodiments, at least one poly(A) sequence of the circular RNA is located downstream of the UTR in the direction of translation and between the UTR and the translation initiation sequence, wherein said at least one poly(A) sequence comprises about 50 to about 150 consecutive adenosine nucleotides, wherein the distance between the at least one poly(A) sequence and the translation initiation sequence as defined herein is less than about 200 nucleotides, preferably less than about 160 nucleotides.

In preferred embodiments, the circular RNA of the invention comprises at least one untranslated region (UTR).

Surprisingly, including at least one UTR sequence derived from linear mRNA molecules was also beneficial in the context of circular RNA of the invention.

The terms “untranslated region” or “UTR” or “UTR element” are intended to refer to a part of an RNA typically located 5′ (“upstream”) or 3′ (“downstream”) of a coding sequence. An UTR is not translated into protein. An UTR typically comprises elements for controlling gene expression, also called regulatory elements. Such regulatory elements may be, e.g., ribosomal binding sites, miRNA binding sites, promotor elements etc. UTRs may harbor regulatory sequence elements that determine RNA turnover, stability, and localization. Moreover, UTRs may harbor sequence elements that enhance translation. Circular RNA harboring said UTRs advantageously enable rapid and transient expression of peptides or proteins after administration to a subject.

In embodiments, the circular RNA comprises at least one UTR located “downstream” of the coding sequence and, optionally, at least one further UTR located “upstream” of the coding sequence.

UTRs may be derived from naturally occurring genes or may be synthetically engineered.

In preferred embodiments, the circular RNA comprises at least one UTR “downstream” of the coding sequence (in a linear mRNA, such an UTR would correspond to a 3′ UTR).

The term “3′-untranslated region” or “3′-UTR” or “3′-UTR element” are intended to refer to a part of an RNA molecule located 3′ (i.e. downstream of the direction of translation) of a coding sequence and which is not translated into protein. Such an UTR may be part of the circular RNA, located between a coding sequence and the poly(A) sequence (“Located between” in that context has to be understood as in the direction of translation of the coding sequence). An UTR “downstream” of the coding sequence typically comprises elements for controlling gene expression, also called regulatory elements. Such regulatory elements may be, e.g., ribosomal binding sites, miRNA binding sites etc. Usually, 3′-UTR sequences are known to regulate translation and/or stability in linear mRNA molecules. The role of UTR sequences “downstream” of the coding sequence (corresponding to 3′-UTR sequences in linear mRNA molecules) is to be explored in the context of circular RNA.

In various embodiments, the circular RNA of the invention comprises at least one UTR, wherein the at least one 20 UTR comprises a nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-168, or a fragment or a variant of any of these.

In preferred embodiments, the at least one UTR comprises or consists of an RNA sequence derived from a 3′-UTR of a gene selected from PSMB3, ALB7, alpha-globin, CASP1, COX6B1, GNAS, NDUFA1, AES-12S and RPS9, or from a homolog, a fragment or a variant of any one of these genes.

In preferred embodiments, the circular RNA of the invention may comprise at least one UTR, wherein the at least one UTR comprises a nucleic acid sequence that is derived or selected from a 3′-UTR of a gene selected from PSMB3, ALB7, alpha-globin (referred to as “muag”), CASP1, COX6B1, GNAS, NDUFA1. AES-12S and RPS9, or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 95-118, or 192 or a fragment or a variant of any of these.

In preferred embodiments, the at least one UTR of the circular RNA comprises or consists of an RNA sequence derived from a 5′-UTR of a gene selected from HSD17B4, RPL32, ASAH1, ATP5A1, MP68, NDUFA4, NOSIP, RPL31, SLC7A3, TUBB4B, HBA1, HBA2 and UBQLN2, or from a homolog, a fragment or variant of any one of these genes.

In other preferred embodiments, the circular RNA comprises at least one UTR, wherein the at least one UTR comprises a nucleic acid sequence that is derived or selected from a 5′-UTR of gene selected from HSD17B4, RPL32, ASAH1, ATP5A1, MP68, NDUFA4, NOSIP, RPL31, SLC7A3, TUBB4B, HBA1, HBA2 and UBQLN2, or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-32, or a fragment or a variant of any of these.

In other embodiments, the circular RNA of the invention may comprise at least one UTR, wherein the at least one UTR comprises a nucleic acid sequence that is derived or selected from nucleic acid sequences being identical or at least 70%, 80%, 85%, 86%, 87%, 88%, 89%, 90%, 91%, 92%, 93%, 94%, 95%, 96%, 97%, 98%, 15 or 99% identical to any one of SEQ ID NOs: 33-94, 119-168 or a fragment or a variant of any of these.

In particularly preferred embodiments, the at least one UTR of the circular RNA comprises or consists of a RNA sequence derived from a 3′-UTR of a gene selected from PSMB3, or from a homolog, a fragment or a variant of that gene. Said UTR derived from a PSMB3 gene may comprise or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 96,136, or 192, or a fragment or a variant of any of these.

Particularly preferred in the context of the invention is an RNA sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 192, or a fragment or a variant thereof. Preferably said at least one UTR is located “downstream” of the coding sequence.

In particularly preferred embodiments, the at least one UTR of the circular RNA comprises or consists of a RNA sequence derived from a 3′-UTR of a gene selected from RPS9, or from a homolog, a fragment or a variant of that gene. Said UTR derived from a RPS9 gene may comprise or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 114 or 158, or a fragment or a variant of any of these. In preferred embodiments, the at least one UTR comprises or consists of an RNA sequence that has a length of less than about 200 nucleotides, preferably less than about 100 nucleotides, e.g. between about 30 nucleotides and about 100 nucleotides, Preferably, the UTR with the specified length is a downstream UTR as defined herein.

Suitably, the at least one UTR consists of an RNA sequence that has a length of between about 50 nucleotides and about 200 nucleotides, between about 70 nucleotides and about 200 nucleotides, between about 60 nucleotides and about 100 nucleotides, between about 70 nucleotides and about 100 nucleotides, between about 70 nucleotides and about 90 nucleotides, between about 70 nucleotides and about 85 nucleotides.

Preferably, the UTR with the specified length is a downstream UTR as defined herein.

In preferred embodiments, the at least one UTR consists of an RNA sequence that has a length of at least about 50 nucleotides and less than about 200 nucleotides. In another preferred embodiment, the at least one UTR consists of an RNA sequence that has a length of at least about 50 nucleotides and less than about 100 nucleotides. Preferably, the UTR with the specified length is a downstream UTR as defined herein. 10 In particularly preferred embodiments, the at least one UTR has a length of less than about 200 nucleotides, less than about 150 nucleotides, less than about 100 nucleotides. In particularly preferred embodiments, the at least one UTR has a length between about 50 and about 200 nucleotides, between about 70 and about 200 nucleotides, between about 70 and about 100 nucleotides. Preferably, the UTR with the specified length is a downstream UTR as defined herein.

Preferably said at least one UTR is located “downstream” of the coding sequence.

In preferred embodiments, the at least one UTR sequence as defined herein is located downstream of the coding sequence (that is in particular downstream of the stop codon) in the direction of translation. Accordingly, such an UTR may be considered as a “downstream UTR”.

In preferred embodiments, the at least one UTR is located between the coding sequence (in particular the stop codon) and the at least one Poly(A) sequence. Suitably, the UTR is followed by at least one Poly(A) sequence. “Located between” in that context has to be understood as in the direction of translation of the coding sequence.

In preferred embodiments, the circular RNA comprises at least one upstream UTR sequence. The upstream UTR sequence may be derived from any UTR as defined herein.

Preferably, the upstream UTR sequence is derived from a 5′-UTR of a gene selected from HSD17B4, RPL32, ASAH1, ATP5A1, MP68, NDUFA4, NOSIP, RPL31, SLC7A3, TUBB4B, HBA1, HBA2 and UBQLN2 as defined herein, or from a 3′-UTR of a gene selected from PSMB3, ALB7, alpha-globin, CASP1, COX6B1, GNAS, NDUFA1, AES-12S and RPS9, or from a homolog, a fragment or a variant of any one of these genes.

In embodiments, the circular RNA comprises at least one upstream UTR and at least one downstream UTR. (e.g. upstream UTR—coding sequence—downstream UTR).

As used herein, the term upstream UTR sequence relates to an UTR that is located upstream of the coding sequence, in particular between translation initiation sequence and coding sequence.

In preferred embodiments, the at least one upstream UTR sequence of the circular RNA is located upstream of the coding sequence in the opposing direction of translation.

In preferred embodiments, the at least one upstream UTR sequence of the circular RNA is located between the translation initiation sequence as defined herein and the coding sequence as defined herein.

In preferred embodiments, the at least one upstream UTR as defined herein comprises or consists of a nucleic acid sequence according to nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-168, or a fragment or a variant of any of these.

In preferred embodiments, the at least one upstream UTR comprises or consists of a nucleic acid sequence that has a length of less than about 200 nucleotides, preferably less than about 100 nucleotides, e.g. between about 30 nucleotides and about 100 nucleotides.

Accordingly, in embodiments, the circular RNA may comprise the following elements:
at least one translation initiation sequence;
optionally, at least one Kozak sequence (upstream of the cds);
optionally, at least one upstream UTR (upstream of the cds);
at least one coding sequence (cds);
at least one UTR sequence (downstream of the cds);
at least one poly(A) sequence.

In embodiments, the circular RNA of the pharmaceutical composition may be monocistronic, bicistronic, or multicistronic.

The term “monocistronic” will be recognized and understood by the person of ordinary skill in the art, and is e.g. intended to refer to an circular RNA that comprises only one coding sequence. The terms “bicistronic”, or “multicistronic” as used herein will be recognized and understood by the person of ordinary skill in the art, and are e.g. intended to refer to a circular RNA that may comprise two (bicistronic) or more (multicistronic) coding sequences.

In preferred embodiments, the circular RNA is monocistronic.

In alternative preferred embodiments, the circular RNA is bicistronic. In such embodiments, the bicistronic circular RNA comprises at least two coding sequences. In such embodiments it is preferred that each of the at least two coding sequences of the circular RNA are operably linked to a translation initiation sequence (e.g. an IRES).

In preferred embodiments, the at least one coding sequence of the circular RNA is a codon modified coding sequence.

Suitably, the amino acid sequence encoded by the at least one codon modified coding sequence of the circular RNA is not being modified compared to the amino acid sequence encoded by the corresponding wild type or reference coding sequence.

The term “codon modified coding sequence” relates to coding sequences that differ in at least one codon (triplets of nucleotides coding for one amino acid) compared to the corresponding wild type or reference coding sequence. In the context of linear mRNA, a codon modified coding sequence may show improved resistance to in vivo degradation and/or improved stability in vivo, and/or improved translatability in vivo and/or improved half-life in vivo. In the context of circular RNA of the invention, the role of codon modified coding sequence is to be explored and not yet understood. Codon modifications in the broadest sense make use of the degeneracy of the genetic code wherein multiple codons may encode the same amino acid and may be used interchangeably to optimize/modify the coding sequence of the circular RNA for in vivo applications.

In preferred embodiments, the at least one codon modified coding sequence of the circular RNA is selected from a C maximized coding sequence (according to WO2015/062738), a codon adaptation index (CAI) maximized coding sequence, a human codon usage adapted coding sequence, a G/C content modified coding sequence, and a G/C optimized coding sequence, or any combination thereof.

In preferred embodiments, the at least one codon modified coding sequence of the circular RNA is a G/C optimized coding sequence, a human codon usage adapted coding sequence, or a G/C content modified coding sequence, preferably a G/C optimized coding sequence.

In preferred embodiments, the circular RNA of the invention comprises a G/C optimized coding sequence, wherein the G/C content of the coding sequence is optimized compared to the G/C content of the corresponding wild type or reference coding sequence. “Optimized” in that context refers to a coding sequence wherein the G/C content is preferably increased to the essentially highest possible G/C content The amino acid sequence encoded by the G/C optimized coding sequence of the circular RNA is preferably not modified as compared to the amino acid sequence encoded by the respective wild type or reference coding sequence. The generation of a G/C content optimized coding sequences may be carried out using a method according to WO2002/098443.

In this context, the disclosure of WO2002/098443 is included in its full scope in the present invention. In the context of circular RNA, coding sequences having an increased G/C content may be more stable or may show a better expression than sequences having an increased A/U.

Suitably, the G/C content of the coding sequence of the circular RNA is increased by at least 10%, 20%, 30%, preferably by at least 40% compared to the G/C content of the corresponding wild type or reference coding sequence.

In various embodiments, the at least one coding sequence of the circular RNA has a G/C content of about 55% to about 80%, preferably of about 60% to about 80%, more preferably of about 65% to about 80%.

In preferred embodiments, the at least one coding sequence of the circular RNA has a G/C content of at least about 55%, 60%, or 65%.

In specific embodiments, the at least one coding sequence of the circular RNA has a G/C content of about 55%, 56%, 57%, 58%, 59%, 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%.

In preferred embodiments, the at least one coding sequence of the circular RNA has a G/C content that is about 5%, about 10%, about 15%, or about 20% increased compared to the corresponding wild type or reference coding sequence.

In preferred embodiments, the at least one coding sequence of the circular RNA is an motif optimized coding sequence, wherein certain motifs have been removed in the motif optimized coding sequence compared to the corresponding wild type or reference coding sequence. In preferred embodiment, the at least one coding sequence of the circular RNA is a TLR7/TLR8 optimized coding sequence, wherein TLR7 motifs or TLR8 motifs have been removedin the TLR7/TLR8 optimized coding sequence compared to the corresponding wild type or reference coding sequence.

In preferred embodiments, the at least one coding sequence comprises more than one stop codon to allow sufficient termination of translation. These more than one stop codons may be positioned in alternative reading frames.

In specific embodiments, the at least one coding sequence comprises one, two or three stop codons.

In preferred embodiments, the at least one coding sequence of the circular RNA encodes at least one peptide or protein suitable for use in treatment or prevention of a disease, disorder or condition.

Accordingly, the circular RNA may provide at least one coding sequence encoding a peptide or protein that is translated into a (functional) peptide or protein after administration (e.g. after administration to a subject, e.g. a human subject).

In preferred embodiments, the coding sequence of the circular RNA encodes at least one peptide or protein, wherein said at least one peptide or protein is selected or derived from a therapeutic peptide or protein.

In various embodiments, the length of the encoded at least one peptide or protein, e.g. the therapeutic peptide or protein, may be at least or greater than about 20, 50, 100,150, 200, 300, 400, 500, 600, 700, 800, 900, 1000, or 1500 amino acids.

In preferred embodiments, the at least one peptide or protein is selected or derived from an antibody, an intrabody, a receptor, a receptor agonist, a receptor antagonist, a binding protein, a CRISPR-associated endonuclease, a chaperone, a chimeric antigen receptor (CAR), a transporter protein, an ion channel, a membrane protein, a toxin, a secreted protein, a transcription factor, an enzyme, a peptide or protein hormone, a growth factor, a structural protein, a cytoplasmic protein, a cytoskeletal protein, an allergen, a tumor antigen, a neoantigen, a proto-oncogene, an oncogene, a tumor-suppressor gene, a mutated antigen, an antigen of a pathogen, or fragments, epitopes, variants, or combinations of any of these.

In particularly preferred embodiments, the at least one peptide or protein is selected or derived from an antigen of a pathogen.

In particularly preferred embodiments, the antigen of a pathogen, is selected or derived from a viral antigen, a bacterial antigen, a protozoan antigen, a fungal antigen, or fragments, variants, or combinations of any of these.

In particularly preferred embodiments, the at least one peptide or protein is selected or derived from an antigen of a tumor.

In particularly preferred embodiments, the antigen of a tumor, is selected or derived from a tumor antigen, a neoantigen, a proto-oncogene, an oncogene, a tumor-suppressor gene, a mutated antigen, or fragments, variants, or combinations of any of these.

In preferred embodiments, the at least one coding sequence of the circular RNA is selected or derived from a domain, fragment, an epitope, a region, or a portion of monovalent (monoepitopic) or polyvalent (poly or multiepitopic) of the antigen of a tumor, neoantigen, proto-oncogene, oncogene, tumor-suppressor gene and/or mutated antigen.

In preferred embodiments, the at least one coding sequence of the circular RNA encodes at least two peptide or proteins as defined herein, wherein said at least two peptide or proteins are separated by a self-cleaving peptide.

The term self-cleaving peptide, 2A self-cleaving peptides, or 2A peptides as used herein relates to a class of 18-22 aa-long peptides, which can induce ribosomal skipping during translation of a protein in a cell. These peptides may share a core sequence motif of DxExNPGP, and are typically found in a wide range of viral families. The members of 2A peptides are named after the virus in which they have been first described. For example, F2A, the first described 2A peptide, is derived from foot-and-mouth disease virus. The name “2A” itself comes from the gene numbering scheme of this virus.

Accordingly, the circular RNA may provide one coding sequence encoding two peptides or proteins separated by a self-cleaving peptide, that is translated into two (functional) peptide or protein after administration (e.g. after administration to a cell or subject, e.g. a human subject).

In preferred embodiments, the at least one coding sequence of the circular RNA encodes at least two peptide or proteins as defined herein, wherein said at least two peptide or proteins are separated by at least one linker, preferably a GS linker, e.g. GGGGS, SGGGG or any variant thereof.

In preferred embodiments, the circular RNA comprises at least one translation initiation sequence.

The term “translation initiation sequence” has to be understood as a sequence element that facilitates binding or recruitment of translation initiation factors and/or ribosomes to promote translation of the at least one coding sequence into a peptide or protein. Accordingly, the translation initiation sequence is capable of engaging a ribosome, preferably an eukaryotic ribosome. Suitably, the translation initiation sequence promotes translation of the at least one coding sequence of the circular RNA into protein upon administration to a cell (e.g. eukaryotic cell) or a subject (e.g. human subject). Recruitment and/or binding of translation initiation factors and/or ribosomes may be facilitated due to a specific sequence motif comprised in the translation initiation sequence and/or due to the secondary structure comprised in the translation initiation sequence.

In particularly preferred embodiments, the translation initiation sequence of the circular RNA is a cap-independent translation initiation element. Accordingly, the translation initiation sequence of the circular RNA facilitates translation initiation in the absence of a 5′ Cap structure.

In preferred embodiments, the translation initiation sequence comprises at least one secondary structure for recruitment of translation initiation factors and/or ribosomes and/or at least one sequence motif for recruitment translation initiation factors and/or of ribosomes.

The term “recruitment of ribosomes” in that context is intended to be understood as a direct binding of ribosomes to the translation initiation sequence or as an indirect engagement of ribosomes to the translation initiation sequence. Accordingly, the recruitment may involve the binding of translation initiation factors to the translation initiation sequence. For example, HCV-like internal ribosomal entry site (IRES) directly bind the 40S ribosomal subunit to position their initiator codons are located in ribosomal P-site without mRNA scanning. These IRESs typically use the eukaryotic initiation factors (eIFs) eIF2, eIF3, eIF5, and eIF5B, but typically do not require the factors eIF1, eIF1A, and the eIF4F complex. In contrast, picomavirus IRESs typically do not bind the 40S subunit directly, but are typically recruited through the eIF4G-binding site.

In preferred embodiments, the translation initiation sequence is selected from an internal ribosomal entry site (IRES), an aptamer, or a CITE element.

In preferred embodiments, the translation initiation sequence of the circular RNA may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 210-505,513, 514 or a fragment or a variant of any of these.

In embodiments, the circular RNA additionally comprises at least one Kozak sequence. Suitably, such a Kozak sequence is located directly upstream of the start codon (the initiation codon where translation starts) of the at least one coding sequence.

In various embodiments, the translation initiation sequence is selected from an IRES, and the circular RNA additionally comprises at least one Kozak sequence. (e.g. IRES-Kozak-cds). In preferred embodiments the at least one Kozak sequence is located downstream of the IRES sequence. In preferred embodiments the at least one Kozak sequence is located directly downstream of the IRES sequence. “Directly downstrem” in that specific context means that less than 5 spacer nucleotides (e.g. nucleotides that do not correspond to the Kozak or the IRES sequence) are located between Kozak sequence and IRES sequence.

In the context of the invention, the at least one IRES sequence and the at least one Kozak sequence of the circular RNA may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 518-520, or a fragment or a variant of any of these.

Accordingly, in embodiments, the circular RNA may comprise the following elements:
at least one translation initiation sequence (upstream of the cds);
at least one Kozak sequence (upstream of the cds);
at least one coding sequence (cds);
at least one UTR sequence (downstream of the cds);
at least one poly(A) sequence.

In various embodiments, the translation initiation sequence is selected from an aptamer, and the circular RNA additionally comprises at least one Kozak sequence. (e.g. aptamer-Kozak-cds or aptamer-UTR-Kozak-cds).

In various embodiments, the translation initiation sequence is selected from a CITE, and the circular RNA additionally comprises at least one Kozak sequence. (e.g. CITE-Kozak-cds or CITE-UTR-Kozak-cds).

In the context of the invention, the at least one Kozak sequence of the circular RNA may comprise or consist of a 5 nucleic acid sequence being identical or at least 70%, 80%, 85%, 86%, 87%, 88%, 89%, 90%, 91%, 92%, 93%, 94%, 95%, 96%, 97%, 98%, or 99% identical to any one of sequence GCCGCCACCAUGG, GCCGCCACC, GCCACC or ACC (SEQ ID NOs: 169-176), or a fragment or a variant of any of these.

In preferred embodiments, the translation initiation sequence is selected from an aptamer.

Suitably, the aptamer is an RNA aptamer that has a length of less than 200 nucleotides, preferably less than 100 nucleotides. Typically, the aptamer has a length of between about 20 to about 60 nucleotides.

In embodiments, the aptamer is configured or selected to allow the incorporation of modilled nucleotides into the aptamer sequence without negatively impacting the efficiency of translation initiation.

According to preferred embodiments, the circular RNA comprises an aptamer as a translation initiation sequence, and comprises modified nucleotides.

Suitably, the aptamer is characterized by a high affinity to the Eukaryotic translation initiation factor 4G (eIF4G) In the context of the invention, the at least one aptamer sequence of the circular RNA may comprise or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 496-503, or a fragment or a variant of any of these.

In preferred embodiments, the translation initiation sequence is selected from an Cap-independent Translation Element (CITE).

CITEs that may be used in the context of the invention comprise TED (translation enhancer domain) CITE, for example derived from Satellite tobacco necrosis virus (STNV), BTE (BYDV-like translation element) CITE, for example derived from Barley yellow dwarf virus (BYDV), PTE (PMV-like translation element) CITE, for example derived from Hirse-Mosaik-Virus (en. Panicum mosaic virus, PMV), TSS (T-shaped structure) CITE, for example derived from Tumip crinkle virus (TCV), Y-shaped (YSS) CITE, for example derived from Tomato bushy stunt virus (TBSV), or I-shaped (ISS) CITE, for example derived from Maize necrotic streak virus (MNeSV).

A CITE may be positioned directly in front of the coding sequence. Alternatively, a CITE may be located at or in a UTR as defined herein. Furthermore, a CITE may be combined with an IRES or an aptamer as defined herein.

Suitably, the CITE is an RNA sequence that has a length of less than 200 nucleotides.

In embodiments, the CITE is configured or selected to allow the incorporation of modified nucleotides into the CITE sequence without negatively impacting the efficiency of translation initiation.

In the context of the invention, the at least one CITE sequence of the circular RNA may comprise or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 504 or 505, or a fragment or a variant of any of these.

In preferred embodiments, the translation initiation sequence is selected from an IRES.

In preferred embodiments, the IRES is selected or derived from a viral IRES, a cellular IRES (e.g. eukaryotic IRES), or a synthetic IRES.

In embodiments, the IRES element has a length of about 20 to about 1000 nucleotides. In preferred embodiments, the IRES element has a length of about 100 to about 1000 nucleotides. In specific embodiments, the IRES element has a length of about 100, about 150, about 200, about 250, about 300, about 350, about 400, about 450, about 500, about 550, about 600, about 650, about 700, about 750, about 800, about 850, about 900, about 950, or about 1000 nucleotides. In preferred embodiments, the IRES element has a length of about 740 nucleotides.

In embodiments, the IRES element is derived from the DNA of an organism including, but not limited to, a virus, a mammal, and a Drosophila. A viral DNA may be derived from, but is not limited to, picomavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster. In some embodiments, the IRES element is at least partially derived from a virus, for instance, it can be selected or derived from a viral IRES element, such as ABPV IGRpred, AEV, ALPV, IGRpred, BQCV IGRpred, BVDV1 1-385, BVDV1 29-391, CrPV 5NCR, CrPV IGR, crTMV IREScp, crTMV_IRESmp75, crTMV_lRESmp228, crTMV IREScp, crTMV IREScp, CSFV, CVB3, DCV IGR, EMCV-R, EoPV_5NTR, ERAV 245-961, ECMV, ERBV 162-920, EV71 1-748, FeLV-Notch2, FMDV type C, GBV-A, GBV-B, GBV-C, gypsy_env, gypsyD5, gypsyD2, HAV HM175, HCV type 1a, HiPV IGRpred, HIV-1, HoCVIJGRpred, HRV-2, IAPV IGRpred, idefix, KBV IGRpred, LINE-1 ORF 1-10 Ito-1. LINE-I_ORF-3)2_to_-202, LINE-1 ORF2-138_to_-86, LINE-I_ORF I_-44_to_-L PSIV IGR, PV typel Mahoney, PV_type3 Leon, REV-A, RhPV 5NCR, RhPV IGR, SINV I IGRpred. SV40 661-830,TMEV, TMV UI IRES mp228, TRV 5NTR, TrV IGR, or TSV IGR. In some embodiments, the IRES element is at least partially derived from a cellular IRES, such as AML1/RUNX1, Antp-D, Antp-DE, Antp-CDE, Apaf-1, Apaf-1, AQP4, ATIR vari, ATIR_var2, ATIR_var3, ATiR_var4, BAGI_p36delta236nt, BAGI_p36, BCL2, BiP_-222_-3, C-IAP1 285-1399, c-IAP1 1313-1462, c-jun, c-rnyc, Cat-1 224, CCND1, DAP5, eIF4G, eIF4Gl-exdiestert, eIF4GIl, eIF4Gll-ong, ELG1, ELH, FGF1A, FMR1, Gtx-133-141, Gtx-1-166, Gtx-1-120, Gtx-1-196, hairless, HAP4, HIFIa, hSNMI, HsplOI, hsp70, hsp70, Hsp90, IGF2_leader2, Kvl.4_1.2, L-myc, LamBl_-335_-, LEF1, MNT 75-267, MNT_36-160, MTG8a, MYB, MYT2_997-1152, n-MYC, NDST1, NDST2, NDST3, NDST4L, NDST4S, NRF-653-17, NtHSFI, ODC1, p27kipl, p53_128-269, PDGF2/c-sis, Pim-1, PITSLRE_p58, Rbm3, reaper, Scamper, TFIID, TIF4631, Ubx_I-966, Ubx_373-961, UNR, Ure2, UtrA, VEGF-A_-133_-1, XIAP_5-464, XIAP 305-466, SaV (Salivirus) or AiV(Aichivirus) or YAP1. In some embodiments, the IRES element comprises a synthetic IRES, for instance, (GAAA)16, (PPT19)4, KM11, KMI1, KMI2, KM12, KMIX, XI, orX2.

In particularly preferred embodiments, the IRES is selected or derived from a viral IRES.

In preferred embodiments, the IRES is a chimeric IRES. In preferred embodiments the chimeric IRES is a chimere of encephalomyocarditis virus (ECMV) IRES and foot-and-mouth-disease virus (FMDV) IRES. In preferred embodiments the chimeric IRES is a chimere of hepatitis C virus (HCV) IRES and classical swine fever virus (CSFV) IRES.

In preferred embodiments, the IRES sequence may be selected from nucleic acid sequences being identical or at least 70%, 80%, 85%, 86%, 87%, 88%, 89%, 90%, 91%, 92%, 93%, 94%, 95%, 96%, 97%, 98%, or 99% 20 identical to any one of SEQ ID NOs: 1566-1662 of published PCT patent application WO2017081082, said sequences herewith incorporated by reference.

In the context of the invention, the at least one IRES sequence of the circular RNA may comprise or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 393-495, 514 or a fragment or a variant of any of these.

In particularly preferred embodiments, the IRES is selected or derived from a coxsackievirus B3 (CVB3) IRES.

Accordingly, in the context of the invention, the at least one IRES sequence of the circular RNA is selected or derived from a coxsackievirus B3 (CVB3) IRES and comprises or consists of an RNA sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 353, or a fragment or a variant thereof.

In particularly preferred embodiments, the IRES is selected or derived from a Salivirus (SaV) or Aichiviurs (AiV) IRES, preferably selected or derived from a Salivirus IRES.

Accordingly, in the context of the invention, the at least one IRES sequence of the circular RNA is selected or derived from a Salivirus (SaV) or Aichivirus (AiV) IRES and comprises or consists of an RNA sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 370, 381, 417, 450, or a fragment or a variant thereof, preferably SEQ ID NO: 417.

In preferred embodiments, the translation initiation sequence as defined herein is located upstream of the start codon of the at least one coding sequence. Suitably, the translation initiation sequence is located upstream of the start codon of the at least one coding sequence and operably linked to that coding sequence.

In particularly preferred embodiments, a Kozak sequence as defined herein and a translation initiation sequence as defined herein is located upstream of the start codon of the at least one coding sequence. Suitably, the translation initiation sequence is located upstream of the Kozak sequence. The Kozak sequence is located upstream of the start codon of the at least one coding sequence and operably linked to that coding sequence.

In preferred embodiments, the translation initiation sequence is an IRES and is located directly upstream of the start codon of the at least one coding sequence. Accordingly, in such embodiments, the circular RNA may comprise an IRES sequence for translation initiation followed by a coding sequence as defined herein.

In preferred embodiments, the RNA sequence located upstream of the translation initiation sequence (in opposing direction of translation) is an unstructured sequence element (also corresponding to the spacer sequence of the splice junction element as defined herein).

The absence of complex secondary structures upstream of the translation initiation sequence (e.g. the IRES) has the advantage that binding of translation initiation factors and/or ribosomes is not impaired.

The term “unstructured” in that context relates to an RNA sequence element with a low number of secondary structures. As used herein, “unstructured” with regard to RNA refers to an RNA sequence that is not predicted by the RNAFold software or similar predictive tools to form a structure (e.g., a hairpin loop) with itself or other sequences in the same RNA molecule. Suitably, in the context of the invention, the unstructured sequence element does not comprise a stable secondary structure under physiological conditions. Suitably, in the context of the invention, the unstructured sequence element is essentially non-structured. Suitably, in the context of the invention, the unstructured sequence element does not bind RNA binding proteins. Suitably, in the context of the invention, the unstructured sequence element does not bind translation initiation factors and/or ribosomes.

Suitably, in the context of the invention, the unstructured sequence element does impair the binding of translation initiation factors and/or ribosomes to translation initiation sequences (typically located downstream).

In preferred embodiments, the unstructured sequence element has a length of at least about 20 nucleotides, preferably at least about 20 nucleotides to about 100 nucleotides. In particularly preferred embodiments, the unstructured sequence element has a length of about 50 nucleotides.

In preferred embodiments, the at least one unstructured sequence element comprises an AC rich sequence. In alternative embodiments, the at least one unstructured sequence element comprises an UG rich sequence.

In preferred embodiments, the unstructured sequence element comprises a poly AC sequence. In alternative embodiments, the unstructured sequence element comprises a poly UG sequence.

In preferred embodiments, the unstructured sequence element has a length of at least 20 nucleotides, preferably about 20 nucleotides to about 200 nucleotides.

In preferred embodiments, the unstructured sequence may be selected from nucleic acid sequences being 15 identical or at least 70%, 80%, 85%, 86%, 87%, 88%, 89%, 90%, 91%, 92%, 93%, 94%, 95%, 96%, 97%, 98%, or 99% identical to SEQ ID NOs: 186, or fragments or variants thereof.

In preferred embodiments, the circular RNA of the invention additionally comprises at least one poly(C) sequence and/or at least one histone-stem loop sequence and/or at least one miRNA binding site.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides.

In embodiments, the circular RNA of the pharmaceutical composition comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence may preferably be derived from formulae (1) or (II) of WO2012/019780. According to a further preferred embodiment, the circular RNA comprises at least one histone stem-loop sequence derived from at least one of the specific formulae (Ia) or (IIa) of the patent application WO2012/019780.

In preferred embodiments, the circular RNA comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to any one of SEQ ID NOs: 177 or 178, or fragments or variants of any of these.

In embodiments, the circular RNA of the pharmaceutical composition comprises at least one miRNA binding site.

The miRNA binding site sequence is located within and/or immediately 3′ or 5′ of the 3′ UTR to allow a cell type specific expression from the circular RNA within the target organ or organs. In particular, the miRNA binding site sequence comprises at least one, two, three, or four miRNA binding sites, which can be similar, identical or different.

In embodiments the at least one first miRNA binding site sequence comprises one or more of the group consisting of binding sites for miRNA-122, miRNA-142, miRNA-148a, miRNA-101, miRNA-192, miRNA-194, and miRNA-223.

In preferred embodiments, the miRNA binding site sequence comprises one or more miRNA-122 binding sites or miRNA-142 binding sites.

In preferred embodiments, the circular RNA does not comprise chemically modified nucleotides.

Accordingly, the circular RNA of the invention consists of non-modified A, U, G, and C ribonucleotides.

In embodiments where the circular RNA comprises an IRES sequence as translation initiation sequence, the circular RNA of the invention may suitably consist of non-modified A, U, G, and C ribonucleotides and may suitably not comprise chemically modified ribonucleotides.

Without wishing to be bound to theory, the absence of modified ribonucleotides may have the advantage that the function of a translation initiation sequence is not impaired. For example, IRES sequences have complex functionally relevant secondary structures that may be destroyed by the introduction of modified ribonucleotides.

In alternative embodiments, the circular RNA comprises modified nucleotides.

A chemically modified circular RNA may comprise nucleotide analogues/modifications, e.g. backbone modifications, sugar modifications or base modifications. A backbone modification is a chemical modification in which phosphates of the backbone of the nucleotides of the circular RNA are modified. A sugar modification is a chemical modification of the sugar of the nucleotides of the circular RNA. Furthermore, a base modification is a chemical modification of the base moiety of the nucleotides of the circular RNA.

In the context of the invention, chemically modified nucleotides that may be incorporated into the circular RNA should be selected in a way that the function of the translation initiation sequence is not impaired.

In various embodiments in that context, the circular RNA comprises chemically modified nucleotides selected from pseudouridine (ψ), N1-methylpseudouridine (m1ψ), N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouidine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In preferred embodiments in that context, the circular RNA comprises pseudouridine (ψ), N1-methylpseudouridine (m1ψ), 5-methylcytosine, 5-methoxyuridine, Alpha-thio-ATP, Alpha-thio-GTP, Alpha-thio-CTP, Alpha-thio-UTP, N4-acetyl-CTP, N6-methyladenosine, 2′O-methyl-ATP, 2′O-methyl-GTP, 2′O-methyl-CTP, and/or 2′O-methyl-UTP.

In preferred embodiments, the at least one modified nucleotide of the circular RNA is selected from pseudouridine (l), N1-methylpseudouridine (m1ψ), 5-methylcytosine, and/or 5-methoxyuridine.

In particularly preferred embodiments, the at least one modified nucleotide of the circular RNA is N1-methylpseudouridine (mli).

In embodiments where the circular RNA comprises modified nucleotides as specified herein, an RNA aptamer sequence is preferably selected as a translation initiation sequence.

As defined herein, aptamers are typically short and the function of such a short aptamer may not be impaired by the introduction of chemically modified nucleotides.

Accordingly, in preferred embodiments, the circular RNA of the invention comprises an aptamer as a translation initiation sequence, and comprises chemically modified nucleotides as defined herein.

In preferred embodiments, the at least one modified nucleotide the circular RNA is selected from Alpha-thio-ATP, Alpha-thio-GTP, Alpha-thio-CTP, Alpha-thio-UTP, N4-acetyl-CTP, N6-methyladenosine, 2′O-methyl-ATP, 2′O-methyl-GTP, 2′O-methyl-CTP, or 2′O-methyl-UTP

In embodiments where the circular RNA comprises modified nucleotides selected from Alpha-thio-ATP, Alpha-thio-GTP, Alpha-thio-CTP, Alpha-thio-UTP, N4-acetyl-CTP, N6-methyladenosine, 2′O-methyl-ATP, 2′O-methyl-GTP, 2′O-methyl-CTP, or 2′O-methyl-UTP, an IRES sequence is preferably selected as a translation initiation sequence.

Without whishing to be bound to theory, the incorporation of Alpha-thio-ATP, Alpha-thio-GTP, Alpha-thio-CTP, Alpha-thio-UTP, N4-acetyl-CTP, N6-methyladenosine, 2′O-methyl-ATP, 2′O-methyl-GTP, 2′O-methyl-CTP, or 2′O-methyl-UTP may not have a negative effect on the structure and/or function of the IRES.

In particularly preferred embodiments, the circular RNA consists of ribonucleotides linked via phosphodiester-bonds. Accordingly, each ribonucleotide of the circular RNA is linked to the following ribonucleotide via phosphodiester-bonds.

In particularly preferred embodiments, the circular RNA does not comprise ribonucleotides linked via an amide bond or a triazole linkage or a linkage different from a phosphodiester-bond.

In alternative embodiments, the circular RNA does comprise ribonucleotides linked via an amide bond or a triazole linkage or a linkage different from a phosphodiester-bond. 15 In particularly preferred embodiments, the circular RNA comprises at least one splice-junction element (v).

In the context of the invention, the splice-junction element (v) comprises residual sequence elements that result from the circularization of a linear precursor RNA (see second aspect).

Suitably, the splice-junction element (v) is not able to mediate any splicing.

A splice junction element (v) in the context of the present invention comprises sequences of the 3′ permuted intron-exon element (see second aspect) and the 5′ permuted intron-exon element (see second aspect). The splice-junction element (v) relates to parts of the 3′ permuted intron-exon element and parts of the 5′ permuted intron-exon element that stays in the circular RNA after circularization occurred.

Sequence elements typically comprised in the splice-junction element (v) are for example the exon fragment of the 3′ permuted intron-exon element or the exon fragment of the 5′ permuted intron-exon element In preferred embodiments, the splice-junction element (v) comprises at least one exon fragment.

In preferred embodiments, the splice-junction element (v) comprises at least one exon fragment Preferably, the exon fragment is derived from a Cyanobacterium Anabaena sp. pre-tRNA-Leu gene or T4 phage Td gene.

The splice-junction element (v) in the context of the invention relates to a nucleic acid sequence identical or at least 70%, 80%, 90%, 91%, 92%, 93%, 94%, 95%, 96%, 97%, 98%, or 99% identical to SEQ ID NOs: 187 and 189, or fragments or variants thereof. In specific examples, the splice-junction element (v) comprises nucleic acid sequence SEQ ID NO: 187 and nucleic acid sequence SEQ ID NO: 189.

In preferred embodiments, the splice-junction element (v) as defined herein may be followed by a unstructured sequence element as defined herein (e.g. a AC rich sequence). In preferred embodiments, the splice-junction element (v) as defined herein may be followed by a unstructured sequence element as defined herein (e.g. a AC rich sequence) and a translation initiation sequence as defined herein (e.g. IRES).

In preferred embodiments, the splice-junction element (v) is located between the at least one Poly(A) sequence (as defined herein) and the at least one translation initiation sequence (as defined herein). “Located between” in that context has to be understood as in the direction of translation of the coding sequence.

In preferred embodiments, the splice-junction element (v) is located between the at least one Poly(A) sequence (as defined herein) and the at least one spacer sequence (as defined herein) followed by the at least one translation initiation sequence (as defined herein). “Located between” in that context has to be understood as in the direction of translation of the coding sequence.

In preferred embodiments, the splice-junction element (v) has a length of at least about 20 nucleotides, preferably about 20 nucleotides to about 200 nucleotides.

In particularly preferred embodiments, the splice-junction element (v) has a length about 100 nucleotides, e.g. about 104 nucleotides.

In preferred embodiments, the circular RNA has a length of at least 500 ribonucleotides. In other preferred embodiments, the circular RNA has a length of at least 1000 ribonucleotides. In other preferred embodiments, the circular RNA has a length of at least 1500 ribonucleotides. In other preferred embodiments, the circular RNA has a length of at least 2000 ribonucleotides. In other preferred embodiments, the circular RNA has a length of at least 2500 ribonucleotides. In other preferred embodiments, the circular RNA has a length of at least 3000 ribonucleotides. In other preferred embodiments, the circular RNA has a length of at least 3500 ribonucleotides.

In other preferred embodiments, the circular RNA has a length of at least 4000 ribonucleotides. In other preferred embodiments, the circular RNA has a length of at least 4500 ribonucleotides. In other preferred embodiments, the circular RNA has a length of at least 5000 ribonucleotides.

In preferred embodiments, the circular RNA has a length ranging from about 500 ribonucleotides to about 5000 ribonucleotides. In particularly preferred embodiments, the circular RNA has a length ranging from about 1500 ribonucleotides to about 5000 ribonucleotides. In particularly preferred embodiments, the circular RNA has a length ranging from about 1500 ribonucleotides to about 3500 ribonucleotides.

In preferred embodiments, the circular RNA backbone has a length of about 500 ribonucleotides to about 1500 ribonucleotides. In particularly preferred embodiments, the circular RNA backbone has a length of about 500 ribonucleotides to about 1000 ribonucleotides.

In the context of the invention, the term “circular RNA backbone” relates to all components of the circular RNA excluding the coding sequence (e.g. comprising translation initiation sequence, UTR sequences, poly(A) sequences, and splice junction elements, but not comprising the coding sequence).

In preferred embodiments, the circular RNA backbone comprises or consists of an RNA sequence identical or at 10 least 70%, 80%, 90%, 91%, 92%, 93%, 94%, 95%, 96%, 97%, 98%, or 99% identical to any of SEQ ID NOs: 202 to 205, wherein the respective coding sequences (encoding GLuc and PLuc) of SEQ ID NOs: 202-205, 533-544 are not part of the circular RNA backbone as defined herein. The skilled person is of course able to remove the coding sequences (encoding GLuc and PLuc) to derive the respective circular RNA backbone sequence.

In preferred embodiments, the circular RNA is a single stranded circular RNA.

In preferred embodiments, the circular RNA lacks a self-replication element Accordingly, the circular RNA does not comprise a sequence encoding a replicase for self-replication of sequences of the circular RNA.

In preferred embodiments, the circular RNA is an in vitro transcribed RNA as defined herein. In particularly preferred embodiments, the RNA in vitro transcription has been performed in the presence of a sequence optimized nucleotide mixture (as further specified in the context of aspects relating to methods for preparing and/or purifying circular RNA).

In preferred embodiments, the circular RNA has been circularized via self-splicing from a linear precursor RNA.

In particularly preferred embodiments, the circular RNA has been circularized via self-splicing from a linear single stranded precursor RNA. Suitably, the linear precursor RNA is characterized by any of the features provided in the second aspect In some embodiments, the circular RNA has been circularized via enzymatic ligation, splint ligation, self-cleavable elements (e.g., hammerhead, splicing element), cyclase ribozyme, cleavage recruitment sites (e.g., ADAR), a degradable linker (e.g., glycerol), or chemical methods of circularization.

Enzymatic ligation typically involves RNA ligases (e.g. T4 RNA ligase, T4 RNA ligase 2). Using such enzymes, linear RNA can be circularized, provided that the donor contains a 5′-phosphate and the acceptor a 3′-OH. A DNA or RNA ligase may be used to enzymatically link a 5′—phosphorylated nucleic acid molecule to the 3′-hydroxyl group of a linear precursor RNA forming a new phosphodiester linkage. In an example reaction, a linear precursor RNA is incubated at 37° C. for 1 hour with 1-10 units of T4 RNA ligase. The ligation reaction may occur in the presence of a linear precursor RNA capable of base-pairing with both the 5′- and 3′—region in juxtaposition to assist the enzymatic ligation reaction.

RNA transcribed from RNA cyclase ribozyme genes autocatalytically converts the desired RNA sequence it contains into circular form. RNA cyclase genes may be placed into appropriate expression vectors for synthesis of circular RNA in vitro and in vivo. Method for rearrangement of regions that code for RNA splicing elements in such a way that if the 3′ half of an intron is placed first, followed by the 3′ splice site, followed by the sequence of interest inserted between the 3′ splice site and the 5′ splice site, followed by the 5′ splice site and the 5′ half of an intron, then after splicing, the sequence between the 3′ splice site and the 5′ splice site becomes circular.

Splint ligation typically involves a single stranded polynucleotide (splint), like a single stranded RNA, can be designed to hybridize with both termini of a linear precursor RNA, so that the two termini can be juxtaposed upon hybridization with the single-stranded splint. Splint ligase can thus catalyze the ligation of the juxtaposed two termini of the linear precursor RNA, generating a circular RNA. For example, a splint ligase, like SplintR® ligase, can be used for splint ligation.

Chemical methods of circularization may include, but are not limited to click chemistry (e.g., alkyne and azide based methods, or clickable bases), olefin metathesis, phosphoramidate ligation, hemiaminal-imine crosslinking, 20 base modification, and any combination thereof. For example, the 5′-end and the 3′-end of the linear precursor RNA may include chemically reactive groups that, when close together, may form a new (covalent) linkage between the 5′-end and the 3-end of the RNA. In embodiments, the 5′-end may contain an NHS-ester reactive group and the 3′-end may contain a 3′-amino-terminated nucleotide such that under suitable conditions (e.g. in an organic solvent) the 3′-amino-terminated nucleotide on the 3′-end of a linear RNA molecule will undergo a nucleophilic attack on the 5′-NHS-ester moiety forming a new 5′-/3′—amide bond.

In preferred embodiments, the circular RNA has not been circularized via enzymatic ligation, splint ligation, or chemical methods of circularization.

In preferred embodiments, the circular RNA is a purified circular RNA.

The term “purified circular RNA” or as used herein has to be understood a circular RNA which has a higher purity after certain purification steps (e.g. HPLC, TFF, Oligo d(T) purification, precipitation, filtration, AEX, cellulose purification, SEC) than the starting material (e.g. crude circularization reaction). Typical impurities that are essentially not present in purified circular RNA comprise peptides or proteins (e.g. enzymes derived from RNA in vitro transcription, e.g. RNA polymerases, RNases, pyrophosphatase, restriction endonudease, DNase), spermidine, BSA, short abortive RNA sequences, RNA fragments (short double stranded RNA fragments, short single stranded RNA fragments, abortive RNA sequences etc.), free nucleotides (modified nucleotides, conventional NTPs, cap analogue), template DNA fragments, buffer components (HEPES, TRIS, MgCl2, CaCl2), linear precursor RNA or fragments thereof, intronic RNA fragments, RNAse. Other potential impurities may be derived from e.g. fermentation procedures and comprise bacterial impurities (bioburden, bacterial DNA, bacterial RNA) or impurities derived from purification procedures (organic solvents etc.). Accordingly, it is desirable in this regard for the “degree of RNA purity” to be as close as possible to 100%, e.g. 80%, 85%, 90%, 95%.

In preferred embodiments, the purified circular RNA is essentially free of non-circularized linear precursor RNA.

In preferred embodiments, the purified circular RNA is essentially free of intermediates derived from the self-splicing process (e.g. intronic RNA molecules) In preferred embodiments, the circular RNA of the invention has an certain RNA integrity.

The term “RNA integrity” generally describes whether the complete circular RNA sequence with the correct RNA length is present. Low RNA integrity could be due to, amongst others, RNA degradation, RNA cleavage, incorrect or incomplete circularization of the RNA, incorrect base pairing, integration of modified nucleotides or the modification of already integrated nucleotides etc. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the circular RNA of the invention.

The skilled person can choose from a variety of different chromatographic or electrophoretic methods for determining integrity of circular RNA. Chromatographic and electrophoretic (e.g. capillary gel electrophoresis) methods are well-known in the art. In case chromatography is used (e.g. RP-HPLC), the analysis of the integrity 25 of the circular RNA may be based on determining the peak area (or “area under the peak”) of the expected full length circular RNA (the RNA with the correct RNA length) in a corresponding chromatogram.

In embodiments, the circular RNA of the invention has an RNA integrity ranging from about 40% to about 100%.

In embodiments, the circular RNA has an RNA integrity ranging from about 50% to about 100%. In embodiments, the circular RNA has an RNA integrity ranging from about 60% to about 100%. In embodiments, the circular RNA has an RNA integrity ranging from about 70% to about 100%. In embodiments, the circular RNA integrity is for example about 50%, about 60%, about 70%, about 80%, or about 90%. Integrity of circular RNA is suitably determined using analytical HPLC, preferably analytical RP-HPLC.

In preferred embodiments, the circular RNA has an RNA integrity of at least about 50%, preferably of at least about 60%, more preferably of at least about 70%, most preferably of at least about 80%. RNA integrity is suitably determined using analytical HPLC, preferably analytical RP-HPLC. Alternatively, RNA integrity may be measured using a commercially available fragment analyzer.

In particularly preferred embodiments, the (purified) circular RNA has been produced and/or purified using methods as provided in the aspects “method of preparing and/or purifying circular RNA”. Accordingly, certain purity and quality levels (RNA purity, RNA integrity, amount of linear precursor RNA, amount of dsRNA fragments, DNA, protein, and/or abortive IVT fragments) and methods for determining these values are also provided in the aspects “method of preparing and/or purifying circular RNA”. Advantageous features of the circular RNA obtained by the methods of the aspects “method of preparing and/or purifying circular RNA” do of course also relate to the circular RNA of the first aspect.

In preferred embodiments, upon administration of the circular RNA to a cell or subject, the circular RNA has reduced immunostimulatory properties compared to administration of a corresponding reference RNA. In preferred embodiments, the immunostimulatory properties are defined as the induction of an innate immune response which is determined by measuring the induction of cytokines.

A corresponding reference RNA may be defined as a comparable circular RNA encoding the same amino acid sequence, but lacking e.g. the poly(A) sequence element and/or the UTR sequence. Further, a corresponding reference RNA may be defined as a comparable linear RNA (e.g. capped mRNA) encoding the same amino acid sequence. Further, a corresponding reference RNA may be defined as a comparable linear RNA comprising a wild-type coding sequence but encoding the same amino acid sequence.

Accordingly, the circular RNA as defined herein has reduced immunostimulatory properties compared to a corresponding reference RNA.

In this context, it is particularly preferred that the circular RNA has at least 10%, 20% or at least 30% lower immunostimulatory properties compared to a corresponding reference RNA.

In some preferred embodiments, the circular RNA has at least 40%, 50% or at least 60% lower immunostimulatory properties compared to a corresponding reference RNA.

In preferred embodiments, the circular RNA is characterized by a lower affinity to a pattern recognition receptor compared to a corresponding reference RNA. Preferably, a pattern recognition receptor is selected from the group consisting of TLR3, TLR7, TLR8, PKR, MDA5, RIG-1, LGP2 or 2′-5′-oligoadenylate synthetase.

The term, ‘Patteen recognition receptor’ (PRR) will be recognized and understood by the person of ordinary skill in the art, and is e.g. intended to refer to receptors that are part of the innate immune system. Germline-encoded PRRs are responsible for sensing the presence of microbe-specific molecules (such as bacterial or viral DNA or RNA) via recognition of conserved structures, which are called pathogen-associated molecular patterns (PAMPs). Recent evidence indicates that PRRs are also responsible for recognizing endogenous molecules released from damaged cells, termed damage-associated molecular patterns (DAMPs). Currently, four different classes of PRR families have been identified. These families include transmembrane proteins such as the Toll-like receptors (TLRs) and C-type lectin receptors (CLRs), as well as cytoplasmic proteins such as the Retinoic acid-inducible gene (RIG)-I-like receptors (RLRs) and NOD-like receptors (NLRs). Based on their localization, PRRs may be divided into membrane-bound PRRs and cytoplasmic PRRs and are expressed not only in macrophages and DCs but also in various nonprofessional immune cells. Typical Pattern recognition receptor” (PRR) in the context of the invention are Toll-like receptors, NOD-like receptors, RIG-I like receptors, PKR, OAS1, IFIT1 and IFIT5.

In preferred embodiments, the immunostimulatory properties are defined as the induction or activation or stimulation of an innate immune response which is determined by measuring the induction of cytokines.

Preferably, a reduction of the immunostimulatory properties is characterized by a reduced level of at least one cytokine preferably selected from MIG, McP1, Rantes, MIP-1 alpha, IP-10, MIP-1 beta, McP1, TNFalpha, IFNgamma, IFNalpha, IFNbeta, IL-12, IL-6, or IL-8.

The term “reduced level of at least one cytokine” has to be understood as that the administration of the circular RNA of the invention reduces the induction of cytokines to a certain percentage compared to a corresponding reference RNA.

Accordingly, reduction of the immunostimulatory properties in the context of the invention is characterized by a reduced level of at least one cytokine preferably selected from MIG, McP1, Rantes, MIP-1 alpha, IP-10, MIP-1 beta, McP1, TNFalpha, IFNgamma, IFNalpha, IFNbeta, IL-12, IL-6, or IL-8, wherein the reduced level of at least one cytokine is a reduction of at least 10%, 15%, 20%, 25%, 30%, 35%, 40%, 45%, 50%, 55%, 60%, 65%, 25 70%, 75%, 80%, 85%, 90%, or 95%. Preferably, the reduced level of at least one cytokine is a reduction of at least 30%.

Methods to evaluate the (innate) immune stimulation (that is, the induction of e.g. MIG, McP1, Rantes, MIP-1 alpha, IP-10, MIP-1 beta, McP1, TNFalpha, IFNgamma, IFNalpha, IFNbeta, IL-12, IL-6, or IL-8) by the circular RNA of the invention in specific cells/organs/tissues are well known in the art for the skilled artisan.

Methods to evaluate the reduction of immunostimulatory properties (that is, the reduction of the (innate) immune response of e.g. Rantes, MIP-1 alpha, MIP-1 beta, McP1, TNFalpha, IFNgamma, IFNalpha, IFNbeta, IL-12, IL-6, or IL-8) by the circular RNA in specific cells/organs/tissues are well known in the art for the skilled artisan. Typically, the (innate) immune stimulation of the circular RNA is compared with a corresponding reference RNA. The same conditions (e.g. same cell lines, same organism, same application route, same detection method, same amount of RNA, similar RNA sequence etc.) have to be used (if feasible) to allow a valid comparison. The person of skill in the art understands how to perform a comparison of the inventive circular RNA and a corresponding reference RNA.

In the context of the invention, the induction of cytokines is measured after administration of the circular RNA to cells, a tissue or an organism, preferably hPBMCs, Hela cells or HEK cells. Preferred in that context are hPBMCs. Upon administration of the circular RNA (or the corresponding reference RNA) to hPBMCs, Hela cells or HEK cells, an assay for measuring cytokine levels is performed. Cytokines secreted into culture media or supematants can be quantified by techniques such as bead based cytokine assays (e.g. cytometric bead array (CBA), ELISA, and Western blot).

In a preferred embodiment circular RNA of the invention is more stable and/or the encoded peptide or protein is more efficiently expressed compared to a corresponding reference RNA. Suitably, the circular RNA of the invention is more stable and/or the encoded peptide or protein is more efficiently expressed compared to a corresponding reference RNA in liver cells (e.g. after intravenous administration).

Accordingly, in preferred embodiments, upon administration of the circular RNA to a cell or subject, the circular RNA has a prolonged protein expression compared to a corresponding reference RNA.

A more stable and/or more efficiently expression as described herein has to be understood as the additional duration of protein expression wherein expression of the circular RNA is still detectable in comparison to a corresponding reference RNA. The level of protein expression can be determined by various well-established expression assays (e.g. antibody-based detection methods).

In a preferred embodiment circular RNA of the invention has a prolonged protein expression in cells that are characterized by a reduced/lowered eIF4F expression compared to a corresponding reference RNA. A reduced or lowered eIF4F expression is detectable in comparison to the average expression rate of a corresponding unaffected cell type with normal eIF4F expression.

Reduced or lowered eIF4F expression in cells is known in many cellular and preclinical models of cancer or models of obesity related diseases. eIF4F deregulation results in changes in translational efficiency of specific mRNA classes, e.g. in reduced or lowered expression of cap dependent sequence translation.

Accordingly, administration of the circular RNA to a cell, tissue, or organism results in a prolonged protein expression compared to administration of the corresponding reference RNA, wherein the additional duration of protein expression in said cell, tissue, or organism is at least 5 h, 10h, 20 h, 25h, 30h, 35h, 40h, 45h, 50h, 55h, 60h, 65h, 70h, 75h, 80h, 85h, 90h, 95h, or 100h or even longer. Preferably, the additional duration of protein expression is about 20h to about 240h. Suitably, the additional duration of protein expression is in liver cells. In preferred embodiments, the additional duration of protein expression is in muscle cells. In preferred embodiments, the additional duration of protein expression is in adipocytes.

Methods to evaluate the expression (that is, protein expression) of the circular RNA in specific cells/organs/tissues, and methods to determine the duration of expression are well known in the art for the skilled artisan. For example, protein expression can be determined using antibody-based detection methods (western blots, FACS, ELISA) or quantitative mass spectrometry. The same conditions (e.g. same cell lines, same organism, same application route, same detection method, same amount of RNA, similar RNA sequence) have to be used (if feasible) to allow a valid comparison. The person of skill in the art understands how to perform a comparison of the circular RNA and a respective reference RNA.

The “more efficiently expressed” circular RNA comprising has to be understood as percentage increase of expression compared to a corresponding reference RNA which can be determined by various well-established expression assays (e.g. antibody-based detection methods) as described above.

Accordingly, administration of the circular RNA of the invention to a cell, tissue, or organism results in an increased expression as compared to administration of the corresponding reference RNA, wherein the percentage increase in expression in said cell, tissue, or organism is at least 10%, 20%, 30%, 40%, 50%, 60%, 70%, 80%, 90%, 100%, 200%, 300%, 400%, 500% or even more. Preferably, the percentage increase in expression is about 20% to about 100%. Suitably, increase in expression is in liver cells. In preferred embodiments, increase in expression is in muscle cells. Additionally, in preferred embodiments, increase in expression is in adipocytes.

In preferred embodiments, upon administration of the circular RNA to a cell or subject, the circular RNA has a longer RNA half-life as compared to a corresponding reference RNA.

Accordingly, administration of the circular RNA to a cell, tissue, or organism results in a longer half-life of the circular RNA compared to administration of a corresponding reference RNA, wherein the additional duration of half-life in said cell, tissue, or organism is at least 5 h, 10h, 20 h, 25h, 30h, 35h, 40h, 45h, 50h, 55h, 60h, 65h, 70h, 75h, 80h, 85h, 90h, 95h, or 100h or even longer. Preferably, the additional half-life is about 20h to about 240h. Suitably, the additional half-life is observed in liver cells. In preferred embodiments, the additional half-life is observed in adipocytes. In preferred embodiment, the additional half-life is observed in muscle cells.

To further extend the half-life of the circular RNA in vivo, the circular RNA may additionally comprise at least one RNA sequences that serve as protein binding sites. In some embodiments, the protein binding site includes a binding site to the protein such as for example ACINI, AGO, APOBEC3F, APOBEC3G, ATXN2, AUH, BCCIP, CAPRINI, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIPILI, FKBP4, FMR1, FUS, FXR1, FXR2, GNL3, GTF2F1, HNRNPAI, HNRNPA2B1, HNRNPC, HNRNPK, HNRNPL, HNRNPM, HNRNPU, HNRNPULI, IGF2BP1, IGF2BP2, IGF2BP3, ILF3, KHDRBSI, LARP7, LIN28A, LIN28B, m6A, MBNL2, METTL3, MOV10, MS11, MS12, NONO, NONO-, NOP58, NPM1, NUDT21, PCBP2, POLR2A, PRPF8, PTBP1, RBFOX2, RBMIO, RBM22, RBM27, RBM47, RNPSI, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, or AKTI.

Such protein binding sites are preferably not located upstream of the translation initiation sequence to not interfere with an efficient protein translation.

For circular RNA and/or the encoded protein with a long half-life in vivo might be necessary to induce a rapid and complete block in transcription, e.g. to avoid unwanted secondary effects, such as cytotoxicity. One not limiting example are ON and OFF switches for CAR T cells using the clinically approved drug lenalidomide, which mediates the proteasomal degradation of several target proteins by inducing interactions between the CRL4CRBN E3 ubiquitin ligase and a C2H2 zinc finger degron motif.

Accordingly, in preferred embodiments the circular RNA may additionally comprise at least one RNA sequences that serve as small tag (called degron) that induces degradation in the presence or absence of a defined ligand, so that the level of degradation of a degron-fused protein via the ubiquitin-proteasome pathway can be rapidly controlled by ligand administration.

In preferred embodiments, administration of the circular RNA is intravenous, intranasal, intramuscular, intradermal, transdermal, intraocular, subcutaneous, intrapulmonal, intralesional, intrathecal, intracranial, intracardial, intratumoral.

Accordingly, in preferred embodiments, the circular RNA is suitable for intravenous, intranasal, intramuscular, intradermal, transdermal, intraocular, subcutaneous, intrapulmonal, intralesional, intrathecal, intracranial, intracardial, intratumoral administration.

Suitably, upon intravenous, intranasal, intramuscular, intradermal, transdermal, intraocular, subcutaneous, intrapulmonal, intralesional, intrathecal, intracranial, intracardial, intratumoral administration, the circular RNA to a cell or subject, the circular RNA has reduced immunostimulatory properties compared to a corresponding reference RNA.

Suitably, upon intravenous, intranasal, intramuscular, intradermal, transdermal, intraocular, subcutaneous, intrapulmonal, intralesional, intrathecal, intracranial, intracardial, intratumoral administration, the circular RNA to a cell or subject, the circular RNA to a cell or subject, the circular RNA has a prolonged protein expression compared to a corresponding reference RNA.

In preferred embodiments, the circular RNA of the invention comprises the following sequence elements in the following order operably connected to each other:
(i) at least one translation initiation sequence;
(ii) optionally, at least one Kozak sequence
(iii) at least one coding sequence (cds);
(iv) at least one UTR sequence;
(v) at least one poly(A) sequence comprising about 40 to about 150 consecutive adenosine nucleotides;
(vi) a splice-junction element,

wherein the splice-junction element (v) and the at least one translation initiation sequence (i) are connected to form a single stranded circular RNA.

In particularly preferred embodiments, the circular RNA of the invention comprises the following sequence elements a) to e) in the following order:
(a) at least one unstructured sequence element as defined herein, optionally selected or derived from SEQ ID NO 186, preferably directly followed by
(b) at least one IRES as defined herein, preferably selected or derived from SEQ ID NO: 353, preferably operably linked to
(c) at least one coding sequence (cds) as defined herein, preferably a GC optimized coding sequence;
(d) at least one UTR sequence as defined herein, preferably selected or derived from SEQ ID NO: 192;
(e) at least one poly(A) sequence comprising about 40 to about 150 consecutive adenosine nucleotides as defined herein,

preferably, wherein the elements are connected via phosphodiester bonds to form a circular RNA.

In very particularly preferred embodiments, the circular RNA of the invention comprises the following sequence elements a) to e) in the following order
(a) at least one unstructured sequence element as defined herein, optionally selected or derived from SEQ ID NO 186, preferably directly followed by
(b) at least one IRES as defined herein, preferably selected or derived from SEQ ID NO: 353, preferably operably linked to
(c) at least one Kozak sequence as defined herein, preferably selected or derived from GCCACC,
(d) at least one coding sequence (cds) as defined herein, preferably a GC optimized coding sequence;
(e) at least one UTR sequence as defined herein, preferably selected or derived from SEQ ID NO: 192;
(f) at least one poly(A) sequence comprising about 30 to about 150 consecutive adenosine nucleotides as defined herein,

preferably, wherein the elements are connected via phosphodiester bonds to form a circular RNA.

In preferred embodiments, the circular RNA of the invention comprises the following sequence elements a) to e) in the following order:
(a) at least one unstructured sequence element as defined herein, optionally selected or derived from SEQ ID NO 186, preferably directly followed by
(b) at least one IRES as defined herein, preferably selected or derived from SEQ ID NO: 353, preferably operably linked to
(c) at least one Kozak sequence as defined herein, preferably selected or derived from GCCACC,
(d) at least one coding sequence (cds) as defined herein, preferably a GC optimized coding sequence;
(e) at least one UTR sequence as defined herein, preferably selected or derived from SEQ ID NO: 158;
(f) at least one poly(A) sequence comprising about 30 to about 150 consecutive adenosine nucleotides as defined herein,

preferably, wherein the elements are connected via phosphodiester bonds to form a circular RNA.

In preferred embodiments, the circular RNA of the invention comprises the following sequence elements a) to e) in the following order
(a) at least one unstructured sequence element as defined herein, optionally selected or derived from SEQ ID NO 186, preferably directly followed by
(b) at least one IRES as defined herein, preferably selected or derived from SEQ ID NO: 417, preferably operably linked to
(c) at least one coding sequence (cds) as defined herein, preferably a GC optimized coding sequence;
(d) at least one UTR sequence as defined herein, preferably selected or derived from SEQ ID NO: 192;
(e) at least one poly(A) sequence comprising about 30 to about 150 consecutive adenosine nucleotides as defined herein,

preferably, wherein the elements are connected via phosphodiester bonds to form a circular RNA.

In particularly preferred embodiments, the circular RNA of the invention comprises the following sequence elements a) to e) in the following order
(a) at least one unstructured sequence element as defined herein, optionally selected or derived from SEQ ID NO 186, preferably directly followed by
(b) at least one IRES as defined herein, preferably selected or derived from SEQ ID NO: 417, preferably operably linked to
(c) at least one Kozak sequence as defined herein, preferably selected or derived from GCCACC,
(d) at least one coding sequence (cds) as defined herein, preferably a GC optimized coding sequence;
(e) at least one UTR sequence as defined herein, preferably selected or derived from SEQ ID NO: 192;
(f) at least one poly(A) sequence comprising about 30 to about 150 consecutive adenosine nucleotides as defined herein, preferably, wherein the elements are connected via phosphodiester bonds to form a circular RNA.

In particularly preferred embodiments, the circular RNA of the invention comprises the following sequence elements a) to f) in the following order
(a) at least one splice-junction element (v) that comprises a nucleic acid sequence SEQ ID NO: 187;
(b) at least one unstructured sequence element selected or derived from SEQ ID NO 186;
(c) at least one IRES selected or derived from SEQ ID NO: 353, operably linked to
(d) at least one coding sequence (cds) as defined herein, preferably a GC optimized coding sequence;
(e) at least one UTR sequence selected or derived from SEQ ID NO: 192;
(f) at least one poly(A) sequence comprising about 40 to about 150 consecutive adenosine nucleotides as defined herein, preferably selected from SEQ ID NOs: 193 to 195;
(g) at least one splice-junction element (v) that comprises nucleic acid sequence SEQ ID NO: 189, wherein the elements are connected via phosphodiester bonds to form a circular RNA.

In very particularly preferred embodiments, the circular RNA of the invention comprises the following sequence elements a) to f) in the following order:
(a) at least one splice-junction element (v) that comprises a nucleic acid sequence SEQ ID NO: 187;
(b) at least one unstructured sequence element selected or derived from SEQ ID NO 186;
(c) at least one IRES selected or derived from SEQ ID NO: 353, operably linked to
(d) at least one Kozak sequence as defined herein, preferably selected or derived from GCCACC,
(e) at least one coding sequence (cds) as defined herein, preferably a GC optimized coding sequence;
(f) at least one UTR sequence selected or derived from SEQ ID NO: 192;
(g) at least one poly(A) sequence comprising about 30 to about 150 consecutive adenosine nucleotides as defined herein, preferably selected from SEQ ID NOs: 193 to 195;
(h) at least one splice-junction element (v) that comprises nucleic acid sequence SEQ ID NO: 189, wherein the elements are connected via phosphodiester bonds to form a circular RNA.

In preferred embodiments, the circular RNA of the invention comprises the following sequence elements in the following order
(a) optionally, at least one unstructured sequence element as defined herein, preferab/ydirectly followed by
(b) at least one aptamer for translation initiation as defined herein preferably selected or derived from SEQ ID NOs: 393-495;
(c) optionally, at least one upstream UTR;
(d) at least one Kozak sequence preferably selected or derived from SEQ ID NO 169-176 (GCCGCCACCAUGG, GCCGCCACC, GCCACC, ACC), operably linked to
(e) at least one coding sequence (cds) as defined herein, preferably a GC optimized coding sequence;
(f) at least one UTR sequence, preferably selected or derived from SEQ ID NO: 192;
(g) at least one poly(A) sequence comprising about 40 to about 150 consecutive adenosine nucleotides as defined herein,
(h) optionally, at least one splice-junction element that comprises nucleic acid sequence SEQ ID NO: 189 and nucleic acid sequence SEQ ID NO: 187, preferably wherein the elements are connected via phosphodiester bonds to form a circular RNA, optionally wherein the circular RNA comprises chemically modified nucleotides, e.g. pseudouridine (y), N1-methylpseudouridine (m1ψ), 5-methylcytosine, and/or 5-methoxyuridine.

In particularly preferred embodiments, the circular RNA of the invention comprises or consists an RNA sequence identical or at least 70%, 80%, 90%, 91%, 92%, 93%, 94%, 95%, 96%, 97%, 98%, or 99% identical to any one of SEQ ID NOs: 202-205, 533-544 or a fragment or a variant of any of these, wherein the coding sequence (encoding Gluc or Ppluc) in any one of SEQ ID NOs: 202-205, 533-544 is exchanged by at least one coding sequence as defined herein, preferably a coding sequence encoding a therapeutic peptide or protein as defined herein.

### 2: Linear Precursor RNA for Making a Circular RNA

In a second aspect, the invention provides linear precursor RNA for making circular RNA.

As used herein, “linear precursor RNA” refers to a linear RNA molecule typically created by RNA in vitro transcription (e.g., from a DNA template). This linear precursor RNA molecule may contain the entirety of the sequences of the circular RNA of the first aspect, plus splicing sequences (intron fragments and homology arms) necessary to circularize the linear precursor RNA. These splicing sequences (intron fragments and homology arms) are removed from the linear precursor RNA during circularization, yielding circular RNA plus two intron/homology arm linear RNA fragments (“intronic splice products”).

Notably, embodiments relating to the circular RNA of the first aspect may likewise be read on and be understood as suitable embodiments of the linear precursor RNA of the second aspect In embodiments, the linear precursor RNA comprises at least one translation initiation sequence operably linked to at least one coding sequence.

In preferred embodiments, the linear precursor RNA comprising the following elements operably connected to each other and preferably arranged in the following sequence:
at least one translation initiation sequence preferably as defined in the first aspect;
at least one coding sequence, preferably as defined in the first aspect;
at least one UTR sequence, preferably as defined in the first aspect; and
at least one poly(A) sequence, preferably as defined in the first aspect

In preferred embodiments, the linear precursor RNA comprising the following elements operably connected to each other and preferably arranged in the following sequence:
at least one translation initiation sequence preferably as defined in the first aspect;
at least one Kozak sequence preferably as defined in the first aspect;
at least one coding sequence, preferably as defined in the first aspect;
at least one UTR sequence, preferably as defined in the first aspect; and
at least one poly(A) sequence, preferably as defined in the first aspect

In embodiments, the linear precursor RNA comprises at least one 3′ permuted intron-exon element and at least one 5′ permuted intron-exon element, both elements arranged to allow circularization of the RNA.

Preferably, such a circularization event results in generating a circular RNA of the first aspect For example, a linear precursor RNA according to SEQ ID NO: 198 may lead, after circularization reaction, to the two intronic splice products according to SEQ ID NO: 184 and according to SEQ ID NO: 185, and to the desired circular RNA according to SEQ ID NO: 203.

As another example, a linear precursor RNA according to SEQ ID NO: 199 may lead, after circularization reaction, to the two intronic splice products according to SEQ ID NO: 184 and according to SEQ ID NO: 185, and to the desired circular RNA according to SEQ ID NO: 204.

In preferred embodiments, the linear precursor RNA comprises the following elements preferably operably connected to each other and arranged in the following sequence:
a 3′ permuted intron-exon element;
at least one translation initiation sequence;
at least one coding sequence;
at least one UTR sequence;
at least one poly(A) sequence;
a 5′ permuted intron-exon element;

In preferred embodiments, the linear precursor RNA comprises the following elements preferably operably connected to each other and arranged in the following sequence:
a 3′ permuted intron-exon element;
at least one translation initiation sequence;
at least one Kozak sequence;
at least one coding sequence;
at least one UTR sequence;
at least one poly(A) sequence;
a 5′ permuted intron-exon element; In preferred embodiments, the linear precursor RNA comprises a 5′ terminal triphosphate group and/or a 3′ terminal OH group. In alternative embodiments, the linear precursor RNA comprises a 5′ terminal diphosphate group. In alternative embodiments, the linear precursor RNA comprises a 5′ terminal monophosphate group. In alternative embodiments, the linear precursor RNA comprises a 5′ terminal cap structure.

In preferred embodiments, the 3′ permuted intron-exon element of the linear precursor RNA comprises a 5′ homology arm, a 3′ Group I intron fragment containing a 3′ splice site dinucleotide, and an optional 5′ spacer sequence (or unstructured sequence). In alternative embodiments, the 3′ permuted intron-exon element of the linear precursor RNA comprises a 3′ Group II or Group 1I intron fragment.

In preferred embodiments, the 5′ permuted intron-exon element of the linear precursor RNA comprises an 3′ spacer sequence (which corresponds to the unstructured sequence as defined in the context of the first aspect), a 5′ Group I intron fragment containing a 5′ splice site dinucleotide, and a 3′ homology arm. In alternative embodiments, the 3′ permuted intron-exon element of the linear precursor RNA comprises a 5′ Group II or Group III intron fragment.

As used herein, a “homology arm” is any contiguous sequence that is 1) predicted to form base pairs with at least about 75% (e.g., at least about 80%, at least about 85%, at least about 90%, at least about 95%, about 100%) of another sequence in the RNA, such as another homology arm 2) at least 7nt long and no longer than 250nt 3) located before and adjacent to, or included within, the 3′ intron fragment and/or after and adjacent to, or included within, the 5′ intron fragment and, optionally, 4) predicted to have less than 50% (e.g., less than 45%, less than 40%, less than 35%, less than 30%, less than 25%) base pairing with unintended sequences in the RNA (e.g., non-homology arm sequences). A “strong homology arm” refers to a homology arm with a Tm of greater than 50 degrees Celsius when base paired with another homology arm in the RNA.

As used herein, a 3′ group I intron fragment is a contiguous sequence that is at least 75% (e.g., at least 80%, at least 85%, at least 90%, at least 95%, 100%) homologous to a 3′ proximal fragment of a natural group I intron, including the 3′ splice site dinucleotide, and, optionally, the adjacent exon sequence at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length). In one embodiment, the included adjacent exon sequence is about the length of the natural exon. In some embodiments, a 5′ group I intron fragment is a contiguous sequence that is at least 75% (e.g., at least 80%, at least 85%, at least 90%, at least 95%, 100%) homologous to a 5′ proximal fragment of a natural group I intron, including the 5′ splice site dinucleotide and, optionally, the adjacent exon sequence at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length). In one embodiment, the included adjacent exon sequence is about the length of the natural exon.

As used herein, a “spacer” refers to any contiguous nucleotide sequence that is 1) predicted to avoid interfering with proximal structures, for example, from the translation initiation sequence, coding or noncoding region, or intron 2) at least 7 nucleotides long (and optionally no longer than 100 nucleotides) 3) located downstream of and adjacent to the 3′ intron fragment and/or upstream of and adjacent to the 5′ intron fragment and/or 4) contains one or more of the following: a) an unstructured region at least 5nt long b) a region predicted base pairing at least 5nt long to a distal (i.e., non-adjacent) sequence, including another spacer, and/or c) a structured region at least 7nt long limited in scope to the sequence of the spacer.

In preferred embodiments, the spacer sequences can be a poly AC sequences, poly C sequences, or poly U sequences, or the spacer sequences can be specifically engineered depending on the used translation initiation sequence. Spacer sequences as described herein may have two functions: (1) promote circularization and (2) promote functionality by allowing the introns and the translation initiation sequence to fold correctly. More specifically, the spacer sequences as described herein were engineered with three priorities: 1) to be inert with regards to the folding of proximal intron and translation initiation sequence structures; 2) to sufficiently separate intron and translation initiation sequence secondary structures; and 3) to contain a region of spacer-spacer complementarity to promote the formation of a “splicing bubble”.

In various embodiments, the 5′ homology arm is about 5-50 nucleotides in length. In another embodiment, the 5′ homology arm is about 9-19 nucleotides in length.

In various embodiments, the 3′ homology arm is about 5-50 nucleotides in length. In another embodiment, the 3′ homology arm is about 9-19 nucleotides in length.

As used herein, the term “splice site dinucleotide” refers to the two nucleotides that border a splice site.

In preferred embodiments, the 3′ Group I intron fragment and the 5′ Group I intron fragment are from a Cyanobacterium Anabaena sp. pre-tRNA-Leu gene or T4 phage Td gene.

In preferred embodiments, the 3′ Group I intron fragment and the 5′ Group I intron fragment are from a Cyanobacterium Anabaena sp. pre-tRNA-Leu gene.

In preferred embodiments, each homology arm of the 3′ permuted intron-exon element and the 5′ permuted intron-exon element is about 5-50 nucleotides in length, In particularly preferred embodiments, each homology arm is about 20-50 nucleotides in length.

In preferred embodiments, each spacer sequence of the 3′ permuted intron-exon element and the 5′ permuted intron-exon element is at least 10 nucleotides in length, at least 15 nucleotides in length, or at least 30 nucleotides in length, e.g. about 50 nucleotides in length.

In preferred embodiments, the 3′ intron fragment comprises a 3-proximal Group I intron-derived sequence including the 3′ splice site dinucleotide and optionally sequence corresponding to the adjacent natural exon, In preferred embodiments, the 5′ intron fragment comprises a 5′—proximal Group I intron-derived sequence including the 5′ splice site dinucleotide and optionally sequence corresponding to the adjacent exon.

In preferred embodiments, the 3′ permuted intron-exon element may be selected or derived from nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 508, or fragments or variants thereof.

In preferred embodiments, the 5′ permuted intron-exon element may be selected or derived from nucleic acid sequences being identical or at least 70%, 80%, 85%, 86%, 87%, 88%, 89%, 90%, 91%, 92%, 93%, 94%, 95%, 96%, 97%, 98%, or 99% identical to any of SEQ ID NOs: 506 or 507, or fragments or variants of these.

In preferred embodiments, the at least one poly(A) sequence of the linear precursor RNA is characterized by any of the features relating to Poly(A) sequences as defined in the first aspect

In preferred embodiments, the at least one translation initiation sequence is characterized by any of the features relating to translation initiation sequences as defined in the first aspect

In preferred embodiments, the at least one coding sequence is characterized by any of the features relating to coding sequence as defined in the first aspect. Suitable peptide or proteins (encoded by said coding sequence) may be selected from peptides or proteins as defined in the first aspect

In preferred embodiments, the at least one UTR sequence of the linear precursor RNA is characterized by any of the features relating to UTR sequences as defined in the first aspect.

In preferred embodiments, the linear precursor RNA comprises at least one upstream UTR sequence characterized by any of the features relating to an upstream UTR sequence as defined in the first aspect In embodiments, the linear precursor RNA additionally comprises a poly(C) sequence as defined herein and/or a histone-stem loop sequence as defined herein and/or a miRNA binding sites as defined herein.

In preferred embodiments, the linear precursor RNA does not comprise chemically modified nucleotides.

In alternative embodiments, the circular RNA comprises modified nucleotides. Suitably modified nucleotides are defined in the context of the first aspect.

In preferred embodiments, the linear precursor RNA is an in vitro transcribed RNA.

In particularly preferred embodiments, the RNA in vitro transcription has been performed in the presence of a sequence optimized nucleotide mixture (as further specified in the context of the aspects “method of preparing and/or purifying circular RNA”).

In preferred embodiments, the linear precursor RNA of the invention comprises at least one purification tag.

A purification tag in the context of the invention may be any moiety or sequence that can be integrated into an RNA molecule. Preferably, a purification tag in the context of the invention may be any moiety or sequence that can be integrated into an RNA molecule via chemical RNA synthesis or RNA in vitro transcription.

In preferred embodiments, the at least one purification tag of the linear precursor RNA is located at the 3′ or the 5′ terminus of the linear precursor RNA.

This feature is particularly important in embodiments where linear precursor RNA serves as a template for RNA circularization via self splicing. As the 5′ terminus and the 3′ terminus of the linear precursor RNA is spliced out during the circularization reaction, a respective purification tag in said the 3′ or the 5′ of the linear precursor RNA may serve as a specific tag for purifying the resulting circular RNA preparation by specifically binding of non-circularized linear precursor RNA (that of course still carries the 3′ and/or the 5′ terminal tag), incomplete circular by-products (such by-products may still carry the 3′ and/or the 5′ terminal tag), or short intron fragments (that will still harbour these tags).

In particularly preferred embodiments, the purification tag in the context of the invention may be selected from a moiety or sequence that is integrated into the RNA sequence by means of RNA in vitro transcription. For example, the moiety may be a modified nucleotide that allows for affinity-based purification or chemical coupling. Suitably, as described above, the moiety or sequence should be integrated into the 3′ and/or the 5′ terminus.

In preferred embodiments, the at least one purification tag of the linear precursor RNA is an RNA sequence tag element. Suitably, the RNA sequence tag element is located at the 3′ and/or the 5′ terminus. This RNA sequence tag element should suitably be configured to allow specific affinity-based binding of the linear precursor RNA.

The RNA sequence tag element should suitably be configured not to interfere with the circularization reaction, in particular the self-splicing reaction. For example, the RNA sequence tag element should suitably be configured not to interfere with the 3′ permuted intron-exon element sequences or the 5′ permuted intron-exon element Preferably, the RNA sequence tag element may comprise or consist of a unstructured RNA sequence. This may be advantageous for the affinity-based capture of linear precursor RNA as further outlined in the context of the aspects “method of preparing and/or purifying circular RNA”.

In preferred embodiments, the RNA sequence tag element of the linear precursor RNA has a length ranging from about 5 nucleotides to about 50 nucleotides, preferably ranging from about 5 nucleotides to about 30 nucleotides, more preferably ranging from about 10 nucleotides to about 30 nucleotides.

In preferred embodiments, the at least one purification tag allows for an affinity-based binding of the linear precursor RNA in the presence of a corresponding circularized RNA. “Corresponding circularized RNA” has to be understood as the circular RNA product that is generated by self-splicing of the linear precursor RNA. As a result of the self-splicing, a corresponding circularized RNA does not comprise the 3′ and/or the 5′ terminal purification tag any more.

Accordingly, in preferred embodiments, the linear precursor RNA comprises a 3′ terminal RNA sequence tag element that allows for the specific affinity-based binding of said linear precursor RNA, wherein the 3′terminal RNA sequence tag element has a length ranging from about 5 nucleotides to about 50 nucleotides. Additionally or alternatively, the linear precursor RNA may comprise a 5′ terminal RNA sequence tag element that allows for the specific affinity-based binding of said linear precursor RNA, wherein the 5′ terminal RNA sequence tag element has a length ranging from about 5 nucleotides to about 50 nucleotides.

In various preferred embodiments, the affinity-based binding is performed using antisense oligonucleotide. (further specified in the context of the aspects “method of preparing and/or purifying circular RNA”)

In preferred embodiments, linear precursor RNA is configured for circularization via self-splicing. As outlined herein, the self-splicing event is triggered by the splicing sequences (3′ permuted intron-exon element and 5′ permuted intron-exon element) of the linear precursor RNA, yielding circular RNA and in addition two intron/homology arm linear RNA fragments (intronic splice products). For example, the two intronic splice products may resemble a nucleic acid sequence according to SEQ ID NO: 184 and according to SEQ ID NO: 185

Notably, RNA circularization takes place during and/or directly following RNA in vitro transcription. The respective circularization conditions are further specified in the context of the aspects “method of preparing and/or purifying circular RNA”.

In particularly preferred embodiments, the linear precursor RNA of the second aspect is for making a circular RNA, wherein the circular RNA (that is obtained by circularization of the linear precursor RNA) is characterized by any of the features according to the first aspect

In particularly preferred embodiments, the linear precursor RNA of the invention comprises or consists an RNA sequence identical or at least 70%, 80%, 90%, 91%, 92%, 93%, 94%, 95%, 96%, 97%, 98%, or 99% identical to any one of SEQ ID NOs: 197-200, 512-532 or a fragment or a variant of any of these, wherein the coding sequence (encoding Gluc or Ppluc) in any one of SEQ ID NOs: 197-200, 512-532 is exchanged by at least one coding sequence as defined herein, preferably a coding sequence encoding a therapeutic peptide or protein as defined herein.

In a further aspect, the invention provides a DNA template for producing the linear precursor RNA (e.g. via transcribing the DNA into an RNA by RNA in vitro transcription) as defined in the second aspect. Suitably, the DNA template may be a PCR product or a plasmid DNA. Suitably, the DNA template carries the sequence elements specified in the context of the first aspect, or specified in conjunction with the linear precursor RNA as specified in the context of the second aspect

### 3: A Pharmaceutical Composition Comprising Circular RNA

In a third aspect, the invention provides a pharmaceutical composition comprising circular RNA.

Notably, embodiments relating to the circular RNA of the first aspect may likewise be read on and be understood as suitable embodiments of the circular RNA comprised in the composition of the third aspect.

In preferred embodiments, the third aspect relates to a pharmaceutical composition comprising circular RNA, wherein the composition optionally comprises at least one pharmaceutically acceptable carrier.

Preferably, the circular RNA of the composition is as defined in any of the embodiments of the first aspect.

In particularly preferred embodiments, the circular RNA of the pharmaceutical composition comprises or consists an RNA sequence identical or at least 70%, 80%, 90%, 91%, 92%, 93%, 94%, 95%, 96%, 97%, 98%, or 99% identical to any one of SEQ ID NOs: 202-205, 533-544 or a fragment or a variant of any of these, wherein the coding sequence (encoding Gluc or Ppluc) in any one of SEQ ID NOs: 202-205, 533-544 is exchanged by at least one coding sequence as defined herein, preferably a coding sequence encoding a therapeutic peptide or protein as defined herein.

In preferred embodiments, the pharmaceutical composition comprises a plurality or at least more than one circular RNA species, preferably wherein each circular RNA species encodes a different peptide or protein.

Preferably, the pharmaceutical composition as defined herein may comprise 2, 3, 4, 5, 6, 7, 8, 9, or 10 circular RNA species each as defined herein, wherein each of the 2, 3, 4, 5, 6, 7, 8, 9, or 10 circular RNA species encode a different peptide or protein, wherein the at least one different peptide or protein differs in at least one amino acid.

In preferred embodiments, the circular RNA of the pharmaceutical composition is complexed or associated with at least one further compound to obtain a formulated composition. A formulation in that context may have the function of a transfection agent. A formulation in that context may also have the function of protecting the circular RNA from degradation, e.g. to allow storage, shipment, etc.

In various embodiments, the circular RNA of the pharmaceutical composition is formulated with a pharmaceutically acceptable carrier or excipient.

In preferred embodiments, the circular RNA of the pharmaceutical composition is formulated with at least one compound, e.g. peptides, proteins, lipids, polysaccharides, and/or polymers.

In preferred embodiments, the circular RNA of the pharmaceutical composition is formulated with at least one cationic (cationic or preferably ionizable) or polycationic compound (cationic or preferably ionizable).

In preferred embodiments, the circular RNA of the pharmaceutical composition is complexed or associated with or at least partially complexed or partially associated with one or more cationic (cationic or preferably ionizable) or polycationic compound.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Om. Accordingly, “polycationic” components are also within the scope exhibiting more than one positive charge under the given conditions.

In preferred embodiments, the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

In preferred embodiments, the at least one cationic or polycationic compound is selected from a cationic or polycationic peptide or protein.

Suitable cationic or polycationic proteins or peptides that may be used for complexation of the circular RNA can be derived from formula (Arg)I;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In preferred embodiments, the at least one circular RNA of the pharmaceutical composition is complexed, or at least partially complexed, with at least one cationic or polycationic proteins or peptides preferably selected from 15 an one of SEQ ID NOs: 179-183 or fragments or variants of any of these, or any combinations thereof.

According to various embodiments, the pharmaceutical composition comprises at least one circular RNA as defined herein, and a polymeric carrier.

The term “polymeric carrier” as used herein will be recognized and understood by the person of ordinary skill in the art, and are e.g. intended to refer to a compound that facilitates transport and/or complexation of another compound. A polymeric carrier is typically a carrier that is formed of a polymer. A polymeric carrier may be associated to its cargo (e.g. circular RNA) by covalent or non-covalent interaction. A polymer may be based on different subunits, such as a copolymer.

Suitable polymeric carriers in that context may include, for example, polyethyleneimine (PEI).

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are cross-linked by disulfide bonds (via —SH groups).

In this context, polymeric carriers according to formula {(Arg)I;(Lys)m;(His)n;(Om)o;(Xaa)x(Cys)y} and formula Cys,{(Arg)l;(Lys)m;(His)n;(Om)o;(Xaa)x)Cys2 of the patent application WO2012/013326 are preferred, the disclosure of WO2012/013326 relating thereto incorporated herewith by reference.

In embodiments, the polymeric carrier used to complex the at least one circular RNA may be derived from a polymeric carrier molecule according formula (L-P1-S-fS-P2-S]n-S-P3-L) of the patent application WO2011/026641, the disclosure of WO2011/026641 relating thereto incorporated herewith by reference.

In embodiments, the polymeric carrier compound is formed by, or comprises or consists of the peptide elements CysArg12Cys (SEQ ID NO: 179) or CysArg12 (SEQ ID NO: 180) or TrpArg12Cys (SEQ ID NO: 181). In particularly preferred embodiments, the polymeric carrier compound consists of a (R12C)-(R12C) dimer, a (WR12C)-(WR12C) dimer, or a (CR12)-(CR12C)-(CR12) trimer, wherein the individual peptide elements in the dimer (e.g. (WR12C)), or the trimer (e.g. (CR12)), are connected via —SH groups.

In embodiments, the pharmaceutical composition comprises at least one circular RNA that is complexed or associated with polymeric carriers and, optionally, with at least one lipid component as described in WO2017/212008A1, WO2017/212006A1, WO2017/212007A1, and WO2017/212009A1. In this context, the disclosures of WO2017/212008A1, WO2017/212006A1, WO2017/212007A1, and WO2017/212009A1 are herewith incorporated by reference.

In a particularly preferred embodiment, the polymeric carrier is a peptide polymer, preferably a polyethylene glycol/peptide polymer as defined above, and a lipid component, preferably a lipidoid component.

A lipidoid (or lipidoit) is a lipid-like compound, i.e. an amphiphilic compound with lipid-like physical properties. The lipidoid is preferably a compound, which comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention, the term lipid is considered to also encompass lipidoids.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In preferred embodiments, the lipidoid component of the polymeric carrier may be any one selected from the table of lipidoid structures of published PCT patent application WO2017/212009A1 (pages 50-54).

In particularly preferred embodiments, the circular RNA is formulated in lipid-based carriers.

In the context of the invention, the term “lipid-based carriers” encompass lipid based delivery systems for circular RNA that comprise a lipid component. A lipid-based carrier may additionally comprise other components suitable for encapsulating/incorporating/complexing a circular RNA including a cationic or polycationic polymer, a cationic or polycationic polysaccharide, a cationic or polycationic protein, a cationic or polycationic peptide, or any combinations thereof.

In the context of the invention, a typical “lipid-based carrier” is selected from liposomes, lipid nanoparticles (LNPs), lipoplexes, and/or nanoliposomes. Preferably, the lipid-based carrier is a lipid nanoparticle. The circular RNA of the pharmaceutical composition may completely or partially incorporated or encapsulated in a lipid-based carrier, wherein the circular RNA may be located in the interior space of the lipid-based carrier, within the lipid layer/membrane of the lipid-based carrier, or associated with the exterior surface of the lipid-based carrier.

The incorporation of circular RNA into lipid-based carriers may be referred to as “encapsulation”. A “lipid-based carrier” is not restricted to any particular morphology, and include any morphology generated when e.g. an aggregation reducing lipid and at least one further lipid are combined, e.g. in an aqueous environment in the presence of circular RNA. For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the circular RNA is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanopartides (LNPs), a specific type of lipid-based carrier, are characterized as microscopic lipid particles having a solid core or partially solid core. Typically, an LNP does not comprise an interior aqua space sequestered from an outer medium by a bilayer. In an LNP, the circular RNA may be encapsulated or incorporated in the lipid portion of the LNP enveloped by some or the entire lipid portion of the LNP. An LNP may comprise any lipid capable of forming a particle to which the circular RNA may be attached, or in which the circular RNA may be encapsulated.

In preferred embodiments, the lipid-based carriers of the pharmaceutical composition are selected from liposomes, lipid nanoparticles, lipoplexes, and/or nanoliposomes.

In preferred embodiments, the lipid-based carriers of the pharmaceutical composition are lipid nanoparticles (LNPs). In particularly preferred embodiments, the lipid nanopartides of the pharmaceutical composition encapsulate the circular RNA of the invention.

The term “encapsulated”, e.g. incorporated, complexed, encapsulated, partially encapsulated, associated, partially associated, refers to the essentially stable combination of circular RNA with one or more lipids into lipid-based carriers (e.g. larger complexes or assemblies) preferably without covalent binding of the circular RNA. The lipid-based carriers—encapsulated RNA may be completely or partially located in the interior of the lipid-based carrier (e.g. the lipid portion and/or an interior space) and/or within the lipid layer/membrane of the lipid-based carriers. The encapsulation of a circular RNA into lipid-based carriers is also referred to herein as “incorporation” as the circular RNA is preferably contained within the interior of the lipid-based carriers. Without wishing to be bound to theory, the purpose of incorporating or encapsulating circular RNA into lipid-based carriers may be to protect the circular RNA from an environment which may contain enzymes, chemicals, or conditions that degrade the RNA. Moreover, incorporating circular RNA into lipid-based carriers may promote the uptake of the circular RNA, and hence, may enhance the therapeutic effect of the circular RNA when administered to a cell or a subject.

In preferred embodiments, the lipid-based carriers of the pharmaceutical composition comprise at least one or more lipids selected from at least one aggregation-reducing lipid, at least one cationic lipid, at least one neutral lipid or phospholipid, or at least one steroid or steroid analog.

In preferred embodiments, the lipid-based carriers of the pharmaceutical composition comprise an aggregation-reducing lipid, a cationic lipid or ionizable lipid, a neutral lipid or phospholipid, and a steroid or steroid analog.

The term “aggregation reducing lipid” refers to a molecule comprising both a lipid portion and a moiety suitable of reducing or preventing aggregation of the lipid-based carriers in a composition. Under storage conditions or during formulation, the lipid-based carriers may undergo charge-induced aggregation, a condition which can be undesirable for the stability of the composition. Therefore, it can be desirable to include a lipid compound which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the invention, stabilization of the lipid-based carriers is achieved by including lipids which may comprise a lipid bearing a sterically bulky group which, after formation of the lipid-based carrier, is preferably located on the exterior of the lipid-based carrier. Suitable aggregation reducing groups include hydrophilic groups, e.g. polymers, such as poly(oxyalkylenes), e.g., a poly(ethylene glycol) or poly(propylene glycol). Lipids comprising a polymer as aggregation reducing group are herein referred to as “polymer conjugated lipid”.

The term “polymer conjugated lipid” refers to a molecule comprising both a lipid portion and a polymer portion, wherein the polymer is suitable of reducing or preventing aggregation of lipid-based carriers comprising the RNA. A polymer has to be understood as a substance or material consisting of very large molecules, or macromolecules, composed of many repeating subunits. A suitable polymer in the context of the invention may be a hydrophilic polymer. An example of a polymer conjugated lipid is a PEGylated or PEG-conjugated lipid.

In preferred embodiments, the lipid-based carriers of the pharmaceutical composition comprise an aggregation reducing lipid selected from a polymer conjugated lipid. As used herein, the terms “aggregation reducing lipid” and “polymer conjugated lipid” may be used interchangeably.

In preferred embodiments, the polymer conjugated lipid is a PEG-conjugated lipid (or PEGylated lipid, PEG lipid).

In other preferred embodiments, the polymer conjugated lipid is a POZ or preferably PMOZ lipid.

In preferred embodiments, the polymer conjugated lipid, e.g. the PEG-conjugated lipid, is selected or derived from 1,2-dimyristoyl-rac-glycero-3-methoxypolyethylene glycol-2000 (PEG2000 DMG or DMG-PEG 2000). As used in the art, “DMG-PEG 2000” is typically considered a mixture of 1,2-DMG PEG2000 and 1,3-DMG PEG2000 in-97:3 ratio.

In other embodiments, the polymer conjugated lipid, e.g. the PEG-conjugated lipid, is selected or derived from C10-PEG2K, or Cer8-PEG2K.

In other preferred embodiments, the polymer conjugated lipid, e.g. the PEG-conjugated lipid, is selected or derived from a lipid with the chemical term 2[(polyethylene glycol)-2000]-N,N-ditetradecylacetamide, also referred to as ALC-0159.

Accordingly, in preferred embodiments, the polymer conjugated lipid is a PEG-conjugated lipid selected or derived from DMG-PEG 2000, C10-PEG2K, Cer8-PEG2K, or ALC-0159.

In preferred embodiments, the lipid-based carriers of the pharmaceutical composition comprise an aggregation reducing lipid, wherein the aggregation reducing lipid is not a PEG-conjugated lipid.

In preferred embodiments, the lipid-based carriers of the pharmaceutical composition comprise a polymer conjugated lipid, wherein the polymer conjugated lipid is not a PEG-conjugated lipid.

In preferred embodiments, the lipid-based carriers of the pharmaceutical composition do not comprise a PEG-conjugated lipid. Accordingly, in further preferred embodiments, the polymer conjugated lipid is a POZ-lipid, which is defined as a compound according to formula (POZ):

[H]—[linker]—[M] formula (POZ)

wherein
[H] is a homopolymer moiety comprising at least one polyoxazoline (POZ) monomer unit

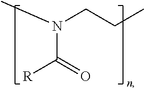

wherein R is C₁₋₆ alkyl or C₂₋₉ alkenyl and n has a mean value ranging from 2 to 200, preferably from 20 to 100, more preferably from 24 to 26 or 45 to 50
[linker] is an optional linker group, and
[M] is a lipid moiety.

In an embodiment, [H] is a heteropolymer moiety or homopolymer moiety comprising multiple monomer units selected from the group consisting of

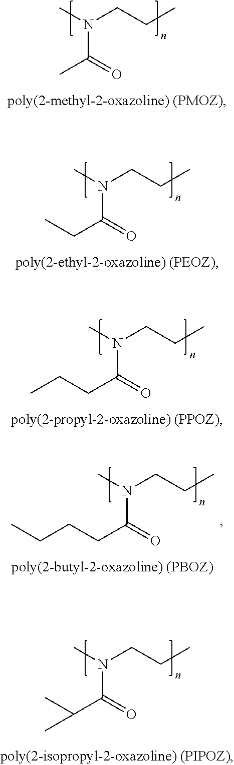

poly(2-methoxymethyl-2-oxazoline) (PMeOMeOx), and
poly(2-dimethylamino-2-oxazoline) (PDMAOx),

preferably wherein [H] is a homopolymer moiety comprising multiple PMOZ or PEOZ monomer units, more preferably wherein [H] comprises or preferably consists of multiple PMOZ monomer units, wherein
(i) n has a mean value ranging from 2 to 200, preferably from 20 to 100, more preferably from 24 to 26 or 45 to 50 or wherein
(ii) n is selected such that the [H] moiety has an average molecular weight of 1,5 to 22 kDa, more preferably of 2 to 19 kDa, even more preferably of about 7.5 kDa or of about 15 kDa, preferably from 1 to 15 kDa, more preferably of 2 to 12.5 kDa, even more preferably of about 5 kDa or of about 10 kDa.

In another embodiment, [H] is a heteropolymer moiety or homopolymer moiety comprising multiple monomer units selected from the group consisting of

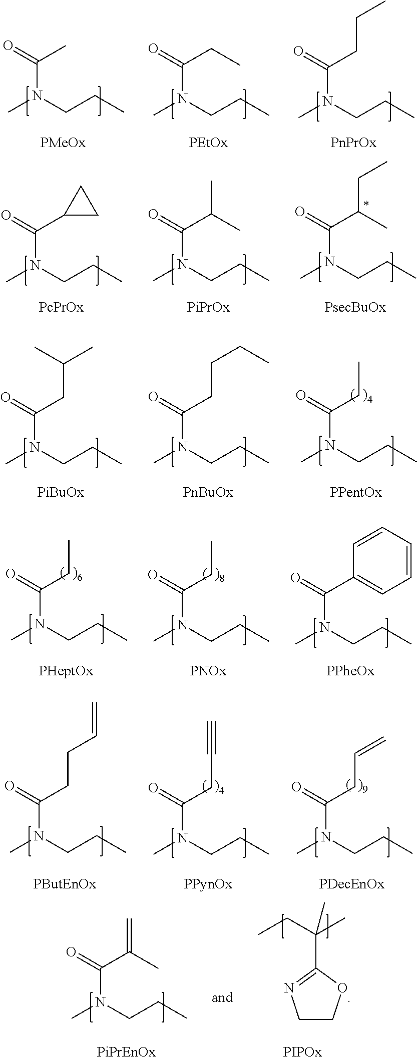

In yet another embodiment, the [H] from the polymer conjugated lipid according to formula (POZ) is selected from the group consisting of poly(2-methoxymethyl-2-oxazoline) (PMeOMeOx) and poly(2-dimethylamino-2-oxazoline) (PDMAOx).

In one embodiment, the lipid moiety [M] as shown in formula (POZ) comprises at least one straight or branched, saturated or unsaturated alkyl chain containing from 6 to 30 carbon atoms, preferably wherein the lipid moiety [M] comprises at least one straight or branched saturated alkyl chain, wherein the alkyl chain is optionally interrupted by one or more biodegradable group(s) and/or optionally comprises one terminal biodegradable group, wherein the biodegradable group is selected from the group consisting of but not limited to a pH-sensitive moiety, a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)═N—O— or —O— N═C(H)—), carbamate (—NHC(O)O—), urea (—NHC(O)NH—), succinyl (—(O)CCH2CH2C(O)—), succinamidyl (—NHC(O)CH2CH2C(O)NH—), —C(R⁵)═N—, —N═C(R⁵)—, —C(R⁵)═N—O—, —O—N═C(R⁵)—, —O—C(O)O—, —C(O)N(R⁵), —N(RW)C(O)—, —C(S)(NR⁵—, (NR⁵)C(S)—, —N(RW)C(O)N(R⁵)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—, carbonate (—OOC(O)—), succinoyl, phosphate esters (—O—(O)POH—O—), cyclic compound, heterocyclic compound, piperidine, pyrazine, pyridine, piperazine, and sulfonate esters, as well as combinations thereof, wherein R³, R⁴ and R⁵ are, independently H or alkyl (e.g. C₁-C₄ alkyl).

In another embodiment, the the lipid moiety [M] comprises at least one straight or branched, saturated or unsaturated alkyl chain comprising 6, 7, 8, 9, 10, 11,12, 13, 14, 15, 16,17, 18,19, 20, 21, 22, 23, 24, 25, 26, 27, 28, 29, or 30 carbon atoms, preferably in the range of 10 to 20 carbon atoms, more preferably in the range of 12 to 18 carbon atoms, even more preferably 14, 16 or 18 carbon atoms, even more preferably 16 or 18 carbon atoms, most preferably 14 carbon atoms, wherein all selections are independent of one another.

In one embodiment, the linker group [linker] as shown in formula (POZ) is selected from the group consisting of but not limited to a pH-sensitive moiety, a zwitterionic linker, non-ester containing linker moieties and ester-containing linker moieties (—C(O)O— or —OC(O)—), amido (—C(O)NH—), disulfide (—S—), carbonyl (—C(O)—), ether (—O—), thioether (—S—), oxime (e.g., —C(H)═N—O— or —O— N═C(H)—), carbamate (—NHC(O)O—), urea (—NHC(O)NH—), succinyl (—(O)CCH2CH2C(O)—), succinamidyl (—NHC(O)CH2CH2C(O)NH—), —C(R⁵)═N—, —N═C(R⁵)—, —C(R⁵)═N—O—, —O—N═C(R⁵)—, —O—C(O)O—, —C(O)N(R⁵), —N(R⁵)C(O)—, —C(S)(NR⁵)—, (NR⁵)C(S)—, —N(R⁵)C(O)N(R⁵)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—, carbonate (—OC(O)O—), succinoyl, phosphate esters (—O—{O)POH—O—), and sulfonate esters, as well as combinations thereof, wherein R³, R⁴ and R⁵ are, independently H or alkyl (e.g. C₁-C₄ alkyl).

In a very preferred embodiment, the polymer conjugated lipid has the structure of

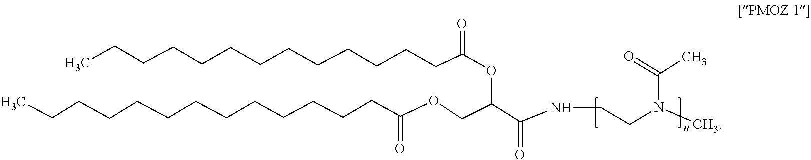

In another very preferred embodiment, the polymer conjugated lipid has the structure of

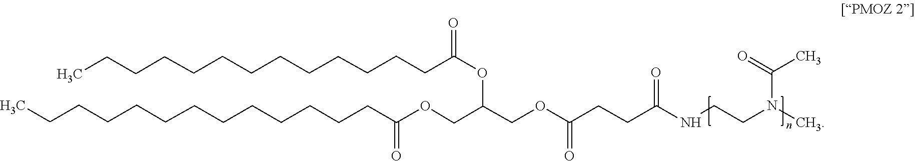

In another very preferred embodiment, the polymer conjugated lipid has the structure of “PMOZ 2” with n=50 i.e. having 50 monomer repeats.

In an even further preferred embodiment, the polymer conjugated lipid has the structure of

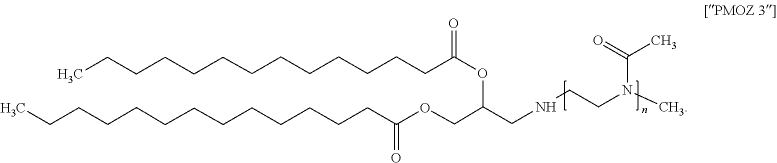

In another preferred embodiment, the polymer conjugated lipid has the structure of

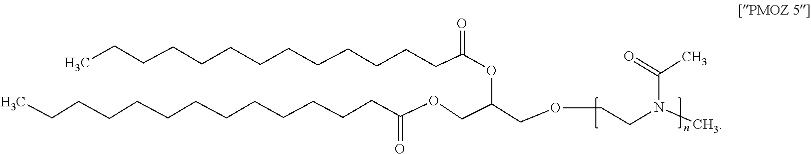

In an even further very preferred embodiment, the polymer conjugated lipid has the structure of

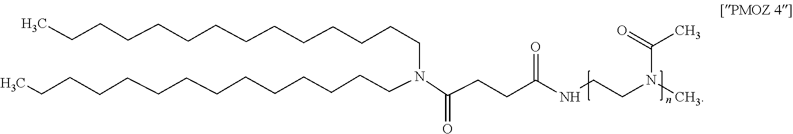

preferably with n=50 i.e. having 50 monomer repeats, i.e.

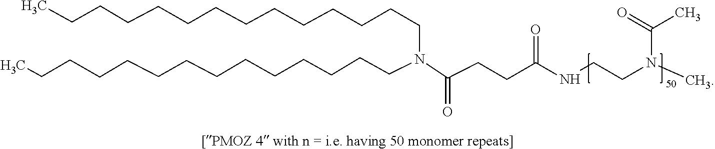

For “PMOZ 1” to “PMOZ 5”, preferably n has a mean value ranging from 2 to 200, preferably from 20 to 100, more preferably from 24 to 26, even more preferably about 100, or further even more preferably from 45 to 50, most preferably 50 or wherein n is selected such that the [P] moiety has an average molecular weight of about 4.2 kDa to about 4.4 kDa, or most preferably about 4.3 kDa.

In another very preferred embodiment, the linker group [linker] comprises preferably an amide linker moiety.

In a further very preferred embodiment, the linker group [linker] comprises preferably an ester linker moiety.

In a further very preferred embodiment, the linker group [linker] comprises preferably a succinate linker moiety.

In another very preferred embodiment, the linker group [linker] comprises both an ester linker and an amid linker moiety. In another preferred embodiment, the linker group [linker] comprises both an ester linker, an amine linker and an amid linker moiety.

It is noted herein, that all chemical compounds mentioned throughout the whole specification may be produced via processes known to a skilled worker; starting materials and/or reagents used in the processes are obtainable through routine knowledge of a skilled worker on the basis of common general knowledge (e.g. from text books or from e.g. patent applications WO2022173667, WO2009043027, WO2013067199, WO2010006282, WO2009089542, WO2016019340, WO2008106186, WO2020264505, and WO2020023947, the complete disclosure of said patent applications is incorporated by reference herein).

In yet a further embodiment, the lipid nanoparticle does not comprise a polyethylene glycol-(PEG)-lipid conjugate or a conjugate of PEG and a lipid-like material, and preferably do not comprise PEG and/or (ii) the polymer conjugated lipid of the invention does not comprise a sulphur group (—S—), a terminating nucleophile, and/or is covalently coupled to a biologically active ingredient is a nucleic acid compound selected from the group consisting of RNA, an artificial mRNA, chemically modified or unmodified messenger RNA (mRNA) comprising at least one coding sequence, self-replicating RNA, circular RNA, viral RNA, and replicon RNA; or any combination thereof, preferably wherein the biologically active ingredient is chemically modified mRNA or chemically unmodified mRNA, more preferably wherein the biologically active ingredient is chemically unmodified mRNA.

In another very preferred embodiment, the polymer conjugated lipid of the invention does not comprise sulphur (S) or a sulphur group (—S—).

In further preferred embodiments, lipid nanopartices and/or polymer conjugated lipids may be selected from the lipid nanopartides and/or lipids as disclosed in PCT/EP2022/074439 (i.e. lipids derived from formula I, II, and III of PCT/EP2022/074439, or lipid nanoparticles and/or lipids as specified in Claims 1 to 46 of PCT/EP2022/074439), the disclosure of PCT/EP2022/074439 hereby incorporated by reference in its entirety.

In preferred embodiments, the lipid-based carriers comprise a cationic or ionizable lipid.

The cationic lipid of the lipid-based carriers may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Suitable cationic lipids or cationisable lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3}-dioxolane) HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d] [1,3]dioxol-5-amine)), 1,1′-(2-(4-2(2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N,N 16-diundecyl-4,7, 10,13-tetraazahexadecane-1,16-diamide), (6Z,9Z,28Z,31Z)-heptatiaconta-6,9,28,31-tetraen-19-yl4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN© (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from 30 GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic or cationizable lipids include those described in international patent publications WO2010/053572 (and particularly, CI2-200 described at paragraph [00225]) and WO2012/170930, both of which are incorporated herein by reference, HGT4003, HGT5000, HGTS001, HGT5001, HGT5002 (see US20150140070A1), 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120), lipids as disclosed in Tables 1, 2 and 3 and as defined in claims 1-24 of WO2017/075531A1, lipids selected from compounds as disclosed in WO2015/074085A1 (i.e. ATX-001 to ATX-032 or the compounds as specified in claims 1-26), lipids selected from compounds as disclosed in WO2017/117530A1 (i.e. lipids 13, 14, 15, 16, 17, 18, 19, 20, or the compounds as specified in the claims).

In preferred embodiments, the lipid-based carriers comprise a cationic or ionizable lipid that preferably carries a net positive charge at physiological pH, more preferably wherein the cationic or ionizable lipid comprises a tertiary nitrogen group or quatemary nitrogen group.

In embodiments, cationic or cationizable lipids may be selected from the lipids disclosed in WO2018/078053A1 (i.e. lipids derived from formula 1, 11, and III of WO2018/078053A1, or lipids as specified in Claims 1 to 12 of WO2018/078053A1), the disclosure of WO2018/078053A1 hereby incorporated by reference in its entirety. In preferred embodiments, the lipid-based carriers of the pharmaceutical composition comprise a cationic lipid selected or derived from structures Ill-1 to 111-36 of Table 9 of published PCT patent application WO2018/078053A1. Accordingly, formula III-1 to 111-36 of WO2018/078053A1, and the specific disclosure relating thereto, are herewith incorporated by reference.

In preferred embodiments, the lipid-based carriers (e.g. LNPs) of the pharmaceutical composition comprise a cationic lipid selected or derived from [(4-Hydroxybutyl)azandiyl]bis(hexan-6,1-diyl)bis(2-hexyldecanoat), also referred to as ALC-0315.

Further suitable cationic lipids may be selected or derived from cationic lipids according to PCT claims 1 to 14 of published patent application WO2021123332, or table 1 of WO2021123332, the disclosure relating to claims 1 to 14 or table 1 of WO2021123332 herewith incorporated by reference.

Accordingly, suitable cationic lipids may be selected or derived from cationic lipids according Compound 1 to Compound 27 (C1-C27) of Table 1 of WO2021123332.

In preferred embodiments, the lipid-based carriers (e.g. LNPs) of the pharmaceutical composition comprise a cationic lipid selected or derived from (COATSOME® SS-EC)SS-33/4PE-15 (see C23 in Table 1 of WO2021123332)

In preferred embodiments, the lipid-based carriers (e.g. LNPs) of the pharmaceutical composition comprise a cationic lipid selected or derived from HEXA-C5DE-PipSS (see C2 in Table 1 of WO2021123332) Other suitable cationic or ionizable, neutral, steroid/sterol or aggregation reducing lipids are disclosed in WO2010053572, WO2011068810, WO2012170889, WO2012170930, WO2013052523, WO2013090648, WO2013149140, WO2013149141, WO2013151663, WO2013151664, WO2013151665, WO2013151666, WO2013151667, WO2013151668, WO2013151669, WO2013151670, WO2013151671, WO2013151672, WO2013151736, WO2013185069, WO2014081507, WO2014089486, WO2014093924, WO2014144196, WO2014152211, WO2014152774, WO2014152940, WO2014159813, WO2014164253, WO2015061461, WO2015061467, WO2015061500, WO2015074085, WO2015105926, WO2015148247, WO2015164674, WO2015184256, WO2015199952, WO2015200465, WO2016004318, WO2016022914, WO2016036902, WO2016081029, WO2016118724, WO2016118725, WO2016176330, WO2017004143, WO2017019935, WO2017023817, WO2017031232, WO2017049074, WO2017049245, WO2017070601, WO2017070613, WO2017070616, WO2017070618, WO2017070620, WO2017070622, WO2017070623, WO2017070624, WO2017070626, WO2017075038, WO2017075531, WO2017099823, WO2017106799, WO2017112865, WO2017117528, WO2017117530, WO2017180917, WO2017201325, WO2017201340, WO2017201350, WO2017201352, WO2017218704, WO2017223135, WO2018013525, WO2018081480, WO2018081638, WO2018089540, WO2018089790, WO2018089801, WO2018089851, WO2018107026, WO2018118102, WO2018119163, WO2018157009, WO2018165257, WO2018170245, WO2018170306, WO2018170322, WO2018170336, WO2018183901, WO2018187590, WO2018191657, WO2018191719, WO2018200943, WO2018231709, WO2018231990, WO2018232120, WO2018232357, WO2019036000, WO2019036008, WO2019036028, WO2019036030, WO2019040590, WO2019089818, WO2019089828, WO2019140102, WO2019152557, WO2019152802, WO2019191780, WO2019222277, WO2019222424, WO2019226650, WO2019226925, WO2019232095, WO2019232097, WO2019232103, WO2019232208, WO2020061284, WO2020061295, WO2020061332, WO2020061367, WO2020081938, WO2020097376, WO2020097379, WO2020097384, WO2020102172, WO2020106903, WO2020146805, WO2020214946, WO2020219427, WO2020227085, WO2020232276, WO2020243540, WO2020257611, WO2020257716, WO2021007278, WO2021016430, WO2021022173, WO2021026358, WO2021030701, WO2021046260, WO2021050986, WO2021055833, WO2021055835, WO2021055849, WO2021127394, WO2021127641, WO2021202694, WO2021231697, WO2021231901, WO2008103276, WO2009086558, WO2009127060, WO2010048536, WO2010054406, WO2010080724, WO2010088537, WO2010129709, WO201021865, WO2011022460, WO2011043913, WO2011090965, WO2011149733, WO2011153120, WO2011153493, WO2012040184, WO2012044638, WO2012054365, WO2012061259, WO2013063468, WO2013086354, WO2013086373, U.S. Pat. No. 7,893,302B2, U.S. Pat. No. 7,404,969B2, U.S. Pat. No. 8,158,601B2, U.S. Pat. No. 8,283,333B2, U.S. Pat. No. 8,466,122B2, U.S. Pat. No. 8,569,256B2, US20100036115, US20110256175, US20120202871, US20120027803, US20120128760, US20130064894, US20130129785, US20130150625, US20130178541, US20130225836, and US20140039032; the disclosures specifically relating to cationic or ionizable, neutral, sterol or aggregation reducing lipids suitable for lipid-based carriers of the foregoing publications are incorporated herewith by reference.

In some embodiments, the lipid-based carriers (e.g. LNPs) of the pharmaceutical composition comprise a cationic lipid selected or derived from Heptadecan-9-yl 8-((2-hydroxyethyl)(6-oxo-6-(undecyloxy)hexyl)amino)octanoate, also referred to as SM-102.

In further preferred embodiment of the second aspect, the at least one RNA, preferably the at least one mRNA is complexed with one or more lipids thereby forming LNPs, wherein the LNPs comprises a cationic lipid according to formula X-1:

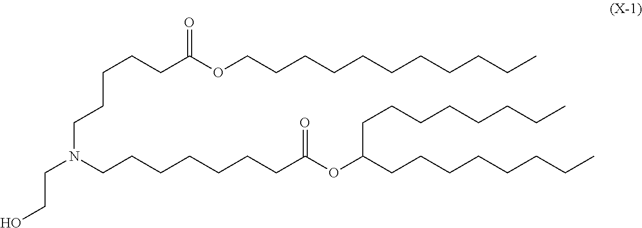 [SMILES: CCCCCCCCCCCOC(=O)CCCCCN(CCO)CCCCCCCC(=O)OC(CCCCCCCC)CCCCCCCC]

The lipid of formula X-1 as suitably used herein has the chemical term (9-Heptadecanyl 8-{(2-hydroxyethyl)[6-oxo-6-(undecyloxy)hexyl]amino}octanoate) or “Heptadecan-9-yl 8-((2-hydroxyethyl)(6-oxo-6-(undecyloxy)hexyl)amino)octanoate”, also referred to as SM-102.

In certain embodiments, the cationic lipid as defined herein, more preferably cationic lipid compound X-1, is present in the LNP in an amount from about 30 to about 95 mole percent, relative to the total lipid content of the LNP. If more than one cationic lipid is incorporated within the LNP, such percentages apply to the combined cationic lipids.

In embodiments, the cationic lipid is present in the LNP in an amount from about 30 to about 70 mole percent. In one embodiment, the cationic lipid is present in the LNP in an amount from about 40 to about 60 mole percent, such as from about 45 to about 55 mole percent or about 47 to about 50 mole percent. In embodiments, the cationic lipid is present in the LNP in an amount from about 48 to about 49 mole percent, such as about 48.0, 48.1, 48.2, 48.3, 48.4, 48.5, 48.6, 48.7, 48.8, 48.9, 49.0 mole percent, respectively, wherein 48.5 mole percent are particularly preferred.

In other preferred embodiments, the lipid-based carriers (e.g. LNPs) of the pharmaceutical composition comprise a cationic lipid selected or derived from HEXA-C5DE-PipSS (see C2 in Table 1 of WO2021123332). In other preferred embodiments, the lipid-based carriers (e.g. LNPs) of the pharmaceutical composition comprise a cationic lipid selected or derived from compound C26 as disclosed in Table 1 of WO2021123332 or herein:

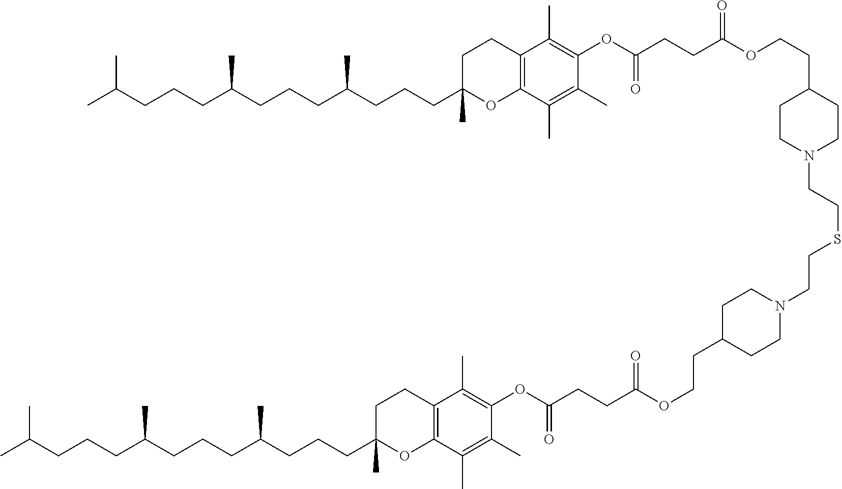 [SMILES: Cc1c(C)c2c(c(C)c1OC(=O)CCC(=O)OCCC1CCN(CCSCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2]

Other preferred lipid-based carriers (e.g. LNPs) of the pharmaceutical composition comprise a squaramide ionizable amino lipid, more preferably a cationic lipid selected from the group consisting of formulas (M1) and (M2):

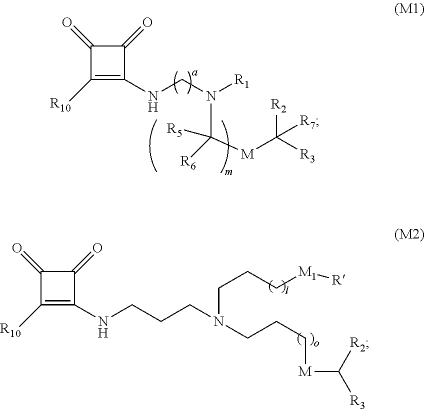

wherein the substituents (e.g. R₁, R₂, R₃, R₅, R₆, R₇, R₁₀, M, M₁, m, n, o, 1) are defined in claims 1 to 13 of U.S. Ser. No. 10/392,341B2; U.S. Ser. No. 10/392,341B2 being incorporated herein in its entirety.

Accordingly, in preferred embodiments, the lipid-based carriers (e.g. LNPs) of the pharmaceutical composition comprise a cationic lipid selected or derived from ALC-0315, SM-102, SS-33/4PE-15, HEXA-C5DE-PipSS or compound C26 (according to C26 in Table 1 of WO2021123332 or as disclosed herein).

In specific embodiments, the lipid-based carriers of the invention comprise two or more (different) cationic lipids as defined herein.

In preferred embodiments, the lipid-based carriers (e.g. LNPs) comprise a neutral lipid or phospholipid.

The term “neutral lipid” refers to any one of a number of lipid species that exist in either an uncharged or neutral zwitterionic form at physiological pH. Suitable neutral lipids include diacylphosphatidylcholines, diacylphosphatidylethanolamines, ceramides, sphingomyelins, dihydro sphingomyelins, cephalins, and cerebrosides.

In preferred embodiments, the neutral lipid of the lipid-based carriers (e.g. LNPs) of the pharmaceutical composition is selected or derived from 1,2-distearoyl-sn-glycero-3-phosphocholine (DSPC).

In other preferred embodiments, the neutral lipid of the lipid-based carriers (e.g. LNPs) of the pharmaceutical composition is selected or derived from 1,2-diheptanoyl-sn-glycero-3-phosphocholine (DHPC).

In other preferred embodiments, the neutral lipid of the lipid-based carriers (e.g. LNPs) of the pharmaceutical composition is selected or derived from 1,2-diphytanoyl-sn-glycero-3-phosphoethanolamine (DPhyPE).

Accordingly, in preferred embodiments, the lipid-based carriers (e.g. LNPs) of the pharmaceutical composition comprise a neutral lipid selected or derived from DSPC, DHPC, or DPhyPE.

In preferred embodiments, the lipid-based carriers of the pharmaceutical composition comprise a steroid or steroid analog.

Suitably, the steroid or steroid analog may be derived or selected from cholesterol, cholesteryl hemisuccinate (CHEMS) and a derivate thereof.

In particularly preferred embodiments, the lipid-based carriers of the pharmaceutical composition comprise cholesterol.

In some embodiments, the cholesterol is a polymer conjugated cholesterol or a PEGylated cholesterol.

In preferred embodiments, the lipid-based carriers of the pharmaceutical composition, preferably the LNPs, comprise the circular RNA as defined herein, a cationic lipid as defined herein, an aggregation reducing lipid as defined herein, optionally, a neutral lipid as defined herein, and, optionally, a steroid or steroid analog as defined herein.

In preferred embodiments, the lipid-based carriers comprising the circular RNA comprise comprise
(i) at least one cationic lipid or ionizable lipid, preferably as defined herein;
(ii) at least one neutral lipid or phospholipid, preferably as defined herein;
(iii) at least one steroid or steroid analogue, preferably as defined herein; and
(iv) at least one aggregation reducing lipid, preferably as defined herein; In preferred embodiments, the lipid-based carriers comprising the circular RNA comprise
(i) at least one cationic lipid selected or derived from ALC-0315, SM-102, SS-33/4PE-15, HEXA-C5DE-PipSS or compound C26 (according to C26 in Table 1 of WO2021123332);
(ii) at least one neutral lipid selected or derived from DSPC, DHPC, or DPhyPE;
(iii) at least one steroid or steroid analog selected or derived from cholesterol; and
(iv) at least one aggregation reducing lipid selected or derived from DMG-PEG 2000, C10-PEG2K, Cer8-PEG2K, or ALC-0159; and wherein the lipid-based carriers encapsulate the circular RNA.

In preferred embodiments, the cationic lipids (as defined herein), neutral lipid (as defined above), steroid or steroid analog (as defined above), and/or aggregation reducing lipid (as defined above) may be combined at various relative molar ratios.

In preferred embodiments, the lipid-based carriers comprise (i) to (iv) in a molar ratio of about 20-60% cationic lipid or ionizable lipid, about 5-25% neutral lipid, about 25-55% steroid or steroid analogue, and about 0.5-15% aggregation reducing lipid e.g. polymer conjugated lipid, preferably wherein the lipid-based carriers encapsulate the circular RNA.

In specific embodiments, the lipid-based carriers comprise or consist (i) to (iv) in a molar ratio of about 47.4% cationic lipid, about 10% neutral lipid, about 40.9% steroid or steroid analogue, and about 1.7% aggregation reducing lipid, e.g. polymer conjugated lipid, preferably wherein the lipid-based carriers encapsulate the circular RNA.

In specific embodiments, the lipid-based carriers comprise or consist (i) to (iv) in a weight ratio of about 56.28% cationic lipid, about 12.24% neutral lipid, about 24.51% steroid or steroid analogue, and about 6.97% aggregation reducing lipid, preferably wherein the lipid-based carriers encapsulate the circular RNA.

In preferred embodiments, the lipid-based carriers comprising the circular RNA comprise
(i) at least one cationic lipid selected from SM-102;
(ii) at least one neutral lipid selected from DSPC;
(iii) at least one steroid or steroid analog selected from cholesterol; and
(iv) at least one aggregation reducing lipid selected from PEG2000 DMG; and wherein the lipid-based carriers encapsulate the circular RNA, preferably wherein i) to (iv) are n a weight ratio of about 50% cationic lipid, about 10% neutral lipid, about 38.5% steroid or steroid analogue, and about 1.5% aggregation reducing lipid, preferably wherein the lipid-based carriers encapsulate the circular RNA.

In preferred embodiments, the lipid-based carriers comprising the circular RNA comprise
(i) at least one cationic lipid selected from SS-33/4PE-15 or HEXA-C5DE-PipSS;
(ii) at least one neutral lipid selected from DPhyPE;
(iii) at least one steroid or steroid analog selected from cholesterol; and
(iv) at least one aggregation reducing lipid selected from DMG-PEG 2000; and wherein the lipid-based carriers encapsulate the circular RNA, preferably wherein i) to (iv) are in a weight ratio of about 56.28% cationic lipid, about 12.24% neutral lipid, about 24.51% steroid or steroid analogue, and about 6.97% aggregation reducing lipid, preferably wherein the lipid-based carriers encapsulate the circular RNA.

In another preferred embodiment, lipid-based carriers comprising the circular RNA comprise 59 mol % HEXA-C5DE-PipSS lipid (see compound C2 in Table 1 of WO2021123332) as cationic lipid, 10 mol % DPhyPE as neutral lipid, 29.3 mol % cholesterol as steroid and 1.7 mol % DMG-PEG 2000 as aggregation reducing lipid.

In further preferred embodiment, lipid-based carriers comprising the circular RNA comprise 59 mol % compound C26 as cationic lipid, 10 mol % DPhyPE as neutral lipid, 28.5 mol % cholesterol as steroid and 2.5 mol % polymer conjugated lipid. In a very preferred embodiment, lipid-based carriers comprising the circular RNA comprise 59 mol % compound C26 as cationic lipid, 10 mol % DPhyPE as neutral lipid, 28.5 mol % cholesterol as steroid and 2.5 mol % polymer conjugated lipid having the structure

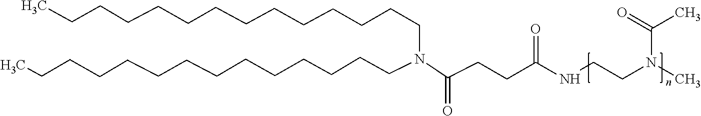

[“PMOZ 4” ],

more preferably with n=50 i.e. having 50 monomer repeats, i.e.

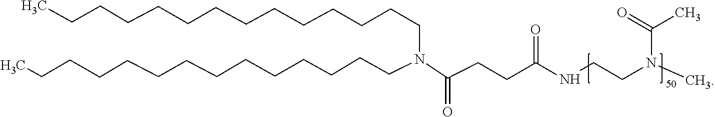

[“PMOZ 4” with n=50 i.e. having 50 monomer repeats].

In other preferred embodiments, the lipid-based carriers comprising the circular RNA comprise
(i) at least one cationic lipid selected from ALC-0315;
(ii) at least one neutral lipid selected from DSPC;
(iii) at least one steroid or steroid analog selected from cholesterol; and
(iv) at least one aggregation reducing lipid selected from ALC-0159; and wherein the lipid-based carriers encapsulate the circular RNA, preferably wherein i) to (iv) are n a molar ratio of about 47.4% cationic lipid, about 10% neutral lipid, about 40.9% steroid or steroid analogue, and about 1.7% aggregation reducing lipid, preferably wherein the lipid-based carriers encapsulate the circular RNA.

In preferred embodiments, the wt/wt ratio of lipid to circular RNA in the lipid-based carrier is from about 10:1 to about 60:1. In particularly preferred embodiments, the wVwt ratio of lipid to circular RNA is from about 20:1 to about 30:1, e.g. about 25:1.

The amount of lipid comprised in the lipid-based carriers may be selected taking the amount of the circular RNA cargo into account. In one embodiment, these amounts are selected such as to result in an N/P ratio of the lipid-based carriers encapsulating the circular RNA in the range of about 0.1 to about 20. The N/P ratio is defined as the mole ratio of the nitrogen atoms (“N”) of the basic nitrogen-containing groups of the lipid to the phosphate groups (“P”) of the circular RNA which is used as cargo. The N/P ratio may be calculated on the basis that, for example, 1ug circular RNA typically contains about 3nmol phosphate residues, provided that the circular RNA exhibits a statistical distribution of bases. The “N”—value of the lipid or lipidoid may be calculated on the basis of its molecular weight and the relative content of permanently cationic and—if present—cationisable groups.

In preferred embodiments, the N/P ratio of the lipid-based carriers encapsulating the circular RNA is in a range from about 1 to about 10, preferably in a range from about 5 to about 7, e.g. about 6.

In various embodiments, the pharmaceutical composition comprises lipid-based carriers (encapsulating circular RNA) that have a defined size (particle size, homogeneous size distribution).

The size of the lipid-based carriers of the pharmaceutical composition is typically described herein as Z-average size. The terms “average diameter”, “mean diameter”, “diameter” or “size” for particles (e.g. lipid-based carrier) are used synonymously with the value of the Z-average. The term “Z-average size” refers to the mean diameter of particles as measured by dynamic light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321).

The term “dynamic light scattering” or “DLS” refers to a method for analyzing particles in a liquid, wherein the liquid is typically illuminated with a monochromatic light source and wherein the light scattered by particles in the liquid is detected. DLS can thus be used to measure particle sizes in a liquid. Suitable DLS protocols are known in the art DLS instruments are commercially available (such as the Zetasizer Nano Series, Malvern Instruments, Worcestershire, UK). DLS instruments employ either a detector at 90° (e.g., DynaPro® NanoStar® from Wyatt Technology or Zetasizer Nano S90® from Malvem Instruments) or a backscatter detection system at 173° (e.g., Zetasizer Nano S® from Malvem Instruments) and at 1580 (DynaPro Plate Reader® from Malvem Instruments) close to the incident light of 180°. Typically, DLS measurements are performed at a temperature of about 25° C.

DLS is also used in the context of the present invention to determine the polydispersity index (PDI) and/or the main peak diameter of the lipid-based carriers incorporating RNA.

In various embodiments, the lipid-based carriers of the pharmaceutical composition encapsulating circular RNA have a Z-average size ranging from about 50 nm to about 200 nm, from about 50 nm to about 190 nm, from about 50 nm to about 180 nm, from about 50 nm to about 170 nm, from about 50 nm to about 160 nm, 50 nm to about 150 nm, 50 nm to about 140 nm, 50 nm to about 130 nm, 50 nm to about 120 nm, 50 nm to about 110 nm, 50 nm to about 100 nm, 50 nm to about 90 nm, 50 nm to about 80 nm, 50 nm to about 70 nm, 50 nm to about 60 nm, 60 nm to about 200 nm, from about 60 nm to about 190 nm, from about 60 nm to about 180 nm, from about 60 nm to about 170 nm, from about 60 nm to about 160 nm, 60 nm to about 150 nm, 60 nm to about 140 nm, 60 nm to about 130 nm, 60 nm to about 120 nm, 60 nm to about 110 nm, 60 nm to about 100 nm, 60 nm to about 90 nm, 60 nm to about 80 nm, or 60 nm to about 70 nm, for example about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm.

In preferred embodiments, the lipid-based carriers of the pharmaceutical composition encapsulating circular RNA have a Z-average size ranging from about 50 nm to about 150 nm, preferably in a range from about 50 nm to about 120 nm, more preferably in a range from about 60 nm to about 115 nm. In particularly preferred embodiments, the lipid-based carriers have a Z-average size in a range of about 50 nm to about 120 nm.

In preferred embodiments, the composition comprises at least one linear 5′ capped messenger RNA comprising at least one coding sequence encodes at least one peptide or protein.

It may be advantageous to combine a circular RNA of the first aspect and a linear 5′ capped messenger RNA as both molecules may have different translation profiles. E.g. it is envisaged that such a composition may lead to a fast protein translation (provided by the mRNA) and a long lasting protein translation (provided by the circular RNA).

Typically, a messenger RNA (mRNA) comprises a 5′-cap, optionally a 5′UTR, a coding sequence, optionally a 3′UTR and a poly(A) sequence. If RNA molecules are of synthetic origin, the RNA molecules are meant not to be produced in vivo, i.e. inside a cell or purified from a cell, but in an in vitro method. An examples for a suitable in vitro method is in vitro transcription as defined herein.

In preferred embodiments, the linear 5′ capped linear messenger RNA pharmaceutical composition is an artificial messenger RNA.

The term “artificial messenger RNA” as used herein is intended to refer to a messenger RNA that does not occur naturally. In other words, an artificial messenger RNA may be understood as a non-natural RNA molecule. Such RNA molecules may be non-natural due to its individual sequence (e.g. G/C content modified coding sequence, UTRs) and/or due to other modifications, e.g. structural modifications of nucleotides. Typically, artificial mRNA may be designed and/or generated by genetic engineering to correspond to a desired artificial sequence of nucleotides. In this context, an artificial mRNA is a sequence that may not occur naturally, i.e. a sequence that differs from the wild type sequence/the naturally occurring sequence by at least one nucleotide (via e.g. codon modification as further specified below). The term “artificial messenger RNA” is not restricted to mean “one single molecule” but is understood to comprise an ensemble of essentially identical RNA molecules. Accordingly, the term may relate to a plurality of essentially identical messenger RNA molecules.

Suitably, the at least one peptide or protein of the coding sequence of the messenger RNA is selected or derived from an antibody, an intrabody, a receptor, a receptor agonist, a receptor antagonist, a binding protein, a CRISPR-associated endonuclease, a chaperone, a transporter protein, an ion channel, a membrane protein, a toxin, a secreted protein, a chimeric antigen receptor (CAR), a transcription factor, an enzyme, a peptide or protein hormone, a growth factor, a structural protein, a cytoplasmic protein, a cytoskeletal protein, an allergen, a antigen, a neoantigen, a proto-oncogene, an oncogene, a tumor-suppressor gene, a mutated antigen, an antigen of a pathogen, or fragments, epitopes, variants, or combinations of any of these.

Suitably, the at least one peptide or protein of the coding sequence of the messenger RNA is selected or derived from an antigen of a pathogen.

Suitably, the antigen of a pathogen of the coding sequence of the messenger RNA is selected or derived from a viral antigen, a bacterial antigen, a protozoan antigen, a fungal antigen, or fragments, variants, or combinations of any of these.

In preferred embodiments, the at least one linear 5′ capped messenger RNA of the pharmaceutical composition comprises at least one 5′ UTR and/or at least one 3′ UTR.

Preferably, the at least one 3′-UTR of the messenger RNA of the pharmaceutical composition comprises or consists of a nucleic acid sequence derived from a 3′-UTR of a gene selected from PSMB3, ALB7, alpha-globin, CASP1, COX6B1, GNAS, NDUFA1, AES-12S and RPS9, or from a homolog, a fragment ora variant of any one of these genes. Further details in that context regarding e.g. suitable nucleic acid sequences are provided in the context of the first aspect Preferably, the at least one 5′-UTR of the messenger RNA of the pharmaceutical composition comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B, HBA1, HBA2 and UBQLN2, or from a homolog, a fragment or variant of any one of these genes. Further details in that context regarding e.g. suitable nucleic acid sequences are provided in the context of the first aspect.

In preferred embodiments, the at least one linear 5′ capped messenger RNA of the pharmaceutical composition comprises at least one 5′ UTR derived from HSD17B4 and/or at least one 3′ UTR derived from PSMB3.

In preferred embodiments, the at least one linear 5′ capped messenger RNA of the pharmaceutical composition comprises at least one poly(A) sequence and, optionally, at least one histone stem loop and/or at least one poly(C) sequence. Further details in that context regarding e.g. suitable nucleic acid sequences are provided in the context of the first aspect.

In preferred embodiments, the at least one poly(A) sequence of the messenger RNA of the pharmaceutical composition comprises about 30 to about 500 consecutive adenosine nucleotides.

Suitably, the at least one poly(A) sequence of the messenger RNA of the pharmaceutical composition comprises about 100 consecutive adenosine nucleotides, preferably wherein poly(A) sequence represents the 3′ terminus.

In embodiments, the messenger RNA of the pharmaceutical composition comprises at least two, three, or more poly(A) sequences.

In various embodiments, the messenger RNA of the pharmaceutical composition comprises a 5-cap structure, preferably a cap1 structure.

Accordingly, in preferred embodiments, the RNA of the pharmaceutical composition comprises a 5′-cap structure, preferably m7G, cap0, cap1, cap2, a modified cap0 or a modified cap1 structure.

The term “5′-cap structure” as used herein is intended to refer to the 5′ structure of a messenger RNA, particularly a guanine nucleotide, positioned at the 5′-end of messenger RNA. Preferably, the 5′-cap structure is 30 connected via a 5′-5′-triphosphate linkage to the mRNA. Notably, a “5-cap structure” or a “cap analogue” is not considered to be a “modified nucleotide” or “chemically modified nucleotides” in the context of the invention. 5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g. phosphothioate modARCA), inosine, N1-methyl-guanosine, 2′-luoro-guanosine, 7-deaza-guanosine, 8-oxo-guanosine, 2-amino-guanosine, LNA-guanosine, and 2-azido-guanosine.

A 5-cap (cap0 or cap1) structure may be formed in chemical RNA synthesis, using capping enzymes, or in RNA in vitro transcription (co-transcriptional capping) using cap analogs.

The term “cap analog” as used herein is intended to refer to a non-polymerizable di-nucleotide or tri-nucleotide that has cap functionality in that it facilitates translation or localization, and/or prevents degradation the RNA when incorporated at the 5′-end of the mRNA. Non-polymerizable means that the cap analogue will be incorporated only at the 5′-terminus because it does not have a 5′ triphosphate and therefore cannot be extended in the 3′-direction by a template-dependent polymerase, (e.g. a DNA-dependent RNA polymerase).

In preferred embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap analog derivable from the structure disclosed in claim 1-5 of WO2017/053297 may be suitably used to co-transcriptionally generate a cap1 structure. Further, any cap analog derivable from the structure defined in claim 1 to claim 21 of WO2018/075827 may be suitably used to co-transcriptionally generate a cap1 structure.

In preferred embodiments, the mRNA of the pharmaceutical composition comprises a cap1 structure.

In preferred embodiments, the cap1 structure of the mRNA is formed using co-transcriptional capping using tri-nucleotide cap analog m7G(5′)ppp(5′)(2′OMeA)pG or m7G(5′)ppp(5′)(2′OMeG)pG. A preferred cap1 analog in that context is m7G(5′)ppp(5′)(2′OMeA)pG.

In alternative embodiments, the cap1 structure is formed using enzymatic capping.

In preferred embodiments, the at least one coding sequence of the linear 5′ capped messenger RNA is a codon modified coding sequence, wherein the amino acid sequence encoded by the at least one codon modified coding sequence is preferably not being modified compared to the amino acid sequence encoded by the corresponding wild type or reference coding sequence.

In preferred embodiments, the at least one codon modified coding sequence of the linear 5′ capped messenger RNA is selected from a C maximized coding sequence, a CAI maximized coding sequence, a human codon usage adapted coding sequence, a G/C content modified coding sequence, and a G/C optimized coding sequence, or any combination thereof.

In preferred embodiments, the at least one codon modified coding sequence of the linear 5′ capped messenger RNA is a G/C optimized coding sequence, a human codon usage adapted coding sequence, or a G/C content modified coding sequence.

In preferred embodiments, the at least one coding sequence of the linear 5′ capped messenger RNA has a G/C content of at least about 50%, 55%, 60%, or 65%.

In preferred embodiments, the messenger RNA of the pharmaceutical composition does not comprise chemically modified nucleotides.

In alternative embodiments, the messenger RNA of the pharmaceutical composition comprises modified nucleotides. Suitably, the mRNA comprises chemically modified nucleotides selected from those defined in the context of the first aspect or second aspect.

In preferred embodiments, the at least one modified nucleotide of the linear 5′ capped messenger RNA of the pharmaceutical composition is selected from pseudouridine (ψ), N1-methylpseudouridine (m1ψ), 5-methylcytosine, and/or 5-methoxyuridine.

In preferred embodiments, at least one coding sequence of the linear 5′ capped messenger RNA encodes at least one peptide or protein suitable for use in treatment or prevention of a disease, disorder or condition.

In preferred embodiments, the at least one peptide or protein encoded by the linear 5′ capped messenger RNA is selected or derived from an antibody, an intrabody, a receptor, a receptor agonist, a receptor antagonist, a binding protein, a CRISPR-associated endonuclease, a chimeric antigen receptor (CAR), a chaperone, a transporter protein, a toxin, an ion channel, a membrane protein, a secreted protein, a transcription factor, an enzyme, a peptide or protein hormone, a growth factor, a structural protein, a cytoplasmic protein, a cytoskeletal protein, an allergen, a tumor antigen, a neoantigen, a proto-oncogene, an oncogene, a tumor-suppressor gene, a mutated antigen, an antigen of a pathogen, or fragments, epitopes, variants, or combinations of any of these.

In preferred embodiments, the at least one peptide or protein encoded by the linear 5′ capped messenger RNA is selected or derived from an antigen of a pathogen.

In preferred embodiments, the antigen of a pathogen is selected or derived from a viral antigen, a bacterial antigen, a protozoan antigen, a fungal antigen, or fragments, variants, or combinations of any of these.

In preferred embodiments, the at least one peptide or protein encoded by the linear 5′ capped messenger RNA is selected or derived from an antigen of a tumor.

In preferred embodiments, the antigen of a pathogen is selected or derived from a tumor antigen, a neoantigen, a proto-oncogene, an oncogene, a tumor-suppressor gene, a mutated antigen, or fragments, variants, or combinations of any of these.

In preferred embodiments, at least one messenger RNA of the pharmaceutical composition is a purified mRNA.

Accordingly, “purified mRNA” as used herein has a degree of purity of more than 75%, 80%, 85%, very 5 particularly 90%, 91%, 92%, 93%, 94%, 95%, 96%, 97%, 98% and most favourably 99% or more. The degree of purity may for example be determined by an analytical HPLC, wherein the percentages provided above correspond to the ratio between the area of the peak for the target RNA and the total area of all peaks representing all the by-products. Alternatively, the degree of purity may for example be determined by an analytical agarose gel electrophoresis or capillary gel electrophoresis.

Suitably, purification of the at least one messenger RNA of the pharmaceutical composition may be performed by means of RP-HPLC, AEX, size exclusion chromatography, hydroxyapatite chromatography, TFF, filtration, precipitation, core-bead flow through chromatography, oligo(dT) purification, spin column and/or cellulose-based purification, preferably by RP-HPLC and/or TFF.

In embodiments, the messenger RNA of the pharmaceutical composition is an RP-HPLC purified mRNA and/or a tangential flow filtration (TFF) purified mRNA.

In various embodiments the at least one linear 5 capped messenger RNA comprises, preferably in 5′- to 3′-direction, the following elements:
A) 5′-cap structure, preferably as specified herein;
B) optionally, a 5′-UTR, preferably as specified herein;
C) at least one coding sequence, preferably as specified herein;
D) 3′-UTR, preferably as specified herein;
E) optionally, poly(A) sequence, preferably as specified herein;
F) optionally, poly(C) sequence, preferably as specified herein;
G) optionally, histone stem-loop preferably as specified herein; In particularly preferred embodiments the at least one linear 5′ capped messenger RNA comprises the following elements in 5′- to 3′-direction:
A) 5′ cap1 structure as defined herein;
B) 5′-UTR derived from a HSD17B4 gene as defined herein;
C) at least one coding sequence;
D) 3′-UTR derived from a 3′-UTR of a PSMB3 gene as defined herein;
E) optionally, a histone stem-loop selected preferably as specified herein;
F) poly(A) sequence, preferably comprising about 100 A nucleotides, optionally representing the 3′ terminus.

In preferred embodiments, the linear 5′ capped messenger RNA as defined herein is separately formulated as defined herein, e.g. separately formulated in lipid-based carriers as defined herein.

In alternative preferred embodiments, the linear 5 capped messenger RNA as defined herein is co-formulated with the at least one circular RNA as defined, e.g. co-formulated in lipid-based carriers as defined herein.

In preferred embodiments, the pharmaceutical composition is lyophilized, spray-dried or spray-freeze dried.

In preferred embodiments, the pharmaceutical composition of the invention is a vaccine comprising circular RNA.

In preferred embodiments, the pharmaceutical composition e.g. the vaccine elicits an adaptive immune response, preferably a protective adaptive immune response against a pathogen, wherein the at least one pathogen may be selected from a bacterium, a protozoan, or a virus.

In preferred embodiments, the pharmaceutical composition e.g. the vaccine elicits an adaptive immune response, preferably a protective adaptive immune response against a tumor antigen, wherein the at least one tumor antigen may be selected from a proto-oncogene, an oncogene, a tumor suppressor gene, a neoantigen, or a mutated antigen.

In preferred embodiments, administration of the vaccine elicits neutralizing antibody titers against at least one pathogen, wherein the at least one pathogen may be selected from a bacterium, a protozoan, or a virus.

In preferred embodiments, administration of the vaccine elicits neutralizing antibody titers against at least one tumor antigen, wherein the at least one tumor antigen may be selected from a proto-oncogene, an oncogene, a tumor suppressor gene, a neoantigen, or a mutated antigen.

Accordingly, the pharmaceutical composition e.g. the vaccine is against a pathogen, for example against a virus, against a bacterium, or against a protozoan.

Accordingly, the pharmaceutical composition e.g. the vaccine is against a tumor antigen, for example against a proto-oncogene, an oncogene, a tumor suppressor gene, a neoantigen, or a mutated antigen.

The pharmaceutical composition or vaccine may be used according to the invention for human medical purposes and also for veterinary medical purposes (mammals, vertebrates, or avian species).

Suitable administration routes for the pharmaceutical composition or vaccine comprise intravenous, intranasal, intramuscular, intradermal, transdermal, intraocular, subcutaneous, intrapulmonal, intralesional, intrathecal, intracranial, intracardial, intratumoral.

Accordingly, in preferred embodiments, the pharmaceutical composition or vaccine is suitable for intravenous, intranasal, intramuscular, intradermal, transdermal, intraocular, subcutaneous, intrapulmonal, intralesional, intrathecal, intracranial, intracardial, intratumoral administration.

Preferred in the context of a vaccine is an intramuscular injection.

Preferred in the context of a protein replacement therapy is an intramuscular or subcutaneous injection.

In this context, it is particularly preferred that the pharmaceutical composition has at least 10%, 20% or at least 30% lower immunostimulatory properties compared to a composition comprising a corresponding reference RNA. In some preferred embodiments, the pharmaceutical composition has at least 40%, 50% or at least 60% lower immunostimulatory properties compared to a composition comprising a corresponding reference RNA.

Accordingly, reduction of the immunostimulatory properties in the context of the invention is characterized by a reduced level of at least one cytokine preferably selected from MIG, McP1, Rantes, MIP-1 alpha, IP-10, MIP-1 beta, McP1, TNFalpha, IFNgamma, IFNalpha, IFNbeta, IL-12, IL-6, or IL-8, wherein the reduced level of at least one cytokine is a reduction of at least 10%, 15%, 20%, 25%, 30%, 35%, 40%, 45%, 50%, 55%, 60%, 65%, 70%, 75%, 80%, 85%, 90%, or 95%. Preferably, the reduced level of at least one cytokine is a reduction of at least 30%.

In preferred embodiments, administration pharmaceutical composition to a cell, tissue, or organism results in a prolonged protein expression compared to administration of a composition comprising a corresponding reference RNA, wherein the additional duration of protein expression in said cell, tissue, or organism is at least 5 h, 10h, 20 h, 25h, 30h, 35h, 40h, 45h, 50h, 55h, 60h, 65h, 70h, 75h, 80h, 85h, 90h, 95h, or 100h or even longer. Preferably, the additional duration of protein expression is about 20h to about 240h. Suitably, the additional duration of protein expression is in liver cells. In preferred embodiments the additional duration of protein expression is in adipocytes. In particularly embodiments the additional duration of protein expression is in muscle cells.

In preferred embodiments, administration of the pharmaceutical composition to a cell, tissue, or organism results in an increased expression as compared to administration of a composition comprising a corresponding reference RNA, wherein the percentage increase in expression in said cell, tissue, or organism is at least 10%, 20%, 30%, 40%, 50%, 60%, 70%, 80%, 90%, 100%, 200%, 300%, 400%, 500% or even more. Preferably, the percentage increase in expression is about 20% to about 100%. Suitably, increase in expression is in liver cells. In preferred embodiments the increase in expression is in adipocytes. In particularly embodiments increase in expression is in muscle cells.

In preferred embodiments, administration of the pharmaceutical composition to a cell, tissue, or organism results in a longer half-life of the circular RNA compared to administration of a composition comprising a corresponding reference RNA, wherein the additional duration of half-life in said cell, tissue, or organism is at least 5 h, 10h, 20 h, 25h, 30h, 35h, 40h, 45h, 50h, 55h, 60h, 65h, 70h, 75h, 80h, 85h, 90h, 95h, or 100h or even longer. Preferably, the additional half-life is about 20h to about 240h. Suitably, the additional half-life is observed in liver cells. In preferred embodiments the additional half-life is observed is in adipocytes. In particularly embodiments additional half-life is observed is in muscle cells.

### 4: A Combination Comprising Circular RNA

In a fourth aspect, the invention provides a combination comprising circular RNA.

Notably, embodiments relating to the circular RNA of the first aspect may likewise be read on and be understood as suitable embodiments of the combination comprising circular RNA of the fourth aspect. Also, embodiments relating to the pharmaceutical composition of the third aspect may likewise be read on and be understood as suitable embodiments of the combination of the fourth aspect and vice versa.

In the context of the present invention, the term “combination” preferably means a combined occurrence of the at 20 least one circular RNA (herein referred to as “component A”) and of the at least one further linear coding RNA (herein referred to as “component B”). Therefore, said combination may occur either as one composition (as outlined e.g. in the context of the third aspect), comprising all these components in one and the same composition or mixture (but as separate entities), or may occur as a kit of parts, wherein the different components form different parts of such a kit of parts (as outlined e.g. in the context of the fifth aspect). Thus, the administration of the components of the combination may occur either simultaneously or timely staggered, either at the same site of administration or at different sites of administration, as further outlined below. The components may be formulated together as a co-formulation, or may be formulated as different separate formulations (and optionally combined after formulation).

As further specified herein, the combination comprises at least one circular RNA (component A) and at least one linear coding RNA (component B).

It may be advantageous to combine a circular RNA of the first aspect and a linear coding RNA as both molecules may have different translation profiles. E.g. it is envisaged that such a composition may lead to a fast protein translation (provided by the linear coding RNA) and a long lasting protein translation (provided by the circular RNA).

In preferred embodiments, the fourth aspect relates to a combination comprising the following components
A) at least one circular RNA comprising at least one coding sequence; and
B) at least one linear coding RNA.

A linear coding RNA can be any type of linear RNA construct characterized in that said linear coding RNA comprises at least one sequence (cds) that is translated into at least one amino-acid sequence (upon administration to e.g. a cell). Notably, the term linear coding RNA does explicitly not comprise a linear precursor RNA (for example as defined in the context of the second aspect) that is used to generate the circular RNA of the combination.

Preferably, said linear coding RNA may be selected from an mRNA, a (coding) self-replicating RNA, a (coding) viral RNA, or a (coding) replicon RNA.

In preferred embodiments of the combination, the at least one circular RNA (component A) is further characterized by any one of the features of the first aspect In particularly preferred embodiments of the combination, the circular RNA (component A) comprises or consists an RNA sequence identical or at least 70%, 80%, 90%, 91%, 92%, 93%, 94%, 95%, 96%, 97%, 98%, or 99% identical to any one of SEQ ID NOs: 202-205, 533-544 or a fragment or a variant of any of these, wherein the coding sequence (encoding Gluc or Ppluc) in any one of SEQ ID NOs: 202-205, 533-544 is exchanged by at least one coding sequence as defined herein, preferably a coding sequence encoding a therapeutic peptide or protein as defined herein.

In particularly preferred embodiments of the combination, the at least one linear coding RNA (component B) is a linear 5′ capped messenger RNA.

Accordingly, in particularly preferred embodiments, the combination comprises the following components
A) at least one circular RNA of the first aspect comprising at least one coding sequence; and
B) at least one linear 5′ capped messenger RNA; In preferred embodiments of the combination, the at least one linear 5 capped messenger RNA (component B) is further characterized by any one of the features relating to mRNA as provided in the third aspect

Suitably, the at least one linear 5′ capped mRNA of the combination comprises, preferably in 5′- to 3-direction, the following elements:
A) 5′-cap structure, preferably as specified in the third aspect;
B) optionally, a 5′-UTR, preferably as specified in the third aspect;
C) at least one coding sequence, preferably as specified in the third aspect;
D) 3′-UTR, preferably as specified in the third aspect;
E) optionally, poly(A) sequence, preferably as specified in the third aspect;
F) optionally, poly(C) sequence, preferably as specified in the third aspect;
G) optionally, histone stem-loop preferably as specified in the third aspect;

Suitably, the at least one linear 5′ capped mRNA of the combination comprises the following elements in 5′- to 3′-direction:
A) 5′ cap1 structure as specified in the third aspect;
B) 5′-UTR derived from a HSD17B4 gene as specified in the third aspect;
C) at least one coding sequence as specified in the third aspect;
D) 3′-UTR derived from a 3′-UTR of a PSMB3 gene as specified in the third aspect;
E) optionally, a histone stem-loop selected preferably as specified in the third aspect;
F) poly(A) sequence, preferably comprising about 100 A nucleotides, optionally representing the 3′ terminus.

In preferred embodiments, component A and/or component B are separately formulated.

In particularly preferred embodiments, component A and/or component B are separately formulated in lipid-based carriers, preferably wherein the lipid-based carriers are as defined in the third aspect.

In preferred embodiments, component A and/or component B are co-formulated.

In particularly preferred embodiments, component A and/or component B are co-formulated in lipid-based carriers, preferably wherein the lipid-based carriers are as defined in the third aspect. The obtained composition may be characterized by any of the features of the third aspect.

In various embodiments of the combination, the molar ratio of component A to component B ranges from about 1:1 to about 10:1, or ranges from about 1:1 to about 1:10. In specific embodiments of the combination, the molar ratio of component A to component B is about 1:1.

In various embodiments of the combination, the weight to weight ratio of component A to component B ranges from about 1:1 to about 10:1, or ranges from about 1:1 to about 1:10. In specific embodiments of the combination, the weight to weight ratio of component A to component B is about 1:1.

In particularly preferred embodiments, upon administration of the combination to a cell or subject, the combination has reduced immunostimulatory properties (as defined herein) compared to an administration of component B alone.

In this context, it is particularly preferred that the combination has at least 10%, 20% or at least 30% lower immunostimulatory properties (as defined herein) compared to component B alone.

In some preferred embodiments, the combination has at least 40%, 50% or at least 60% lower immunostimulatory properties (as defined herein) compared to component B alone.

In particularly preferred embodiments, upon administration of the combination to a cell or subject, the combination has a prolonged protein expression (that is an additional duration of protein expression) compared to an administration of component B alone.

Suitably, the additional duration of protein expression is at least 5 h, 10h, 20 h, 25h, 30h, 35h, 40h, 45h, 50h, 55h, 60h, 65h, 70h, 75h, 80h, 85h, 90h, 95h, or 100h or even longer. Preferably, the additional duration of protein expression is about 20h to about 240h.

In particularly preferred embodiments, upon administration of the combination to a cell or subject, the combination has a faster onset of protein expression compared to an administration of component A alone.

Suitably, the faster onset of protein expression is to be understood as a detectable protein expression that starts at least 1 h, 2h, 3 h, 4h, 5 h, 5h, 10 h, 20h, 25h earlier. Suitably, the faster onset of protein expression is to be understood as the peak protein expression that is achieved at least 1 h, 2h, 3 h, 4h, 5 h, 5h, 10 h, 20h, 25h earlier.

In embodiments, the administration of the components of the combination may occur either simultaneously or timely staggered, either at the same site of administration or at different sites of administration, In preferred embodiments, administration of the combination is intravenous, intranasal, intramuscular, intradermal, transdermal, intraocular, subcutaneous, intrapulmonal, intralesional, intrathecal, intracranial, intracardial, intratumoral.

5: A Kit or Kit of Parts Comprising Circular RNA In a fifth aspect, the invention provides a kit or kit of parts comprising circular RNA.

Notably, embodiments relating to the circular RNA of the first aspect may likewise be read on and be understood as suitable embodiments of the kit or kit of parts comprising circular RNA of the fifth aspect Also, embodiments relating to the pharmaceutical composition of the third aspect or the combination of the fourth aspect may likewise be read on and be understood as suitable embodiments of the kit or kit of parts comprising circular RNA of the fifth aspect

In preferred embodiments, the fifth aspect relates to a kit or kit of parts, comprising
at least one circular RNA characterized by any one of the features of the first aspect, and/or
at least one pharmaceutical composition characterized by any one of the features of the third aspect, and/or
at least one vaccine characterized by any one of the features of the third aspect In particularly preferred embodiments, the kit or kit of parts comprises a liquid vehicle for solubilising, and/or, technical instructions providing information on administration and dosage of the components.

In particularly preferred embodiments, the kit or kit of parts comprises at least one further linear coding RNA, preferably a linear 5′ capped messenger RNA, preferably as defined herein (e.g. in the context of the composition or combination).

In embodiments where the pharmaceutical composition is provided as a lyophilized or spray-freeze dried or spray dried composition, the kit or kit of parts may suitably comprise a buffer for re-constitution of lyophilized or spray-freeze dried or spray dried composition.

Accordingly, the kit or kit of parts additionally comprises a buffer for re-constitution and/or dilution.

In preferred embodiments, the buffer for re-constitution and/or dilution is a sterile buffer. In preferred embodiments, the buffer comprises a salt, preferably NaCl, optionally in a concentration of about 0.9%.

6: First, Second, and Further Medical Uses: In a further aspect, the present invention relates to the medical use of the circular RNA as defined herein, the pharmaceutical composition as defined herein, the vaccine as defined herein, the combination as defined herein, or the kit or kit of parts as defined herein.

Notably, embodiments relating to the previous aspects may likewise be read on and be understood as suitable embodiments of medical uses of the invention (and vice versa).

Accordingly, the invention provides the circular RNA of the invention, and/or the pharmaceutical composition of the invention, and/or the vaccine of the invention, and/or the combination of the invention, and/or the kit or kit of parts of the invention for use as a medicament In preferred embodiments, the use may be for human medical purposes and also for veterinary medical purposes, preferably for human medical purposes.

In other preferred embodiments, the use may for human medical purposes, in particular for young infants, newborns, immunocompromised recipients, pregnant and breast-feeding women, and elderly people.

In a further aspect, the present invention relates to second medical uses of the circular RNA as defined herein, the pharmaceutical composition as defined herein, the vaccine as defined herein, the combination as defined herein, or the kit or kit of parts as defined herein.

Accordingly, the invention provides the circular RNA of the invention, and/or the pharmaceutical composition of the invention, and/or the vaccine of the invention, and/or the combination of the invention, and/or the kit or kit of parts of the invention for use in treating or preventing an infectious disease, a tumour disease, or a genetic disorder or condition.

In that context, an infection may be caused by a pathogen selected from a bacterium, a protozoan, or a virus.

In preferred aspects, the invention relates to the medical use of the circular RNA of the invention, and/or the pharmaceutical composition of the invention, and/or the vaccine of the invention, and/or the combination of the invention, and/or the kit or kit of parts of the invention in the treatment or prophylaxis of a tumour disease, or of a disorder related to such tumour disease.

Accordingly, in said embodiments, the circular RNA of the pharmaceutical composition may encode at least one tumour or cancer antigen and/or at least one therapeutic antibody (e.g. checkpoint inhibitor).

In preferred aspects, the invention relates to the medical use of the circular RNA of the invention, and/or the pharmaceutical composition o of the invention, and/or the vaccine of the invention, and/or the combination of the invention, and/or the kit or kit of parts of the invention in the treatment or prophylaxis of a genetic disorder or condition.

Such a genetic disorder or condition may be a monogenetic disease, i.e. (hereditary) disease, or a genetic disease in general, diseases which have a genetic inherited background and which are typically caused by a defined gene defect and are inherited according to Mendel's laws.

Accordingly, in said embodiments, the circular RNA of the pharmaceutical composition may encode a CRISPR-associated endonuclease or another protein or enzyme suitable for genetic engineering. Such a composition may also comprise a guide RNA.

In other preferred aspects, the invention relates to the medical use of the circular RNA of the invention, and/or the pharmaceutical composition of the invention, and/or the vaccine of the invention, and/or the combination of the invention, and/or the kit or kit of parts of the invention in the treatment or prophylaxis of a protein or enzyme deficiency or protein replacement.

Accordingly, in said embodiments, the circular RNA of the pharmaceutical composition may encode at least one protein or enzyme. “Protein or enzyme deficiency” in that context has to be understood as a disease or deficiency where at least one protein is deficient, e.g. A1AT deficiency.

In yet another aspect, the invention relates to the medical use of the circular RNA of the invention, and/or the pharmaceutical composition of the invention, and/or the vaccine of the invention, and/or the combination of the invention, and/or the kit or kit of parts of the invention in the treatment or prophylaxis of autoimmune diseases, allergies or allergic diseases, cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, and ear diseases.

In the context of a medical use, the circular RNA of the invention, and/or the pharmaceutical composition of the invention, and/or the vaccine of the invention, and/or the combination of the invention, and/or the kit or kit of parts of the invention may preferably be administered locally or systemically.

In that context, administration may be by an intravenous, intranasal, intramuscular, intradermal, transdermal, intraocular, subcutaneous, intrapulmonal, intralesional, intrathecal, intracranial, intracardial, intratumoral.

Accordingly, in preferred embodiments, the circular RNA is suitable for intravenous, intramuscular, intraarticular, sublingual, intraocular, intrapulmonal, intrathecal, intratumoral route.

In embodiments, administration may be by conventional needle injection. In embodiments, administration may be via inhalation. In embodiments, administration may be a topical administration.

### 7: Method of Treatment:

In a further aspect, the present invention relates to a method of treating or preventing a disease, disorder or condition.

Notably, embodiments relating to the previous aspects may likewise be read on and be understood as suitable embodiments of medical uses of the invention.

Furthermore, specific features and embodiments relating to method of treatments as provided herein may also apply for medical uses of the invention and vice versa.

Preventing (inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the present invention relates to a method of treating or preventing a disease, disorder or condition, wherein the method comprises applying or administering to a subject in need thereof the circular RNA of the invention, and/or the pharmaceutical composition of the invention, and/or the vaccine of the invention, and/or the combination of the invention, and/or the kit or kit of parts of the invention.

In preferred embodiments, the disease, disorder or condition is an infectious disease or a disorder related to such an infectious disease, a tumour disease or a disorder related to such tumour disease, a genetic disorder or condition, a protein or enzyme deficiency.

In other embodiments, the disorder is an autoimmune disease, an allergy or allergic disease, cardiovascular disease, neuronal disease, disease of the respiratory system, disease of the digestive system, disease of the skin, musculoskeletal disorder, disorders of the connective tissue, neoplasm, immune deficiencies, endocrine, nutritional and metabolic disease, eye disease, and ear disease.

In particularly preferred embodiments, the subject in need is a mammalian subject, preferably a human subject, e.g. new-born human subject, pregnant human subject, immunocompromised human subject, and/or elderly human subject.

In preferred embodiments, applying or administering is performed via intramuscular injection, intradermal injection, transdermal injection, intradermal injection, intralesional injection, intracranial injection, subcutaneous injection, intracardial injection, intratumoral injection, intravenous injection, or intraocular injection, intrapulmonal inhalation, intraarticular injection, sublingual injection.

Preferably, the disease, disorder or condition is a disease, disorder or condition that requires a long lasting protein expression and/or that requires a repetitive administration.

Further preferably, the disease, disorder or condition is a disease, disorder or condition of the liver.

### 8: A Method for Preparing Circular RNA

In a further aspect, the invention provides a method for preparing circular RNA.

Notably, embodiments relating to a method of purifying a circular RNA as defined herein (see next aspect in paragraph 9) may likewise be read on and be understood as suitable embodiments of the method for preparing circular RNA of the present aspect In addition, embodiments of the first aspect defining the circular RNA of the composition (that is, the circular RNA that is to be produced) may likewise be read on and be understood as suitable embodiments of the method of preparing a circular RNA of the present aspect. In addition, embodiments of the second aspect defining the linear precursor RNA of the composition (that is, the (non-circularized) linear precursor RNA) may likewise be read on and be understood as suitable embodiments of the method of preparing a circular RNA of the present aspect.

In preferred embodiments of the method for preparing circular RNA, the method comprises the steps of
A) providing a linear precursor RNA comprising a 3′ permuted intron-exon element and a 5′ permuted intron-exon element for circularization;
B) Incubating the precursor RNA in a buffer to allow circularization of the RNA; and
C) obtaining the circular RNA product In preferred embodiments, the linear precursor RNA comprises at least one moiety or at least one sequence element configured for circularizing the linear precursor RNA.

For example, a linear precursor RNA according to SEQ ID NO: 198 may lead, after circularization reaction, to the two intronic splice products according to SEQ ID NO: 184 and according to SEQ ID NO: 185, and to the desired circular RNA according to SEQ ID NO: 203.

As another example, a linear precursor RNA according to SEQ ID NO: 199 may lead, after circularization reaction, to the two intronic splice products according to SEQ ID NO: 184 and according to SEQ ID NO: 185, and to the desired circular RNA according to SEQ ID NO: 204.

In particularly preferred embodiments, the linear precursor RNA comprises a 3′ permuted intron-exon element and a 5′ permuted intron-exon element for circularization (as defined in the context of the second aspect).

In preferred embodiments, the providing step A) comprises a step A1) RNA in vitro transcription as defined herein.

In preferred embodiments, the RNA in vitro transcription (step A1) is performed in the presence of a sequence optimized nucleotide mixture (IVT-mix), preferably as described in WO2015/188933. Such a procedure may reduce the formation of by-products during IVT.

Accordingly, the linear precursor RNA is preferably produced by RNA in vitro transcription in a sequence-optimized IVT-mix, preferably as described in WO2015/188933. Accordingly, the sequence-optimized IVT-mix may comprise the four ribonucleoside triphosphates (NTPs) GTP, ATP, CTP and UTP, wherein the fraction (1) 5 of each of the four ribonucleoside triphosphates in the sequence-optimized reaction mix corresponds to the fraction (2) of the respective nucleotide in said RNA molecule, a buffer, a DNA template, and an RNA polymerase. In that context, fraction (1) and fraction (2) may differ by not more than 25%, 20%, 15%, 10%, 7%, 5% or by a value between 0.1% and 5%.

Furthermore, the RNA in vitro transcription process may comprise at least one feeding step, preferably wherein the feeding step provided further sequence-optimized IVT-mix.

In preferred embodiments, the RNA in vitro transcription (step A1) is performed in the absence of modified nucleotides. In embodiments, the RNA in vitro transcription (step A1) is performed in the absence of cap analogs. Accordingly, in preferred embodiments, the vitro transcription mix, preferably the sequence-optimized IVT-mix, is composed of (chemically) non-modified ribonucleoside triphosphates (NTPs) GTP, ATP, CTP and UTP.

In alternatively preferred embodiments, the RNA in vitro transcription (step A1) is performed in the presence of modified nucleotides. Suitably modified nucleotides in that context may be selected from pseudouridine (y), N1-methylpseudouridine (m1ψ), 5-methylcytosine, and/or 5-methoxyuridine. Further suitably modified nucleotides in that context may be selected from Alpha-thio-ATP, Alpha-thio-GTP, Alpha-thio-CTP, Alpha-thio-UTP, N4-acetyl-CTP, N6-methyladenosine, 2′O-methyl-ATP, 2′O-methyl-GTP, 2′O-methyl-CTP, and/or 2′O-methyl-UTP.

In some embodiments, the RNA in vitro transcription is performed in the presence of a cap analog (e.g. (e.g. a cap1 analog). Generating capped linear precursor RNA may have the advantage that potential (non-circularized) linear RNA constructs lack an immunostimulatory 5′ terminal triphosphate which may be advantageous in generating a circular RNA product with reduced immunostimulatory properties.

In some embodiments, the RNA in vitro transcription is performed in the presence of a GDP that is incorporated as a starting nucleotide during IVT. Generating a linear precursor RNA under these conditions may have the advantage that potential (non-circularized) linear RNA constructs lack an immunostimulatory 5′ terminal triphosphate which may be advantageous in generating a circular RNA product with reduced immunostimulatory properties.

In some embodiments, the RNA in vitro transcription process may comprise at least one feeding step, preferably wherein the feeding step provided further vitro transcription mix, preferably the sequence-optimized IVT-mix as defined herein.

Already during RNA in vitro transcription, circularization of the linear precursor RNA takes place. The circularization event via self-splicing introns typically requires the presence of GTP and MgCl₂-components that are typically already comprised in an RNA in vitro transcription reaction.

To ensure sufficient circularization of the linear precursor RNA, the method comprises a step of adding GTP to the RNA in vitro transcription reaction to start the incubation step B, preferably after completion of the RNA in vitro transcription process. The addition of GTP is suitably configured to obtain a final concentration of at least about 2 mM GTP.

Accordingly, in preferred embodiments, the incubation step B) is performed in a buffer comprising GTP and MgCl₂. As described above, the GTP may be already present in the RNA in vitro transcription reaction or may be added in addition to the GTP that has not been integrated into the in vitro transcribed RNA products.

In embodiments, the GTP concentration of incubation step B) is at least about 1 mM, at least about 2 mM, at least about 3 mM, at least about 4 mM, at least about 5 mM, at least about 6 mM, at least about 7 mM, at least about 8 mM, at least about 9 mM, at least about 10 mM. In embodiments, the GTP concentration may be in a range from about 1 mM to about 20 mM, preferably from about 1 mM to about 10 mM, more preferably from about 1 mM to about 5 mM.

In particularly preferred embodiments, the incubation step B) is performed at a final GTP concentration of at least 2 mM GTP (which is achieved by adding the respective amount of GTP to the RNA in vitro transcription reaction).

In preferred embodiments in that context, at the end of the RNA in vitro transcription when the linear precursor RNA is produced in sufficient amounts, fresh GTP is added to the reaction to ensure a complete circularization of the linear precursor RNA, wherein the amount of added GTP is sufficient to obtain a final GTP concentration of at least 2 mM GTP.

In embodiments, the MgCl2 concentration of incubation step B) is at least about 2 mM, at least about 3 mM, at 30 least about 4 mM, at least about 5 mM, at least about 15 mM, at least about 20 mM, at least about 25 mM, at least about 30 mM, at least about 35 mM, at least about 40 mM. In embodiments, the MgCl₂ concentration may be in a range from about 2 mM to about 40 mM, preferably from about 2 mM to about 30 mM, more preferably from about 10 mM to about 30 mM.

In particularly preferred embodiments, the incubation step B) is performed at a final MgCl2 concentration of at least about 10 mM MgCl₂ (which typically corresponds to the amount of MgCl2 already present in the RNA in vitro transcription reaction).

In particularly preferred embodiments, the incubation step B) is performed in a buffer comprising GTP and MgCl₂, preferably wherein GTP is in a final concentration of at least about 2 mM (e.g. about 2 mM) and MgCL₂ is in a final concentration of about 10 mM (e.g. about 24 mM).

In preferred embodiments, the incubation step B) is performed for at least 5 minutes, for at least 10 minutes, for at least 15 minutes, for at least 20 minutes, for at least 25 minutes, for at least 30 minutes. In preferred embodiments, the incubation step B) is performed for about 5 minutes to about 30 minutes.

In that context, the “starting point” of the incubation step is defined as the time point when the reaction conditions of the IVT are adjusted to 2 mM GTP (by the addition of e.g. about 2 mM GTP).

In preferred embodiments, the incubation step B) is performed for at least about 5 minutes, e.g. about 15 minutes. Of course, the duration of incubation also depends on the selected incubation temperature. 15 In preferred embodiments, the incubation step B) is performed at a temperature of at least about 30° C., at least about 35° C., at least about 40° C., at least about 45° C., at least about 50° C., at least about 55° C., at least about 60° C., at least about 65° C., at least about 70° C. In preferred embodiments, the incubation step B) is performed at a temperature ranging from about 35° C. to about 70° C., e.g. about 55° C.

In the context of the invention, the starting point of the incubation step is the time point when the reaction conditions of the IVT are adjusted to 2 mM GTP (by the addition of e.g. about 2 mM GTP). Accordingly, the temperature of the in vitro transcription reaction has to be adjusted to the desired reaction temperature, which is e.g. 55° C.

In preferred embodiments, the incubation step B) is performed at a temperature ranging from about 35° C. to about 70° C., e.g. about 55° C.

In specific embodiments, the incubation step B) is performed for at least 5 minutes (e.g. 15 Minutes) at a temperature of about 55° C.

In preferred embodiments, the method does not involve a step of enzymatic RNA ligation or a step of adding chemical compounds required for chemical circularization or a step of splint ligation.

In preferred embodiments, the incubation step B) is configured to produce more than about 60%(w/w), 65%(w/w), 70%(w/w), 75%(w/w), 80%(w/w), 85%(w/w), 90%(w/w), or 95% (w/w) circular RNA molecules (in relation to (non-circularized) linear precursor RNA). Preferably, the incubation step B) is configured to produce at least 80%(w/w) circular RNA molecules.

In preferred embodiments, the incubation step B) is configured to produce less than 40% (w/w). 30%(w/w), 25%(w/w), 20%(w/w), 15%(w/w), 10%(w/w), 5%(w/w) non-circularized linear precursor RNA (in relation to total RNA). Preferably, the incubation step B) is configured to produce less than 20%(w/w) non-circularized precursor RNA (in relation to circular RNA molecules).

The amount of circular RNA and (non-circularized) linear precursor RNA may be determined by gel electrophoresis (e.g. capillary gel electrophoresis) or analytical HPLC (e.g. analytical RP-HPLC).

For obtaining a pharmaceutical product, it may be required to perform at least one purification step to remove unwanted impurities from the circular RNA product, such as e.g. non-circularized linear precursor RNA or intermediates derived from the self-splicing process (e.g. intronic RNA molecules, e.g. SEQ ID NO: 184 or SEQ ID NO: 185).

Further impurities may comprise peptides or proteins (e.g. enzymes derived from RNA in vitro transcription, e.g.

RNA polymerases, RNases, pyrophosphatase, restriction endonuclease, DNase), spermidine, BSA, short abortive RNA sequences, RNA fragments (short double stranded RNA fragments, short single stranded RNA fragments, abortive RNA sequences etc.), free nucleotides (modified nucleotides, conventional NTPs, cap analogue), template DNA fragments, buffer components (HEPES, TRIS, MgCl2, CaCl2), linear precursor RNA or fragments thereof, intronic RNA fragments, RNAse. Other potential impurities may be derived from e.g. fermentation procedures and comprise bacterial impurities (bioburden, bacterial DNA, bacterial RNA) or impurities derived from purification procedures (organic solvents etc.).

In preferred embodiments, the obtaining step C) comprises at least one purification step C1) of affinity-based removal of the non-circularized linear precursor RNA and/or intronic splice products.

For example, intronic splice products according to SEQ ID NO: 184 (or fragments thereof) and according to SEQ ID NO: 185 (or fragments thereof) may represent a prominent impurity that needs to be efficiently removed.

In particularly preferred embodiments, step C1) comprises a step of selectively binding non-circularized linear precursor RNA to an antisense oligonucleotide. In addition, step C1) is suitable for binding intronic splice products to an antisense oligonucleotide.

In particularly preferred embodiments, the antisense oligonucleotide is configured to (selectively) bind the 3′ permuted intron-exon element or the 5′ permuted intron-exon element of the non-circularized linear precursor RNA.

In other particularly preferred embodiments, the antisense oligonucleotide is configured to (selectively) bind the 3′ terminal purification tag (e.g. an RNA sequence tag element element) located on the linear precursor RNA.

In other particularly preferred embodiments, the antisense oligonucleotide is configured to (selectively) bind the 5′ terminal purification tag (e.g. an RNA sequence tag element element) located on the linear precursor RNA.

Preferably, the affinity-based removal of linear precursor RNA is essentially performed as described in aspect provided in paragraph 9 “A method of purifying circular RNA”.

In preferred embodiments, the obtaining step C) comprises at least one purification step C2) selected from RP-HPLC, AEX, size exclusion chromatography, hydroxyapatite chromatography, TFF, filtration, precipitation, core-bead flow through chromatography, oligo(dT) purification, cellulose-based purification, spin column or affinity-based capturing of circular RNA, or any combination.

In embodiments, the step C2) is performed before step C1 or after step C1.

In preferred embodiments, step C2 is performed in addition to step C1.

In alternative embodiments, step C2 is performed without performing step C1.

In preferred embodiments, the at least one purification step C2) is RP-HPLC. In a preferred embodiment of the RP-HPLC, a non-alkylated porous polystyrenedivinylbenzene is used that may have a particle size of 8.0±1.5 μm, in particular 8.0±0.5 μm, and a pore size of 3500 to 4500A and most preferably of 4000 Å. In preferred embodiments, the RP-HPLC step is performed as described in WO2008/077592, in particular according to PCT claims 1 to 26. Accordingly, the disclosure of WO2008/077592, in particular the disclosure relating to PCT claims 1 to 26 are herewith incorporated by reference.

In preferred embodiments, the at least one purification step C2) is tangential flow filtration (TFF). In preferred embodiments, the TFF performed after the RP-HPLC is preferably performed as described in published patent application WO2016/193206, the disclosure relating to TFF for conditioning and/or purifying RP-HPLC purified RNA disclosed in WO2016/193206 herewith incorporated by reference. Exemplary parameters for TFF of the RP-HPLC purified RNA are provided in Example 14, e.g. Table 18 of WO2016/193206.

In preferred embodiments, the at least one purification step C2) is cellulose-based purification.

Suitably, the circular RNA preparation is purified by subjecting the preparation comprising circular RNA to a cellulose material (either a column or cellulose particles) under certain conditions. Such a purification step is considered to be particularly suitable for removing dsRNA by products (e.g. produced during RNA in vitro transcription).

The cellulose purification is suitably performed using a buffer comprising 14 to 20% (v/v) ethanol and 15 mM to 70 mM salt which facilitates binding of dsRNA to cellulose. The unbound, single stranded circular RNA may then be subjected to further purification steps. In preferred embodiments, the cellulose-purification is preferably performed as described in published patent application WO2017/182524, in particular according to PCT claims 1 to 45. Accordingly, the disclosure of WO2017/18252, in particular the disclosure relating to PCT claims 1 to 45 are herewith incorporated by reference.

In preferred embodiments, the at least one purification step C2) is core-bead flow through chromatography. An exemplary core bead flow-through chromatography medium is Capto™ Core 700 beads from GE Healthcare.

Preferably, circular RNA is selectively recovered from the capto-core column in the flow-through. Proteins and short RNA fragments or dsRNA are retained in the capto-core beads. Flow-through fractions containing circular RNA may be identified by measuring UV absorption at 260 nm. The composition comprising the circular RNA of interest may be collected in the flow-through and may be subjected to at least one further purification step.

Purification of RNA transcript by core bead chromatography is described in WO 2014140211 Å1, the disclosure relating thereto is herewith incorporated by reference.

In preferred embodiments, step C2) is selected from RP-HPLC and/or TFF.

In other preferred embodiments, the at least one purification step C2) is an affinity-based capturing of the circular RNA.

In preferred embodiments, the affinity-based capturing comprises a step of selectively binding circular RNA to an antisense oligonucleotide.

In preferred embodiments of the affinity-based capturing, the antisense oligonucleotide is configured to bind the splice-junction element (v), in particular, the sequence on the splice-junction element that is unique for the circular RNA. That unique junction sequence basically represents the junction site that is generated by the self-splicing event as described herein. As an example, the splice junction element of the circular RNA of the invention may comprise a unique junction sequence. As an example, in the context of the present invention, such a unique junction sequence comprises the sequence CUUUCC (SEQ ID NO: 511).

Accordingly, in embodiments of the affinity-based capturing, the antisense oligonucleotide comprises or consists a nucleic acid sequence (DNA and/or LNA) identical or at least 70%, 80%, 90%, 91%, 92%, 93%, 94%, 95%, 96%, 97%, 98%, or 99% identical to GGAAAG (SEQ ID NOs: 512), or fragments or variants thereof.

Preferably, the affinity-based capturing of circular RNA is essentially performed as described in aspect provided in paragraph 10 “A method of purifying circular RNA by affinity based capturing”.

In preferred embodiments, the obtaining step C) comprises a step C3) of digesting of linear RNA (impurities).

Linear RNA should be digested to avoid e.g. immunostimulation of the obtained circular RNA product. Linear RNA that may be digested are non-circularized linear precursor RNA or linear RNA splice products.

In preferred embodiments, the step of digesting is performed using an RNAse that is specific for linear RNA molecules.

Accordingly, in embodiments, linear RNA may be digested with an RNase, e.g., RNase R, Exonuclease T, A Exonuclease, Exonuclease I, Exonuclease VII, T7 Exonuclease, or XRN-1; preferably, the RNase is RNase R and/or XRN-1.

In particularly preferred embodiments, linear RNA impurities (e.g. non-circularized linear RNA molecules or linear splice products) are removed by a digestion step using an RNAse specific for linear RNA, preferably wherein the RNAse is selected from RNase R.

In preferred embodiments, the step C3) is performed before step C₁ or after step C₁.

In preferred embodiments, the step C3) is performed before step C2 or after step C2.

In preferred embodiments, the method additionally comprises a step of 5′ dephosphorylation of linear RNA impurities. Linear RNA may carry 5′ triphosphate ends that should be removed to avoid e.g. immunostimulation of the obtained circular RNA product. In that context, linear RNA that may be dephosphorylated are non-circularized linear precursor RNA molecules (the linear precursor RNA) or linear RNA splice products. 25 Accordingly, in preferred embodiments, the step of 5′ dephosphorylation of linear RNA impurities may reduce the immunostimulatory properties of the obtained circular RNA product.

The dephsphorylation may be carried out using an enzyme that converts a 5′ triphosphate of the linear RNA into a 5′ monophosphate. Accordingly, in some embodiments, the circular RNA preparation is contacted with RNA 5′ pyrophosphohydrolase (RppH) or an ATP diphosphohydrolase (apyrase) to convert a 5′ triphosphate of the linear RNA into a 5′ monophosphate.

Preferably, the dephsphorylation may be carried out using an enzyme that removes all three 5′ phosphate groups. Accordingly, in preferred embodiments the circular RNA preparation is contacted with a phosphatase (e.g., Antarctic Phosphatase, Shrimp Alkaline Phosphatase, or Calf Intestinal Phosphatase to remove all three phosphates.

In preferred embodiments, the method additionally comprises a step of DNA digestion, protein digestion, and/or dsRNA digestion.

A step of DNase digestion and/or removal may be performed by contacting the RNA preparation with a DNase.

Preferably, DNase digestion is performed after the incubation step B). Alternatively, DNase digestion is performed after the RNA in vitro transcription step A1) A step of protein digestion and/or removal may be performed by contacting the RNA preparation with a Proteinase, e.g. with Proteinase K Preferably, Proteinase K digestion is performed after the incubation step B) and before the obtaining step C).

A step of dsRNA digestion to remove dsRNA may be performed by contacting the RNA preparation with RNAse III. Preferably, RNAse Ill digestion is performed after the incubation step B) and before the obtaining step C).

In preferred embodiments, the linear precursor RNA or the (non-ircularized) linear precursor RNA is further characterized by any of the features as defined in the second aspect. In preferred embodiments, the circular RNA obtained by the method is further characterized by any of the features as defined in the first aspect.

The method of preparing circular RNA may additionally comprise a step of formulating the obtained circular RNA in a pharmaceutically acceptable carrier, e.g. formulating the obtained circular RNA in lipid-based carriers.

Suitable formulations are provided in the context of the third aspect

### 9: A Method of Purifying Circular RNA

In a further aspect, the invention provides a method of purifying a circular RNA from a composition comprising non-circularized precursor RNA and interalia circular RNA by affinity-based removal of the linear precursor RNA.

The composition may be considered as an “impure composition” obtained from a RNA circularization. As the conversion of a linear precursor RNA to a circular RNA does not happen with a 100% efficiency, the (non-circularized) linear precursor RNA has to be removed in a highly efficient manner. Furthermore, depending on the selected circularization method, the impure composition may also comprise linear RNA splice products (that typically, in the context of the invention, comprise intronic sequences).

Accordingly, for obtaining a pharmaceutical product, it may be required to perform a purification to remove unwanted impurities from the circular RNA product, such as e.g. (non-circularized) linear precursor RNA and/or linear RNA sequences derived from the self-splicing process (e.g. intronic RNA molecules).

Notably, embodiments relating to the aspect “9: A method of purifying circular RNA” may likewise be read on and be understood as suitable embodiments of the method of purifying a circular RNA of the present aspect. In addition, embodiments of the first aspect defining the circular RNA of the composition (that is, the circular RNA that has to be purified) may likewise be read on and be understood as suitable embodiments of the method of purifying a circular RNA of the present aspect. In addition, embodiments of the second aspect defining the linear precursor RNA of the composition (that is, the non-circularized linear precursor RNA that represents an impurity) may likewise be read on and be understood as suitable embodiments of the method of purifying a circular RNA of the present aspect.

In preferred embodiments, the present aspect relates to a method of purifying a circular RNA from an (impure) composition comprising (non-circularized) linear precursor RNA and circular RNA comprising a step of
affinity-based removal of the (non-circularized) linear precursor RNA and/or intronic splice products; and
obtaining a preparation comprising purified circular RNA.

An affinity-based removal of the (non-circularized) linear precursor RNA is associated with technical challenges as the RNA sequences of the circular RNA and the linear precursor RNA are highly similar in sequence and/or length.

To ensure an efficient affinity-based removal of (non-circularized) linear precursor RNA, an element (e.g. an RNA sequence) on the linear precursor RNA has to be selected that is distinct from or not present on the circular RNA. Such a distinct element may then be used to design a specific affinity-based purification process.

In preferred embodiments, the circular RNA (that is to be purified) comprises a splice junction element (v). The splice junction element (v) is further defined in the context of the first aspect and represents the region where the RNA sequence is circularized by a self-splicing event.

In preferred embodiments, the (non-circularized) linear precursor RNA comprises a 3′ permuted intron-exon element and a 5′ permuted intron-exon element for circularization. These elements are further defined in the context of the second aspect and represent the 3′ terminal and 5′ terminal RNA sequence that inter alia comprise homology arms and intron fragments that are spliced out during the RNA circularization event Accordingly, using such elements for a purification is also suitable for removing linear intronic splice products (e.g. SEQ ID NO: 184 and/or SEQ ID NO: 185, or fragments thereof).

Accordingly, in the context of the invention, a 3′ permuted intron-exon element and a 5′ permuted intron-exon element is not present in the circular RNA of the invention.

In preferred embodiments of, the (non-circularized) linear precursor RNA, the linear precursor RNA comprises at least one purification tag, preferably at least one purification tag located at the 3′ and/or the 5′ terminus of the (non-circularized) linear precursor RNA. As such tags represent the 3′ terminal and 5′ terminal RNA sequence, these sequences are spliced out during the RNA circularization event. Accordingly, in the context of the invention, a the at least one purification tag is not present in the circular RNA of the invention. Accordingly, such a purification tag is also suitable for removing linear intronic splice products (e.g. SEQ ID NO: 184 and/or SEQ ID NO: 185, or fragments thereof).

Preferably, the purification tag is not generated by enzymatic Polyadenylation.

In preferred embodiments, the affinity-based removal comprises a step of selectively binding (non-circularized) linear precursor RNA to an antisense oligonucleotide.

In preferred embodiments, the antisense oligonucleotide is configured to bind the 3′ permuted intron-exon element or the 5′ permuted intron-exon element of the (non-circularized) linear precursor RNA.

The selection of a suitable sequence stretch in the 3′ permuted intron-exon element or the 5′ permuted intron-exon element may require bioinformatic analysis as it may be advantageous to select the target sequence (that is the sequence to which the antisense oligonucleotide is supposed to bind to) that is unstructured. Moreover, the target sequence should be dose to the 3′ and/or 5′ terminus to allow an efficient affinity-based removal. Preferably, the distance of the target sequence in the 3′ permuted intron-exon element or the 5′ permuted intron-exon element to the respective 3′ or 5′ terminus should be no more than about 150 nucleotides.

Accordingly, the method of the present aspect may comprise a step of bioinformatic analysis of the structure of the 3′ permuted intron-exon element or the 5′ permuted intron-exon element to identify a suitable target sequence and, based on that sequence, designing a complementary antisense oligonucleotide.

As an example, in case of the 3′ permuted intron-exon element (e.g. SEQ ID NO: 508), the target sequence in 25 the 3′ permuted intron-exon element may be SEQ ID NO: 510. In case of the 5′ permuted intron-exon element (e.g. SEQ ID NO: 506 or 507), the target sequence in the 5′ permuted intron-exon element may be SEQ ID NO: 509.

In preferred embodiments of the present aspect, the antisense oligonucleotide is configured to bind the at least 30 one purification tag located at the 3′ and/or the 5′ terminus of the linear precursor RNA.

In preferred embodiments, the antisense oligonucleotide is configured not to bind to the circular RNA. In preferred embodiments, the antisense oligonucleotide is configured not to bind to linear intronic splice products.

In preferred embodiments, the antisense oligonucleotide comprises RNA, DNA, and/or LNA nucleotides.

In particularly preferred embodiments, the antisense oligonucleotide comprises LNA nucleotides.

In preferred embodiments, the antisense oligonucleotide has a length ranging from about 5 nucleotides to about 50 nucleotides, preferably ranging from about 5 nucleotides to about 30 nucleotides, more preferably ranging from about 10 nucleotides to about 30 nucleotides.

In embodiments where the antisense oligonucleotide is configured to bind to the 3′ permuted intron-exon element, the antisense oligonucleotide comprises or consists a nucleic acid sequence (DNA and/or LNA) identical or at least 70%, 80%, 90%, 91%, 92%, 93%, 94%, 95%, 96%, 97%, 98%, or 99% identical to SEQ ID NOs: 208, or fragments or variants thereof. 10 In embodiments where the antisense oligonucleotide is configured to bind to the 5′ permuted intron-exon element (located on the (non-circularized) precursor RNA), the antisense oligonucleotide comprises or consists a nucleic acid sequence (DNA and/or LNA) identical or at least 70%, 80%, 90%, 91%, 92%, 93%, 94%, 95%, 96%, 97%, 98%, or 99% identical to SEQ ID NOs: 209, or fragments or variants thereof.

In preferred embodiments of the present aspect, the antisense oligonucleotide is immobilized on a solid support, preferably wherein the solid support is a bead or a column.

The shape, form, materials, and modifications of the solid support can be selected from a range of options depending on the desired application or scale. Exemplary materials that can be used as a solid support include, but are not limited to acrylics, carbon (e.g., graphite, carbon-fiber), cellulose (e.g., cellulose acetate), ceramics, controlled-pore glass, cross-linked polysaccharides (e.g., agarose or SEPHAROSE™), gels, glass (e.g., modified or functionalized glass), gold (e.g., atomically smooth Au(111)), graphite, inorganic glasses, inorganic polymers, latex, metal oxides (e.g., SiO2, TiO2, stainless steel), metalloids, metals (e.g., atomically smooth Au(1 111), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™. A single material or mixture of several different materials can form a solid support useful in the context of the invention.

In preferred embodiments, the solid support comprises sepharose. For example, the solid support may be a sepharose bead or a sepharose column.

In preferred embodiments, the solid support comprises silica. For example, the solid support may be a silica bead or a silica column.

In preferred embodiments, the solid support is a monolithic material, e.g. a methacrylate monolith.

The terms “monolith,” “monolithic matrix” and “monolithic column” are used interchangeably herein to refer to a solid support (e.g. a chromatography column) composed of a continuous stationary phase made of a polymer matrix. In contrast to particle-based chromatography columns, monolithic columns are made of a porous polymer material with highly interconnected channels and large pore size. While particle-based columns rely on diffusion through pores, separation by monolithic columns occurs primarily by convective flow through relatively large channels (about 1 micron or more).

A suitable monolithic matrix may be derived from a variety of materials, such as but not limited to, polymethacrylate, polyacrylamide, polystyrene, silica and cryogels.

In one embodiment, the solid support is modified to contain chemically modified sites that can be used to attach, either covalently or non-covalently, the antisense oligonucleotide to discrete sites or locations on the surface. “Chemically modified sites” in this context includes, but is not limited to, the addition of a pattern of chemical functional groups including amino groups, carboxy groups, oxo groups and thiol groups, that can be used to covalently attach the antisense oligonucleotide, which generally also contain corresponding reactive functional groups. Examples of surface functionalizations are: Amino derivatives, Thiol derivatives, Aldehyde derivatives, Formyl derivatives, Azide Derivatives (click chemistry), Biotin derivatives, Alkyne derivatives, Hydroxyl derivatives, Activated hydroxyls or derivatives, Carboxylate derivatives, activated carboxylate derivates, Activated carbonates, Activated esters, NHS Ester (succinimidyl), NHS Carbonate (succinimidyl), Imidoester or derivated, Cyanogen Bromide derivatives, Maleimide derivatives, Haloacteyl derivatives, lodoacetamide/iodoacetyl derivatives, Epoxide derivatives, Streptavidin derivatives, Tresyl derivatives, Diene/conjugated diene derivatives (diels alder type reaction), Alkene derivatives, Substituted phosphate derivatives, Bromohydrin/halohydrin, Substituted disulfides, Pyridyl-disulfide Derivatives, Aryl azides, Acyl azides, Azlactone, Hydrazide derivatives, Halobenzene derivatives, Nucleoside derivatives, Branching/multi functional linkers, Dendrimeric functionalities, and/or Nucleoside derivatives; or any combination thereof.

In some embodiments, the antisense oligonucleotide is linked directly to the solid support. In some embodiments, the antisense oligonucleotide is linked to the solid support via a linker.

In some embodiments, a solid support and/or the antisense oligonucleotide can be attached to a linker.

The term “linker” can refer to a connection between two molecules or entities, for example, the connection between the antisense oligonucleotide and a spacer or the connection between the antisense oligonucleotide and a solid support. The linker can be formed by the formation of a covalent bond or a non-covalent bond.

Suitable covalent linkers can include, but are not limited to the formation of an amide bond, an oxime bond, a hydrazone bond, a triazole bond, a sulfide bond, an ether bond, an enol ether bond, an ester bond, or a disulfide bond.

Suitable linkers include alkyl and aryl groups, including heteroalkyl and heteroaryl, and substituted derivatives of these. In some instances, linkers can be amino acid based and/or contain amide linkages. Examples of linkers are: Amino derivatives, Thiol derivatives, Aldehyde derivatives, Formyl derivatives, Azide Derivatives (click chemistry), Biotin derivatives, Alkyne derivatives, Hydroxyl derivatives, Activated hydroxyls or derivatives, Carboxylate derivatives, activated carboxylate derivates, Activated carbonates, Activated esters, NHS Ester (succinimidyl), NHS Carbonate (succinimidyl), Imidoester or derivated, Cyanogen Bromide derivatives, Maleimide derivatives, Haloacteyl derivatives, lodoacetamide/iodoacetyl derivatives, Epoxide derivatives, Streptavidin derivatives, Tresyl derivatives, Diene/conjugated diene derivatives (diels alder type reaction), Alkene derivatives, Substituted phosphate derivatives, Bromohydrin/halohydrin, Substituted disulfides, Pyridyl-disulfide Derivatives, Aryl azides, Acyl azides, Azlactone, Hydrazide derivatives, Halobenzene derivatives, Nucleotide derivatives, Branching/multi functional linkers, Dendrimeric funcationalities, and/or Nucleoside derivatives; or any combination thereof.

In particularly preferred embodiments, the antisense oligonucleotide is linked to a sepharose bead that comprises streptavidin.

In preferred embodiments, the method comprises a step of subjecting the composition comprising linear precursor RNA and circular RNA comprising to the antisense oligonucleotide (as defined herein) under conditions that allow nucleic acid hybridization.

In some embodiments, the conditions that allow nucleic acid hybridization is a temperature of about 20° C. to about 60° C., preferably about 30° C.

In some embodiments, the conditions that allow nucleic acid hybridization is at a pH of about 7.0.

In some embodiments, the conditions that allow nucleic acid hybridization is a buffer condition, wherein the buffer is a hybridization buffer, e.g. an saline sodium citrate buffer (SSC).

Suitably, the hybridization buffer comprises 100 mM to 1M sodium chloride and 10 mM to 100 mM trisodium citrate. For example, the hybridization buffer comprises 300 mM sodium chloride, 30 mM trisodium citrate.

In particularly preferred embodiments, the linear RNA precursor and the antisense oligonucleotide bind one another via non-covalent bonding, e.g. nucleic acid hybridization.

In embodiments, several affinity-based purification steps are performed in sequence, for example at least one, two, three, or even four affinity-based purification steps may be performed, e.g. a first step using an antisense oligonucleotide against the 3′ permuted intron-exon element (Affi 3′ PIE), and/or a second step using an antisense oligonucleotide against the 5′ permuted intron-exon element (Affi 5′ PIE), and/or a third step using 5 antisense oligonucleotide against a 3′ purification tag (Affi 3′ PT), and/or a fourth step using antisense oligonucleotide against a 5′ purification tag (Affi 5′ PT).

In preferred embodiments of the present aspect, after binding of the linear precursor RNA (and/or the linear intronic splice products) to an antisense oligonucleotide, the purified circular RNA is collected in a flow through or a supernatant. Suitably, the linear RNA impurity stays hybridized to the antisense oligonucleotide.

In preferred embodiments of the present aspect, the affinity-based purification method is operated in a batch mode, or as a chromatography. Preferably, the chromatography is a liquid chromatography, e.g. LC, FPLC, or HPLC, In preferred embodiments of the present aspect, the composition is subjected to the affinity-based removal of linear precursor RNA is additionally purified using a method described herein to remove additional impurities.

Accordingly, a further purification step is performed before or after the affinity-based removal of linear precursor RNA to remove at least one further impurity.

In preferred embodiments, the method additionally comprises a step of digesting linear RNA using an RNAse specific for linear RNA, preferably wherein the RNAse is selected from RNase R. 25 In preferred embodiments, the method of purifying additionally comprises a step of 5′ dephosphorylation of (non-circularized) liner precursor RNA as defined herein.

In preferred embodiments, the method of purifying additionally comprises a step of DNA digestion, protein digestion, and/or dsRNA digestion as defined herein.

In preferred embodiments of, the method of purifying additionally comprises at least one step of purifying by means of RP-HPLC, AEX, size exclusion chromatography, hydroxyapatite chromatography, TFF, filtration, precipitation, core-bead flow through chromatography, oligo(dT) purification, spin column, cellulose-based purification, and/or affinity-based capturing of the circular RNA.

Further embodiments and details relating to said methods that may be used to additionally purify the composition are provided in the context of aspect provided in paragraph 8 “A method for preparing circular RNA”.

In preferred embodiments, the at least additionally purification is an affinity-based capturing of the circular RNA, essentially performed as described in the aspect of paragraph 10.

In preferred embodiments, the at least one step of purifying is performed before and/or after the affinity-based removal of linear precursor RNA.

In preferred embodiments, the obtained preparation comprises more than about 60%(w/w), 65%(w/w), 70%(w/w), 75%(w/w), 80%(w/w), 85%(w/w), 90%(w/w), or 95% (w/w) circular RNA molecules (in relation to total RNA). Preferably, the obtained preparation comprises at least 80%(w/w) circular RNA molecules.

In particularly preferred embodiments, the obtained preparation comprises more than about 96%(w/w), 97%(w/w), 98%(w/w), 99%(w/w), 99.5%(w/w), 99.9%(w/w) circular RNA molecules (in relation to total RNA).

Preferably, the obtained preparation comprises at least 80%(w/w) circular RNA molecules. 15 In preferred embodiments, the obtained preparation comprises less than 40% (w/w). 30%(w/w), 25%(w/w), 20%(w/w), 15%(w/w), 10%(w/w), 5%(w/w) non-circularized linear precursor RNA (in relation to total RNA).

Preferably, the obtained preparation comprises less than 20%(w/w) non-circularized linear precursor RNA (in relation to total RNA). 20 In particularly preferred embodiments, the obtained preparation comprises less than 5% (w/w). 4%(w/w), 3%(w/w), 2%(w/w), 1%(w/w), or 0.5%(w/w) non-circularized linear precursor RNA (in relation to total RNA).

In preferred embodiments, the obtained preparation comprises less than 5% (w/w), 4%(w/w), 3%(w/w), 2%(w/w), 1%(w/w), 0.5%(w/w), 0.1%(w/w) linear intronic splice products (in relation to total RNA). Preferably, the obtained preparation comprises less than 1%(w/w) linear intronic splice products (in relation to total RNA).

In preferred embodiments, the method leads to a reduction of linear RNA (that is linear precursor RNA and/or linear intronic splice products) of 50%(w/w), 60%(w/w), 70%(w/w), 80%(w/w), 90%(w/w), 95%(w/w) in the obtained preparation (in relation to total RNA), preferably compared to the level linear RNA molecules in the composition prior to the purification step(s).

In preferred embodiments, the obtained preparation comprises purified circular RNA.

Accordingly, “purified circular RNA” as used herein has a degree of purity of more than 75%, 80%, 85%, very particularly 90%, 91%, 92%, 93%, 94%, 95%, 96%, 97%, 98% and most favourably 99% or more. The degree of purity may for example be determined by an analytical HPLC, wherein the percentages provided above correspond to the ratio between the area of the peak for the target RNA and the total area of all peaks representing all the by-products. Alternatively, the degree of purity may for example be determined by an analytical agarose gel electrophoresis or capillary gel electrophoresis.

In preferred embodiments, the purified circular RNA preparation has a purity of at least 70%(w/w), 75%(w/w), 80%(w/w), 85%(w/w), 90%(w/w), 95%(w/w), 96%(w/w), 97%(w/w), 98%(w/w), or 99%(w/w).

In preferred embodiments, the purified circular RNA preparation comprising less than 5%(w/w) dsRNA fragments, less than 5%(w/w) DNA, less than 5%(w/w) protein, and less than 5%(w/w) abortive IVT fragments.

In particularly preferred embodiments, the purified circular RNA comprising less than 1%(w/w) dsRNA fragments, less than 1%(w/w) DNA, less than 1%(w/w) protein, and less than 1%(w/w) abortive IVT fragments.

In even more preferred embodiments, the purified circular RNA comprising less than 0.5%(w/w) dsRNA fragments, less than 0.5%(w/w) DNA, less than 0.5%(w/w) protein, and less than 0.5%(w/w) abortive IVT fragments.

In preferred embodiments, the purified circular RNA preparation comprises no more than 5% (w/w) nicked circular RNA molecules of the total ribonucleotide molecules in the preparation. In some embodiments, the purified circular RNA preparation comprises no more than 9% (w/w), 8% (w/w), 7% (w/w), 6% (w/w), 5% (w/w), 4% (w/w), 3% (w/w), 2% (w/w), 1% (w/w), or 0.5% (w/w) nicked circular RNA molecules of the total ribonucleotide molecules in the preparation.

In preferred embodiments, the purified circular RNA preparation comprises circular RNA and no more than 0.5% (w/w), 1% (w/w), 2% (w/w), 3% (w/w), 4% (w/w), 5% (w/w), 6% (w/w), 7% (w/w), 8% (w/w), 9% (w/w), or 10% (w/w) linear RNA molecules of the total ribonucleotide molecules in the preparation.

In preferred embodiments, the purified circular RNA has an RNA integrity of at least 70%, 75%, 80%, 85%, 90%, 95%. RNA integrity is suitably determined using analytical HPLC, preferably analytical RP-HPLC.

In preferred embodiments of the method of purifying, the (non-circularized) linear precursor RNA is further characterized by any of the features as defined in the second aspect. In preferred embodiments of the method of purifying, the circular RNA is further characterized by any of the features as defined in the first aspect.

10: A Method of Purifying Circular RNA by Affinity-Based Capturing In a further aspect, the invention provides a method of purifying a circular RNA from a composition comprising non-circularized precursor RNA and inter alia circular RNA by affinity-based capturing of the circular RNA.

Notably, embodiments relating to the previous aspects may also apply to the present aspect.

In preferred embodiments, the present aspect relates to a method of purifying a circular RNA from an (impure) composition comprising (non-circularized) linear precursor RNA and circular RNA comprising a step of
affinity-based capturing of the circular RNA; and
obtaining a preparation comprising purified circular RNA.

In preferred embodiments, the affinity-based capturing comprises a step of selectively binding circular RNA to an antisense oligonucleotide.

In preferred embodiments of the affinity-based capturing, the antisense oligonucleotide is configured to bind the 10 splice-junction element (v), in particular, the sequence on the splice-junction element that is unique for the circular RNA. That unique splice junction sequence basically represents the junction site that is generated by the self-splicing event as described herein.

As an example, the splice junction element of the circular RNA of the invention may comprise a unique splice junction sequence. As an example, in the context of the present invention, such a unique junction sequence comprises the sequence CTTTCC (SEQ ID NO: 511).

In preferred embodiments of the affinity-based capturing, an antisense oligonucleotide is configured to bind to a unique junction sequence of the circular RNA, preferably to the unique junction sequence of CTTTCC (SEQ ID NO: 511).

In preferred embodiments of the affinity-based capturing, the antisense oligonucleotide is configured not to bind to the linear precursor RNA. In preferred embodiments of the affinity-based capturing, the antisense oligonucleotide is configured not to bind to linear intronic splice products.

In preferred embodiments of the affinity-based capturing, the antisense oligonucleotide comprises RNA, DNA, and/or LNA nucleotides.

In particularly preferred embodiments of the affinity-based capturing, the antisense oligonucleotide comprises LNA nucleotides.

In preferred embodiments of the affinity-based capturing, the antisense oligonucleotide has a length ranging from about 5 nucleotides to about 50 nucleotides, preferably ranging from about 5 nucleotides to about 30 nucleotides, more preferably ranging from about 10 nucleotides to about 30 nucleotides.

In embodiments of the affinity-based capturing, the antisense oligonucleotide comprises or consists a nucleic acid sequence (DNA and/or LNA) identical or at least 70%, 80%, 90%, 91%, 92%, 93%, 94%, 95%, 96%, 97%, 98%, or 99% identical to GGAAAG (SEQ ID NOs: 512), or fragments or variants thereof.

In preferred embodiments of the affinity-based capturing, the antisense oligonucleotide is immobilized on a solid support, preferably wherein the solid support is a bead or a column.

Suitable shape, form, materials, and modifications of the solid support can be selected from a range of options depending on the desired application or scale and can be selected from those provided in the aspect of paragraph 9.

In one embodiment of the affinity-based capturing, the solid support is modified to contain chemically modified sites that can be used to attach, either covalentiy or non-covalently, the antisense oligonucleotide to discrete sites or locations on the surface as described in the aspect of paragraph 9.

In some embodiments of the affinity-based capturing, the antisense oligonucleotide is linked directly to the solid support. In some embodiments of the affinity-based capturing, the antisense oligonucleotide is linked to the solid support via a linker.

In some embodiments of the affinity-based capturing, a solid support and/or the antisense oligonucleotide can be attached to a linker, preferably a linker as described in the aspect of paragraph 9.

In particulady preferred embodiments of the affinity-based capturing, the antisense oligonucleotide is linked to a sepharose bead that comprises streptavidin.

In preferred embodiments of the affinity-based capturing, the method comprises a step of subjecting the composition comprising linear precursor RNA and circular RNA comprising to the antisense oligonucleotide (as defined herein) under conditions that allow nucleic acid hybridization, preferably a linker as described in the aspect of paragraph 9.

In preferred embodiments of the affinity-based capturing, capturing the circular RNA to an antisense oligonucleotide, the purified circular RNA is collected by eluting the circular RNA and discarding the flow through or supernatant.

In preferred embodiments of the affinity-based capturing, the affinity-based capturing of circular RNA is operated in a batch mode, or as a chromatography. Preferably, the chromatography is a liquid chromatography, e.g. LC, FPLC, or HPLC.

In preferred embodiments of the affinity-based capturing, the method additionally comprises at least one step of purifying selected from RP-HPLC, AEX, size exclusion chromatography, hydroxyapatite chromatography, TFF, filtration, precipitation, core-bead flow through chromatography, spin column, oligo(dT) purification, cellulose-based purification, and affinity-based removal of linear RNA (see aspect of paragraph 9).

In preferred embodiments of the affinity-based capturing, the obtained preparation comprises more than about 60%(w/w), 65%(w/w), 70%(w/w), 75%(w/w), 80%(w/w), 85%(w/w), 90%(w/w), or 95% (w/w) circular RNA molecules (in relation to total RNA). Preferably, the obtained preparation comprises at least 80%(w/w) circular RNA molecules.

In particularly preferred embodiments of the affinity-based capturing, the obtained preparation comprises more 10 than about 96%(w/w), 97%(w/w), 98%(w/w), 99%(w/w), 99.5%(w/w), 99.9%(w/w) circular RNA molecules (in relation to total RNA). Preferably, the obtained preparation comprises at least 80%(w/w) circular RNA molecules.

In preferred embodiments of the affinity-based capturing, the obtained preparation comprises less than 40% (w/w). 30%(w/w), 25%(w/w), 20%(w/w), 15%(w/w), 10%(w/w), 5%(w/w) non-circularized linear precursor RNA (in relation to total RNA). Preferably, the obtained preparation comprises less than 20%(w/w) non-circularized linear precursor RNA (in relation to total RNA).

In particularly preferred embodiments of the affinity-based capturing, the obtained preparation comprises less than 5% (w/w). 4%(w/w), 3%(w/w), 2%(w/w), 1%(w/w), or 0.5%(w/w) non-circularized linear precursor RNA (in relation to total RNA).

In preferred embodiments of the affinity-based capturing, the obtained preparation comprises less than 5% (w/w), 4%(w/w), 3%(w/w), 2%(w/w), 1%(w/w), 0.5%(w/w), 0.1%(w/w) linear intronic splice products (in relation to total RNA). Preferably, the obtained preparation comprises less than 1%(w/w) linear intronic splice products (in relation to total RNA).

In preferred embodiments of the affinity-based capturing, the method leads to a reduction of linear RNA (that is linear precursor RNA and/or linear intronic splice products) of 50%(w/w), 60%(w/w), 70%(w/w), 80%(w/w), 90%(w/w), 95%(w/w) in the obtained preparation (in relation to total RNA), preferably compared to the level linear RNA molecules in the composition prior to the purification step(s).

In preferred embodiments of the affinity-based capturing, the purified circular RNA preparation has a purity of at least 70%(w/w), 75%(w/w), 80%(w/w), 85%(w/w), 90%(w/w), 95%(w/w), 96%(w/w), 97%(w/w), 98%(w/w), or 99%(w/w).

In preferred embodiments of the affinity-based capturing, the purified circular RNA preparation comprising less than 5%(w/w) dsRNA fragments, less than 5%(w/w) DNA, less than 5%(w/w) protein, and less than 5%(w/w) abortive IVT fragments. In particularly preferred embodiments, the purified circular RNA comprising less than 1%(w/v) dsRNA fragments, less than 1%(w/w) DNA, less than 1%(w/w) protein, and less than 1%(w/w) abortive IVT fragments. In even more preferred embodiments, the purified circular RNA comprising less than 0.5%(w/w) dsRNA fragments, less than 0.5%(w/w) DNA, less than 0.5%(w/w) protein, and less than 0.5%(w/w) abortive IVT fragments.

In preferred embodiments of the affinity-based capturing, the purified circular RNA preparation comprises no more than 5% (w/w) nicked circular RNA molecules of the total ribonucleotide molecules in the preparation. In some embodiments, the purified circular RNA preparation comprises no more than 9% (w/w), 8% (w/w), 7% (w/w), 6% (w/w), 5% (w/w), 4% (w/w), 3% (w/w), 2% (w/w), 1% (w/w), or 0.5% (w/w) nicked circular RNA molecules of the total ribonucleotide molecules in the preparation.

In preferred embodiments of the affinity-based capturing, the purified circular RNA preparation comprises circular RNA and no more than 0.5% (w/w), 1% (w/w), 2% (w/w), 3% (w/w), 4% (w/w), 5% (w/w), 6% (w/w), 7% (w/w), 8% (w/w), 9% (w/w), or 10% (w/w) linear RNA molecules of the total ribonucleotide molecules in the preparation.

In preferred embodiments of the affinity-based capturing, the purified circular RNA has an RNA integrity of at least 70%, 75%, 80%, 85%, 90%, 95%. RNA integrity is suitably determined using analytical HPLC, preferably analytical RP-HPLC.

In preferred embodiments of the method of the affinity-based capturing, the (non-circularized) linear precursor RNA is further characterized by any of the features as defined in the second aspect. In preferred embodiments of the method of purifying, the circular RNA is further characterized by any of the features as defined in the first aspect.

11: Preparation Comprising Circular RNA Obtained by the Methods of Producing an/or Purifying

In a further aspect, the invention provides a preparation comprising circular RNA, wherein said preparation is produced by the method of paragraph 8, or wherein said preparation is purified by the method of paragraph 9, or wherein said preparation is purified by the method of paragraph 10. Accordingly, said further aspect relates to a preparation comprising circular RNA obtainable by the method for preparing circular RNA as provided herein or the methods of purifying a circular RNA.

Said obtained preparation comprising circular RNA is characterized by advantageous quality features e.g. high purity, reduced immunostimulation, increased expression etc. as specified in detail in conjunction with the previously described aspects of the invention.

### Brief Description of Tables:

Table 1: Linear precursor RNA constructs used for RNA circularization
Table 2: Circular RNA constructs used in experiments
Table 3: Linear capped mRNA constructs used in experiments

### BRIEF DESCRIPTION OF THE FIGURES

FIG. 1: shows the relative GLuc activity of two circular RNA constructs compared to a linear capped mRNA over 7 days in a human liver cell line (HepG2) (see FIG. 1A) and lung epithelial cell line (A549) (see FIG. 1B). The improved circular RNA (CircRNA-02) shows a significant longer protein expression compared to linear capped mRNA (linear) and higher and longer protein expression compared to the standard circular RNA (CircRNA-01). Further details are provided in Example 4.

FIG. 2: shows in vivo luciferase protein production in the liver after i.v. injection of LNP formulated improved circular RNA (CircRNA-05). Further details are provided in Example 5.

FIG. 3: shows IFNa titer in the serum after i.v. injection of LNP formulated improved circular RNA (CircRNA-05) and a corresponding linear capped mRNA (linear). CircRNA-05 reduces IFNa level in serum compared to linear capped RNA in vivo after i.v. injection (see Example 6).

FIG. 4: shows GLuc expression of different improved circular RNA constructs comprising different adenosine stretches (CircRNA-02, CircRNA-03, CircRNA-04) and a corresponding linear capped mRNA (linear) in primary human skeletal muscle cells. Adenosine stretches had an effect on protein expression and/or RNA stability. Further details are provided in Example 7.

FIG. 5: shows the in vivo kinetic of PpLuc expression in muscle cells after i.m. injection of LNP formulated improved circular RNA (CircRNA-05) and linear capped mRNA. Further details are provided in Example 8.

FIG. 6: shows an agarose gel of different fractions of the antisense oligo purification of circular RNA constructs (see Example 9). Circular RNA is strongly enriched in the supernatant fraction whereas non-circularized precursor RNA and spliced introns were removed. Column A: fraction of non-purified in vitro transcription product precursor RNA; Column B: fraction of circular RNA incubated with 5′ end biotinylated AS2 LNA oligo (“+3′AS oligo”) in splicing reaction condition; Column C: purified supernatant fraction enriched circular RNA after pull down with streptavidin sepharose beads; Column D: fraction of circular RNA incubated with 5′ end biotinylated AS1 LNA oligo (“+5′AS oligo”) in splicing reaction condition; Column E: purified supernatant fraction enriched circular RNA after pull down with streptavidin Sepharose beads. Further details are provided in Example 9.

FIG. 7: shows specific humoral immune responses (IgG titer) in two different timepoints (day 21 and day 35) after vaccination with a formulated circRNA construct (CircRNA-09, see Table 2) encoding for an HA antigen in three different doses. Even with a very low concentration (0.5pg circRNA) a specific humoral immune response was detected after first vaccination and after second vaccination total IgG titer increased significantly. Further details are provided in Example 11.

FIG. 8: shows specific cellular immune responses after vaccination with a formulated circRNA construct (CircRNA-09, see Table 2) encoding for an HA antigen. The antigen specific polyfunctional T cell responses were detected for CD4 (FIG. 8A) and CD8 (FIG. 8B) specific T cells. Additionally, the vaccination with the circRNA construct induced a strong effector memory T cell immune response in CD4 positive (FIG. 8C) and CD8 positive (FIG. 8D) positive memory T cells (TEM). Further details are provided in Example 11.

FIG. 9: shows dose dependent IFNalpha levels in the serum 18 hours after vaccination with a formulated circRNA construct (CircRNA-09, see Table 2) encoding for an HA antigen.

The lowest dose (0.5pg) showed INFalpha levels comparable with the PBS control group. Further details are provided in Example 11.

FIG. 10: shows increased PpLuc expression in vitro of an improved circRNA construct with an additional Kozak sequence (CircRNA-10, see Table 2) in HeLa (FIG. 10A) and HepG2 (FIG. 10B) cells. The increased expression is shown over 3 days in two different concentrations (5ng and 50ng). Further details are provided in Example 12.

FIG. 11: shows increased (CircRNA-11, see table 2) or comparable (CircRNA-12, see Table 2) PpLuc expression in vitro of circRNA constructs with two alternative IRES sequences in HeLa (FIG. 11A) and HepG2 (FIG. 11B) cells. The expression is shown over 3 days in two different concentrations (5ng and 50ng). Further details are provided in Example 13.

### EXAMPLES

In the following, examples illustrating various embodiments and aspects of the invention are presented. However, the present invention shall not to be limited in scope by the specific embodiments presented herein, and should rather be understood as being applicable to other compositions and/or vaccines and/or uses as for example defined in the specification. Accordingly, the following preparations and examples are given to enable those skilled in the art to more clearly understand and to practice the present invention. Indeed, various modifications of the invention in addition to those described herein will become readily apparent to those skilled in the art from the foregoing description, accompanying figures and the examples below.

### Example 1: Preparation and Purification of Circular RNA

The present example inter alia provides methods of producing and purifying the circular RNA of the invention.

### 1.1. Preparation of DNA Templates for Linear RNA Precursor Constructs

A DNA sequence for the production of linear precursor RNA was prepared to serve as a template for RNA in vitro transcription. The DNA template comprised inter alia a coxsackievirus B3 (CVB3) IRES, a G/C optimized coding sequence encoding Gaussia luciferase (GLuc) or Photinus pyralis luciferase (PpLuc), optionally a PSMB-3′UTR, optionally 36× or 60× or 100× adenosine stretches, and two permuted intron-exon sequences (3′ PIE and 5′ PIE) suitable for circularization of the linear precursor RNA into circular RNA via self-splicing.

Obtained plasmid DNA was transformed and propagated in bacteria using common protocols and plasmid DNA was extracted, purified, and used for subsequent RNA in vitro transcription as outlined below.

### 1.2. RNA In Vitro Transcription of Linear Precursor RNA

DNA plasmids prepared according to Example 1.1. were enzymatically linearized using a restriction enzyme and used for DNA dependent RNA in vitro transcription using T7 RNA polymerase in the presence of a sequence optimized nucleotide mixture (ATP/GTP/CTP/UTP), essentially preformed according to WO2015188933, under suitable buffer conditions. The obtained linear precursor RNA was used for circularization. The generated linear precursor RNA sequences are provided in Table 1.

TABLE 1
Linear precursor RNA constructs used for RNA circularization

| | SEQ | | | | | Poly(A) |
|---|---|---|---|---|---|---|
| ID | ID NO: | IRES | Kozak | coding sequence | UTR | sequence |
| PreCircRNA-01 | 196 | CVB3 | − | GLuc | — | — |
| PreCircRNA-02 | 197 | CVB3 | − | G/C optimized GLuc | PSMB3 | A36 |
| PreCircRNA-03 | 198 | CVB3 | − | G/C optimized GLuc | PSMB3 | A60 |
| PreCircRNA-04 | 199 | CVB3 | − | G/C optimized GLuc | PSMB3 | A100 |
| PreCircRNA-05 | 200 | CVB3 | − | G/C optimized | PSMB3 | A36 |
| | | | | PpLuc | | |
| PreCircRNA-06 | 521 | CVB3 | − | G/C optimized anti- | PSMB3 | A36 |
| | | | | Rabies IgG(HC) | | |
| PreCircRNA-07 | 522 | CVB3 | − | G/C optimized anti- | PSMB3 | A36 |
| | | | | Rabies IgG(LC) | | |
| PreCircRNA-08 | 523 | CVB3 | − | G/C optimized anti- | PSMB3 | A36 |
| | | | | Rabies IgG(HC + LC) | | |
| PreCircRNA- | 524 | CVB3 | − | G/C optimized | PSMB3 | A36 |
| 09 | | | | HA(NL2009) | | |
| PreCircRNA- | 525 | CVB3 | + | G/C optimized | PSMB3 | A36 |
| 10 | | | | PpLuc | | |
| PreCircRNA-11 | 526 | SaV | − | G/C optimized | PSMB3 | A36 |
| | | IRES | | PpLuc | | |
| PreCircRNA- | 527 | AiV | − | G/C optimized | PSMB3 | A36 |
| 12 | | IRES | | PpLuc | | |
| PreCircRNA-13 | 528 | SaV | + | G/C optimized | PSMB3 | A36 |
| | | IRES | | PpLuc | | |
| PreCircRNA- | 529 | AiV | + | G/C optimized | PSMB3 | A36 |
| 14 | | IRES | | PpLuc | | |
| PreCircRNA-15 | 530 | CVB3 | − | G/C optimized GLuc | RPS9 | A36 |
| PreCircRNA-16 | 531 | CVB3 | + | GC optimized | PSMB3 | A36 |
| | | | | CTLA4(2-35); mus musculus (Mm); | | |
| | | | | Trp2 (24-472, | | |
| | | | | D260E, W261L); | | |
| | | | | CTLA4(162-223) | | |
| PreCircRNA-17 | 532 | CVB3 | + | GC optimized | PSMB3 | A36 |
| | | | | CTLA4(2-35); Mm | | |
| | | | | Pmel(14-43); Mm | | |
| | | | | Dct(170-198); Mm | | |
| | | | | Pbk(131-159, | | |
| | | | | (V145D); Mm | | |
| | | | | Trp1(455-473, | | |
| | | | | A463M); Mm | | |
| | | | | Obsl1(1750-1778; | | |
| | | | | T1764M); PADRE; | | |
| | | | | CTLA4(162-223) | | |

### 1.3. Circularization Reaction:

During and following the RNA in vitro transcription of the linear precursor RNA, the circularization of the linear precursor RNA takes place. External homology regions of the 3′ PIE and 5′ PIE sequences allow for circularization of the linear precursor RNA using the permuted intron exon (PIE) circularization strategy.

To increase the circularization efficiency (that is, the conversion of linear precursor RNA to circular RNA) GTP was added at a concentration of 2 mM to the unpurified in vitro transcription product and the reaction mixture was incubated at 55° C. for 15 min. Following that, RNA was precipitated with LiCl and subsequently purified using RP-HPLC (PureMessenger®; according to WO2008/077592) to e.g. remove linear intronic splice products.

Circular RNA enriched RP-HPLC fractions were subjected to RNaseR digestion (0.8U RNaseR/pg RNA) for 30 min at 37° C. to digest non-circularized linear RNA and linear intronic splice products. Reaction product was purified using spin column (Monarch® RNA Cleanup Kit; NEB T2050). RP-HPLC purified circular RNA was treated with a phosphatase (QuickCIP, 0.3U/pmol RNA) for 20 min at 37° C. to remove 5′ triphosphates of potential linear RNA impurities. Following that, the phosphatase treated preparation comprising circular RNA was purified using spin columns.

The generated improved circular RNA sequences (CircRNA-02, CircRNA-03, CircRNA-04, CircRNA-05) as well as a standard circular RNA sequence (CircRNA-01) are provided in Table 2.

TABLE 2
Circular RNA constructs used in experiments

| | SEQ | | | | | Poly(A) |
|---|---|---|---|---|---|---|
| ID | ID NO: | IRES | Kozak | coding sequence | UTR | sequence |
| CircRNA-01 | 201 | CVB3 | − | GLuc | — | — |
| CircRNA-02 | 202 | CVB3 | − | G/C optimized GLuc | PSMB3 | A36 |
| CircRNA-03 | 203 | CVB3 | − | G/C optimized GLuc | PSMB3 | A60 |
| CircRNA-04 | 204 | CVB3 | − | G/C optimized GLuc | PSMB3 | A100 |
| CircRNA-05 | 205 | CVB3 | − | G/C optimized PpLuc | PSMB3 | A36 |
| CircRNA- 06 | 533 | CVB3 | − | G/C optimized anti- | PSMB3 | A36 |
| | | | | Rabies IgG(HC) | | |
| CircRNA- 07 | 534 | CVB3 | − | G/C optimized anti- | PSMB3 | A36 |
| | | | | Rabies IgG(LC) | | |
| CircRNA- 08 | 535 | CVB3 | − | G/C optimized anti- | PSMB3 | A36 |
| | | | | Rabies IgG(HC + LC) | | |
| CircRNA-09 | 536 | CVB3 | − | G/C optimized | PSMB3 | A36 |
| | | | | HA(NL2009) | | |
| CircRNA-10 | 537 | CVB3 | + | G/C optimized PpLuc | PSMB3 | A36 |
| CircRNA-11 | 538 | SaV IRES | − | G/C optimized PpLuc | PSMB3 | A36 |
| CircRNA-12 | 539 | AIV IRES | − | G/C optimized PpLuc | PSMB3 | A36 |
| CircRNA-13 | 540 | SaV IRES | + | G/C optimized PpLuc | PSMB3 | A36 |
| CircRNA-14 | 541 | AiV IRES | + | G/C optimized PpLuc | PSMB3 | A36 |
| CircRNA-15 | 542 | CVB3 | − | G/C optimized PpLuc | RPS9 | A36 |
| CircRNA-16 | 531 | CVB3 | + | GC optimized | PSMB3 | A36 |
| | | | | mus musculus (Mm) | | |
| | | | | Trp2 (24-472, D260E, | | |
| | | | | W261L) | | |
| CircRNA-17 | 544 | CVB3 | − | GC optimized CTLA4(2- | PSMB3 | A36 |
| | | | | 35); Mm Pmel(14-43); | | |
| | | | | Mm Dct(170-198); Mm | | |
| | | | | Pbk(131-159, (V145D); | | |
| | | | | Mm Trp1(455-473, | | |
| | | | | A463M); Mm | | |
| | | | | Obsl1(1750-1778; | | |
| | | | | T1764M); PADRE; | | |
| | | | | CTLA4(162-223) | | |

### Example 2: Preparation and Purification of Linear Capped mRNA

The present example inter alia provides methods of producing and purifying linear capped mRNA.

2.1. Preparation of DNA Templates for Linear Capped mRNA:

A DNA sequence for the production of linear capped mRNA was prepared to serve as a template for RNA in vitro transcription. DNA sequences were prepared by modifying the wild type or reference encoding DNA sequences by introducing a G/C optimized coding for stabilization and expression optimization. Sequences were introduced into a pUC derived DNA vector to comprise stabilizing 3′-UTR sequences and 5′-UTR sequences, additionally comprising a stretch of adenosines (A100) and an histone stem-loop (HSL) as a 3′ tail.

The obtained plasmid DNA constructs were transformed and propagated in bacteria using common protocols known in the art. Eventually, the plasmid DNA constructs were extracted, purified, and used for subsequent RNA in vitro transcription.

2.2. RNA In Vitro Transcription of Linear Capped mRNA: DNA plasmids prepared according to Example 2.1. were enzymatically linearized using a restriction enzyme and used for DNA dependent RNA in vitro transcription using T7 RNA polymerase in the presence of a sequence optimized nucleotide mixture (ATP/GTP/CTP/UTP) (essentially according to WO2015/188933) and cap analogue (m7G(5′)ppp(5′)(2′OMeA)pG)) under suitable buffer conditions.

The obtained mRNA was purified using RP-HPLC (PureMessenger®; according to WO2008/077592).

The generated mRNA sequences are provided in Table 3.

TABLE 3
Linear capped mRNA constructs used in experiments

| | | SEQ | Description | | | |
|---|---|---|---|---|---|---|
| ID | coding sequence | ID NO: | 5′cap | 5′UTR | 3′UTR | 3′ tail |
| R8732 | G/C optimized GLuc | 206 | yes | HSD17B4 | PSMB3 | HSL + |
| | | | | | | A100 |
| R8730 | G/C optimized PpLuc | 207 | yes | HSD17B4 | PSMB3 | HSL + |
| | | | | | | A100 |

### Example 3: Formulation of Circular RNA and mRNA in Lipid-Based Carriers

The present example inter alia provides methods of producing lipid-based carriers encapsulating circular RNA or lipid-based carriers encapsulating linear capped mRNA.

In short, LNP-formulated RNA (circular RNA or mRNA) was prepared using 59 mol % HEXA-C5DE-PipSS lipid (see compound C2 in Table 1 of WO2021123332) as cationic lipid, 10 mol % DPhyPE as neutral lipid, 29.3 mol % cholesterol as steroid and 1.7 mol % DMG-PEG 2000 as aggregation reducing lipid.

For vaccination experiment (Example 11) circRNA construct was formulated by using 47,4% mol ionizable amino lipid (cationic lipid), 10% mol phospholipid, 40.9 mol % cholesterol and 1,7% mol PEGylated lipid. In short, lipid nanoparticles were prepared and tested according to the general procedures described in PCT Pub. Nos. WO2015199952, WO 2017004143 and WO2017075531, the full disclosures of which are incorporated herein by reference.

The LNPs were prepared using the NanoAssembr™ microfluidic system (Precision NanoSystems Inc., Vancouver, BC) according to standard protocols that enable millisecond mixing of lipid-based carriers at a nanolitre scale. The LNPs comprising circular RNA formed uniform structures with an average size and encapsulation efficiency similar to that of particles containing linear mRNA. Obtained LNPs were re-buffered in a carbohydrate buffer via dialysis, and optionally up-concentrated to a target concentration using ultracentrifugation tubes. The obtained formulation comprising circular RNA and mRNA in lipid-based carriers were used in the in vivo experiments described in the following (see e.g. Examples 5, 6, 8).

Lipid-based carriers (lipid nanoparticles) are also prepared and tested according to general procedures described in PCT Pub. Nos. WO2021123332, WO2015/199952, WO2017/004143 and WO2017/075531, the full disclosures of which are incorporated herein by reference.

### Example 4: Circular RNA Constructs Improved Protein Production in HepG2 or A549 Cells

The present example inter alia shows that circular RNA was more efficiently expressed in cells compared to a corresponding linear capped mRNA. Furthermore, an improved circular RNA constructs showed a higher and longer lasting protein expression compared to a non-improved circular RNA constructs.

Standard circular RNA constructs CircRNA-01 (Table 2; SEQ ID NO: 201) and improved circular RNA constructs CircRNA-02 (SEQ ID NO: 202) were compared with a linear capped GLuc mRNA (Table 3; SEQ ID NO: 206). The circular RNA was produced and purified according to Example 1, and the linear capped mRNA was produced and purified according to Example 2.

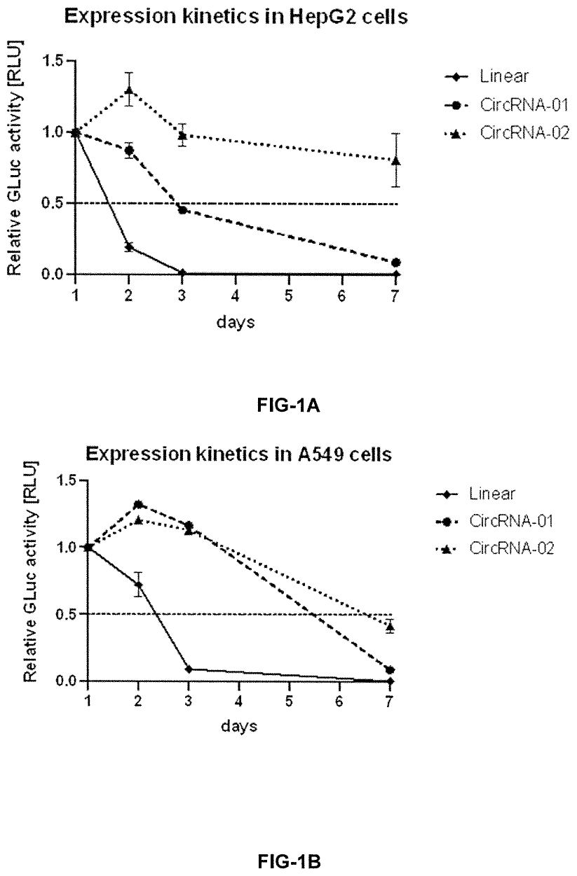

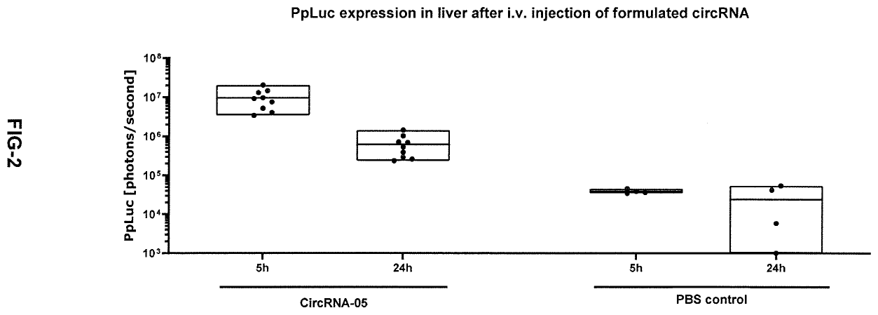

HepG2 or A549 cells were seeded in a 96-well plate at a density of 20,000 cells/well in cell culture medium (RPMI, 10% FCS, 1% L-Glutamine, 1% Pen/Strep), 24h prior to transfection. Cells were transfected with 25ng circular RNA or 25ng linear mRNA using Lipofectamine 2000 (Invitrogen). GLuc luminescence was measured at day 1, 2, 3 and 7 post-transfection using standard procedures. Luminescence data was collected over 7 days. In HepG2 and A549 cells, circular RNA transfection resulted in a prolonged reporter protein expression compared to a linear capped mRNA (see FIG. 1A and FIG. 1B).

Notably, as shown FIG. 1A and FIG. 1B, the improved CircRNA-02 construct (e.g. additionally comprising a Poly(A) stretch and a UTR and codon optimized coding sequence) showed significant higher (2 to 3 times more at initial time point) and longer protein expression in both cell types compared to non-improved CircRNA-01.

### Example 5: Circular RNA can be Effectively Delivered In Vivo Via Lipid-Based Carriers

The present example shows that improved circular RNA can be efficiently delivered using lipid-based carriers.

Improved circular RNA construct CircRNA-05 (Table 2; SEQ ID NO: 205) was produced and purified according to Example 1. The obtained purified circular RNA construct was formulated into lipid-based carriers (LNPs) as described in Example 3.

In BALB/c mice, 10pg LNP formulated circular RNA was delivered by intravenous injection to the liver. As a control composition, PBS was administered. Luminescence from the mice livers was imaged at different time points with a standard in vivo imaging procedure. Injection of LNP formulated CircRNA-05 led to a strong luciferase protein production in the liver (see FIG. 2).

The data clearly shows that the improved circular RNA showed a strong in vivo expression.

### Example 6: Circular RNA Shows Reduced Immunogenicity In Vivo Compared to Linear Capped mRNA

The present example inter alia shows that improved circular RNA shows reduced immunostimulatory properties compared to a corresponding linear capped mRNA.

Improved PpLuc-coding circular RNA construct CircRNA-05 (Table 2; SEQ ID NO: 205) and a corresponding linear capped mRNA (Table 3; SEQ ID NO: 207) were formulated into lipid nanoparticles (LNPs) as described in Example 3. The circular RNA was produced and purified according to Example 1, and the linear capped mRNA was produced and purified according to Example 2.

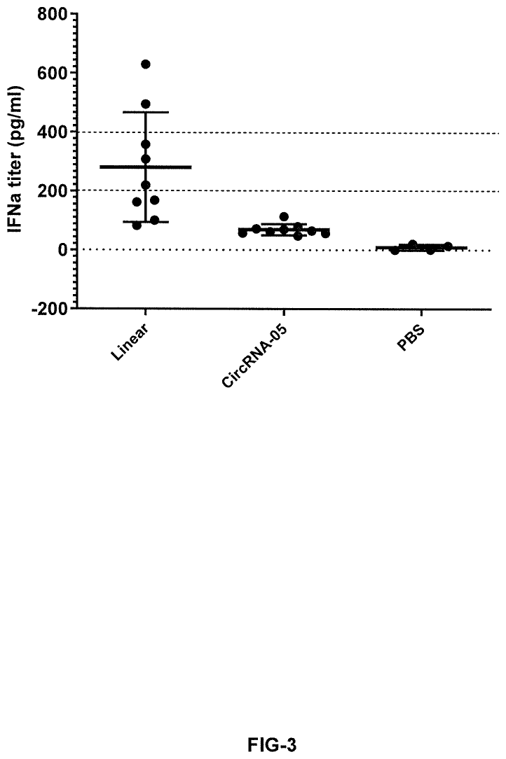

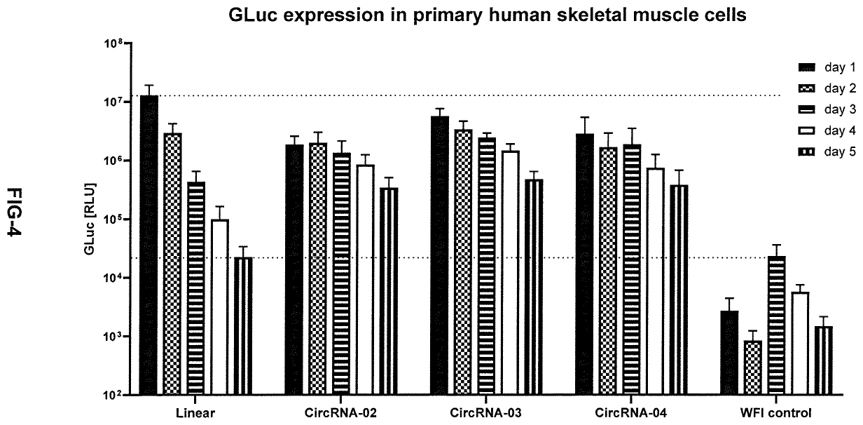

In BALB/c mice, 10pg LNP formulated circular RNA and 10pg LNP formulated linear capped mRNA were delivered by intravenous injection to the liver. As a control composition, PBS was administered. IFNa levels were measured by an IFNa ELISA as commonly known in the art. IFNa was measured from serum sample collected at 5h time point. The data shown in FIG. 3 demonstrates that the improved circular RNA construct CircRNA-05 is characterized by a strongly reduced immunogenicity compared to linear capped mRNA (see FIG. 3).

Notably, a low immunostimulation upon in vivo administration is a particularly important feature of the improved circular RNA and is advantageous in the context of various medical applications.

### Example 7: Circular RNA Prolongs and Increases Protein Expression in Human Muscle Cells

The present example inter alia shows that improved circular RNA constructs showed a stronger and a longer lasting protein expression compared to a corresponding linear capped mRNA in muscle cells.

Improved circular RNA constructs CircRNA-02, CircRNA-03, CircRNA-04 (Table 2; SEQ ID NO: 202-204) were compared with a corresponding linear capped GLuc mRNA (Table 3; SEQ ID NO: 206). The circular RNA was produced and purified according to Example 1, and the linear capped mRNA was produced and purified according to Example 2.

Human skeletal muscle cells were seeded in a 96-well plate at a density of 48,000 cells/well in cell culture medium (low glucose containing DMEM, 2% horse serum) 2 days prior to transfection. Cells were transfected with 50ng circular RNA CircRNA-02, CircRNA-03, CircRNA-04, linear capped mRNA, or water as a control using Lipofectamine 3000 (Invitrogen) as a transfection agent. GLuc reading was performed using standard procedures at day 1, 2, 3, 4 and 5 (see FIG. 4).

As shown in FIG. 4, the improved circular RNA constructs stabilizes GLuc expression (longer lasting protein production) in primary human skeletal muscle cells dependent on the length of the adenosine stretches. Unexpectedly, the insertion of different Poly(A)sequences had an effect on protein expression and/or circular RNA stability.

### Example 8: Circular RNA Stabilizes Protein Expression in Muscle Cells In Vivo

The present example inter alia shows that the improved circular RNA was more efficiently expressed in vivo compared to a corresponding linear capped mRNA.

Improved PpLuc-coding circular RNA construct CircRNA-05 (Table 2; SEQ ID NO: 205) and linear capped mRNA (Table 3; SEQ ID NO: 207) was formulated into a lipid nanoparticles (LNPs) as described in Example 3. The circular RNA was produced and purified according to Example 1, and the linear capped mRNA was produced and purified according to Example 2.

In BALB/c mice, 1pg LNP formulated circular RNA or 1pg LNP formulated linear capped mRNA were delivered by intramuscular injection into each leg muscle. Luminescence data were collected using standard procedures at 18 h and 48h.

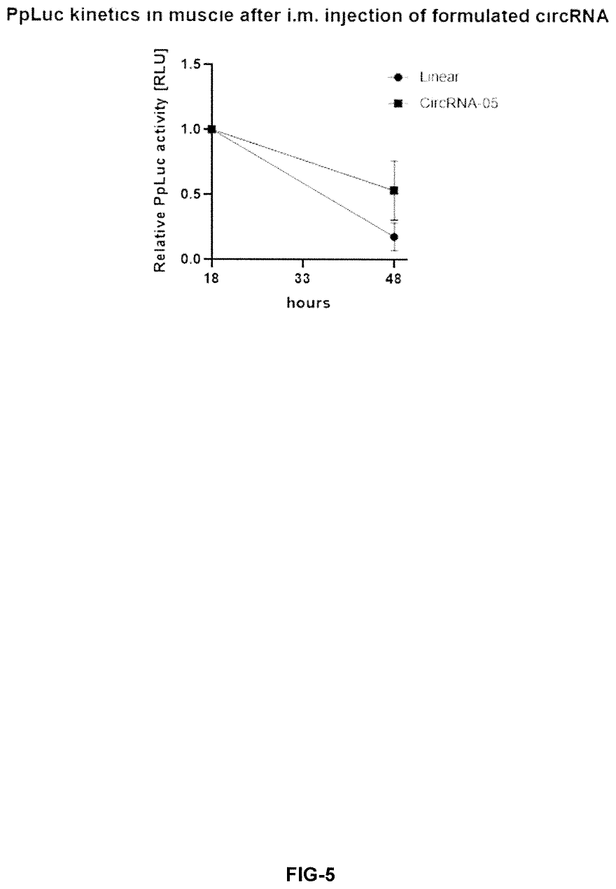

As shown in FIG. 5, injection of the improved circular RNA construct led to a stabilized Luciferase protein production in muscle cells in comparison to a corresponding linear capped mRNA.

### Example 9: Affinity-Based Removal of (Non-Circularized) Linear Precursor RNA

The present example shows a method for the selective depletion of (non-circularized) linear precursor RNA and, optionally, linear intronic splice products from a composition comprising circular RNA.

Intronic sequences or other sequence features that are only present in the linear precursor RNA were exploited to separate non-circularized linear precursor RNA and circular RNA products.

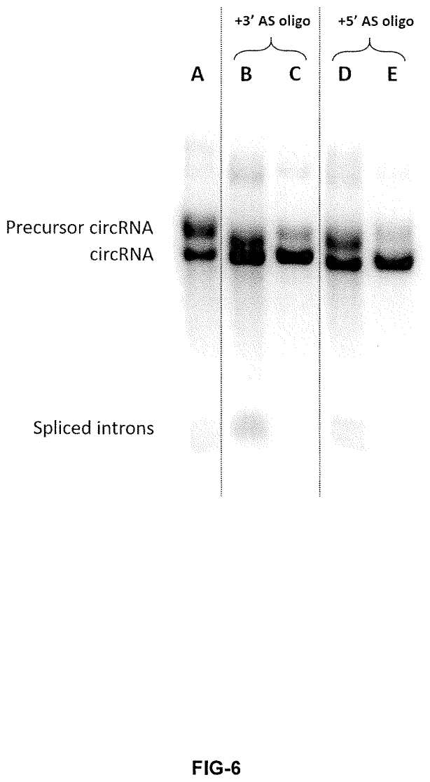

DNA plasmids prepared according to Example 1.1. were enzymatically linearized using a restriction enzyme and used for DNA dependent RNA in vitro transcription using T7 RNA polymerase in the presence of a sequence optimized nucleotide mixture (ATP/GTP/CTP/UTP), essentially preformed according to WO2015188933, under suitable buffer conditions. 50pg CircRNA-01 non-purified in vitro transcription product was incubated either with 5′ end biotinylated 150pmol AS2 LNA oligo (3′ end binding oligo) (SEQ ID NO: 209) or with 5′ end biotinylated 150pmol AS1 LNA oligo (5′ end binding oligo) (SEQ ID NO: 208) in a buffer (50 mM Tris-HCl pH 7.5, 2 mM GTP, 1 mM DTT and 10 mM MgCl2) at 55° C. for 8 min to enhanced in vitro splicing (RNA circularization). After this splicing step, the reaction volume was adjusted to 500 μl with 2×SSC buffer. Next, 50 μl of equilibrated (in 2×SSC) high performance streptavidin sepharose beads were added to the reaction and incubated on a rotating wheel for 1 hr at 30° C. to allow hybridization of antisense oligonucleotides to non-circularized linear precursor RNA and circular RNA products and to allow streptavidin-biotin interaction. After 1 h incubation, bead bound fractions and supernatant fractions were collected by centrifugation. Oligonucleotide bound non-circularized linear precursor RNA and linear intronic splice products remained in the bead fraction, while the desired circular RNA product was strongly enriched in the supernatant fraction (see FIG. 6).

The data shows that an affinity-based removal of (non-circularized) linear precursor RNA as described herein can be used to improve purification of circular RNA. The exemplified procedure can of course be adapted to larger scales, e.g. to liquid chromatography scales using columns or beads comprising immobilized antisense oligonucleotides that specifically bind to (non-circularized) linear precursor RNA species that represent a major impurity of circular RNA preparations.

### Example 10: In Vitro and In Vivo Expression of Circular RNA-Encoded Anti-Rabies Antibodies

In this experiment, the in vitro and in vivo expression and binding to the native antigen of circular RNA-encoded anti-RAV G antibody is evaluated. Three improved circular RNA constructs are used for the experiments. One circular RNA encodes both heavy and light chain of the antibody (CircRNA-08, see Table 2). In other two circular RNA constructs heavy and light chain will be expressed separately (CircRNA-06 and CircRNA-07, see Table 2). The circular RNA is produced and purified according to Example 1 and formulated according to Example 3.

To demonstrate functionality of in vitro-produced circular RNA-encoded antibody, the supernatant obtained after transfection of BHK cells is used for staining of RAV G expressing HeLa cells. To this end, BHK cells are transfected with a specific amount of antibody-encoding circular RNA and HeLa cells are transfected with a specific amount of RAV G-expressing mRNA, respectively, using the Lipofectamine2000 reagent. After staining, HeLa cells are analyzed by flow cytometry (FACS). The expression level of the anti-RAV G antibody is presented as median fluorescence intensity.

To demonstrate in vivo expression of circular RNA encoded anti RAV G antibody, BALB/c mice are injected intravenous (i.v.) with a specific amount of circular RNA in a liposomal formulation (see Example 3). 24h after administration sera are collected and tested by ELISA to determine the expression of human antibodies.

### Example 11: Vaccination of Mice with LNP Formulated circRNA Vaccine

The present example inter alia shows that circular RNA encoding for Hemagglutinin (HA) was able to induce specific humoral and cellular immune responses in vivo after intramuscular injection.

Preparation of LNP Formulated circRNA Vaccine:

CircRNA construct for the HA vaccine (CircRNA-09, see Table 2) was prepared as described in Example 1 (Preparation and purification of circular RNA). Purified circRNA (Example 1.3) was formulated with LNPs according to Example 3 (Formulation of circular RNA and mRNA in lipid-based carriers) prior to use in vivo vaccination experiments.

### Immunization:

Female BALB/c mice (6-8 weeks old) were injected intramuscularly (i.m.) with circRNA vaccine (respective RNA identifiers see Table 2 with 0.5pg, 1pg and 4pg doses. As a negative control, one group of mice was vaccinated with PBS. All animals were vaccinated on day 0 and 21. Blood samples were collected after 18h of first vaccination and on day 21 (post prime) and 35 (post boost) for the determination of antibody titers. After vaccination experiments, the efficiency of the vaccines was antigen specific antibody titer and T cell response were determined.

### Determination of HA Specific Immune Responses

Detection of an HA-specific immune response (B-cell immune response) was carried out by detecting IgG antibodies directed against the active trimeric HA antigen. Therefore, blood samples were taken from the vaccinated mice on day 21 and 35 after vaccination with boost on day 21 and sera was prepared. MaxiSorb plates (Nalgene Nunc International) were coated with the recombinant HA protein (Hemagglutinin HA Trimer (H1N1) (IA-H1-11SWt, E-Enzyme). After blocking with 1×PBS containing 0.05% Tween-20, 0.02% NaN3 and 1% BSA the plates were incubated with diluted mice serum. After washing with 1×PBS containing 0.05% Tween-20, the plates were incubated with peroxidase-conjugated detection antibody total IgG (Anti-mouse total IgG (H+L), 11-035-003, jacksonimmuno) followed by addition of the Amplex UltraRed Reagent (A36006, Invitrogen) and subsequent quantification of the fluorescent product.

### Intracellular Cytokine Staininq:

Splenocytes were seeded into 96-well plates (2×106 cells per well). Cells were stimulated with a mixture of protein specific peptide epitopes (0.5 μg/ml of peptide library) in the presence of 2.5 μg/ml of an anti-CD28 antibody (BD Biosciences) and CD107a-BV421 (Biolegend) for 1 hour at 37° C. and additional 5 hours at 37° C. in the presence of a protein transport inhibitor. After stimulation, cells were washed and stained for viability, washed followed by Fc-block and subsequently stained for surface markers. Fixation and permeabilization were performed using the Cytofix/Cytoperm reagent (BD Biosciences) according to the manufacturer's instructions. Staining for intracellular cytokines followed. The antibodies were used for staining: Thy1.2-af700 (1:200, Biolegend), CD8-PerCpCy5.5 (1:100, Invitrogen), TNF-PE (1:400, Invitrogen), IFNy-APC (1:200, eBioscience), CD4-BV786 (1:400,BD Biosciences), CD107a-BV421 (1:100, Biolegend), CD44-BV605 (1:200, Biolegend), CD62L-PE-Dazzle594 (1:100, Biolegend), Fc-block (1:100, Invitrogen). Aqua Dye was used to distinguish live/dead cells (1:1000, Invitrogen). Cells were acquired using a Fortessa (Beckton Dickinson). Flow cytometry data were analyzed using FlowJo software package (Tree Star, Inc.).

Determination of immunogenicity after circRNA vaccination

IFNa levels were measured by an IFNa ELISA as commonly known in the art. IFNa was measured from serum sample collected at 18h after first vaccination.

### Results of Humoral Immune Response

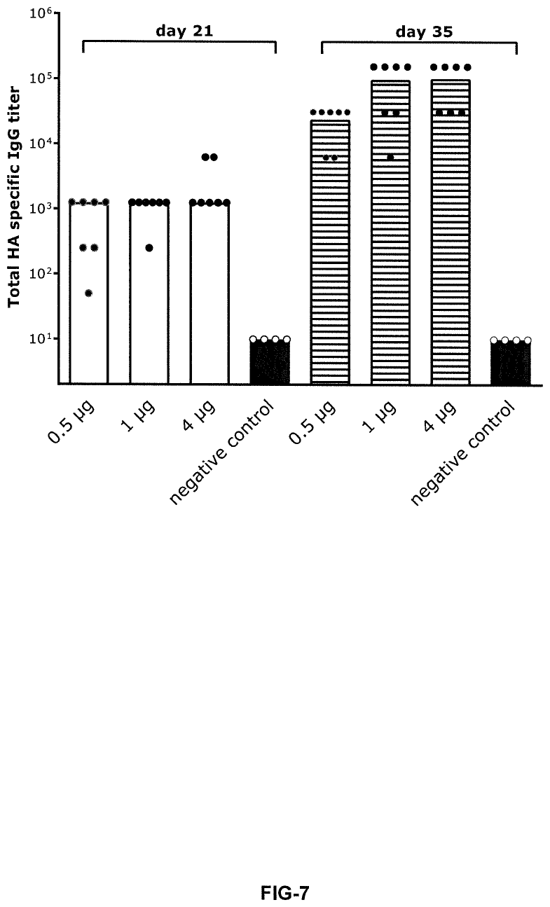

Results of FIG. 7 shows that all mice vaccinated with formulated circRNA constructs developed an HA specific humoral immune response after the first vaccination (day 21). The levels of the specific antibody titers were further increased after boost vaccination (day 35). Even with a very low dose (0.5 μg circRNA) a specific humoral immune response was detected after first vaccination.

### Results of Cellular Immune Response

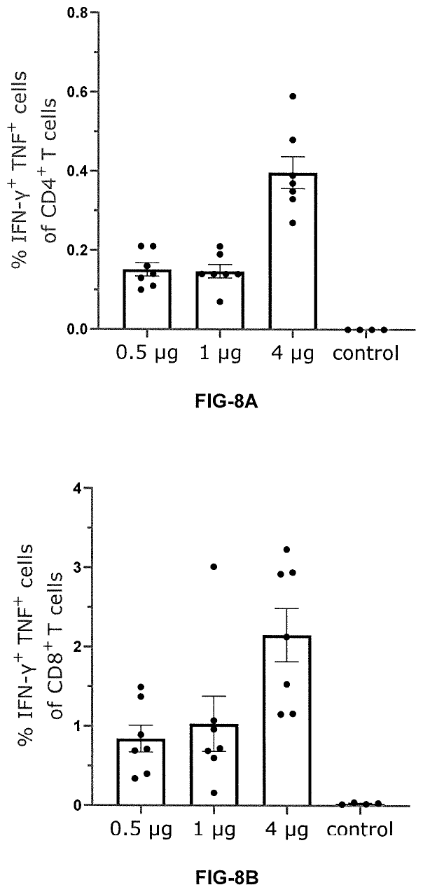

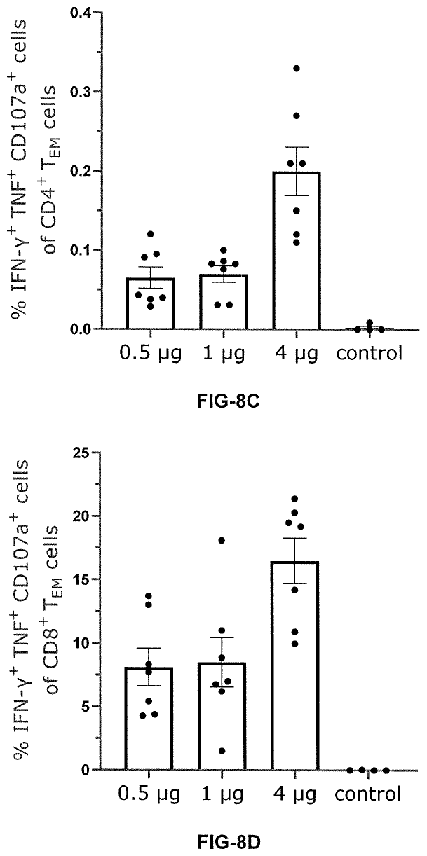

Results of FIG. 8 shows that all mice vaccinated with formulated circRNA constructs developed an antigen specific polyfunctional cellular T cell response. The highest T cell population was detected in the group with the dose of 4 μg injected circular RNA construct. Additionally, FIGS. 8C and 8D show that the vaccination with the circRNA constructs induced a strong effector memory T cell (TEM) subset immune response for both, CD4 and CD8 positive cells.

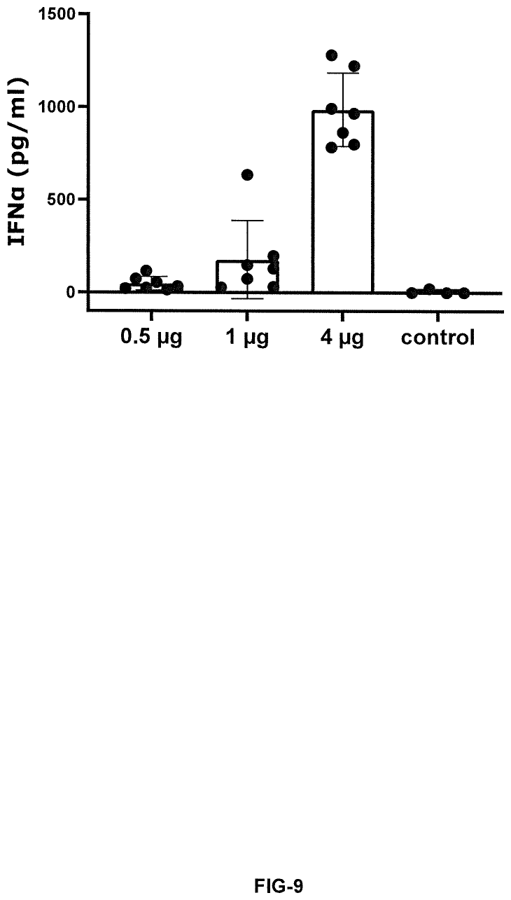

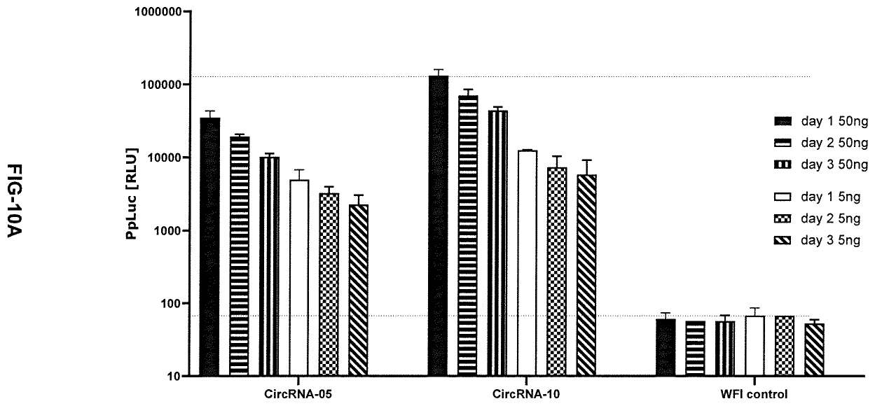

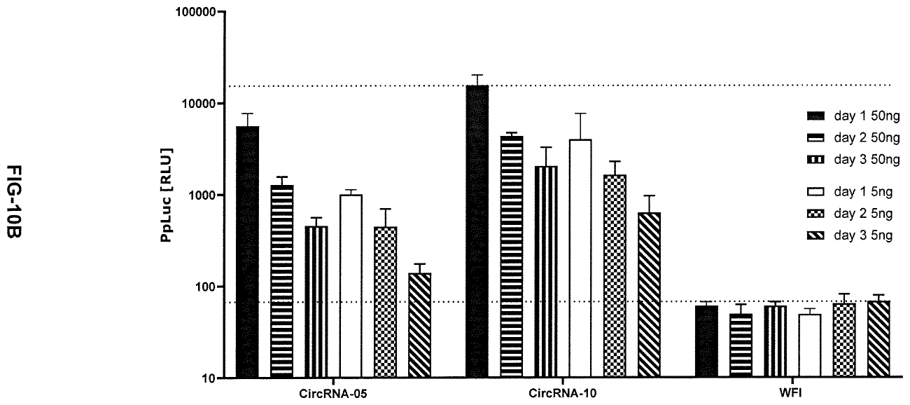

Results of FIG. 9 shows dose dependent INFalpha induction of circular RNA constructs 18 hours after first vaccination. The group with the lowest dose (0.5 μg) circular RNA construct was comparable with the buffer control group.

The data shows that circular RNA constructs can be used as a vaccine to induce high humoral and cellular immune responses with low side effects (e.g. immunogenicity, shown as INFalpha levels) even for very low doses.

### Example 12: In Vitro Expression of Circular RNA with Additional Kozak Sequence in HeLa and HepG2 Cells

The present example inter alia shows that circular RNA construct with additional Kozak sequence was able to induce higher protein expression over time.

Preparation of circRNA Constructs:

Circular RNA construct without additional Kozak sequence (CircRNA-05, see Table 2) and circular RNA construct with additional Kozak sequence (CircRNA-10, see Table 2) were produced and purified according to Example 1.

### Cell Assay

HepG2 or HeLa cells were seeded in a 96-well plate at a density of 20,000 cells/well in cell culture medium (RPMI, 10% FCS, 1% L-Glutamine, 1% Pen/Strep), 24h prior to transfection. Cells were transfected with 5 and 50ng circular RNA using Lipofectamine 2000 (Invitrogen). PLuc luminescence was measured at day 1, 2 and 3 post-transfection using standard procedures.

### Results

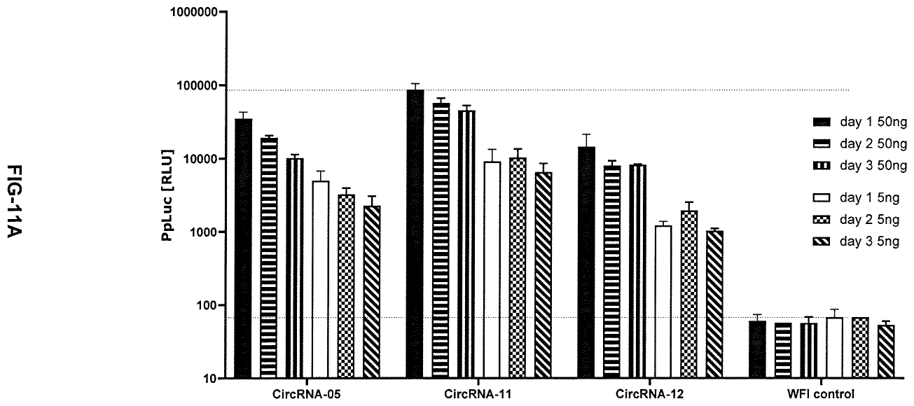

In both cell lines, Kozak improved circular RNA transfection resulted in a higher reporter protein expression over time and independent of transfected circRNA concentration (see FIG. 10A and FIG. 10B).

### Example 13 In Vitro Expression of circRNA Constructs with Alternative IRES in HeLa and HepG2 Cells

The present example inter alia shows that circular RNA constructs with alternative IRES sequences were able to induce high protein expression in two cell lines.

Preparation of circRNA Constructs:

Circular RNA construct with CVB3 (CircRNA-05, see Table 2) and circular RNA constructs with alternative IRES of a Salivirus (CircRNA-11, see Table 2) and of a Aichivirus (CircRNA-12, see table 2) were produced and purified according to Example 1.

### Cell Assay

HepG2 or HeLa cells were seeded in a 96-well plate at a density of 20,000 cells/well in cell culture medium (RPMI, 10% FCS, 1% L-Glutamine, 1% Pen/Strep), 24h prior to transfection. Cells were transfected with 5 and 50ng circular RNA using Lipofectamine 2000 (Invitrogen). PLuc luminescence was measured at day 1, 2 and 3 post-transfection using standard procedures.

### Results

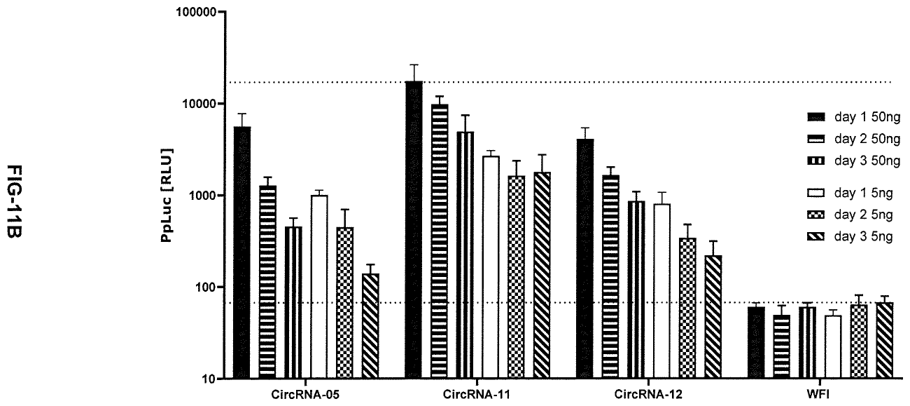

In both cell lines, transfection of circular RNA constructs with alternative IRES resulted in a comparable (CircRNA-12) or higher (CircRNA-11) reporter protein expression (FIG. 11A and FIG. 11B). These data show that the improved circular RNA construct of the invention is functional with alternative IRES sequences and could be improved further.

### Example 14 In Vitro Expression of circRNA Constructs with Alternative IRES and Additional Kozak Sequences in HeLa and HepG2 Cells

The goal of the present example is inter alia to show that an additional Kozak sequence improves the performance of alternative IRES in circular RNA constructs.

Preparation of circRNA Constructs:

Circular RNA constructs (CircRNA-11 and CircRNA-12, see Table 2) and circular RNA constructs with an additional Kozak sequence ((CircRNA-13 and CircRNA-14, see Table 2) are produced and purified according to Example 1.

### Cell Assay

HepG2 or HeLa cells are seeded in a 96-well plate at a density of 20,000 cells/well in cell culture medium (RPMI, 10% FCS, 1% L-Glutamine, 1% Pen/Strep), 24h prior to transfection. Cells are transfected with 5 and 50ng circular RNA using Lipofectamine 2000 (Invitrogen). PLuc luminescence are measured at day 1, 2 and 3 post-transfection using standard procedures.

### Example 15: In Vitro Expression of Circular RNA Construct with Alternative 3′UTR (RSP9) in HeLa and HepG2 Cells

The goal of the present example is inter alia to show that an alternative 3′UTR (RSP9) in circular RNA constructs showed comparable protein expression compared to the circular RNA construct with a PSMB3 3′UTR.

Preparation of circRNA Constructs:

Circular RNA construct with alternative RSP9 3′UTR (CircRNA-15, see Table 2) and circular RNA construct with PSMB3 3′UTR (CircRNA-05, see Table 2) are produced and purified according to Example 1.

### Cell Assay

HepG2 or HeLa cells are seeded in a 96-well plate at a density of 20,000 cells/well in cell culture medium (RPMI, 10% FCS, 1% L-Glutamine, 1% Pen/Strep), 24h prior to transfection. Cells are transfected with 5 and 50ng circular RNA using Lipofectamine 2000 (Invitrogen). PLuc luminescence are measured at day 1, 2 and 3 post-transfection using standard procedures.

### Example 16: Vaccination of Mice with LNP Formulated circRNA Tumor Vaccine

The goal of the the present example is inter alia to shows that circular RNA constructs encoding for tumor antigens and epitopes (mutated and unmutated) are able to induce specific cellular immune responses in vivo after intramuscular injection. To induce significant anti-tumor responses the antigen and the epitopes are designed with the CTLA4 targeting approach as described in WO2019/008001, the full disclosures of which are incorporated herein by reference.

Preparation of circRNA Constructs:

Circular RNA construct encoding a single tumor antigen (CircRNA-16, see Table 2) and circular RNA construct encoding multiple epitopes (CircRNA-17, see Table 2) are produced and purified according to Example 1. Spin column purified circRNA (Example 1.3) is formulated with LNPs according to Example 3 (Formulation of circular RNA and mRNA in lipid-based carriers) prior to use in vivo vaccination experiments.

### Determination of Tumor Specific Immune Responses

C57BL/6 mice are injected intramuscular (i.m.) at 2 sites with circular RNA constructs encoding a tumor antigen (Trp2, see Table 2) or a multiepitope variant (see Table 2). On days 0, 7, 14, 21 and 28 of the experiment mice are injected i.m. with circular RNA constructs solved in Ringer Lactate buffer. The total volume for i.m. vaccination is 20 μl and is distributed to 2 sites of injection. 7 days after the last vaccination an ICS is performed to evaluate epitope-specific CD8 positive and CD4 positive T cell responses.

### Example 17: CircRNA Constructs Express in Adipocytes

The goal of the present example is inter alia to show that circular RNA constructs are able to express the encoded protein in adipocytes. Adipose tissue and its biology is becoming increasingly important as obesity and its related comorbidities, including type 2 diabetes, cardiovascular disease, certain cancers and possible restricted success in vaccination, are threatening the health of a growing number of people worldwide.

Previous studies have shown that genes involved in de novo lipogenesis use IRES elements in their 5′ UTR to initiate translation. Therefore, the circular RNA of the invention is suitable for protein expression in adipocytes.

Preparation of circRNA Constructs:

Circular RNA constructs (CircRNA-10, se Table 2) are produced and purified according to Example 1 and the linear RNA as a control group according to Example 2. Purified circRNA (Example 1.3) is used unformulated or formulated with LNPs according to Example 3 (Formulation of circular RNA and mRNA in lipid-based carriers) prior to use in vitro experiments.

### Murine Adipocyte Cell Assay

Murine 3T3-L1 adipocytes are a well-characterized cell culture model that is widely used to study the role of adipocyte biology in obesity and type 2 diabetes. These properties make 3T3-L1 adipocytes an attractive model for in vitro assays.

Murine 3T3-L1 preadipocytes are cultured and differentiated as described in the literature/common knowledge. The 3T3-L1 adipocytes are used for transfection at day 4-6 post-induction when lipid droplets were readily apparent. The differentiated adipocytes are washed and plated at collagen-coated 48 well plates (5.4×104 cells/cm2 or 1.16×105 cells/cm2).

Adipocyte cells are transfected with a specific amount of unformulated circular RNA using the Lipofectamine2000 reagent or formulated circular RNA, as described in Example 3. PLuc luminescence are measured at day 1, 2 and 3 post-transfection using standard procedures.

### Human Adipocyte Cell Assay

Primary human subcutaneous adipocytes (e.g. SGBS preadipocytes) are obtained at day 7 post induction and are maintained in DMEM/F-12 with 3% FBS, 33 μM biotin, 17 μM pantothenate, 1 μM bovine insulin, 1 μM dexamethasone, and 100 U penicillin/100 μg streptomycin/0.25 μg Fungizone. At day 9 post-induction, the adipocytes are plated on collagen-coated 48 well plates at 4.1×104 cells/cm2 and transfected with a specific amount of unformulated circular and linear RNA using the Lipofectamine2000 reagent or formulated circular and linear RNA, as described in Example 3. PLuc luminescence are measured at day 1, 2 and 3 post-transfection using standard procedures.

1. A circular RNA comprising the following sequence elements in the following order
(i) at least one translation initiation sequence;
(ii) at least one coding sequence (cds);
(iii) at least one UTR sequence; and
(iv) at least one poly(A) sequence.

2. The circular RNA of claim 1, wherein the at least one poly(A) sequence comprises at least about 30 consecutive adenosine nucleotides, at least about 40 consecutive adenosine nucleotides, at least about 50 consecutive adenosine nucleotides, at least about 60 consecutive adenosine nucleotides, at least about 70 consecutive adenosine nucleotides, at least about 80 consecutive adenosine nucleotides, at least about 90 consecutive adenosine nucleotides, or at least about 100 consecutive adenosine nucleotides.

3. The circular RNA of claim 1 or 2, wherein the at least one poly(A) sequence comprises about 40 to about 150 consecutive adenosine nucleotides.

4. The circular RNA of claims 1 to 3, wherein the circular RNA comprises at least two, three, or more poly(A) sequences.

5. The circular RNA of claims 1 to 4, wherein the at least one poly(A) sequence is located downstream of the coding sequence in the direction of translation.

6. The circular RNA of any one of the preceding claims, wherein the at least one UTR comprises or consists of a nucleic acid sequence derived from a 3′-UTR of a gene selected from PSMB3, ALB7, alpha-globin, CASP1, COX6B1, GNAS, NDUFA1, AES-12S and RPS9, or from a homolog, a fragment or a variant of any one of these genes, or wherein the at least one UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B, HBA1, HBA2 and UBQLN2, or from a homolog, a fragment or a variant of any one of these genes.

7. The circular RNA of any one of the preceding claims, wherein the at least one UTR has a length of less than about 200 nucleotides or a length between 50 and 200 nucleotides.

8. The circular RNA of any one of the preceding claims, wherein the at least one UTR comprises or consists of an RNA sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 192, or a fragment or a variant thereof.

9. The circular RNA of any one of the preceding claims, wherein the at least one UTR is a downstream UTR located between the coding sequence and the at least one Poly(A) sequence.

10. The circular RNA of any one of the preceding claims, additionally comprising at least one upstream UTR sequence, preferably derived from a 5′-UTR of a gene selected from HSD17B4, RPL32, ASAH1, ATP5A1, MP68, NDUFA4, NOSIP, RPL31, SLC7A3, TUBB4B, HBA1, HBA2 and UBQLN2.

11. The circular RNA of claim 10, wherein the at least one upstream UTR sequence is located between the translation initiation sequence and the coding sequence.

12. The circular RNA of any one of the preceding claims, wherein the at least one coding sequence is a codon modified coding sequence, wherein the amino acid sequence encoded by the at least one codon modified coding sequence is preferably not being modified compared to the amino acid sequence encoded by the corresponding wild type or reference coding sequence.

13. The circular RNA of claim 12, wherein the at least one codon modified coding sequence is selected from a C maximized coding sequence, a CAI maximized coding sequence, a human codon usage adapted coding sequence, a G/C content modified coding sequence, and a G/C optimized coding sequence, or any combination thereof.

14. The circular RNA of claim 12 or 13, wherein the at least one codon modified coding sequence is a G/C optimized coding sequence, a human codon usage adapted coding sequence, or a G/C content modified coding sequence, preferably a G/C optimized coding sequence.

15. The circular RNA of any one of the preceding claims, wherein the at least one coding sequence has a G/C content of at least about 55%, 60%, or 65%.

16. The circular RNA of any one of the preceding claims, wherein coding sequence encodes at least one peptide or protein suitable for use in treatment or prevention of a disease, disorder or condition.

17. The circular RNA of claim 16, wherein the at least one peptide or protein is selected or derived from an antibody, an intrabody, a receptor, a receptor agonist, a receptor antagonist, a binding protein, a CRISPR-associated endonuclease, a chimeric antigen receptor (CAR), a chaperone, a transporter protein, an ion channel, a membrane protein, a secreted protein, a transcription factor, a toxin, an enzyme, a peptide or protein hormone, a growth factor, a structural protein, a cytoplasmic protein, a cytoskeletal protein, an allergen, a tumor antigen, a proto-oncogene, an oncogene, a tumor suppressor gene, a neoantigen, a mutated antigen, an antigen of a pathogen, or fragments, epitopes, variants, or combinations of any of these.

18. The circular RNA of any one of the preceding claims, wherein the at least one translation initiation sequence is a cap-independent translation initiation element.

19. The circular RNA of any one of the preceding claims, wherein the at least one translation initiation sequence is selected from an internal ribosomal entry site (IRES), an aptamer, and a CITE element.

20. The circular RNA of claim 19, wherein the IRES is selected or derived from a viral IRES, a cellular IRES, or a synthetic IRES.

21. The circular RNA of claim 19 or 20, wherein IRES sequence comprises or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 393-495, 514, or a fragment or a variant of any of these.

22. The circular RNA of claim 19 to 21, wherein the IRES is selected or derived from a coxsackievirus B3 (CVB3) IRES or a salivirus (SaV) IRES.

23. The circular RNA of claim 19, wherein the aptamer sequence comprises or consist of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 496-503, or a fragment or a variant of any of these.

24. The circular RNA of any one of the preceding claims, wherein the translation initiation sequence, preferably the IRES, is located upstream of the start codon of the at least one coding sequence.

25. The circular RNA of any one of the preceding claims, wherein the RNA sequence located upstream of the translation initiation sequence (in opposing direction of translation) is a unstructured sequence element, e.g. an AC rich sequence.

26. The circular RNA of claim 25, wherein unstructured sequence element comprises or consists of a sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 186, or a fragment or a variant of any of these.

27. The circular RNA of any one of the preceding claims, wherein the circular RNA additionally comprises at least one Kozak sequence, preferably selected or derived from any one of SEQ ID NOs:169-176 (GCCGCCACCAUGG, GCCGCCACC, GCCACC, ACC), or a fragment or a variant of any of these, more preferably selected or derived from GCCACC, or a fragment or a variant.

28. The circular RNA of any one of the preceding claims, wherein the at least one Kozak sequence is located directly upstream of the start codon of the at least one coding sequence.

29. The circular RNA of any one of the preceding claims, wherein the at least one Kozak sequence is located downstream of the at least one IRES sequence.

30. The circular RNA of any one of the preceding claims, wherein the circular RNA comprises an IRES sequence, as defined in claim 20 to 22 and 24, and a Kozak sequence, as defined in claim 27 to 29, preferably selected or derived from any one of SEQ ID NOs: 518-520.

31. The circular RNA of any one of the preceding claims, additionally comprising at least one poly(C) sequence and/or at least one histone-stem loop sequence and/or at least one miRNA binding site.

32. The circular RNA of claim 31, wherein the at least one first miRNA binding site sequence comprises one or more of the group consisting of binding sites for miRNA-122, miRNA-142, miRNA-148a, miRNA-101, miRNA-192, miRNA-194, and miRNA-223.

33. The circular RNA of claims 1 to 32, wherein the circular RNA does not comprise modified nucleotides.

34. The circular RNA of claims 1 to 32, wherein the circular RNA comprises modified nucleotides.

35. The circular RNA of claim 34, wherein at least one modified nucleotide is selected from pseudouridine (ψ), N1-methylpseudouridine (m1ψ), 5-methylcytosine, and/or 5-methoxyuridine.

36. The circular RNA of claim 34, wherein at least one modified nucleotide is selected from Alpha-thio-ATP, Alpha-thio-GTP, Alpha-thio-CTP, Alpha-thio-UTP, N4-acetyl-CTP, N6-methyladenosine, 2′O-methyl-ATP, 2′O-methyl-GTP, 2′O-methyl-CTP, or 2′O-methyl-UTP.

37. The circular RNA of any one of the preceding claims, wherein the circular RNA consists of ribonucleotides linked via phosphodiester-bonds.

38. The circular RNA of any one of the preceding claims, wherein the circular RNA comprises at least one splice-junction element.

39. The circular RNA of claim 38, wherein the splice-junction element is located between the at least one Poly(A) sequence and the at least one translation initiation sequence.

40. The circular RNA of any one of the preceding claims, wherein the circular RNA has a length of at least 500 ribonucleotides, preferably wherein the circular RNA has a length ranging from about 500 ribonucleotides to about 5000 ribonucleotides.

41. The circular RNA of any one of the preceding claims, wherein the circular RNA has been circularized via self-splicing from a linear single stranded precursor RNA.

42. The circular RNA of any one of the preceding claims, wherein the circular RNA has an RNA integrity of at least about 50%, preferably of at least about 60%, more preferably of at least about 70%, most preferably of at least about 80%.

43. The circular RNA of any one of the preceding claims, wherein upon administration of the circular RNA to a cell or subject, the circular RNA has reduced immunostimulatory properties compared to a corresponding reference RNA.

44. The circular RNA of any one of the preceding claims, wherein upon administration of the circular RNA to a cell or subject, the circular RNA has a prolonged protein expression compared to a corresponding reference RNA.

45. The circular RNA of any one of the preceding claims, wherein upon administration of the circular RNA to a cell or subject, the circular RNA has a prolonged protein expression in cells that are characterized by a reduced/lowered eIF4F expression compared to a corresponding reference RNA.

46. The circular RNA of claim 44 and 45, wherein the prolonged protein expression is an additional duration of protein expression in said cell, tissue, or organism is at least 5 h, 10h, 20 h, 25h, 30h, 35h, 40h, 45h, 50h, 55h, 60h, 65h, 70h, 75h, 80h, 85h, 90h, 95h, or 100h or even longer.

47. The circular RNA of claims 1 to 46, comprising the following sequence elements in the following order:
at least one unstructured sequence element as defined in claim 25 or 26;
at least one translation initiation sequence defined in claims 18 to 24;
at least one coding sequence (cds) as defined in claims 12 to 17;
at least one UTR sequence as defined in claim 6 to 8;
at least one poly(A) sequence as defined in claims 2 to 5, preferably comprising about 40 to about 150 consecutive adenosine nucleotides.

48. The circular RNA of claims 1 to 47, wherein the circular RNA comprises or consists of an RNA sequence identical or at least 70%, 80%, 90%, 91%, 92%, 93%, 94%, 95%, 96%, 97%, 98%, or 99% identical to any one of SEQ ID NOs: 202-205, 533-536, 538, 539, 542 or a fragment or a variant of any of these, wherein the coding sequence in any one of SEQ ID NOs: 202-205, 533-536, 538, 539, 542 is exchanged by at least one coding sequence encoding a therapeutic peptide or protein, preferably by a coding sequence as defined in claims 12 to 17.

49. The circular RNA of claims 1 to 46, comprising the following sequence elements in the following order:
at least one unstructured sequence element as defined in claim 25 or 26;
at least one translation initiation sequence defined in claims 18 to 24;
at least one Kozak sequence as defined in claim 27 to 29;
at least one coding sequence (cds) as defined in claims 12 to 17;
at least one UTR sequence as defined in claim 6 to 8;
at least one poly(A) sequence as defined in claims 2 to 5, preferably comprising about 30 to about 150 consecutive adenosine nucleotides.

50. The circular RNA of claims 1 to 46 and 49, wherein the circular RNA comprises or consists of an RNA sequence identical or at least 70%, 80%, 90%, 91%, 92%, 93%, 94%, 95%, 96%, 97%, 98%, or 99% identical to any one of SEQ ID NOs: 537, 540, 541, 543, 544 or a fragment or a variant of any of these, wherein the coding sequence in any one of SEQ ID NOs: 537, 540, 541, 543, 544 is exchanged by at least one coding sequence encoding a therapeutic peptide or protein, preferably by a coding sequence as defined in claims 12 to 17.

51. The circular RNA of claims 1 to 46, comprising the following sequence elements in the following order:
optionally, at least one unstructured sequence element as defined in claim 25 or 26;
at least one aptamer for translation initiation defined in claims 18 to 24;
optionally, at least one upstream UTR as defined in claim 10 or 11;
at least one Kozak sequence as defined in claim 27 to 29;
at least one coding sequence (cds) as defined in claims 12 to 17;
at least one UTR sequence as defined in claim 6 to 8;
at least one poly(A) sequence as defined in claims 2 to 5, preferably comprising about 40 to about 150 consecutive adenosine nucleotides.

52. A linear precursor RNA for making a circular RNA of claims 1 to 51, said linear precursor RNA comprising the following elements preferably operably connected to each other and arranged in the following sequence:
a 3′ permuted intron-exon element;
at least one translation initiation sequence;
at least one coding sequence;
at least one UTR sequence;
at least one poly(A) sequence;
a 5′ permuted intron-exon element.

53. A linear precursor RNA for making a circular RNA of claims 1 to 51, said linear precursor RNA comprising the following elements preferably operably connected to each other and arranged in the following sequence:
a 3′ permuted intron-exon element;
at least one translation initiation sequence;
at least one Kozak sequence;
at least one coding sequence;
at least one UTR sequence;
at least one poly(A) sequence;
a 5′ permuted intron-exon element.

54. The linear precursor RNA of claim 52 and 53, wherein the 3′ permuted intron-exon element comprises a 5′ homology arm, a 3′ Group I intron fragment containing a 3′ splice site dinucleotide, and a 5′ spacer sequence, and wherein the 5′ permuted intron-exon element comprises a 3′ spacer sequence, a 5′ Group I intron fragment containing a 5′ splice site dinucleotide, and a 3′ homology arm.

55. The linear precursor RNA of claims 52 to 54, wherein the at least one poly(A) sequence is characterized by any of the features as defined in claims 2 to 5.

56. The linear precursor RNA of claims 52 to 55, wherein the at least one translation initiation sequence is characterized by any of the features as defined in claims 18 to 24.

57. The linear precursor RNA of claims 52 to 56, wherein the at least one coding sequence is characterized by any of the features as defined in claims 12 to 17.

58. The linear precursor RNA of claims 52 to 57, wherein the at least one UTR sequence is characterized by any of the features as defined in claims 6 to 8.

59. The linear precursor RNA of claims 52 to 58, comprising at least one upstream UTR sequence, wherein the at least one upstream UTR sequence is characterized by any of the features as defined in claim 10 or 11.

60. The linear precursor RNA of claims 52 to 59, comprising at least one purification tag.

61. The linear precursor RNA of claim 60, wherein the at least one purification tag is located at the 3′ and/or the 5′ terminus of the linear precursor RNA.

62. The linear precursor RNA of claim 60 or 61, comprising at least one purification tag is an RNA sequence tag element.

63. The linear precursor RNA of claims 52 to 62, wherein the linear precursor RNA is configured for circularization via self-splicing.

64. The linear precursor RNA of claims 52 to 63, wherein the linear precursor RNA comprises or consists of an RNA sequence identical or at least 70%, 80%, 90%, 91%, 92%, 93%, 94%, 95%, 96%, 97%, 98%, or 99% identical to any one of SEQ ID NOs: 197-200, 512-532 or a fragment or a variant of any of these, wherein the coding sequence in any one of SEQ ID NOs: 197-200, 512-532 is exchanged by at least one coding sequence encoding a therapeutic peptide or protein, preferably by a coding sequence as defined in claims 12 to 17.

65. A pharmaceutical composition comprising at least one circular RNA as defined in any of the claims 1 to 51, wherein the composition optionally comprises at least one pharmaceutically acceptable carrier.

66. The pharmaceutical composition of claim 65, wherein the composition comprises a plurality or at least more than one circular RNA species, preferably wherein each circular RNA species encodes a different peptide or protein.

67. The pharmaceutical composition of claim 65 or 66, wherein the circular RNA is formulated in at least one cationic or polycationic compound.

68. The pharmaceutical composition of claim 67, wherein the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

69. The pharmaceutical composition of claims 65 to 68, wherein the circular RNA is formulated in lipid-based carriers.

70. The pharmaceutical composition of claim 69, wherein the lipid-based carriers are selected from liposomes, lipid nanoparticles, lipoplexes, and/or nanoliposomes.

71. The pharmaceutical composition of claim 69 or 70, wherein the lipid-based carriers are lipid nanoparticles, preferably wherein the lipid nanoparticles encapsulate the circular RNA.

72. The pharmaceutical composition of claims 69 to 71, wherein the lipid-based carriers comprise an aggregation-reducing lipid, a cationic lipid, a neutral lipid, and a steroid or steroid analog.

73. The pharmaceutical composition of claims 69 to 71, wherein the lipid-based carriers comprise an aggregation reducing lipid selected from a polymer conjugated lipid.

74. The pharmaceutical composition of claim 73, wherein the polymer conjugated lipid is a PEG-conjugated lipid preferably selected or derived from DMG-PEG 2000, C10-PEG2K, Cer8-PEG2K, or ALC-0159, more preferably wherein the polymer conjugated lipid is a POZ-lipid, which is defined as a compound according to formula (POZ):
[H]—[linker]—[M] formula (POZ)
wherein
[H] is a homopolymer moiety comprising at least one polyoxazoline (POZ) monomer unit 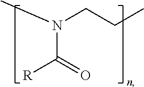
wherein R is C₁₋₉ alkyl or C₂₋₉ alkenyl and n has a mean value ranging from 2 to 200, preferably from 20 to 100, more preferably from 24 to 26 or 45 to 50
[linker] is an optional linker group, and
[M] is a lipid moiety.

75. The pharmaceutical composition of claim 73, wherein the polymer conjugated lipid is not a PEG-conjugated lipid.

76. The pharmaceutical composition of claims 69 to 75, wherein the lipid-based carriers comprise a cationic lipid selected or derived from ALC-0315, SM-102, SS-33/4PE-15, HEXA-C5DE-PipSS or compound C26

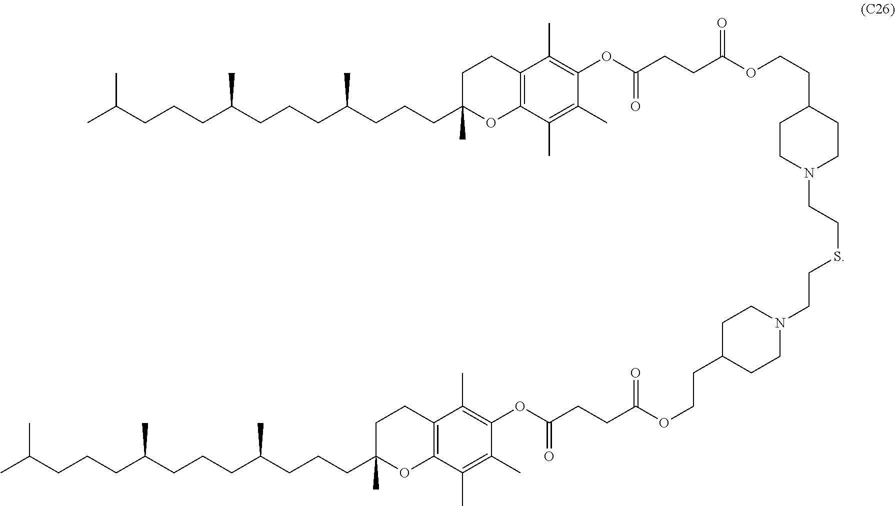 [SMILES: Cc1c(C)c2c(c(C)c1OC(=O)CCC(=O)OCCC1CCN(CCSCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2]

77. The pharmaceutical composition of claims 69 to 76, wherein the lipid-based carriers comprise a neutral lipid selected or derived from DSPC, DHPC, or DphyPE.

78. The pharmaceutical composition of claims 69 to 77, wherein the lipid-based carriers comprise a steroid or steroid analog selected or derived from cholesterol, cholesteryl hemisuccinate (CHEMS), preferably cholesterol.

79. The pharmaceutical composition of claims 69 to 79, wherein the lipid-based carriers comprise
(i) at least one cationic lipid, preferably as defined in claim 76;
(ii) at least one neutral lipid, preferably as defined in claim 77;
(iii) at least one steroid or steroid analogue, preferably as defined in claim 78; and
(iv) at least one aggregation reducing lipid, preferably as defined in claims 73 to 75.

80. The pharmaceutical composition of claims 69 to 79, wherein the lipid-based carriers comprise about 20-60% cationic lipid, about 5-25% neutral lipid, about 25-55% steroid or steroid analogue, and about 0.5-15% aggregation reducing lipid, preferably (i) about 47.4% cationic lipid, about 10% neutral lipid, about 40.9% steroid, and about 1.7% aggregation reducing lipid or (ii) about 59 mol % cationic lipid, 10 mol % neutral lipid, 28.5 mol % steroid and about 2.5 mol % aggregation reducing lipid.

81. The pharmaceutical composition of claims 69 to 80, wherein the wt/wt ratio of lipid to circular RNA in the lipid-based carrier is from about 10:1 to about 60:1, preferably from about 20:1 to about 30:1.

82. The pharmaceutical composition of claims 69 to 81, wherein the N/P ratio of the lipid-based carriers encapsulating the circular RNA is in a range from about 1 to about 10, preferably in a range from about 5 to about 7.

83. The pharmaceutical composition of claims 69 to 82, wherein the lipid-based carriers have a Z-average size in a range of about 50 nm to about 120 nm.

84. The pharmaceutical composition of claims 64 to 83, wherein the pharmaceutical composition comprises at least one linear 5′ capped messenger RNA comprising at least one coding sequence encoding a peptide or protein.

85. The pharmaceutical composition of claim 84, wherein the at least one linear 5′ capped messenger RNA comprises at least one 5′ UTR and/or at least one 3′ UTR.

86. The pharmaceutical composition of claim 84 or 85, wherein the at least one linear 5′ capped messenger RNA comprises at least one poly(A) sequence and, optionally, at least one histone stem loop and/or at least one poly(C) sequence.

87. The pharmaceutical composition of claims 84 to 86, wherein the at least one linear 5′ capped messenger RNA comprises a cap1 structure.

88. The pharmaceutical composition of claims 84 to 87, wherein the at least one coding sequence of the linear 5′ capped messenger RNA is a codon modified coding sequence selected from a C maximized coding sequence, a CAI maximized coding sequence, a human codon usage adapted coding sequence, a G/C content modified coding sequence, and a G/C optimized coding sequence, or any combination thereof.

89. The pharmaceutical composition of claims 84 to 88, wherein the at least one linear 5′ capped messenger RNA comprises modified nucleotides selected from pseudouridine (ψ), N1-methylpseudouridine (m1ψ), 5-methylcytosine, and/or 5-methoxyuridine.

90. The pharmaceutical composition of claims 84 to 89, wherein at least one coding sequence of the linear 5′ capped messenger RNA encodes at least one peptide or protein suitable for use in treatment or prevention of a disease, disorder or condition.

91. The pharmaceutical composition of claims 84 to 90, wherein the at least one peptide or protein encoded by the linear 5′ capped messenger RNA is selected or derived from an antibody, an intrabody, a receptor, a receptor agonist, a chimeric antigen receptor (CAR), a receptor antagonist, a binding protein, a CRISPR-associated endonuclease, a chaperone, a transporter protein, a toxin, an ion channel, a membrane protein, a secreted protein, a transcription factor, an enzyme, a peptide or protein hormone, a growth factor, a structural protein, a cytoplasmic protein, a cytoskeletal protein, an allergen, a tumor antigen, a proto-oncogene, an oncogene, a tumor suppressor gene, a neoantigen, a mutated antigen, an antigen of a pathogen, or fragments, epitopes, variants, or combinations of any of these.

92. The pharmaceutical composition of claims 84 to 91, wherein the at least one peptide or protein encoded by the linear 5′ capped messenger RNA is selected or derived from an antigen of a pathogen.

93. The pharmaceutical composition of claims 84 to 92, wherein the at least one peptide or protein encoded by the linear 5′ capped messenger RNA is selected or derived from an antigen of a tumor.

94. The pharmaceutical composition of claims 84 to 93, wherein the linear 5′ capped messenger RNA is separately formulated or co-formulated with the at least one circular RNA.

95. The pharmaceutical composition of claims 65 to 94, wherein the pharmaceutical composition is a vaccine.

96. The pharmaceutical composition of claims 65 to 95, wherein the pharmaceutical composition is a protein replacement therapy.

97. A combination comprising the following components
A) at least one circular RNA comprising at least one coding sequence; and
B) at least one linear coding RNA.

98. The combination of claim 97, wherein the at least one circular RNA is further characterized by any one of the features of claims 1 to 51.

99. The combination of claim 97 or 98, wherein the at least one linear coding RNA is a linear 5′ capped messenger RNA.

100. The combination of claim 99, wherein the linear 5′ capped messenger RNA is further characterized by any one of the features of claims 84 to 94.

101. The combination of claims 97 to 100, wherein component A and/or component B are separately formulated, preferably separately formulated in lipid-based carriers as defined in any one of the claims 69 to 83.

102. The combination of claims 97 to 101, wherein component A and/or component B are co-formulated, preferably co-formulated formulated in lipid-based carriers as defined in any one of the claims 69 to 83.

103. The combination of claims 97 to 102, wherein upon administration of the combination to a cell or subject, the combination has reduced immunostimulatory properties compared to an administration of component B alone.

104. The combination of claims 97 to 103, wherein upon administration of the combination to a cell or subject, the combination has a prolonged protein expression compared to an administration of component B alone.

105. The combination of claims 97 to 104, wherein upon administration of the combination to a cell or subject, the combination has a faster onset of protein expression compared to an administration of component A alone.

106. A Kit or kit of parts, comprising at least one circular RNA of any one of claims 1 to 51, and/or at least one pharmaceutical composition of any one of claims 65 to 96, and/or at least on combination of any one of claims 97 to 105, optionally comprising a liquid vehicle for solubilising, and, optionally, technical instructions providing information on administration and dosage of the components.

107. The circular RNA of claims 1 to 51, and/or the pharmaceutical composition of claims 65 to 96, and/or the combination of claims 97 to 105, and/or the kit or kit of parts of claim 106 for use as a medicament.

108. The circular RNA of claims 1 to 51 and/or the pharmaceutical composition of claims 65 to 96, and/or the combination of claims 97 to 105, and/or the kit or kit of parts of claim 106 for use in treating or preventing an infectious, a tumour, a disorder related to obesity, or a genetic disease, disorder or condition.

109. A method of treating or preventing a disease, disorder or condition, wherein the method comprises applying or administering to a subject in need thereof the circular RNA of claims 1 to 51, and/or the pharmaceutical composition of claims 65 to 96, and/or the combination of claims 97 to 105, and/or the kit or kit of parts of claim 106.

110. The method of treating or preventing a disease, disorder or condition of claim 109, wherein the disease, disorder or condition is an infectious, a tumour, a disorder related to obesity, or a genetic disease, disorder or condition.

111. The method of treating or preventing a disorder of claim 109 or 110, wherein the subject in need is a mammalian subject, preferably a human subject.

112. The method of treating or preventing a disorder of claims 109 to 111, wherein applying or administering is performed via intramuscular injection, intradermal injection, transdermal injection, intradermal injection, intralesional injection, intracranial injection, subcutaneous injection, intracardial injection, intratumoral injection, intravenous injection, or intraocular injection, intrapulmonal inhalation, intraarticular injection, sublingual injection.

113. A method of purifying a circular RNA from a composition comprising linear precursor RNA and circular RNA comprising a step of
affinity-based removal of linear precursor RNA and/or intronic splice products
obtaining a preparation comprising purified circular RNA.

114. The method of purifying of claim 113, wherein the linear precursor RNA comprises a 3′ permuted intron-exon element and a 5′ permuted intron-exon element for circularization.

115. The method of purifying of claim 113 or 114, wherein the linear precursor RNA comprises at least one purification tag, preferably at least one purification tag located at the 3′ and/or the 5′ terminus of the (non-circularized) linear precursor RNA.

116. The method of purifying of claims 113 to 115, wherein the affinity-based removal comprises a step of selectively binding linear precursor RNA to an antisense oligonucleotide.

117. The method of purifying of claim 116, wherein the antisense oligonucleotide is configured to bind the 3′ permuted intron-exon element or the 5′ permuted intron-exon element of the linear precursor RNA.

118. The method of purifying of claims 116 to 117, wherein the antisense oligonucleotide is configured to bind the at least one purification tag located at the 3′ and/or the 5′ terminus of the linear precursor RNA.

119. The method of purifying of claims 116 to 118, wherein the antisense oligonucleotide is configured not to bind to the circular RNA.

120. The method of purifying of claims 116 to 119, wherein the antisense oligonucleotide comprises RNA, DNA, and/or LNA nucleotides, preferably wherein the antisense oligonucleotide comprises LNA nucleotides.

121. The method of purifying of claims 116 to 120, wherein the antisense oligonucleotide is immobilized on a solid support, preferably wherein the solid support is a bead or a column.

122. The method of purifying of claims 116 to 121, wherein the affinity-based removal comprises a step of subjecting the composition to the antisense oligonucleotide under conditions that allow nucleic acid hybridization.

123. The method of purifying of claims 116 to 122, wherein after binding of the linear precursor RNA to an antisense oligonucleotide, the purified circular RNA is collected in a flow through or a supernatant.

124. The method of purifying of claims 116 to 123, additionally comprising at least one step of purifying selected from RP-HPLC, AEX, size exclusion chromatography, hydroxyapatite chromatography, TFF, filtration, precipitation, core-bead flow through chromatography, oligo(dT) purification, spin column, cellulose-based purification, and affinity-based capturing of circular RNA.

125. The method of purifying of claims 116 to 124, additionally comprising a step of 5′ dephosphorylation of linear RNA, DNA digestion, protein digestion, and/or dsRNA digestion.

126. The method of purifying of claims 113 to 125, wherein the obtained preparation comprises more than about 70%, 75%, 80%, 85%, 90%, 95% circular RNA species in relation to total RNA.

127. The method of purifying of claims 113 to 126, wherein the obtained preparation comprises less than about 30%, 25%, 20%, 15%, 10%, 5% linear precursor RNA in relation to total RNA.

128. The method of purifying of claims 113 to 127, wherein the obtained preparation comprises less than about 5%, 4%, 3%, 2%, 1% linear intronic splice products in relation to total RNA.

129. The method of purifying of claims 113 to 128, wherein the method leads to a reduction of linear RNA in the obtained preparation of about 50%, 60%, 70%, 80%, 90%, 95%.

130. The method of purifying of claims 113 to 129, wherein the obtained purified circular RNA has a purity of at least about 70%, 75%, 80%, 85%, 90%, 95%.

131. The method of purifying of claims 113 to 130, wherein the linear precursor RNA is further characterized by any of the features as defined in claims 52 to 64 and/or wherein circular RNA is further characterized by any of the features as defined in claims 1 to 51.

132. A method for preparing circular RNA comprising the steps of
A) providing a linear precursor RNA;
B) incubating the precursor RNA in a buffer to allow circularization of the RNA;
C) obtaining the circular RNA product.

133. The method of claim 132, wherein the linear precursor RNA comprises a 3′ permuted intron-exon element and a 5′ permuted intron-exon element for circularization.

134. The method of claim 132 or 133, wherein the providing step A) comprises a step A1) RNA in vitro transcription.

135. The method of claim 134, wherein step A1) is performed in the presence of a sequence optimized nucleotide mixture.

136. The method of claim 134 or 135, wherein step A1) is performed in the absence of modified nucleotides and/or cap analogs.

137. The method of claim 134 or 135, wherein step A1) is performed in the presence of modified nucleotides and/or cap analogs, preferably wherein at least one modified nucleotide is selected from pseudouridine (ψ), N1-methylpseudouridine (m1ψ), 5-methylcytosine, and/or 5-methoxyuridine.

138. The method of claims 132 to 137, wherein the method comprises a step of adding GTP to the RNA in vitro transcription reaction to start the incubation step B), preferably to obtain a final concentration of at least about 2 mM GTP.

139. The method of claims 132 to 138, wherein the incubation step B) is performed in a buffer comprising GTP and MgCl2, preferably wherein GTP is in a final concentration of at least about 2 mM.

140. The method of claims 132 to 139, wherein the incubation step B) is performed for at least about 5 minutes at a temperature of about 55° C.

141. The method of claims 132 to 140, wherein the obtaining step C) comprises at least one purification step C1) of affinity-based removal of linear precursor RNA and/or intronic splice products preferably as defined in claims 113 to 131.

142. The method of claims 132 to 141, wherein the obtaining step C) comprises at least one purification step C2) selected from RP-HPLC, AEX, size exclusion chromatography, hydroxyapatite chromatography, TFF, filtration, precipitation, core-bead flow through chromatography, oligo(dT) purification, spin column, cellulose-based purification, and affinity-based capturing of circular RNA.

143. The method of claim 142, wherein step C2) is selected from RP-HPLC and/or TFF.

144. The method of claims 132 to 143, wherein the obtaining step C) comprises a step C3) of digesting linear RNA impurities, preferably wherein the step of digesting is performed using an RNAse specific for linear RNA.

145. The method of claims 132 to 144, wherein the obtaining step C) comprises a step of 5′ dephosphorylation of linear RNA impurities.

146. The method of claims 132 to 145, wherein the obtaining step C) comprises a step of DNA digestion, protein digestion, and/or dsRNA digestion.

147. The method of claims 132 to 146, wherein the linear precursor RNA is further characterized by any of the features as defined in claims 52 to 64 and/or wherein circular RNA is further characterized by any of the features as defined in claims 1 to 51.
